SEQUENCE LISTING (18 sequences)

SEQ ID NO: 1 - DNA, 199 bp, Homo sapiens
gggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccggg

SEQ ID NO: 2 - protein, 1152 aa, Homo sapiens
MREPEELMPDSGAVFTFGKSKFAENNPGKFWFKNDVPVHLSCGDEHSAVVTGNNKLYMFGSNNWGQLGLGSKSAISKPTCVKALKPEKVKLAACGRNHTLVSTEGGNVYATGGNNEGQLGLGDTEERNTFHVISFFTSEHKIKQLSAGSNTSAALTEDGRLFMWGDNSEGQIGLKNVSNVCVPQQVTIGKPVSWISCGYYHSAFVTTDGELYVFGEPENGKLGLPNQLLGNHRTPQLVSEIPEKVIQVACGGEHTVVLTENAVYTFGLGQFGQLGLGTFLFETSEPKVIENIRDQTISYISCGENHTALITDIGLMYTFGDGRHGKLGLGLENFTNHFIPTLCSNFLRFIVKLVACGGCHMVVFAAPHRGVAKEIEFDEINDTCLSVATFLPYSSLTSGNVLQRTLSARMRRRERERSPDSFSMRRTLPPIEGTLGLSACFLPNSVFPRCSERNLQESVLSEQDLMQPEEPDYLLDEMTKEAEIDNSSTVESLGETTDILNMTHIMSLNSNEKSLKLSPVQKQKKQQTIGELTQDTALTENDDSDEYEEMSEMKEGKACKQHVSQGIFMTQPATTIEAFSDEEVEIPEEKEGAEDSKGNGIEEQEVEANEENVKVHGGRKEKTEILSDDLTDKAEVSEGKAKSVGEAEDGPEGRGDGTCEEGSSGAEHWQDEEREKGEKDKGRGEMERPGEGEKELAEKEEWKKRDGEEQEQKEREQGHQKERNQEMEEGGEEEHGEGEEEEGDREEEEEKEGEGKEEGEGEEVEGEREKEEGERKKEERAGKEEKGEEEGDQGEGEEEETEGRGEEKEEGGEVEGGEVEEGKGEREEEEEEGEGEEEEGEGEEEEGEGEEEEGEGKGEEEGEEGEGEEEGEEGEGEGEEEEGEGEGEEEGEGEGEEEEGEGEGEEEGEGEGEEEEGEGKGEEEGEEGEGEGEEEEGEGEGEDGEGEGEEEEGEWEGEEEEGEGEGEEEGEGEGEEGEGEGEEEEGEGEGEEEEGEEEGEEEGEGEEEGEGEGEEEEEGEVEGEVEGEEGEGEGEEEEGEEEGEEREKEGEGEENRRNREEEEEEEGKYQETGEEENERQDGEEYKKVSKIKGSVKYGKHKTYQKKSVTNTQGNGKEQRSKMPVQSKRLLKNGPSGSKKFWNNVLPHYLELK

SEQ ID NO: 3 - DNA, 3459 bp, Artificial Sequence
  Made in Lab - Codon optimized RPGR ORF15
atgagagagccagaggagctgatgccagacagtggagcagtgtttacattcggaaaatctaagttcgctgaaaataacccaggaaagttctggtttaaaaacgacgtgcccgtccacctgtcttgtggcgatgagcatagtgccgtggtcactgggaacaataagctgtacatgttcgggtccaacaactggggacagctggggctgggatccaaatctgctatctctaagccaacctgcgtgaaggcactgaaacccgagaaggtcaaactggccgcttgtggcagaaaccacactctggtgagcaccgagggcgggaatgtctatgccaccggaggcaacaatgagggacagctgggactgggggacactgaggaaaggaatacctttcacgtgatctccttctttacatctgagcataagatcaagcagctgagcgctggctccaacacatctgcagccctgactgaggacgggcgcctgttcatgtggggagataattcagagggccagattgggctgaaaaacgtgagcaatgtgtgcgtccctcagcaggtgaccatcggaaagccagtcagttggatttcatgtggctactatcatagcgccttcgtgaccacagatggcgagctgtacgtctttggggagcccgaaaacggaaaactgggcctgcctaaccagctgctgggcaatcaccggacaccccagctggtgtccgagatccctgaaaaagtgatccaggtcgcctgcgggggagagcatacagtggtcctgactgagaatgctgtgtataccttcggactgggccagtttggccagctggggctgggaaccttcctgtttgagacatccgaaccaaaagtgatcgagaacattcgcgaccagactatcagctacatttcctgcggagagaatcacaccgcactgatcacagacattggcctgatgtatacctttggcgatggacgacacgggaagctgggactgggactggagaacttcactaatcattttatccccaccctgtgttctaacttcctgcggttcatcgtgaaactggtcgcttgcggcgggtgtcacatggtggtcttcgctgcacctcataggggcgtggctaaggagatcgaatttgacgagattaacgatacatgcctgagcgtggcaactttcctgccatacagctccctgacttctggcaatgtgctgcagagaaccctgagtgcaaggatgcggagaagggagagggaacgctctcctgacagtttctcaatgcgacgaaccctgccacctatcgagggaacactgggactgagtgcctgcttcctgcctaactcagtgtttccacgatgtagcgagcggaatctgcaggagtctgtcctgagtgagcaggatctgatgcagccagaggaacccgactacctgctggatgagatgaccaaggaggccgaaatcgacaactctagtacagtggagtccctgggcgagactaccgatatcctgaatatgacacacattatgtcactgaacagcaatgagaagagtctgaaactgtcaccagtgcagaagcagaagaaacagcagactattggcgagctgactcaggacaccgccctgacagagaacgacgatagcgatgagtatgaggaaatgtccgagatgaaggaaggcaaagcttgtaagcagcatgtcagtcaggggatcttcatgacacagccagccacaactattgaggctttttcagacgaggaagtggagatccccgaggaaaaagagggcgcagaagattccaaggggaatggaattgaggaacaggaggtggaagccaacgaggaaaatgtgaaagtccacggaggcaggaaggagaaaacagaaatcctgtctgacgatctgactgacaaggccgaggtgtccgaaggcaaggcaaaatctgtcggagaggcagaagacggaccagagggacgaggggatggaacctgcgaggaaggctcaagcggggctgagcattggcaggacgaggaacgagagaagggcgaaaaggataaaggccgcggggagatggaacgacctggagagggcgaaaaagagctggcagagaaggaggaatggaagaaaagggacggcgaggaacaggagcagaaagaaagggagcagggccaccagaaggagcgcaaccaggagatggaagagggcggcgaggaagagcatggcgagggagaagaggaagagggcgatagagaagaggaagaggaaaaagaaggcgaagggaaggaggaaggagagggcgaggaagtggaaggcgagagggaaaaggaggaaggagaacggaagaaagaggaaagagccggcaaagaggaaaagggcgaggaagagggcgatcagggcgaaggcgaggaggaagagaccgagggccgcggggaagagaaagaggagggaggagaggtggagggcggagaggtcgaagagggaaagggcgagcgcgaagaggaagaggaagagggcgagggcgaggaagaagagggcgagggggaagaagaggagggagagggcgaagaggaagagggggagggaaagggcgaagaggaaggagaggaaggggagggagaggaagagggggaggagggcgagggggaaggcgaggaggaagaaggagagggggaaggcgaagaggaaggcgagggggaaggagaggaggaagaaggggaaggcgaaggcgaagaggagggagaaggagagggggaggaagaggaaggagaagggaagggcgaggaggaaggcgaagagggagagggggaaggcgaggaagaggaaggcgagggcgaaggagaggacggcgagggcgagggagaagaggaggaaggggaatgggaaggcgaagaagaggaaggcgaaggcgaaggcgaagaagagggcgaaggggagggcgaggagggcgaaggcgaaggggaggaagaggaaggcgaaggagaaggcgaggaagaagagggagaggaggaaggcgaggaggaaggagagggggaggaggagggagaaggcgagggcgaagaagaagaagagggagaagtggagggcgaagtcgagggggaggagggagaaggggaaggggaggaagaagagggcgaagaagaaggcgaggaaagagaaaaagagggagaaggcgaggaaaaccggagaaatagggaagaggaggaagaggaagagggaaagtaccaggagacaggcgaagaggaaaacgagcggcaggatggcgaggaatataagaaagtgagcaagatcaaaggatccgtcaagtacggcaagcacaaaacctatcagaagaaaagcgtgaccaacacacaggggaatggaaaagagcagaggagtaagatgcctgtgcagtcaaaacggctgctgaagaatggcccatctggaagtaaaaaattctggaacaatgtgctgccccactatctggaactgaaataa

SEQ ID NO: 4 - DNA, 270 bp, Bos taurus
tcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctgggg

SEQ ID NO: 5 - DNA, 119 bp, Adeno-associated virus 2
ctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 6 - DNA, 130 bp, Adeno-associated virus 2
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 7 - DNA, 10 bp, Artificial Sequence
  Consensus Kozak sequence
ggccaccatg

SEQ ID NO: 8 - DNA, 4450 bp, Artificial Sequence
  Plasmid construct
ctgcgcgctcgctcgctcactgaggccgcccgggcgtcgggcgacctttggtcgcccggcctcagtgagcgagcgagcgcgcagagagggagtggccaactccatcactaggggttcctgcggcaattcagtcgataactataacggtcctaaggtagcgatttaaatacgcgctctcttaaggtagccccgggacgcgtcaattggggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccggggccaccatgagagagccagaggagctgatgccagacagtggagcagtgtttacattcggaaaatctaagttcgctgaaaataacccaggaaagttctggtttaaaaacgacgtgcccgtccacctgtcttgtggcgatgagcatagtgccgtggtcactgggaacaataagctgtacatgttcgggtccaacaactggggacagctggggctgggatccaaatctgctatctctaagccaacctgcgtgaaggcactgaaacccgagaaggtcaaactggccgcttgtggcagaaaccacactctggtgagcaccgagggcgggaatgtctatgccaccggaggcaacaatgagggacagctgggactgggggacactgaggaaaggaatacctttcacgtgatctccttctttacatctgagcataagatcaagcagctgagcgctggctccaacacatctgcagccctgactgaggacgggcgcctgttcatgtggggagataattcagagggccagattgggctgaaaaacgtgagcaatgtgtgcgtccctcagcaggtgaccatcggaaagccagtcagttggatttcatgtggctactatcatagcgccttcgtgaccacagatggcgagctgtacgtctttggggagcccgaaaacggaaaactgggcctgcctaaccagctgctgggcaatcaccggacaccccagctggtgtccgagatccctgaaaaagtgatccaggtcgcctgcgggggagagcatacagtggtcctgactgagaatgctgtgtataccttcggactgggccagtttggccagctggggctgggaaccttcctgtttgagacatccgaaccaaaagtgatcgagaacattcgcgaccagactatcagctacatttcctgcggagagaatcacaccgcactgatcacagacattggcctgatgtatacctttggcgatggacgacacgggaagctgggactgggactggagaacttcactaatcattttatccccaccctgtgttctaacttcctgcggttcatcgtgaaactggtcgcttgcggcgggtgtcacatggtggtcttcgctgcacctcataggggcgtggctaaggagatcgaatttgacgagattaacgatacatgcctgagcgtggcaactttcctgccatacagctccctgacttctggcaatgtgctgcagagaaccctgagtgcaaggatgcggagaagggagagggaacgctctcctgacagtttctcaatgcgacgaaccctgccacctatcgagggaacactgggactgagtgcctgcttcctgcctaactcagtgtttccacgatgtagcgagcggaatctgcaggagtctgtcctgagtgagcaggatctgatgcagccagaggaacccgactacctgctggatgagatgaccaaggaggccgaaatcgacaactctagtacagtggagtccctgggcgagactaccgatatcctgaatatgacacacattatgtcactgaacagcaatgagaagagtctgaaactgtcaccagtgcagaagcagaagaaacagcagactattggcgagctgactcaggacaccgccctgacagagaacgacgatagcgatgagtatgaggaaatgtccgagatgaaggaaggcaaagcttgtaagcagcatgtcagtcaggggatcttcatgacacagccagccacaactattgaggctttttcagacgaggaagtggagatccccgaggaaaaagagggcgcagaagattccaaggggaatggaattgaggaacaggaggtggaagccaacgaggaaaatgtgaaagtccacggaggcaggaaggagaaaacagaaatcctgtctgacgatctgactgacaaggccgaggtgtccgaaggcaaggcaaaatctgtcggagaggcagaagacggaccagagggacgaggggatggaacctgcgaggaaggctcaagcggggctgagcattggcaggacgaggaacgagagaagggcgaaaaggataaaggccgcggggagatggaacgacctggagagggcgaaaaagagctggcagagaaggaggaatggaagaaaagggacggcgaggaacaggagcagaaagaaagggagcagggccaccagaaggagcgcaaccaggagatggaagagggcggcgaggaagagcatggcgagggagaagaggaagagggcgatagagaagaggaagaggaaaaagaaggcgaagggaaggaggaaggagagggcgaggaagtggaaggcgagagggaaaaggaggaaggagaacggaagaaagaggaaagagccggcaaagaggaaaagggcgaggaagagggcgatcagggcgaaggcgaggaggaagagaccgagggccgcggggaagagaaagaggagggaggagaggtggagggcggagaggtcgaagagggaaagggcgagcgcgaagaggaagaggaagagggcgagggcgaggaagaagagggcgagggggaagaagaggagggagagggcgaagaggaagagggggagggaaagggcgaagaggaaggagaggaaggggagggagaggaagagggggaggagggcgagggggaaggcgaggaggaagaaggagagggggaaggcgaagaggaaggcgagggggaaggagaggaggaagaaggggaaggcgaaggcgaagaggagggagaaggagagggggaggaagaggaaggagaagggaagggcgaggaggaaggcgaagagggagagggggaaggcgaggaagaggaaggcgagggcgaaggagaggacggcgagggcgagggagaagaggaggaaggggaatgggaaggcgaagaagaggaaggcgaaggcgaaggcgaagaagagggcgaaggggagggcgaggagggcgaaggcgaaggggaggaagaggaaggcgaaggagaaggcgaggaagaagagggagaggaggaaggcgaggaggaaggagagggggaggaggagggagaaggcgagggcgaagaagaagaagagggagaagtggagggcgaagtcgagggggaggagggagaaggggaaggggaggaagaagagggcgaagaagaaggcgaggaaagagaaaaagagggagaaggcgaggaaaaccggagaaatagggaagaggaggaagaggaagagggaaagtaccaggagacaggcgaagaggaaaacgagcggcaggatggcgaggaatataagaaagtgagcaagatcaaaggatccgtcaagtacggcaagcacaaaacctatcagaagaaaagcgtgaccaacacacaggggaatggaaaagagcagaggagtaagatgcctgtgcagtcaaaacggctgctgaagaatggcccatctggaagtaaaaaattctggaacaatgtgctgccccactatctggaactgaaataagagctcctcgaggcggcccgctcgagtctagagggcccttcgaaggtaagcctatccctaaccctctcctcggtctcgattctacgcgtaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagatcctctcttaaggtagcatcgagatttaaattagggataacagggtaatggcgcgggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 9 - DNA, 588 bp, Woodchuck hepatitis virus
atcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 10 - DNA, 3459 bp, Homo sapiens
atgagggagccggaagagctgatgcccgattcgggtgctgtgtttacatttgggaaaagtaaatttgctgaaaataatcccggtaaattctggtttaaaaatgatgtccctgtacatctttcatgtggagatgaacattctgctgttgttaccggaaataataaactttacatgtttggcagtaacaactggggtcagttaggattaggatcaaagtcagccatcagcaagccaacatgtgtcaaagctctaaaacctgaaaaagtgaaattagctgcctgtggaaggaaccacaccctggtgtcaacagaaggaggcaatgtatatgcaactggtggaaataatgaaggacagttggggcttggtgacaccgaagaaagaaacacttttcatgtaattagcttttttacatccgagcataagattaagcagctgtctgctggatctaatacttcagctgccctaactgaggatggaagactttttatgtggggtgacaattccgaagggcaaattggtttaaaaaatgtaagtaatgtctgtgtccctcagcaagtgaccattgggaaacctgtctcctggatctcttgtggatattaccattcagcttttgtaacaacagatggtgagctatatgtgtttggagaacctgagaatgggaagttaggtcttcccaatcagctcctgggcaatcacagaacaccccagctggtgtctgaaattccggagaaggtgatccaagtagcctgtggtggagagcatactgtggttctcacggagaatgctgtgtatacctttgggctgggacaatttggtcagctgggtcttggcacttttctttttgaaacttcagaacccaaagtcattgagaatattagggatcaaacaataagttatatttcttgtggagaaaatcacacagctttgataacagatatcggccttatgtatacttttggagatggtcgccacggaaaattaggacttggactggagaattttaccaatcacttcattcctactttgtgctctaattttttgaggtttatagttaaattggttgcttgtggtggatgtcacatggtagtttttgctgctcctcatcgtggtgtggcaaaagaaattgaattcgatgaaataaatgatacttgcttatctgtggcgacttttctgccgtatagcagtttaacctcaggaaatgtactgcagaggactctatcagcacgtatgcggcgaagagagagggagaggtctccagattctttttcaatgaggagaacactacctccaatagaagggactcttggcctttctgcttgttttctccccaattcagtctttccacgatgttctgagagaaacctccaagagagtgtcttatctgaacaggacctcatgcagccagaggaaccagattatttgctagatgaaatgaccaaagaagcagagatagataattcttcaactgtagaaagccttggagaaactactgatatcttaaacatgacacacatcatgagcctgaattccaatgaaaagtcattaaaattatcaccagttcagaaacaaaagaaacaacaaacaattggggaactgacgcaggatacagctcttactgaaaacgatgatagtgatgaatatgaagaaatgtcagaaatgaaagaagggaaagcatgtaaacaacatgtgtcacaagggattttcatgacgcagccagctacgactatcgaagcattttcagatgaggaagtagagatcccagaggagaaggaaggagcagaggattcaaaaggaaatggaatagaggagcaagaggtagaagcaaatgaggaaaatgtgaaggtgcatggaggaagaaaggagaaaacagagatcctatcagatgaccttacagacaaagcagaggtgagtgaaggcaaggcaaaatcagtgggagaagcagaggatgggcctgaaggtagaggggatggaacctgtgaggaaggtagttcaggagcagaacactggcaagatgaggagagggagaagggggagaaagacaagggtagaggagaaatggagaggccaggagagggagagaaggaactagcagagaaggaagaatggaagaagagggatggggaagagcaggagcaaaaggagagggagcagggccatcagaaggaaagaaaccaagagatggaggagggaggggaggaggagcatggagaaggagaagaagaggagggagacagagaagaggaagaagagaaggagggagaagggaaagaggaaggagaaggggaagaagtggagggagaacgtgaaaaggaggaaggagagaggaaaaaggaggaaagagcggggaaggaggagaaaggagaggaagaaggagaccaaggagagggggaagaggaggaaacagaggggagaggggaggaaaaagaggagggaggggaagtagagggaggggaagtagaggaggggaaaggagagagggaagaggaagaggaggagggtgagggggaagaggaggaaggggagggggaagaggaggaaggggagggggaagaggaggaaggagaagggaaaggggaggaagaaggggaagaaggagaaggggaggaagaaggggaggaaggagaaggggagggggaagaggaggaaggagaaggggagggagaagaggaaggagaaggggagggagaagaggaggaaggagaaggggagggagaagaggaaggagaaggggagggagaagaggaggaaggagaagggaaaggggaggaggaaggagaggaaggagaaggggagggggaagaggaggaaggagaaggggaaggggaggatggagaaggggagggggaagaggaggaaggagaatgggagggggaagaggaggaaggagaaggggagggggaagaggaaggagaaggggaaggggaggaaggagaaggggagggggaagaggaggaaggagaaggggagggggaagaggaggaaggggaagaagaaggggaggaagaaggagagggagaggaagaaggggagggagaaggggaggaagaagaggaaggggaagtggaaggggaggtggaaggggaggaaggagagggggaaggagaggaagaggaaggagaggaggaaggagaagaaagggaaaaggagggggaaggagaagaaaacaggaggaacagagaagaggaggaggaagaagaggggaagtatcaggagacaggcgaagaagagaatgaaaggcaggatggagaggagtacaaaaaagtgagcaaaataaaaggatctgtgaaatatggcaaacataaaacatatcaaaaaaagtcagttactaacacacagggaaatgggaaagagcagaggtccaaaatgccagtccagtcaaaacgacttttaaaaaacgggccatcaggttccaaaaagttctggaataatgtattaccacattacttggaattgaagtaa

SEQ ID NO: 11 - skipped

SEQ ID NO: 12 - protein, 20 aa, Homo sapiens
EKSLKLSPVQKQKKQQTIGE

SEQ ID NO: 13 - protein, 17 aa, Homo sapiens
KSKFAENNPGKFWFKND

SEQ ID NO: 14 - protein, 17 aa, Homo sapiens
GNNEGQLGLGDTEERNT

SEQ ID NO: 15 - protein, 131 aa, Homo sapiens
EINDTCLSVATFLPYSSLTSGNVLQRTLSARMRRRERERSPDSFSMRRTLPPIEGTLGLSACFLPNSVFPRCSERNLQESVLSEQDLMQPEEPDYLLDEMTKEAEIDNSSTVESLGETTDILNMTHIMSLN

SEQ ID NO: 16 - DNA, 225 bp, Homo sapiens
gaggaggaaggagaagggaaaggggaggaagaaggggaagaaggagaaggggaggaagaaggggaggaaggagaaggggagggggaagaggaggaaggagaaggggagggagaagaggaaggagaaggggagggagaagaggaggaaggagaaggggagggagaagaggaaggagaaggggagggagaagaggaggaaggagaagggaaaggggaggaggaagga

SEQ ID NO: 17 - DNA, 225 bp, Artificial Sequence
  Made in Lab - Codon optimized RPGR ORF15
gaggaagagggggagggaaagggcgaagaggaaggagaggaaggggagggagaggaagagggggaggagggcgagggggaaggcgaggaggaagaaggagagggggaaggcgaagaggaaggcgagggggaaggagaggaggaagaaggggaaggcgaaggcgaagaggagggagaaggagagggggaggaagaggaaggagaagggaagggcgaggaggaaggc

SEQ ID NO: 18 - DNA, 944 bp, Homo sapiens
gaggagggagacagagaagaggaagaagagaaggagggagaagggaaagaggaaggagaaggggaagaagtggagggagaacgtgaaaaggaggaaggagagaggaaaaaggaggaaagagcggggaaggaggagaaaggagaggaagaaggagaccaaggagagggggaagaggaggaaacagaggggagaggggaggaaaaagaggagggaggggaagtagagggaggggaagtagaggaggggaaaggagagagggaagaggaagaggaggagggtgagggggaagaggaggaaggggagggggaagaggaggaaggggagggggaagaggaggaaggagaagggaaaggggaggaagaaggggaagaaggagaaggggaggaagaaggggaggaaggagaaggggagggggaagaggaggaaggagaaggggagggagaagaggaaggagaaggggagggagaagaggaggaaggagaaggggagggagaagaggaaggagaaggggagggagaagaggaggaaggagaagggaaaggggaggaggaaggagaggaaggagaaggggagggggaagaggaggaaggagaaggggaaggggaggatggagaaggggagggggaagaggaggaaggagaatgggagggggaagaggaggaaggagaaggggagggggaagaggaaggagaaggggaaggggaggaaggagaaggggagggggaagaggaggaaggagaaggggagggggaagaggaggaaggggaagaagaaggggaggaagaaggagagggagaggaagaaggggagggagaaggggaggaagaagaggaaggggaagtggaaggggaggtggaaggggaggaaggagagggggaaggagaggaagaggaaggagaggaggaaggagaagaaagggaaaaggagggggaaggagaagaaaa

(19) United States
(12) Patent Application Publication
(10) Pub. No.: US 2021/0348193 A1
(43) Pub. Date: Nov. 11, 2021

(54) COMPOSITIONS AND METHODS FOR TREATING RETINITIS PIGMENTOSA

(71) Applicant: NightstaRx Limited, Maidenhead, Berkshire (GB)

(72) Inventors: Gregory S. Robinson, Waltham, MA (US); Tuyen Ong, London (GB)

(21) Appl. No.: 17/277,842

(22) PCT Filed: Sep. 23, 2019
(86) PCT No.: PCT/US2019/052471

§ 371 Date: Mar. 19, 2021

Related U.S. Application Data
(60) Provisional application No. 62/830,106, filed on Apr. 5, 2019.
(60) Provisional application No. 62/734,746, filed on Sep. 21, 2018.

(51) Int. Cl. C12N 15/86; A61K 48/00; A61P 27/02

(52) U.S. Cl. CPC C12N 15/86; A61P 27/02; A61K 48/0058

(57) ABSTRACT

The disclosure relates to compositions and methods for the treatment of Retinitis Pigmentosa through the administration of a rAAV vector comprising an RPGR^{ORF15} sequence

### RELATED APPLICATIONS

This application claims priority to U.S. Provisional Patent Application No. 62/830,106, filed Apr. 5, 2019, and U.S. Provisional Patent Application No. 62/734,746, filed Sep. 21, 2018, the contents of which are incorporated herein in their entirety.

### STATEMENT REGARDING THE SEQUENCE LISTING

The Sequence Listing associated with this application is provided in text format in lieu of a paper copy, and is hereby incorporated by reference into the specification. The name of the text file containing the Sequence Listing is NIGH-016_N01WO_ST25.txt. The text file is about 32 KB, created on Sep. 19, 2019, and is being submitted electronically via EFS-Web.

### FIELD OF THE DISCLOSURE

The disclosure relates to the fields of human therapeutics, biologic drug products, viral delivery of human DNA sequences and methods of manufacturing same.

### BACKGROUND

Retinitis Pigmentosa is a rare genetic disease that is estimated to affect 1 in 4,000 people world wide. Retinitis Pigmentosa involves the progressive degeneration of the retina, leading to visual symptoms that include loss of night vision, loss of peripheral vision, decreased color perception, decreased visual acuity, loss of central vision and eventual blindness. There is currently no cure for Retinitis Pigmentosa. There thus exists a pressing need in the art for treatments for Retinitis Pigmentosa. This invention provides compositions and methods for treating Retinitis Pigmentosa.

### SUMMARY

The disclosure provides a composition comprising a plurality of recombinant adeno associated virus of serotype 8 (rAAV8) particles, wherein each rAAV8 of the plurality of rAAV8 particles is non-replicating, and wherein each rAAV8 of the plurality of rAAV8 particles comprises a polynucleotide comprising, from 5′ to 3′: (a) a sequence encoding a 5′ inverted terminal repeat (ITR); (b) a sequence encoding a G protein-coupled receptor kinase 1 (GRK1) promoter; (c) a sequence encoding a retinitis pigmentosa GTPase regulator ORF15 isoform (RPGR^{ORF15}); (d) a sequence encoding a polyadenylation (polyA) signal; (e) a sequence encoding a 3′ ITR; and wherein the composition comprises between 5×10⁹ vector genomes (vg) per milliliter (mL) and 2×10¹³ vg/mL, inclusive of the endpoints.

In some embodiments, the composition comprises between 1.0×10¹⁰ vector genomes (vg) per milliliter (mL) and 1×10¹³ vg/mL, inclusive of the endpoints. In some embodiments, the composition comprises between 5×10¹⁰ genome particles (gp) and 5×10¹² g. In some embodiments, the composition comprises between 1.25×10¹² vg/mL and 1×10¹³ vg/mL, inclusive of the endpoints. In some embodiments, the composition comprises 1×10¹² vg/mL. In some embodiments, the composition comprises 2.5×10¹² vg/mL. In some embodiments, the composition comprises 5×10¹² vg/mL. In some embodiments, the composition comprises 5×10⁹ gp, 1×10¹⁰ gp, 5×10¹⁰ gp, 1×10¹¹ gp, 2.5×10¹¹ gp 5×10¹¹ gp, 1.25×10¹² gp, 2.5×10¹² gp, 5×10¹² gp, or 1×10¹³.

In some embodiments of the compositions of the disclosure, the composition comprises between 0.5×10¹¹ vg/mL and 1×10¹² vg/mL, inclusive of the endpoints. In some embodiments, the composition comprises 0.5×10¹¹ vg/mL. In some embodiments, the composition comprises 5×10⁹ vg/mL. In some embodiments, the composition comprises 1×10¹⁰ vg/mL. In some embodiments, the composition comprises 5×10¹⁰ vg/mL. In some embodiments, the composition comprises 1×10¹¹ vg/mL. In some embodiments, the composition comprises 2.5×10¹¹ vg/mL. In some embodiments, the composition comprises 5×10¹¹ vg/mL. In some embodiments, the composition comprises 5×10¹² vg/mL. In some embodiments, the composition comprises 1×10¹³ vg/mL. In some embodiments, the composition comprises 2×10¹³ vg/mL.

In some embodiments of the compositions of the disclosure, the composition comprises between 5×10⁹ genome particles (gp) and 5×10¹¹ gp, inclusive of the endpoints. In some embodiments, the composition comprises 5×10⁹ gp. In some embodiments, the composition comprises 1×10¹⁰ gp. In some embodiments, the composition comprises 5×10¹⁰ gp. In some embodiments, the composition comprises 1×10¹¹ gp. In some embodiments, the composition comprises 2.5×10¹¹ gp. In some embodiments, the composition comprises 5×10¹¹ gp.

In some embodiments of the compositions of the disclosure, the composition further comprises a pharmaceutically acceptable carrier. In some embodiments, the pharmaceutically acceptable carrier comprises Tris, MgCl₂, and NaCl. In some embodiments, the pharmaceutically acceptable carrier comprises 20 mM Tris, 1 mM MgCl₂, and 200 mM NaCl at pH 8.0. In some embodiments, the pharmaceutically acceptable carrier further comprises poloxamer 188 at 0.001%.

In some embodiments of the compositions of the disclosure, the sequence encoding the GRK1 promoter comprises or consists of the sequence of:

| (SEQ ID NO: 1) | |
|---|---|
| 1 | gggccccaga agcctggtgg ttgtttgtcc ttctcagggg aaaagtgagg cggccccttg |
| 61 | gaggaagggg ccgggcagaa tgatctaatc ggattccaag cagctcaggg gattgtcttt |
| 121 | ttctagcacc ttcttgccac tcctaagcgt cctccgtgac cccggctggg atttagcctg |
| 181 | gtgctgtgtc agccccggg. |

In some embodiments of the compositions of the disclosure, the sequence encoding RPGR^{ORF15} comprises or consists of a nucleotide sequence encoding the RPGR^{ORF15} amino acid sequence of:

| (SEQ ID NO: 2) | |
|---|---|
| 1 | MREPEELMPD SGAVFTFGKS KFAENNPGKF WFKNDVPVHL SCGDEHSAVV TGNNKLYMFG |
| 61 | SNNWGQLGLG SKSAISKPTC VKALKPEKVK LAACGRNHTL VSTEGGNVYA TGGNNEGQLG |
| 121 | LGDTEERNTF HVISFFTSEH KIKQLSAGSN TSAALTEDGR LFMWGDNSEG QIGLKNVSNV |
| 181 | CVPQQVTIGK PVSWISCGYY HSAFVTTDGE LYVFGEPENG KLGLPNQLLG NHRTPQLVSE |
| 241 | IPEKVIQVAC GGEHTVVLTE NAVYTFGLGQ FGQLGLGTFL FETSEPKVIE NIRDQTISYI |
| 301 | SCGENHTALI TDIGLMYTFG DGRHGKLGLG LENFTNHFIP TLCSNFLRFI VKLVACGGCH |
| 361 | MVVFAAPHRG VAKEIEFDEI NDTCLSVATF LPYSSLTSGN VLQRTLSARM RRRERERSPD |
| 421 | SFSMRRTLPP IEGTLGLSAC FLPNSVFPRC SERNLQESVL SEQDLMQPEE PDYLLDEMTK |
| 481 | EAEIDNSSTV ESLGETTDIL NMTHIMSLNS NEKSLKLSPV QKQKKQQTIG ELTQDTALTE |
| 541 | NDDSDEYEEM SEMKEGKACK QHVSQGIFMT QPATTIEAFS DEEVEIPEEK EGAEDSKGNG |
| 601 | IEEQEVEANE ENVKVHGGRK EKTEILSDDL TDKAEVSEGK AKSVGEAEDG PEGRGDGTCE |
| 661 | EGSSGAEHWQ DEEREKGEKD KGRGEMERPG EGEKELAEKE EWKKRDGEEQ EQKEREQGHQ |
| 721 | KERNQEMEEG GEEEHGEGEE EEGDREEEEE KEGEGKEEGE GEEVEGEREK EEGERKKEER |
| 781 | AGKEEKGEEE GDQGEGEEEE TEGRGEEKEE GGEVEGGEVE EGKGEREEEE EEGEGEEEEG |
| 841 | EGEEEEGEGE EEEGEGKGEE EGEEGEGEEE GEEGEGEGEE EEGEGEGEEE GEGEGEEEEG |
| 901 | EGEGEEEGEG EGEEEEGEGK GEEEGEEGEG EGEEEEGEGE GEDGEGEGEE EEGEWEGEEE |
| 961 | EGEGEGEEEG EGEGEEGEGE GEEEEGEGEG EEEEGEEEGE EEGEGEEEGE GEGEEEEEGE |
| 1021 | VEGEVEGEEG EGEGEEEEGE EEGEEREKEG EGEENRRNRE EEEEEEGKYQ ETGEEENERQ |
| 1081 | DGEEYKKVSK IKGSVKYGKH KTYQKKSVTN TQGNGKEQRS KMPVQSKRLL KNGPSGSKKF |
| 1141 | WNNVLPHYLE LK. |

In some embodiments of the compositions of the disclosure, the sequence encoding the RPGR^{ORF15} amino acid sequence comprises a codon optimized sequence. In some embodiments, the sequence encoding RPGR^{ORF15} comprises or consists of the nucleotide sequence of:

| (SEQ ID NO: 3) | |
|---|---|
| 1 | atgagagagc cagaggagct gatgccagac agtggagcag tgtttacatt cggaaaatct |
| 61 | aagttcgctg aaaataaccc aggaaagttc tggtttaaaa acgacgtgcc cgtccacctg |
| 121 | tcttgtggcg atgagcatag tgccgtggtc actgggaaca ataagctgta catgttcggg |
| 181 | tccaacaact ggggacagct ggggctggga tccaaatctg ctatctctaa gccaacctgc |
| 241 | gtgaaggcac tgaaacccga gaaggtcaaa ctggccgctt gtggcagaaa ccacactctg |
| 301 | gtgagcaccg agggcgggaa tgtctatgcc accggaggca acaatgaggg acagctggga |
| 361 | ctgggggaca ctgaggaaag gaataccttt cacgtgatct ccttctttac atctgagcat |
| 421 | aagatcaagc agctgagcgc tggctccaac acatctgcag ccctgactga ggacgggcgc |
| 481 | ctgttcatgt ggggagataa ttcagagggc cagattgggc tgaaaaacgt gagcaatgtg |
| 541 | tgcgtccctc agcaggtgac catcggaaag ccagtcagtt ggatttcatg tggctactat |
| 601 | catagcgcct tcgtgaccac agatggcgag ctgtacgtct ttggggagcc cgaaaacgga |
| 661 | aaactgggcc tgcctaacca gctgctgggc aatcaccgga caccccagct ggtgtccgag |
| 721 | atccctgaaa aagtgatcca ggtcgcctgc gggggagagc atacagtggt cctgactgag |
| 781 | aatgctgtgt ataccttcgg actgggccag tttggccagc tggggctggg aaccttcctg |
| 841 | tttgagacat ccgaaccaaa agtgatcgag aacattcgcg accagactat cagctacatt |
| 901 | tcctgcggag agaatcacac cgcactgatc acagacattg gcctgatgta tacctttggc |
| 961 | gatggacgac acgggaagct gggactggga ctggagaact tcactaatca ttttatcccc |
| 1021 | accctgtgtt ctaacttcct gcggttcatc gtgaaactgg tcgcttgcgg cgggtgtcac |
| 1081 | atggtggtct tcgctgcacc tcataggggc gtggctaagg agatcgaatt tgacgagatt |
| 1141 | aacgatacat gcctgagcgt ggcaactttc ctgccataca gctccctgac ttctggcaat |
| 1201 | gtgctgcaga gaaccctgag tgcaaggatg cggagaaggg agagggaacg ctctcctgac |
| 1261 | agtttctcaa tgcgacgaac cctgccacct atcgagggaa cactgggact gagtgcctgc |
| 1321 | ttcctgccta actcagtgtt tccacgatgt agcgagcgga atctgcagga gtctgtcctg |
| 1381 | agtgagcagg atctgatgca gccagaggaa cccgactacc tgctggatga gatgaccaag |
| 1441 | gaggccgaaa tcgacaactc tagtacagtg gagtccctgg gcgagactac cgatatcctg |
| 1501 | aatatgacac acattatgtc actgaacagc aatgagaaga gtctgaaact gtcaccagtg |
| 1561 | cagaagcaga agaaacagca gactattggc gagctgactc aggacaccgc cctgacagag |
| 1621 | aacgacgata gcgatgagta tgaggaaatg tccgagatga aggaaggcaa agcttgtaag |
| 1681 | cagcatgtca gtcaggggat cttcatgaca cagccagcca caactattga ggctttttca |
| 1741 | gacgaggaag tggagatccc cgaggaaaaa gagggcgcag aagattccaa ggggaatgga |
| 1801 | attgaggaac aggaggtgga agccaacgag gaaaatgtga aagtccacgg aggcaggaag |
| 1861 | gagaaaacag aaatcctgtc tgacgatctg actgacaagg ccgaggtgtc cgaaggcaag |
| 1921 | gcaaaatctg tcggagaggc agaagacgga ccagagggac gaggggatgg aacctgcgag |
| 1981 | gaaggctcaa gcggggctga gcattggcag gacgaggaac gagagaaggg cgaaaaggat |
| 2041 | aaaggccgcg gggagatgga acgacctgga gagggcgaaa aagagctggc agagaaggag |
| 2101 | gaatggaaga aaagggacgg cgaggaacag gagcagaaag aaagggagca gggccaccag |
| 2161 | aaggagcgca accaggagat ggaagagggc ggcgaggaag agcatggcga gggagaagag |
| 2221 | gaagagggcg atagagaaga ggaagaggaa aaagaaggcg aagggaagga ggaaggagag |
| 2281 | ggcgaggaag tggaaggcga gagggaaaag gaggaaggag aacggaagaa agaggaaaga |
| 2341 | gccggcaaag aggaaaaggg cgaggaagag ggcgatcagg gcgaaggcga ggaggaagag |
| 2401 | accgagggcc gcggggaaga gaaagaggag ggaggagagg tggagggcgg agaggtcgaa |
| 2461 | gagggaaagg gcgagcgcga agaggaagag gaagagggcg agggcgagga agaagagggc |
| 2521 | gagggggaag aagaggaggg agagggcgaa gaggaagagg gggagggaaa gggcgaagag |
| 2581 | gaaggagagg aaggggaggg agaggaagag ggggaggagg gcgaggggga aggcgaggag |
| 2641 | gaagaaggag agggggaagg cgaagaggaa ggcgaggggg aaggagagga ggaagaaggg |
| 2701 | gaaggcgaag gcgaagagga gggagaagga gagggggagg aagaggaagg agaagggaag |
| 2761 | ggcgaggagg aaggcgaaga gggagagggg gaaggcgagg aagaggaagg cgagggcgaa |
| 2821 | ggagaggacg gcgagggcga gggagaagag gaggaagggg aatgggaagg cgaagaagag |
| 2881 | gaaggcgaag gcgaaggcga agaagagggc gaaggggagg gcgaggaggg cgaaggcgaa |
| 2941 | ggggaggaag aggaaggcga aggagaaggc gaggaagaag agggagagga ggaaggcgag |
| 3001 | gaggaaggag agggggagga ggagggagaa ggcgagggcg aagaagaaga agagggagaa |
| 3061 | gtggagggcg aagtcgaggg ggaggaggga gaaggggaag gggaggaaga agagggcgaa |
| 3121 | gaagaaggcg aggaaagaga aaaagaggga gaaggcgagg aaaaccggag aaatagggaa |
| 3181 | gaggaggaag aggaagaggg aaagtaccag gagacaggcg aagaggaaaa cgagcggcag |
| 3241 | gatggcgagg aatataagaa agtgagcaag atcaaaggat ccgtcaagta cggcaagcac |
| 3301 | aaaacctatc agaagaaaag cgtgaccaac acacagggga atggaaaaga gcagaggagt |
| 3361 | aagatgcctg tgcagtcaaa acggctgctg aagaatggcc catctggaag taaaaaattc |
| 3421 | tggaacaatg tgctgcccca ctatctggaa ctgaaataa. |

In some embodiments of the compositions of the disclosure, the sequence encoding the polyA signal comprises a bovine growth hormone (BGH) polyA sequence. In some embodiments, the sequence encoding the BGH polyA signal comprises the nucleotide sequence of:

| (SEQ ID NO: 4) | |
|---|---|
| 1 | tcgctgatca gcctcgactg tgccttctag ttgccagcca tctgttgttt gcccctcccc |
| 61 | cgtgccttcc ttgaccctgg aaggtgccac tcccactgtc ctttcctaat aaaatgagga |
| 121 | aattgcatcg cattgtctga gtaggtgtca ttctattctg gggggtgggg tggggcagga |
| 181 | cagcaagggg gaggattggg aagacaatag caggcatgct ggggatgcgg tgggctctat |
| 241 | ggcttctgag gcggaaagaa ccagctgggg. |

In some embodiments of the compositions of the disclosure, the sequence encoding the 5′ ITR is derived from a 5′ITR sequence of an AAV of serotype 2 (AAV2). In some embodiments, the sequence encoding the 5′ ITR comprises a sequence that is identical to a sequence of a 5′ITR of an AAV2. In some embodiments, the sequence encoding the 5′ ITR comprises or consists of the nucleotide sequence of:

(SEQ ID NO: 5)
CTGCGCGCTCGCTCGCTCACTGAGGCCGCCCGGGCGTCGGGCGACCTTTGG
TCGCCCGGCCTCAGTGAGCGAGCGAGCGCGCAGAGAGGGAGTGGCCAACTC
CATCACTAGGGGTTCCT.

In some embodiments of the compositions of the disclosure, the sequence encoding the 3′ ITR is derived from a 3′ITR sequence of an AAV2. In some embodiments, the sequence encoding the 3′ ITR comprises a sequence that is identical to a sequence of a 3′ITR of an AAV2. In some embodiments, the sequence encoding the 3′ ITR comprises or consists of the nucleotide sequence of:

(SEQ ID NO: 6)
AGGAACCCCTAGTGATGGAGTTGGCCACTCCCTCTCTGCGCGCTCGCTCGC
TCACTGAGGCCGGGCGACCAAAGGTCGCCCGACGCCCGGGCTTTGCCCGGG
CGGCCTCAGTGAGCGAGCGAGCGCGCAG.

In some embodiments of the compositions of the disclosure, the polynucleotide further comprises a Kozak sequence. In some embodiments, the Kozak sequence comprises or consists of the nucleotide sequence of GGCCACCATG. (SEQ ID NO:7)

In some embodiments of the compositions of the disclosure, the polynucleotide comprises or consists of the sequence of:

| (SEQ ID NO: 8) | |
|---|---|
| 1 | CTGCGCGCTC GCTCGCTCAC TGAGGCCGCC CGGGCGTCGG GCGACCTTTG GTCGCCCGGC |
| 61 | CTCAGTGAGC GAGCGAGCGC GCAGAGAGGG AGTGGCCAAC TCCATCACTA GGGGTTCCTG |
| 121 | CGGCAATTCA GTCGATAACT ATAACGGTCC TAAGGTAGCG ATTTAAATAC GCGCTCTCTT |
| 181 | AAGGTAGCCC CGGGACGCGT CAATTGGGGC CCCAGAAGCC TGGTGGTTGT TTGTCCTTCT |
| 241 | CAGGGGAAAA GTGAGGCGGC CCCTTGGAGG AAGGGGCCGG GCAGAATGAT CTAATCGGAT |
| 301 | TCCAAGCAGC TCAGGGGATT GTCTTTTTCT AGCACCTTCT TGCCACTCCT AAGCGTCCTC |
| 361 | CGTGACCCCG GCTGGGATTT AGCCTGGTGC TGTGTCAGCC CCGGGGCCAC CATGAGAGAG |
| 421 | CCAGAGGAGC TGATGCCAGA CAGTGGAGCA GTGTTTACAT TCGGAAAATC TAAGTTCGCT |
| 481 | GAAAATAACC CAGGAAAGTT CTGGTTTAAA AACGACGTGC CCGTCCACCT GTCTTGTGGC |
| 541 | GATGAGCATA GTGCCGTGGT CACTGGGAAC AATAAGCTGT ACATGTTCGG GTCCAACAAC |
| 601 | TGGGGACAGC TGGGGCTGGG ATCCAAATCT GCTATCTCTA AGCCAACCTG CGTGAAGGCA |
| 661 | CTGAAACCCG AGAAGGTCAA ACTGGCCGCT TGTGGCAGAA ACCACACTCT GGTGAGCACC |
| 721 | GAGGGCGGGA ATGTCTATGC CACCGGAGGC AACAATGAGG GACAGCTGGG ACTGGGGGAC |
| 781 | ACTGAGGAAA GGAATACCTT TCACGTGATC TCCTTCTTTA CATCTGAGCA TAAGATCAAG |
| 841 | CAGCTGAGCG CTGGCTCCAA CACATCTGCA GCCCTGACTG AGGACGGGCG CCTGTTCATG |
| 901 | TGGGGAGATA ATTCAGAGGG CCAGATTGGG CTGAAAAACG TGAGCAATGT GTGCGTCCCT |
| 961 | CAGCAGGTGA CCATCGGAAA GCCAGTCAGT TGGATTTCAT GTGGCTACTA TCATAGCGCC |
| 1021 | TTCGTGACCA CAGATGGCGA GCTGTACGTC TTTGGGGAGC CCGAAAACGG AAAACTGGGC |
| 1081 | CTGCCTAACC AGCTGCTGGG CAATCACCGG ACACCCCAGC TGGTGTCCGA GATCCCTGAA |
| 1141 | AAAGTGATCC AGGTCGCCTG CGGGGGAGAG CATACAGTGG TCCTGACTGA GAATGCTGTG |
| 1201 | TATACCTTCG GACTGGGCCA GTTTGGCCAG CTGGGGCTGG GAACCTTCCT GTTTGAGACA |
| 1261 | TCCGAACCAA AAGTGATCGA GAACATTCGC GACCAGACTA TCAGCTACAT TTCCTGCGGA |
| 1321 | GAGAATCACA CCGCACTGAT CACAGACATT GGCCTGATGT ATACCTTTGG CGATGGACGA |
| 1381 | CACGGGAAGC TGGGACTGGG ACTGGAGAAC TTCACTAATC ATTTTATCCC CACCCTGTGT |
| 1441 | TCTAACTTCC TGCGGTTCAT CGTGAAACTG GTCGCTTGCG GCGGGTGTCA CATGGTGGTC |
| 1501 | TTCGCTGCAC CTCATAGGGG CGTGGCTAAG GAGATCGAAT TTGACGAGAT TAACGATACA |
| 1561 | TGCCTGAGCG TGGCAACTTT CCTGCCATAC AGCTCCCTGA CTTCTGGCAA TGTGCTGCAG |
| 1621 | AGAACCCTGA GTGCAAGGAT GCGGAGAAGG GAGAGGGAAC GCTCTCCTGA CAGTTTCTCA |
| 1681 | ATGCGACGAA CCCTGCCACC TATCGAGGGA ACACTGGGAC TGAGTGCCTG CTTCCTGCCT |
| 1741 | AACTCAGTGT TTCCACGATG TAGCGAGCGG AATCTGCAGG AGTCTGTCCT GAGTGAGCAG |
| 1801 | GATCTGATGC AGCCAGAGGA ACCCGACTAC CTGCTGGATG AGATGACCAA GGAGGCCGAA |
| 1861 | ATCGACAACT CTAGTACAGT GGAGTCCCTG GGCGAGACTA CCGATATCCT GAATATGACA |
| 1921 | CACATTATGT CACTGAACAG CAATGAGAAG AGTCTGAAAC TGTCACCAGT GCAGAAGCAG |
| 1981 | AAGAAACAGC AGACTATTGG CGAGCTGACT CAGGACACCG CCCTGACAGA GAACGACGAT |
| 2041 | AGCGATGAGT ATGAGGAAAT GTCCGAGATG AAGGAAGGCA AAGCTTGTAA GCAGCATGTC |
| 2101 | AGTCAGGGGA TCTTCATGAC ACAGCCAGCC ACAACTATTG AGGCTTTTTC AGACGAGGAA |
| 2161 | GTGGAGATCC CCGAGGAAAA AGAGGGCGCA GAAGATTCCA AGGGGAATGG AATTGAGGAA |
| 2221 | CAGGAGGTGG AAGCCAACGA GGAAAATGTG AAAGTCCACG GAGGCAGGAA GGAGAAAACA |
| 2281 | GAAATCCTGT CTGACGATCT GACTGACAAG GCCGAGGTGT CCGAAGGCAA GGCAAAATCT |
| 2341 | GTCGGAGAGG CAGAAGACGG ACCAGAGGGA CGAGGGGATG GAACCTGCGA GGAAGGCTCA |
| 2401 | AGCGGGGCTG AGCATTGGCA GGACGAGGAA CGAGAGAAGG GCGAAAAGGA TAAAGGCCGC |
| 2461 | GGGGAGATGG AACGACCTGG AGAGGGCGAA AAAGAGCTGG CAGAGAAGGA GGAATGGAAG |
| 2521 | AAAAGGGACG GCGAGGAACA GGAGCAGAAA GAAAGGGAGC AGGGCCACCA GAAGGAGCGC |
| 2581 | AACCAGGAGA TGGAAGAGGG CGGCGAGGAA GAGCATGGCG AGGGAGAAGA GGAAGAGGGC |
| 2641 | GATAGAGAAG AGGAAGAGGA AAAAGAAGGC GAAGGGAAGG AGGAAGGAGA GGGCGAGGAA |
| 2701 | GTGGAAGGCG AGAGGGAAAA GGAGGAAGGA GAACGGAAGA AAGAGGAAAG AGCCGGCAAA |
| 2761 | GAGGAAAAGG GCGAGGAAGA GGGCGATCAG GGCGAAGGCG AGGAGGAAGA GACCGAGGGC |
| 2821 | CGCGGGGAAG AGAAAGAGGA GGGAGGAGAG GTGGAGGGCG GAGAGGTCGA AGAGGGAAAG |
| 2881 | GGCGAGCGCG AAGAGGAAGA GGAAGAGGGC GAGGGCGAGG AAGAAGAGGG CGAGGGGGAA |
| 2941 | GAAGAGGAGG GAGAGGGCGA AGAGGAAGAG GGGGAGGGAA AGGGCGAAGA GGAAGGAGAG |
| 3001 | GAAGGGGAGG GAGAGGAAGA GGGGGAGGAG GGCGAGGGGG AAGGCGAGGA GGAAGAAGGA |
| 3061 | GAGGGGGAAG GCGAAGAGGA AGGCGAGGGG GAAGGAGAGG AGGAAGAAGG GGAAGGCGAA |
| 3121 | GGCGAAGAGG AGGGAGAAGG AGAGGGGGAG GAAGAGGAAG GAGAAGGGAA GGGCGAGGAG |
| 3181 | GAAGGCGAAG AGGGAGAGGG GGAAGGCGAG GAAGAGGAAG GCGAGGGCGA AGGAGAGGAC |
| 3241 | GGCGAGGGCG AGGGAGAAGA GGAGGAAGGG GAATGGGAAG GCGAAGAAGA GGAAGGCGAA |
| 3301 | GGCGAAGGCG AAGAAGAGGG CGAAGGGGAG GGCGAGGAGG GCGAAGGCGA AGGGGAGGAA |
| 3361 | GAGGAAGGCG AAGGAGAAGG CGAGGAAGAA GAGGGAGAGG AGGAAGGCGA GGAGGAAGGA |
| 3421 | GAGGGGGAGG AGGAGGGAGA AGGCGAGGGC GAAGAAGAAG AAGAGGGAGA AGTGGAGGGC |
| 3481 | GAAGTCGAGG GGGAGGAGGG AGAAGGGGAA GGGGAGGAAG AAGAGGGCGA AGAAGAAGGC |
| 3541 | GAGGAAAGAG AAAAAGAGGG AGAAGGCGAG GAAAACCGGA GAAATAGGGA AGAGGAGGAA |
| 3601 | GAGGAAGAGG GAAAGTACCA GGAGACAGGC GAAGAGGAAA ACGAGCGGCA GGATGGCGAG |
| 3661 | GAATATAAGA AAGTGAGCAA GATCAAAGGA TCCGTCAAGT ACGGCAAGCA CAAAACCTAT |
| 3721 | CAGAAGAAAA GCGTGACCAA CACACAGGGG AATGGAAAAG AGCAGAGGAG TAAGATGCCT |
| 3781 | GTGCAGTCAA AACGGCTGCT GAAGAATGGC CCATCTGGAA GTAAAAAATT CTGGAACAAT |
| 3841 | GTGCTGCCCC ACTATCTGGA ACTGAAATAA GAGCTCCTCG AGGCGGCCCG CTCGAGTCTA |
| 3901 | GAGGGCCCTT CGAAGGTAAG CCTATCCCTA ACCCTCTCCT CGGTCTCGAT TCTACGCGTA |
| 3961 | CCGGTCATCA TCACCATCAC CATTGAGTTT AAACCCGCTG ATCAGCCTCG ACTGTGCCTT |
| 4021 | CTAGTTGCCA GCCATCTGTT GTTTGCCCCT CCCCCGTGCC TTCCTTGACC CTGGAAGGTG |
| 4081 | CCACTCCCAC TGTCCTTTCC TAATAAAATG AGGAAATTGC ATCGCATTGT CTGAGTAGGT |
| 4141 | GTCATTCTAT TCTGGGGGGT GGGGTGGGGC AGGACAGCAA GGGGGAGGAT TGGGAAGACA |
| 4201 | ATAGCAGGCA TGCTGGGGAT GCGGTGGGCT CTATGGCTTC TGAGGCGGAA AGAACCAGAT |
| 4261 | CCTCTCTTAA GGTAGCATCG AGATTTAAAT TAGGGATAAC AGGGTAATGG CGCGGGCCGC |
| 4321 | AGGAACCCCT AGTGATGGAG TTGGCCACTC CCTCTCTGCG CGCTCGCTCG CTCACTGAGG |
| 4381 | CCGGGCGACC AAAGGTCGCC CGACGCCCGG GCTTTGCCCG GGCGGCCTCA GTGAGCGAGC |
| 4441 | GAGCGCGCAG. |

In some embodiments of the compositions of the disclosure, the polynucleotide further comprises a sequence encoding a woodchuck posttranslational regulatory element (WPRE). In some embodiments, the WPRE comprises a nucleotide sequence of:

| (SEQ ID NO: 9) | |
|---|---|
| 1 | atcaacctct ggattacaaa atttgtgaaa gattgactgg tattcttaac tatgttgctc |
| 61 | cttttacgct atgtggatac gctgctttaa tgcctttgta tcatgctatt gcttcccgta |
| 121 | tggctttcat tttctcctcc ttgtataaat cctggttgct gtctctttat gaggagttgt |
| 181 | ggcccgttgt caggcaacgt ggcgtggtgt gcactgtgtt tgctgacgca acccccactg |
| 241 | gttggggcat tgccaccacc tgtcagctcc tttccgggac tttcgctttc cccctcccta |
| 301 | ttgccacggc ggaactcatc gccgcctgcc ttgcccgctg ctggacaggg gctcggctgt |
| 361 | tgggcactga caattccgtg gtgttgtcgg ggaaatcatc gtcctttcct tggctgctcg |
| 421 | cctgtgttgc cacctggatt ctgcgcggga cgtccttctg ctacgtccct tcggccctca |
| 481 | atccagcgga ccttccttcc cgcggcctgc tgccggctct gcggcctctt ccgcgtcttc |
| 541 | gccttcgccc tcagacgagt cggatctccc tttgggccgc ctccccgc. |

In some embodiments of the compositions of the disclosure, each of the rAAV8 particles comprise a viral Rep protein isolated or derived from an AAV serotype 8 (AAV8) Rep protein.

In some embodiments of the compositions of the disclosure, each of the rAAV8 particles comprise a viral Cap protein isolated or derived from an AAV serotype 8 (AAV8) Cap protein.

The disclosure provides a device, comprising the composition of the disclosure.

In some embodiments of the devices of the disclosure, the device comprises a microdelivery device. In some embodiments, the microdelivery device comprises a microneedle. In some embodiments, the microneedle is suitable for subretinal delivery. In some embodiments, the device comprises a volume of at least 50 μL. In some embodiments, the device comprises a volume of 5 μL, 10 μL, 15 μL, 20 μL, 25 μL, 50 μL, 75 μL, 100 μL, 150 μL, 200 μL, 250 μL, 300 μL, 350 μL, 400 μL, 450 μL, 500 μL, 550 μL, 600 μL, 650 μL, 700 μL, 750 μL, 800 μL, 850 μL, 900 μL 950 μL, 1000 μL or any number of 4 in between.

In some embodiments of the devices of the disclosure, the device comprises a microdelivery device. In some embodiments, the microdelivery device comprises a microcatheter. In some embodiments, the device is suitable for suprachoroidal delivery. In some embodiments, the device comprises a volume of at least 50 μL. In some embodiments, the device comprises a volume of 5 μL, 10 μL, 15 μL, 20 μL, 25 μL, 50 μL, 75 μL, 100 μL, 150 μL, 200 μL, 250 μL, 300 μL, 350 μL, 400 μL, 450 μL, 500 μL, 550 μL, 600 μL, 650 μL, 700 μL, 750 μL, 800 μL, 850 μL, 900 μL 950 μL, 1000 μL or any number of 4 in between.

The disclosure provides a method of treating Retinitis Pigmentosa in a subject in need thereof, comprising administering to the subject a therapeutically effective amount of the composition of the disclosure.

The disclosure provides a method of treating Retinitis Pigmentosa in a subject in need thereof, comprising administering to the subject a therapeutically effective amount of a composition, wherein the administration is performed using a device of the disclosure.

In some embodiments of the methods of treating Retinitis Pigmentosa of the disclosure, administering the therapeutically effective amount of the composition improves a sign of Retinitis Pigmentosa in the subject.

In some embodiments of the methods of treating Retinitis Pigmentosa of the disclosure, the sign of Retinitis Pigmentosa comprises a degeneration of an ellipsoid zone (EZ) when compared to a healthy EZ. In some embodiments, the degeneration of the EZ comprises a reduction in photoreceptor cell density, a reduction in number of photoreceptor cilia, or a combination thereof, when compared to a healthy EZ. In some embodiments, the degeneration of the EZ comprises a reduction of a width of the EZ when compared to a healthy EZ, wherein the width comprises a distance between an inner photoreceptor segment and an outer photoreceptor segment. In some embodiments, the degeneration of the EZ comprises a reduction of a length of the EZ when compared to a healthy EZ, wherein the length comprises a distance along one or more of the anterior to posterior (A/P) axis, the dorsal to ventral (D/V) axis or the medial to lateral (M/L) axis of the eye. In some embodiments, the degeneration of the EZ comprises a reduction of a area of the EZ when compared to a healthy EZ, wherein the area comprises a π time the square of the distance along one or more of the anterior to posterior (A/P) axis, the dorsal to ventral (D/V) axis or the medial to lateral (M/L) axis of the eye.

In some embodiments of the methods of treating Retinitis Pigmentosa of the disclosure, the healthy EZ comprises an EZ of an age and gender matched individual who does not have either a sign or symptom of Retinitis Pigmentosa. In some embodiments, the age and gender matched individual who does not have either a sign or symptom of Retinitis Pigmentosa does not have a risk factor for developing Retinitis Pigmentosa. In some embodiments, the healthy EZ comprises a predetermined control or threshold. In some embodiments, the predetermined control or threshold comprises an average or mean value determined from measurements of a plurality of healthy EZ from a plurality of individuals. In some embodiments, the plurality of individuals are age and gender matched to the subject. In some embodiments, the healthy EZ comprises an unaffected eye of the subject. In some embodiments, the unaffected eye does not have a detectable sign of Retinitis Pigmentosa. In some embodiments, the unaffected eye does not have detectable degeneration of the EZ.

In some embodiments of the methods of treating Retinitis Pigmentosa of the disclosure, the sign of Retinitis Pigmentosa comprises a degeneration of an ellipsoid zone (EZ) when compared to a baseline EZ. In some embodiments, the baseline EZ comprises a measurement of the degeneration of the subject's EZ prior to administration of the composition. In some embodiments, the measurement of the degeneration of the subject's EZ comprises a determination of a number of living or viable photoreceptors in a portion of the EZ, a number of cilia in a portion of the EZ, a width of a portion of the EZ, a length of the EZ along one or more axes in a portion of the EZ, an area of a portion of the EZ, or any combination thereof.

In some embodiments of the methods of treating Retinitis Pigmentosa of the disclosure, administering the therapeutically effective amount of the composition improves a sign or a symptom of Retinitis Pigmentosa, wherein the sign of Retinitis Pigmentosa comprises the degeneration of an ellipsoid zone (EZ) when compared to a healthy EZ or a baseline EZ and wherein the improvement comprises increasing the width of the EZ between 1 μm and 20 μm, inclusive of the endpoints. In some embodiments, the improvement comprises increasing the width of the EZ between 3 μm and 15 μm, inclusive of the endpoints. In some embodiments, the improvement comprises increasing the width of the EZ between 0.8 μm and 320 μm, inclusive of the endpoints.

In some embodiments of the methods of treating Retinitis Pigmentosa of the disclosure, the improvement comprises increasing the width of the EZ by 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100% or any percentage in between, when compared to a baseline EZ. In some embodiments, the improvement comprises increasing the width of the EZ uniformly across one or more sector(s) of the eye. In some embodiments, the improvement comprises increasing the width of the EZ non-uniformly across one or more sector(s) of the eye, wherein the increased width is maximal at the macula or within one or more central sector(s) and wherein the increased width is minimal at one or more peripheral sector(s).

In some embodiments of the methods of treating Retinitis Pigmentosa of the disclosure, the improvement comprises increasing the length of the EZ along the A/P axis. In some embodiments, the improvement comprises increasing the length of the EZ along the D/V axis. In some embodiments, the improvement comprises increasing the length of the EZ along the M/L axis.

In some embodiments of the methods of treating Retinitis Pigmentosa of the disclosure, the improvement comprises increasing the length of the EZ by 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100% or any percentage in between, when compared to a baseline EZ.

In some embodiments of the methods of treating Retinitis Pigmentosa of the disclosure, administering the therapeutically effective amount of the composition reduces a rate of further degeneration or inhibits further degeneration of the EZ when compared to a baseline EZ. In some embodiments, following administration of the composition, a number of living or viable photoreceptors in a portion of the EZ, a number of cilia in a portion of the EZ, a width of a portion of the EZ, a length of the EZ along one or more axes in a portion of the EZ, an area of a portion of the EZ, or any combination thereof is equal to the number of living or viable photoreceptors in the portion of the EZ, the number of cilia in the portion of the EZ, the width of the portion of the EZ, the length of the EZ along one or more axes in the portion of the EZ or any combination thereof when compared to a baseline EZ.

In some embodiments of the methods of treating Retinitis Pigmentosa of the disclosure, a width or a length of a portion of the EZ of the subject or a width or a length of a portion of a healthy EZ is measured using optical coherence tomography (OCT).

In some embodiments of the methods of treating Retinitis Pigmentosa of the disclosure, the sign of Retinitis Pigmentosa comprises a reduction in retinal thickness and/or in outer nuclear layer (ONL) thickness when compared to a healthy retinal thickness and/or a healthy ONL thickness.

In some embodiments of the methods of treating Retinitis Pigmentosa of the disclosure, a healthy retinal thickness or a healthy ONL thickness is that of an age and gender matched individual who does not have either a sign or symptom of Retinitis Pigmentosa. In some embodiments, the age and gender matched individual who does not have either a sign or symptom of Retinitis Pigmentosa does not have a risk factor for developing Retinitis Pigmentosa. In some embodiments, the healthy retinal thickness or healthy ONL thickness comprises a predetermined control or threshold. In some embodiments, the predetermined control or threshold comprises an average or mean value determined from measurements of a plurality of healthy retinal thicknesses or healthy ONL thicknesses from a plurality of individuals. In some embodiments, the plurality of individuals are age and gender matched to the subject. In some embodiments, the healthy retinal thickness or healthy ONL thickness comprises an unaffected eye of the subject. In some embodiments, the unaffected eye does not have a detectable sign of Retinitis Pigmentosa. In some embodiments, the unaffected eye does not have detectable reduction of retinal thickness or ONL thickness.

In some embodiments of the methods of treating Retinitis Pigmentosa of the disclosure, improvement of a sign of Retinitis Pigmentosa comprises an increase in retinal thickness and/or ONL thickness when compared to a baseline retinal thickness and/or ONL thickness. In some embodiments, the baseline retinal thickness and/or ONL thickness comprises a measurement of the retinal thickness and/or ONL thickness prior to administration of the composition.

In some embodiments of the methods of treating Retinitis Pigmentosa of the disclosure, administering the therapeutically effective amount of the composition improves a sign or a symptom of Retinitis Pigmentosa, wherein the sign of Retinitis Pigmentosa comprises the reduction of retinal thickness and/or ONL thickness when compared to a healthy EZ.

In some embodiments of the methods of treating Retinitis Pigmentosa of the disclosure, the improvement comprises increasing the retinal thickness and/or ONL thickness by 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100% or any percentage in between, when compared to a baseline retinal thickness and/or ONL thickness. In some embodiments, the improvement comprises increasing the retinal thickness and/or ONL thickness uniformly across one or more sector(s) of the eye. In some embodiments, the improvement comprises increasing the retinal thickness and/or ONL thickness non-uniformly across one or more sector(s) of the eye, wherein the increased thickness is maximal at the macula or within one or more central sector(s) and wherein the increased thickness is minimal at one or more peripheral sector(s).

In some embodiments of the methods of treating Retinitis Pigmentosa of the disclosure, administering the therapeutically effective amount of the composition reduces a rate of further degeneration or inhibits further degeneration of the retinal thickness and/or ONL thickness when compared to a baseline retinal thickness and/or ONL thickness.

In some embodiments of the methods of treating Retinitis Pigmentosa of the disclosure, a retinal thickness and/or an ONL thickness of the subject or a retinal thickness and/or an ONL thickness of a healthy individual is measured using OCT.

In some embodiments of the methods of treating Retinitis Pigmentosa of the disclosure, administering the therapeutically effective amount of the composition induces regeneration of photoreceptor outer segments when compared to photoreceptor outer segments of the subject before administration of the composition.

In some embodiments of the methods of treating Retinitis Pigmentosa of the disclosure, the sign of Retinitis Pigmentosa comprises a reduction of a level of retinal sensitivity compared to a healthy level of retinal sensitivity. In some embodiments, the level of retinal sensitivity is measured using microperimetry. In some embodiments, measuring the level of retinal sensitivity comprises: (a) generating an image of a fundus of an eye of the subject; (b) projecting a grid of points onto the image of (a); (c) stimulating the eye at each point on the grid of (b) with light, wherein each subsequent stimulus has a greater intensity than a previous stimulus; (d) repeating step (c) at least once; (e) determining for each point on the grid of (b) a minimum threshold value, wherein the minimum threshold value is an intensity of light from (c) at which the subject can first perceive the light; and (f) converting the minimum threshold value from (e) from asb to decibels (dB), wherein a maximum intensity of light equals 0 dB and a minimum intensity of light equals a maximum dB value of a dB scale, or wherein a maximum intensity of light equals retinal sensitivity of 0 dB and a minimum intensity of light equals a maximum dB value of a dB scale that quantifies retinal sensitivity. In some embodiments, the stimulating step of (c) comprises a light stimulus having a range from approximately 4 to 1000 apostilb (asb). In some embodiments, the grid comprises at least 37 points. In some embodiments, the grid comprises or consists of 68 points. In some embodiments, the points are evenly spaced over a circle having a diameter that covers 10° of the eye. In some embodiments, the circle is centered on the macula. In some embodiments, measuring the level of retinal sensitivity further comprises averaging the minimum threshold value at each point in the grid of (b) to produce a mean retinal sensitivity.

In some embodiments of the methods of treating Retinitis Pigmentosa of the disclosure, the healthy level of retinal sensitivity is determined using an age and gender matched individual who does not have either a sign or symptom of Retinitis Pigmentosa. In some embodiments, the age and gender matched individual who does not have either a sign or symptom of Retinitis Pigmentosa does not have a risk factor for developing Retinitis Pigmentosa. In some embodiments, the healthy level of retinal sensitivity is determined using a predetermined control or threshold. In some embodiments, the predetermined control or threshold comprises an average or mean value determined from measurements of a plurality of healthy levels of retinal sensitivity from a plurality of individuals. In some embodiments, the plurality of individuals are age and gender matched to the subject. In some embodiments, the healthy level of retinal sensitivity is measured from an unaffected eye of the subject. In some embodiments, the unaffected eye does not have a detectable sign of Retinitis Pigmentosa. In some embodiments, the unaffected eye does not have detectable reduction in a level of retinal sensitivity. In some embodiments, the sign of Retinitis Pigmentosa comprises a reduction of a level of retinal sensitivity when compared to a baseline level of retinal sensitivity. In some embodiments, the baseline level of retinal sensitivity comprises a measurement of a level of retinal sensitivity of the subject prior to administration of the composition.

In some embodiments of the methods of treating Retinitis Pigmentosa of the disclosure, administering the therapeutically effective amount of the composition restores retinal sensitivity of the subject when compared to a healthy level of retinal sensitivity. In some embodiments, restoring retinal sensitivity comprises an increase in a mean retinal sensitivity in a portion of the retina when compared to a healthy level of retinal sensitivity. In some embodiments, a mean retinal sensitivity in a portion of the retina of the subject equals a mean retinal sensitivity in the portion of the retina in the healthy level of retinal sensitivity.

In some embodiments of the methods of treating Retinitis Pigmentosa of the disclosure, administering the therapeutically effective amount of the composition improves retinal sensitivity of the subject when compared to a baseline level of retinal sensitivity. In some embodiments, improving retinal sensitivity comprises an increase in a mean retinal sensitivity in a portion of the retina when compared to a baseline level of retinal sensitivity. In some embodiments, improving retinal sensitivity comprises an increase in a level of mean retinal sensitivity of between 1 and 30 decibels (dB), inclusive of the endpoints. In some embodiments, improving retinal sensitivity comprises an increase in a level of mean retinal sensitivity of between 1 and 15 dB, inclusive of the endpoints. In some embodiments, improving retinal sensitivity comprises an increase in a level of mean retinal sensitivity of between 2 to 10 dB, inclusive of the endpoints. In some embodiments, improving retinal sensitivity comprises an increase in a level of mean retinal sensitivity of at least 5 dB, at least 6 dB, at least 7 dB, at least 8 dB, at least 9 dB, or at least 10 dB. In some embodiments, improving retinal sensitivity comprises an increase in a level of mean retinal sensitivity of at least 7 dB.

In some embodiments, improving retinal sensitivity comprises an increase in sensitivity of at least 5 dB, at least 6 dB, at least 7 dB, at least 8 dB, at least 9 dB, or at least 10 dB in between 1-68 points, inclusive of the endpoints. In some embodiments, improving retinal sensitivity comprises an increase in sensitivity of at least 7 dB in at least 2, at least 3, at least 4, at least 5, at least 10, at least 15, at least 20, at least at least 25, at least 30, at least 35, at least 40, at least 45, at least 50, at least 55, at least 60 or at least 65 points. In some embodiments, improving retinal sensitivity comprises an increase in sensitivity of at least 5 dB, at least 6 dB, at least 7 dB, at least 8 dB, at least 9 dB, or at least 10 dB in at least 5 points in the central 16 points of a 68 point grid. In some embodiments, improving retinal sensitivity comprises an increase in sensitivity of at least 7 dB in at least 5 points in the central 16 points of a 68 point grid. In some embodiments, improving retinal sensitivity comprises an increase in sensitivity of at least 7 dB in at least 2, at least 3, at least 4, at least 5, at least 10, at least 15, at least 20, at least at least 25, at least 30, at least 35, at least 40, at least 45, at least 50, at least 55, at least 60 or at least 65 points of a 68 point grid. In some embodiments, improving retinal sensitivity comprises an increase in sensitivity of at least 7 dB in at least 60 or at least 65 points of a 68 point grid. In some embodiments, improving retinal sensitivity comprises an increase in sensitivity of at least 5 dB, at least 6 dB, at least 7 dB, at least 8 dB, at least 9 dB, or at least 10 dB in all points of a 68 point grid. In some embodiments, improving retinal sensitivity comprises an increase in sensitivity of at least 7 dB in all points of a 68 point grid.

In some embodiments, improving retinal sensitivity comprises an increase in a level of mean retinal sensitivity of 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100% or any percentage in between in a level of mean retinal sensitivity when compared to a baseline level of retinal sensitivity. In some embodiments, the increase in a level of mean retinal sensitivity occurs in at least 1, 2, 3, 4, 5, 6, 7, 8, 9, 10, 15, 20, 25, 30, 35 or any number of points in between within a microperimetery grid. In some embodiments, the increase in a level of mean retinal sensitivity occurs in at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100% or any percentage in between in within a microperimetery grid.

In some embodiments of the methods of treating Retinitis Pigmentosa of the disclosure, administering the therapeutically effective amount of the composition inhibits further reduction or prevents loss of retinal sensitivity of the subject when compared to a baseline level of retinal sensitivity. In some embodiments, a level retinal sensitivity in the subject following administration of the composition equals the baseline level of retinal sensitivity

The disclosure provides a method of preventing Retinitis Pigmentosa in a subject, comprising administering to the subject a prophylactically effective amount of the composition of the disclosure, wherein the subject is at risk of developing Retinitis Pigmentosa. In some embodiments, the subject has a risk factor for developing Retinitis Pigmentosa. In some embodiments, the factor comprises one or more of a genetic marker, a family history of Retinitis Pigmentosa, a symptom of Retinitis Pigmentosa or a combination thereof. In some embodiments, the symptom of Retinitis Pigmentosa comprises a reduction or loss of visual acuity. In some embodiments, the visual acuity relates to night vision, peripheral vision, color vision or any combination thereof.

In some embodiments of the methods of the disclosure, the composition is administered by a subretinal route. In some embodiments, the composition is administered by a subretinal injection or infusion. In some embodiments, the composition is administered by a subretinal injection and wherein the injection comprises a volume of between 50 μL and 1000 μL, inclusive of endpoint. In some embodiments, the composition is administered by a subretinal injection and wherein the injection comprises a volume of between 50 μL and 300 μL, inclusive of endpoint. In some embodiments, the composition is administered by a subretinal injection and wherein the injection comprises a volume of 100 μL or up to 100 μL (e.g., 25-100 μL, 50-100 μL, 75-100 μL). In some embodiments, thesubretinal injection comprises two-step injection. In some embodiments, the two-step injection comprises: (a) inserting a microneedle between a photoreceptor cell layer and a retinal pigment epithelial (RPE) layer in an eye of the subject; (b) injecting a solution between the photoreceptor cell layer and a retinal pigment epithelial layer in the eye of the subject in an amount sufficient to partially detach the retina from the RPE to form a bleb; and (c) injecting the composition into the bleb of (b). In some embodiments, the solution comprises a balanced salt solution.

In some embodiments of the methods of the disclosure, the composition is administered by a suprachoroidal route. In some embodiments, the composition is administered by a suprachoroidal injection or infusion. In some embodiments, the composition is administered by a suprachoroidal injection and wherein the injection comprises a volume of between 50 and 1000 μL, inclusive of the endpoints. In some embodiments, the injection comprises a volume of between 50 and 300 μL, inclusive of the endpoints. n some embodiments, the injection comprises a volume of between 50 and 200 μL, inclusive of the endpoints. In some embodiments, the injection comprises a volume of between 50 and 100 μL, inclusive of the endpoints. In some embodiments, the suprachoroidal injection comprises: (a) contacting a hollow end of a microdelivery device and a suprachoroidal space of an eye of the subject, wherein the hollow end comprises an opening; and (b) flowing the composition through the hollow end of the microdelivery device to introduce the composition into the suprachoroidal space. In some embodiments, the suprachoroidal injection comprises wherein the hollow end of the microdelivery device pierced a sclera, wherein the hollow end of the microdelivery device or an extension thereof traversed a portion of a suprachoroidal space, and wherein the hollow end of the microdelivery device traversed a choroid at least once.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a table showing the Best Corrected Visual Acuity (BCVA) measured in 7 subjects who were treated for Retinitis Pigmentosa by injection of an AAV-RPGR^{ORF15} gene therapy vector in one eye. BCVA was evaluated using the Early Treatment Diabetic Retinopathy (EDTRS) letters in each eye at each of baseline (before injection), 1 week, 1 month, 3 months, 6 months and 9 months following injection of the AAV RPGR^{ORF15} vector. 3 month and 6 month changes are indicated at bottom.

FIG. 2 is a table showing microperimetry measurements of mean threshold retinal sensitivity in decibels (dB) from 7 subjects who were treated for Retinitis Pigmentosa by injection of an AAV-RPGR^{ORF15} composition in one eye. Mean threshold was evaluated in both eyes prior to injection (at baseline), and at least at 1 month following injection of the AAV RPGR^{ORF15} vector. In some cases additional measurements were taken at 6 and 9 months. 3 month and 6 month changes are indicated at bottom.

FIG. 3 is a graph of retinal sensitivity change at 3 months. On the Y-axis, retinal sensitivity change from baseline in decibels (dB). On the X-axis, treated and untreated eyes by cohort are shown. TE=treated eye, CE=control eye.

FIG. 4A-4B are each a series of images and graphs showing the microperimetry data for JH90 OD (control eye) at baseline prior to the injection of AAV-RPGR^{ORF15} (FIG. 4A) and 3 months following injection of AAV-RPGR^{ORF15} in the other, treated eye (FIG. 4B). The average threshold (dB) for baseline (FIG. 4A) is 0.9, while the average threshold (dB) for 3 months is 0.8. Fixation stability is stable at baseline (P1=100%, P2=100%, FIG. 4A), and stable at 3 months (P1=100%, P2=100%, FIG. 4B). The Bivareate Contour Ellipse Area (BCEA) for baseline (FIG. 4A) is: 63% BCEA: 0.7°×0.4°, Area=0.2°², angle=−3.1°; 95% BCEA: 1.2°×0.7°, Area=0.7°², angle=−3.1°. The BCEA for 3 months (FIG. 4B) is: 63% BCEA: 0.5°×0.3°, Area=0.1°², angle=0.2°; 95% BVCEA: 0.9°×0.5°, Area=0.3°², angle=0.2°.

FIG. 5A-B are each a series of images and graphs showing the microperimetry data for JH90 OS (treated eye) at baseline prior to the injection of AAV-RPGR^{ORF15} (FIG. 5A) and 3 months following injection of AAV-RPGR^{ORF15} (FIG. 5B). The average threshold (dB) for baseline (FIG. 5A) is 0, while the average threshold (dB) for 3 months is 0.9. Fixation stability is relatively unstable at baseline (P1=37%, P2=82%, FIG. 5A), and is stable at 3 months (P1=99%, P2=100%, FIG. 5B). The BCEA for baseline (FIG. 5A) is: 63% BCEA: 4.4°×2.3°, Area=8.0°², angle=0.2°; 95% BCEA: 7.6°×4.0°, Area=24.0°², angle=0.2°. The BCEA for 3 months (FIG. 5B) is: 63% BCEA: 0.6°×0.2°, Area=8.0°², angle=−5.7°; 95% BCEA: 1.0°×0.7°, Area=0.5°², angle=−5.7°.

FIG. 6A-6B are each a series of images and graphs showing the microperimetry data for AH85 OD (control eye) at baseline prior to the injection of AAV-RPGR^{ORF15} (FIG. 6A) and 3 months following injection of AAV-RPGR^{ORF15} in the other, treated eye (FIG. 6B). The average threshold (dB) for baseline (FIG. 6A) is 0.8, while the average threshold (dB) for 3 months is 1.4. Fixation stability is stable at baseline (P1=83%, P2=88%, FIG. 6A), and is stable at 3 months (P1=96%, P2=100%, FIG. 6B). The BCEA for baseline (FIG. 6A) is: 63% BVCEA: 4.1°×2.3°, Area=5.0°², angle=−13.6°; 95% BVCEA: 7.1°×2.7°, Area=15.0°², angle=−13.6°. The BCEA for 3 months (FIG. 6B) is: 63% BVCEA: 1.1°×0.7°, Area=0.6°², angle=−0.6°; 95% BCEA: 1.9°×1.2°, Area=1.7°², angle=−0.6°.

FIG. 7A-7B are each a series of images and graphs showing the microperimetry data for AH85 OS (treated eye) at baseline prior to the injection of AAV-RPGR^{ORF15} (FIG. 6A) and 3 months following injection of AAV-RPGR^{ORF15} (FIG. 6B). The average threshold (dB) for baseline (FIG. 7A) is 0.9, while the average threshold (dB) for 3 months is 4.3. Fixation stability is stable at baseline (P1=98%, P2=100%, FIG. 7A), and is stable at 3 months (P1=98%, P2=100%, FIG. 7B). The BCEA for baseline (FIG. 7A) is: 63% BCEA: 0.8°×0.9°, Area=0.6°², angle=50.4°; 95% BCEA: 1.4°×1.6°, Area=1.8°², angle=50.4°. The BCEA for 3 months (FIG. 7B) is: 63% BCEA: 0.8°×0.8°, Area=0.5°², angle=−9.0°; 95% BCEA: 1.5°×1.4°, Area=1.6°², angle=−9.0°.

FIG. 8A-8B are each a series of images and graphs showing the microperimetry data for KL94 OS (control eye) at baseline prior to the injection of AAV RPGR^{ORF15} (FIG. 8A) and 1 month following injection of AAV RPGR^{ORF15} in the other, treated eye (FIG. 8B). The average threshold (dB) for baseline (FIG. 8A) is 0.7, while the average threshold (dB) for 1 month is 0.5. Fixation stability is stable at baseline (P1=95%, P2=100%, FIG. 8A), and is stable at 1 month (P1=99%, P2=100%, FIG. 8B). The BCEA for baseline (FIG. 8A) is: 63% BCEA: 1.1°×0.8°, Area=0.7°², angle=12.1°; 95% BCEA: 2.0°×1.4°, Area=2.2°², angle=12.1°. The BCEA for 1 month (FIG. 8B) is: 63% BCEA: 0.9°×0.6°, Area=0.4°², angle=−13.5°; 95% BCEA: 1.6°×1.1°, Area=1.3°², angle=−13.5°.

FIG. 9A-9B are each a series of images and graphs showing the microperimetry data for KL94 OD (treated eye) at baseline prior to the injection of AAV RPGR^{ORF15} (FIG. 9A) and 1 month following injection of AAV RPGR^{ORF15} (FIG. 9B). The average threshold (dB) for baseline (FIG. 9A) is 0.5, while the average threshold (dB) for 1 month is 3.4. Fixation stability is stable at baseline (P1=90%, P2=97%, FIG. 9A), and is stable at 1 month (P1=100%, P2=100%, FIG. 9B). The BCEA for baseline (FIG. 9A) is: 63% BCEA: 1.2°×1.4°, Area=1.3°², angle=51.2°; 95% BCEA: 2.2°×2.4°, Area=4.0°², angle=51.2°. The BCEA for 1 month (FIG. 9B) is: 63% BCEA: 0.7°×0.6°, Area=0.4°², angle=−16.7°; 95% BCEA: 1.3°×1.1°, Area=1.1°², angle=−16.7°.

FIG. 10 is a table describing mean retinal thickness (the mean of the central 1 mm ETDRS circle) measured in 3 subjects who were treated for Retinitis Pigmentosa by injection of an AAV-RPGR^{ORF15} composition in one eye. Mean retinal thickness was measured by optical coherence tomography (OCT). Mean retinal thickness was measured at baseline prior to injection of AAV-RPGR^{ORF15}, and at 1 month and 3 months following injection of AAV-RPGR^{ORF15}. The change 3 months are shown at bottom.

FIG. 11A-11B are each a series of images that show retinal sensitivity and structural changes following gene therapy for X-linked retinitis pigmentosa. (FIG. 11A) Mean retinal sensitivity (decibels, dB) and visual field (represented by sensitivity heat maps) as measured by microperimetry underwent progressive improvement in the treated eye from baseline to 4 months post-treatment, while the untreated eye remained stable. Visual acuity as measured by Early Treatment Diabetic Retinopathy Study chart reading (number of letters) remained stable in both eyes. (FIG. 11B) Complete segmentation (every OCT line scan) of the retinal outer nuclear layer (ONL) over the macula revealed localized thickening of the ONL (shown as red on the heat map) corresponding to areas of sensitivity gain in the treated eye, compared with no significant change in the untreated eye. Middle column: mean sectoral ONL thickness changes (μm) on the 1, 3 and 6 mm ETDRS macula grid. Right column: heat map of ONL thickness changed (red represents increased thickness and green represents reduced thickness).

FIG. 12A-12B are each a series of images and graphs showing raw microperimetry data for a patient following subretinal gene therapy with 1.0×10¹¹ gp AAV8.RPGR to the right eye (FIG. 12A) and no treatment to the left eye (FIG. 12B). For each microperimetry data set, the threshold sensitivity at each of the 68 test loci are color-coded and overlaid on a scanning laser ophthalmoscopy (SLO) image of the retina (top right panel). The threshold sensitivity data are also shown as a heat-map (middle-left panel) and a histogram of sensitivity frequencies with normal reference curve shown in green (middle-right panel). The patient's fixation was assessed by eye tracker in real-time throughout the test and plotted as fine cyan dots in the top-right panel. Fixation stability (as indicated by degrees of excursion from the fovea) during the test is shown in the bottom-right panel. There is no learning effect, as evidenced by the first three pre-treatment baseline field tests which are consistent in both eyes, as is the untreated eye before and after surgery. Only the treated eye shows significant improvement in retinal function, reaching a maximum around 3-4 months after gene therapy.

FIG. 13 is a diagram of an embodiment of the AAV RPGR^{ORF15} polynucleotide. The polynucleotide comprises, from 5′ to 3′, a 5′ inverted terminal repeat (ITR), a rhodopsin kinase (RK) promoter, a codon optimized RPGR^{ORF15} sequence (coRPGR), a bovine growth hormone polyadenylation signal (bGH) and a 3′ ITR.

FIG. 14 is a cross-sectional view of an illustration of the human eye.

FIG. 15 is a cross-sectional view of a portion of the human eye of FIG. 14 taken along the line 2-2.

FIG. 16 is a cross-sectional view of a portion of the human eye of FIG. 14 taken along the line 3.3, illustrating the suprachoroidal space without presence of a fluid.

FIG. 17 is a cross-sectional view of a portion of the human eye of FIG. 14 taken along the line 3-3, illustrating the suprachoroidal space with the presence of a fluid.

FIG. 18A is a schematic diagram depicting a device comprising a microneedle administering a gene therapy composition to the suprachoroidal space.

FIG. 18B is schematic diagram depicting a microneedle crossing the sclera and entering the suprachoroidal space to deliver a gene therapy composition.

FIG. 19 is a photograph of an illustrative microcatheter tip of the disclosure. In some embodiments, a microcatheter such as those shown at devicepharm.net/iscience/US/itrack.htm may be used.

FIG. 20 is a schematic diagram of an illustrative microcannula of the disclosure.

FIG. 21 is a schematic diagram of the escalation scheme used in the AAV8-RPGR dose escalation study. DLT=dose-limiting toxicity; MTD=maximum tolerated dose.

FIG. 22A-22B are schematics depicting sub-retinal injection of an AAV8-RPGR. (FIG. 22A) A standard vitrectomy through the BIOM® operating system to remove the vitreous gel is followed by (FIG. 22B) retinal detachment by injection of BSS if necessary, and injection of 0.1 mL vector suspension through a 41-gauge cannula into the sub-retinal space.

FIG. 23 is a schematic diagram of alternative splicing of the RPGR gene.

FIG. 24A-24C are a series of schematic diagrams showing alternative splicing of the RPGR gene to produce ubiquitous RPGR mRNA.

FIG. 25A-25C are a series of schematic diagrams showing alternative splicing of the RPGR gene to produce photoreceptor specific RPGR mRNA-RPGR^{ORF15}.

FIG. 26A-26D are a series of schematic diagrams showing alternative splicing of the RPGR gene to produce potentially toxic truncated RPGR mRNA.

FIG. 27A-27C are a series of schematic diagrams showing codon optimization and alternative splicing of the RPGR gene to produce a correct full-length RPGR^{ORF15} mRNA from an AAV8 vector.

FIG. 28 is a schematic diagram of a RPGR^{ORF15} codon optimization scheme (SEQ ID NOs: 16 and 17).

FIG. 29A is a Western blot of whole protein lysates from transfected HEK293T cells. Untransfected cells were used as negative control (nc), which only show a positive band at 47 kDa indicating the loading control GAPDH. (FIG. 29B and FIG. 29C) Codon-optimized and wild-type plasmid transfected cells were loaded in an alternating fashion, and signal intensity of bands at 220 kDa (indicating RPGR) were quantified. (FIG. 29B) Boxplot (median, box delineates lower and upper quartile, whiskers minimum and maximum) of intensities in arbitrary units (AU) after normalization to the loading control (GAPDH). (FIG. 29C) Bar graph (mean±SD) after normalization to wild-type levels for a fold change presentation. After confirming the normal distribution of the dataset (n=4), significance was tested by one-tailed t test for paired samples of unequal variance. *p<0.005. See, Fischer et al. Mol Ther. 2017; 25(8):1854-1865.

FIG. 30A-30C are a series of schematic diagrams showing a functional ORF15 region produced after translation of the RPGR gene.

FIG. 31A is a schematic diagram of RPGR glutamylation with TTL5. FIG. 31B is a schematic diagram of glutamylation moving RPGR via tubulin in a photoreceptor cilium to the outer segment of a photoreceptor.

FIG. 32A is a schematic diagram of the effect of RPGR ORF15 deletion on glutamylation of the protein. FIG. 32B is a schematic diagram of a defective RPGR^{ORF15} with reduced glutamylation due to deletion.

FIG. 33A is a Western blot showing that RPGR^{ORF15} expression (black arrow) was detected in HEK293T cells transfected with either codon-optimized RPGR^{ORF15} (coRPGR^{ORF15}; co) or wtRPGR^{ORF15} (wt) containing plasmids compared with untransfected samples (UNT). A truncated 80 kDa protein (white arrowhead) was detected with an N terminus-directed RPGR antibody in cells transfected with the WT plasmid compared with cells transfected with the codon-optimized plasmid. FIG. 33A shows correct splicing in the cells transfected with the codon-optimized plasmid (full length RPGR protein with no splice variants in codon optimized RPGR construct (white arrowhead)). FIG. 33B is a Western blot showing that glutamylated RPGR^{ORF15} was detected with the GT335 antibody in HEK293T cells transfected with the codon-optimized and the WT sequence of RPGR^{ORF15}. FIG. 33B shows correct glutamylation in the cells transfected with the codon-optimized plasmid (full length and fully glutamylated ORF15 seen with GT335 immuno-staining in codon optimized RPGR). The 80 kDa band in (FIG. 33A) was not glutamylated in (FIG. 33B) and may therefore represent a truncated RPGR variant with a C-terminal deletion. See, Fischer et al. Mol Ther. 2017; 25(8):1854-1865.

FIG. 34 is a series of images produced after gene therapy of the human eye with a codon-optimized RPGR^{ORF15}.

FIG. 35A-35D are a series of schematics, immunoblots, graphs and tables showing that RPGR glutamylation in vivo requires both the C-terminal basic domain and the Glu-Gly-rich region. (FIG. 35A) Diagrams of human RPGR^{ORF15} expression constructs packaged into AAV vectors. The Glu-Gly-rich region is marked in red and the C-terminal basic domain in magenta. The position of glutamylation consensus motifs is shown in the schematic for the full-length (FL) construct. (FIG. 35B) Immunoblots of retinal extracts from Rpgr^{-/-} mice injected with RPGR expression constructs. Lanes 1-5 match the construct numbers shown in FIG. 35A, and lane 6 is an uninjected control. Full-length RPGR and to a lesser extent RPGRΔ864-989 are glutamylated as indicated by detection with GT335 (Middle). Probing with an RPGR antibody shows expression levels for recombinant RPGR (Top). Reprobing blots with β-actin provides a loading control (Bottom). (FIG. 35C) Quantification of glutamylation levels by densitometry after normalizing for RPGR levels, with sample 4 level set arbitrarily at 1. These results are summarized in FIG. 35D. ND, not determined. Similar results were obtained from two independent experiments. See, Sun et al. PNAS, 2016, 113 (21) E2925-E2934.

FIG. 36 is a codon frequency table for Homo sapiens used for codon optimisation of RPGR^{ORF15}. Each codon is indicated by the 3 nucleotide sequence (eg, TTT), followed by its frequency per 1000 (eg, 16.9) and the total number (eg, 336,562). The human codon usage table had been calculated from a set of 19,250 human genes from the Ensembl database (Release 57) with UniProtKB/SwissProt ID and is available in the public domain: genomes.urv.cat/CAIcal/CU_human_nature.html.

FIG. 37A-37B is a schematic diagram providing an overview of Sequencing Primer Alignment Along (FIG. 37A) wtRPGR^{ORF15} and (FIG. 37B) coRPGRORF15 Coding Sequences. Additional primers were designed and applied within the ORF15 region of the wtRPGR^{ORF15} cds in order to achieve full coverage of the sequence. This is due to difficulties in primer annealing and due to frequent premature terminations of sequencing reactions because of poly-G runs in the ORF15 region of wtRPGR^{ORF15}.

FIG. 38 is a schematic diagram providing an example of Highly Repetitive and Purine (Adenine/Guanine) Rich Sequence within ORF15 of wtRPGR^{ORF15} (SEQ ID NO:18).

FIG. 39A-39B is a graph and a schematic diagram showing that Codon Optimisation of RPGR^{ORF15} Leads to Significant Changes in the Primary Coding Sequence. FIG. 39A) The GC frequency along the full cds of RPGR^{ORF15} with wtRPGR^{ORF15} indicated on the top (black) and coRPGR^{ORF15} at the bottom (red) with grey breaks indicating the changes from the wild type sequence. FIG. 39B) Full sequence display with coRPGR^{ORF15} (SEQ ID NO:3) on top indicating the silent substitutions in red. The wtRPGR^{ORF15} (SEQ ID NO:10) sequence is displayed as reference below.

FIG. 40A-40C is a series of graphs depicting the results of codon optimisation efficacy experiments. FIG. 40A) coRPGR^{ORF15} Minipreparations containing plasmid DNA: concentration of were significantly higher (n=24, unpaired, 2-tailed t-test: p=0.0004). FIG. 40B) coRPGR^{ORF15} minipreparations: plasmid DNA concentration of 260/280 ratio remained unchanged. FIG. 40C) coRPGR^{ORF15} minipreparations: plasmid DNA concentration of total plasmid in maxipreparations confirmed increased cloning efficacy of coRPGRORF15. Note: no error bars as n=1.

FIG. 41 is a photograph showing RPGR^{ORF15} Transgene Expression in HEK293T Cells. Cells were transfected with wtRPGR^{ORF15} (wt) and coRPGR^{ORF15} (co) plasmid constructs or treated with media only (negative control). Confocal microscopy after immunocytochemistry with anti-RPGR and Hoechst 33342 demonstrate high levels of RPGR^{ORF15} expression in transfected cells.

FIG. 42A-42D is a series of photographs, a series of Western Blots and a pair of graphs providing the results of a Western blot analysis of RPGR^{ORF15} expression. (FIG. 42A) HEK293T cells were transfected with either wtRPGR^{ORF15} (wt) or coRPGR^{ORF15} (co) plasmid constructs. Control plasmid (GFP) was used to control for transfection (top right) and DMEM was used as negative control (nc). Intensity of Western blots bands. (FIG. 42B) Intensity of Western blots bands were quantified. (FIG. 42C) Box plot (median, box delineates lower and upper quartile, whiskers minimum and maximum), of intensities in arbitrary units [AU] after normalisation for loading control (GABDH). (FIG. 42D) Bar graph (mean±standard deviation) after normalisation to wild type levels for a fold change presentation.

FIG. 43A-43C is a photograph and series of graphs providing the results of a flow cytometric analysis of RPGR^{ORF15} expression. (FIG. 43A) HEK293T cells were transfected with either wtRPGR^{ORF15} (wt), coRPGR^{ORF15} (co) plasmid constructs. Control plasmid (GFP) was used to control for transfection (top right) and DMEM was used as negative control (not shown). Scale bar=20 μM. (FIG. 43B) Naïve cells were used to set appropriate sensitivity and specificity thresholds (left graphs). Using these thresholds, test samples transfected with wtRPGR^{ORF15} or coRPGR^{ORF15} were quantified. (FIG. 43C) Box plot (median, box delineates lower and upper quartile, whiskers minimum and maximum) of median fluorescence intensities in arbitrary units [AU] (n=9).

FIG. 44 is a graph showing overall macula sensitivity at month 1 of subjects responsive to treatment with a composition of the disclosure. Sensitivity was determined using the microperimetry methods of the disclosure.

FIG. 45 is a graph showing sensitivity at month 1 of 16 central points within the macula of subjects responsive to treatment with a composition of the disclosure. Sensitivity was determined using the microperimetry methods of the disclosure.

FIG. 46 is a graph showing the number of patients with greater than or equal to 7 decibels of improvement at 5 loci at month 1. The analysis was based the difference in mean sensitivities between baseline and a one-month follow-up following treatment. Sensitivity was determined using the microperimetry methods of the disclosure.

FIG. 47 is a graph showing the number of patients with greater than or equal to 7 decibels of improvement at 5 loci of 16 central loci at month 1. The analysis was based the difference in mean sensitivities between baseline and a one-month follow-up following treatment. Sensitivity was determined using the microperimetry methods of the disclosure.

FIG. 48 is a graph showing sensitivity at month 3 of subjects responsive to treatment with a composition of the disclosure. Sensitivity was determined using the microperimetry methods of the disclosure.

FIG. 49 is a graph showing sensitivity within 16 central loci of the macula at month 3 of subjects responsive to treatment with a composition of the disclosure. Sensitivity was determined using the microperimetry methods of the disclosure.

FIG. 50 is a graph showing the number of patients with greater than or equal to 7 decibels of improvement at 5 loci at month 3. The analysis was based the difference in mean sensitivities between baseline and a three-month follow-up following treatment. Sensitivity was determined using the microperimetry methods of the disclosure.

FIG. 51 is a graph showing the number of patients with greater than or equal to 7 decibels of improvement at 5 loci of 16 central loci at month 3. The analysis was based the difference in mean sensitivities between baseline and a three-month follow-up following treatment. Sensitivity was determined using the microperimetry methods of the disclosure.

FIG. 52 is a table providing a descriptive summary of subjects evaluated by OCT as part of the Xirius clinical trial.

FIG. 53 is a pair of photographs providing a low magnification (left) and high magnification (right) image of a cross section of the retina of subject 1 (as indicated in FIG. 52).

FIG. 54 is a pair of photographs providing a low magnification (left) and high magnification (right) image of a cross section of the retina of subject 2 (as indicated in FIG. 52). Yellow arrow pointing to double line within the retinal corresponding to the inner and outer segments, the appearance or increased thickness of which is a sign of therapeutic efficacy.

FIG. 55 is a pair of photographs providing a low magnification (left) and high magnification (right) image of a cross section of the retina of subject 3 (as indicated in FIG. 52).

FIG. 56 is a pair of photographs providing a low magnification (left) and high magnification (right) image of a cross section of the retina of subject 4 (as indicated in FIG. 52). Yellow arrow pointing to double line within the retinal corresponding to the inner and outer segments, the appearance or increased thickness of which is a sign of therapeutic efficacy.

FIG. 57 is a pair of photographs providing a low magnification (left) and high magnification (right) image of a cross section of the retina of subject 5 (as indicated in FIG. 52).

FIG. 58 is a pair of photographs providing a low magnification (left) and high magnification (right) image of a cross section of the retina of subject 6 (as indicated in FIG. 52). Yellow arrow pointing to double line within the retinal corresponding to the inner and outer segments, the appearance or increased thickness of which is a sign of therapeutic efficacy.

FIG. 59 is a pair of photographs providing a low magnification (left) and high magnification (right) image of a cross section of the retina of subject 7 (as indicated in FIG. 52). Yellow arrow pointing to double line within the retinal corresponding to the inner and outer segments, the appearance or increased thickness of which is a sign of therapeutic efficacy.

FIG. 60 is a pair of photographs providing a low magnification (left) and high magnification (right) image of a cross section of the retina of subject 8 (as indicated in FIG. 52). Yellow arrow pointing to double line within the retinal corresponding to the inner and outer segments, the appearance or increased thickness of which is a sign of therapeutic efficacy.

FIG. 61 is a pair of photographs providing a low magnification (left) and high magnification (right) image of a cross section of the retina of subject 9 (as indicated in FIG. 52). Yellow arrow pointing to double line within the retinal corresponding to the inner and outer segments, the appearance or increased thickness of which is a sign of therapeutic efficacy.

FIG. 62 is a pair of photographs providing a low magnification (left) and high magnification (right) image of a cross section of the retina of subject 10 (as indicated in FIG. 52). Yellow arrow pointing to double line within the retinal corresponding to the inner and outer segments, the appearance or increased thickness of which is a sign of therapeutic efficacy.

FIG. 63 is a pair of photographs providing a low magnification (left) and high magnification (right) image of a cross section of the retina of subject 11 (as indicated in FIG. 52).

FIG. 64 is a pair of photographs providing a low magnification (left) and high magnification (right) image of a cross section of the retina of subject 12 (as indicated in FIG. 52).

FIG. 65 is a pair of photographs providing a low magnification (left) and high magnification (right) image of a cross section of the retina of subject 13 (as indicated in FIG. 52). Yellow arrow pointing to double line within the retinal corresponding to the inner and outer segments, the appearance or increased thickness of which is a sign of therapeutic efficacy.

FIG. 66 is a pair of photographs providing a low magnification (left) and high magnification (right) image of a cross section of the retina of subject 14 (as indicated in FIG. 52). Yellow arrow pointing to double line within the retinal corresponding to the inner and outer segments, the appearance or increased thickness of which is a sign of therapeutic efficacy.

FIG. 67 is a pair of photographs providing a low magnification (left) and high magnification (right) image of a cross section of the retina of subject 15 (as indicated in FIG. 52).

FIG. 68 is a series of photographs showing the various features identified in each of the subjects evaluated by OCT.

### DETAILED DESCRIPTION

The disclosure provides a composition comprising a plurality of recombinant adeno associated virus of serotype 8 (rAAV8) particles, wherein each rAAV8 of the plurality of rAAV8 particles is non-replicating, and wherein each rAAV8 of the plurality of rAAV8 particles comprises a polynucleotide comprising, from 5′ to 3′: (a) a sequence encoding a 5′ inverted terminal repeat (ITR); (b) a sequence encoding a G protein-coupled receptor kinase 1 (GRK1) promoter; (c) a sequence encoding a retinitis pigmentosa GTPase regulator ORF15 isoform (RPGR^{ORF15}); (d) a sequence encoding a polyadenylation (polyA) signal; (e) a sequence encoding a 3′ ITR; and wherein the composition comprises between 5×10¹⁰ vector genomes (vg) per milliliter (mL) and 2×10¹³ vg/mL, inclusive of the endpoints.

In some embodiments of the compositions of the disclosure, the composition comprises between 0.5×10¹¹ vg/mL and 1×10¹² vg/mL, inclusive of the endpoints. In some embodiments, the composition comprises 0.5×10¹¹ vg/mL. In some embodiments, the composition comprises 5×10⁹ vg/mL. In some embodiments, the composition comprises 1×10¹⁰ vg/mL. In some embodiments, the composition comprises 5×10¹⁰ vg/mL. In some embodiments, the composition comprises 1×10¹¹ vg/mL. In some embodiments, the composition comprises 2.5×10¹¹ vg/mL. In some embodiments, the composition comprises 5×10¹¹ vg/mL. In some embodiments, the composition comprises 5×10¹² vg/mL. In some embodiments, the composition comprises 1×10¹³ vg/mL. In some embodiments, the composition comprises 2×10¹³ vg/mL.

In some embodiments, the disclosure provides a composition comprising a plurality of recombinant adeno associated virus of serotype 8 (rAAV8) particles, wherein each rAAV8 of the plurality of rAAV8 particles is non-replicating, and wherein each rAAV8 of the plurality of rAAV8 particles comprises a polynucleotide comprising, from 5′ to 3′: (a) a sequence encoding a 5′ inverted terminal repeat (ITR); (b) a sequence encoding a G protein-coupled receptor kinase 1 (GRK1) promoter; (c) a sequence encoding a retinitis pigmentosa GTPase regulator ORF15 isoform (RPGR^{ORF15}); (d) a sequence encoding a polyadenylation (polyA) signal; and (e) a sequence encoding a 3′ ITR; and wherein the composition comprises between 1.0×10¹⁰ vector genomes (vg) per milliliter (mL) and 1×10¹³ vg/mL, inclusive of the endpoints.

In some embodiments of the compositions of the disclosure, the composition comprises between 5×10⁹ genome particles (gp) and 5×10¹¹ gp, inclusive of the endpoints. In some embodiments, the composition comprises 5×10⁹ gp. In some embodiments, the composition comprises 1×10¹⁰ gp. In some embodiments, the composition comprises 5×10¹⁰ gp. In some embodiments, the composition comprises 1×10¹¹ gp. In some embodiments, the composition comprises 2.5×10¹¹ gp. In some embodiments, the composition comprises 5×10¹¹ gp.

In some embodiments, the composition further comprises a pharmaceutically acceptable carrier. In some embodiments, the pharmaceutically acceptable carrier comprises Tris, MgCl₂, and NaCl, optionally 20 mM Tris, 1 mM MgCl₂, and 200 mM NaCl at pH 8.0. In some embodiments, the pharmaceutically acceptable carrier further comprises poloxamer 188 at 0.001%.

The disclosure provides a device, comprising a composition of the disclosure. In some embodiments, the device comprises a microdelivery device. In some embodiments, the microdelivery device comprises a microneedle and the microneedle is suitable for subretinal injection. In some embodiments, the microdelivery device comprises a microcatheter and the microcatheter is suitable for suprachoroidal injection.

The disclosure provides a method of treating Retinitis Pigmentosa in a subject in need thereof, comprising administering to the subject a therapeutically effective amount of a composition of the disclosure. In some embodiments, administering to the subject the therapeutically effective amount of the composition administered to the subject improves a sign or a symptom of Retinitis Pigmentosa. In some embodiments, the sign of Retinitis Pigmentosa comprises degeneration of the ellipsoid zone (EZ) and/or a reduction of retinal sensitivity when compared to a healthy or control EZ or retinal sensitivity. In some embodiments, the sign of Retinitis Pigmentosa comprises a reduction of visual acuity, retinal thickness and/or outer nuclear layer (ONL) thickness when compared to a healthy or control visual acuity, retinal thickness and/or ONL thickness. In some embodiments, retinal thickness encompasses or comprises ONL thickness. In some embodiments of the methods of the disclosure, treating Retinitis Pigmentosa restores the EZ, retinal sensitivity, visual acuity, retinal thickness and/or ONL thickness. In some embodiments of the methods of the disclosure, treating Retinitis Pigmentosa decreases a severity of a sign or symptom of Retinitis Pigmentosa, including, but not limited to, degeneration of the EZ or reduction of retinal sensitivity, visual acuity, retinal thickness and/or outer nuclear layer (ONL) thickness. In some embodiments of the methods of the disclosure, treating Retinitis Pigmentosa delays the onset of a sign or symptom of Retinitis Pigmentosa, including, but not limited to, degeneration of the EZ or reduction of retinal sensitivity, visual acuity, retinal thickness and/or ONL thickness. In some embodiments of the methods of the disclosure, treating Retinitis Pigmentosa reduces a rate of progression or inhibits the progression of a sign or symptom of Retinitis Pigmentosa, including, but not limited to, degeneration of the EZ or reduction of retinal sensitivity, visual acuity, retinal thickness and/or ONL thickness. Healthy or control EZ, retinal sensitivity, visual acuity, retinal thickness and/or ONL thickness may include experimentally determined population-based thresholds, averages, means or standards of, for example gender and age matched individuals to the subject. Healthy or control EZ, retinal sensitivity, visual acuity, retinal thickness and/or ONL thickness may include those of an unaffected eye of the subject. A control EZ, retinal sensitivity, visual acuity, retinal thickness and/or ONL thickness may include a time point in the subject prior to administration of a composition of the disclosure that forms a baseline for comparison throughout treatment to determine effectiveness of the composition to improve a sign or symptom of Retinitis Pigmentosa.

### AAV Compositions

Compositions of the disclosure may comprise a polynucleotide comprising Retinitis Pigmentosa GTPase Regulator ORF15 (RPGR^{ORF15}) suitable for systemic or local administration to a mammal, and preferable, to a human. Illustrative RPGR^{ORF15} polynucleotides of the disclosure comprise a sequence encoding RPGR^{ORF15} or a portion thereof. Preferably, RPGR^{ORF15} polynucleotides of the disclosure comprise a sequence encoding human RPGR^{ORF15} or a portion thereof. Illustrative RPGR^{ORF15} polynucleotides of the disclosure may further comprise one or more sequence(s) encoding regulatory elements to enable or to enhance expression of the gene or a portion thereof. Illustrative regulatory elements include, but are not limited to, promoters, introns, enhancer elements, response elements (including post-transcriptional response elements or post-transcriptional regulatory elements), polyadenosine (polyA) sequences, and a gene fragment to facilitate efficient termination of transcription (including a β-globin gene fragment and a rabbit β-globin gene fragment).

In some embodiments of the compositions of the disclosure, the RPGR^{ORF15} polynucleotide comprises a human gene or a portion thereof corresponding to a human Retinitis Pigmentosa GTPase Regulator (RPGR) protein or a portion thereof. Human RPGR comprises multiple spliced isoforms. Isoform ORF15 RPGR (RPGR^{ORF15}) localizes to the photoreceptors. In some embodiments, the RPGR protein is RPGR^{ORF15}. In some embodiments, the RPGR^{ORF15} polynucleotide comprises a codon-optimized sequence. In some embodiments, the sequence is codon-optimized for expression in mammals. In some embodiments, the sequence is codon-optimized for expression in humans.

In some embodiments of the compositions of the disclosure, the RPGR^{ORF15} polynucleotide consists of a purified recombinant serotype 2 (rAAV) encoding the cDNA of RPGR^{ORF15}. In some embodiments, each 20 nm AAV virion contains a single stranded DNA insert sequence comprising: a 119 bp AAV2 5′ inverted terminal repeat (ITR), a 199 bp G protein-coupled rhodopsin kinase 1 (GRK1) promoter, a 3459 bp human RPGR^{ORF15} cDNA, a 270 bp Bovine growth hormone polyadenylation sequence (BGH-polyA), and a 130 bp AAV2 3′ ITR, as well a short cloning sequences flanking the elements.

In some embodiments, the RPGR^{ORF15} polynucleotide comprises a sequence encoding RPGR^{OR15}. In some embodiments, the sequence encoding the RPGR^{ORF15} is a human RPGR^{ORF15} sequence. In some embodiments, the sequence encoding RPGR^{ORF15} comprises a nucleotide sequence encoding an amino acid sequence that has at least 80% identity, at least 90% identity, at least 95% identity, at least 97% identity, at least 99% identity or is identical to the amino acid sequence of:

| (SEQ ID NO: 2) | |
|---|---|
| 1 | MREPEELMPD SGAVFTFGKS KFAENNPGKF WFKNDVPVHL SCGDEHSAVV TGNNKLYMFG |
| 61 | SNNWGQLGLG SKSAISKPTC VKALKPEKVK LAACGRNHTL VSTEGGNVYA TGGNNEGQLG |
| 121 | LGDTEERNTF HVISFFTSEH KIKQLSAGSN TSAALTEDGR LFMWGDNSEG QIGLKNVSNV |
| 181 | CVPQQVTIGK PVSWISCGYY HSAFVTTDGE LYVFGEPENG KLGLPNQLLG NHRTPQLVSE |
| 241 | IPEKVIQVAC GGEHTVVLTE NAVYTFGLGQ FGQLGLGTFL FETSEPKVIE NIRDQTISYI |
| 301 | SCGENHTALI TDIGLMYTFG DGRHGKLGLG LENFTNHFIP TLCSNFLRFI VKLVACGGCH |
| 361 | MVVFAAPHRG VAKEIEFDEI NDTCLSVATF LPYSSLTSGN VLQRTLSARM RRRERERSPD |
| 421 | SFSMRRTLPP IEGTLGLSAC FLPNSVFPRC SERNLQESVL SEQDLMQPEE PDYLLDEMTK |
| 481 | EAEIDNSSTV ESLGETTDIL NMTHIMSLNS NEKSLKLSPV QKQKKQQTIG ELTQDTALTE |
| 541 | NDDSDEYEEM SEMKEGKACK QHVSQGIFMT QPATTIEAFS DEEVEIPEEK EGAEDSKGNG |
| 601 | IEEQEVEANE ENVKVHGGRK EKTEILSDDL TDKAEVSEGK AKSVGEAEDG PEGRGDGTCE |
| 661 | EGSSGAEHWQ DEEREKGEKD KGRGEMERPG EGEKELAEKE EWKKRDGEEQ EQKEREQGHQ |
| 721 | KERNQEMEEG GEEEHGEGEE EEGDREEEEE KEGEGKEEGE GEEVEGEREK EEGERKKEER |
| 781 | AGKEEKGEEE GDQGEGEEEE TEGRGEEKEE GGEVEGGEVE EGKGEREEEE EEGEGEEEEG |
| 841 | EGEEEEGEGE EEEGEGKGEE EGEEGEGEEE GEEGEGEGEE EEGEGEGEEE GEGEGEEEEG |
| 901 | EGEGEEEGEG EGEEEEGEGK GEEEGEEGEG EGEEEEGEGE GEDGEGEGEE EEGEWEGEEE |
| 961 | EGEGEGEEEG EGEGEEGEGE GEEEEGEGEG EEEEGEEEGE EEGEGEEEGE GEGEEEEEGE |
| 1021 | VEGEVEGEEG EGEGEEEEGE EEGEEREKEG EGEENRRNRE EEEEEEGKYQ ETGEEENERQ |
| 1081 | DGEEYKKVSK IKGSVKYGKH KTYQKKSVTN TQGNGKEQRS KMPVQSKRLL KNGPSGSKKF |
| 1141 | WNNVLPHYLE LK. |

In some embodiments, the sequence encoding RPGR^{ORF15} comprises a wild type nucleotide sequence. In some embodiments, the sequence encoding RPGR^{ORF15} comprises a nucleotide sequence that has at least 70%, at least 75%, at least 80%, at least 85%, at least 90%, at least 95%, at least 97%, at least 99% or any percentage in between of identity to the nucleotide sequence of:

| (SEQ ID NO: 10) | |
|---|---|
| 1 | atgagggagc cggaagagct gatgcccgat tcgggtgctg tgtttacatt tgggaaaagt |
| 61 | aaatttgctg aaaataatcc cggtaaattc tggtttaaaa atgatgtccc tgtacatctt |
| 121 | tcatgtggag atgaacattc tgctgttgtt accggaaata ataaacttta catgtttggc |
| 181 | agtaacaact ggggtcagtt aggattagga tcaaagtcag ccatcagcaa gccaacatgt |
| 241 | gtcaaagctc taaaacctga aaaagtgaaa ttagctgcct gtggaaggaa ccacaccctg |
| 301 | gtgtcaacag aaggaggcaa tgtatatgca actggtggaa ataatgaagg acagttgggg |
| 361 | cttggtgaca ccgaagaaag aaacactttt catgtaatta gcttttttac atccgagcat |
| 421 | aagattaagc agctgtctgc tggatctaat acttcagctg ccctaactga ggatggaaga |
| 481 | ctttttatgt ggggtgacaa ttccgaaggg caaattggtt taaaaaatgt aagtaatgtc |
| 541 | tgtgtccctc agcaagtgac cattgggaaa cctgtctcct ggatctcttg tggatattac |
| 601 | cattcagctt ttgtaacaac agatggtgag ctatatgtgt ttggagaacc tgagaatggg |
| 661 | aagttaggtc ttcccaatca gctcctgggc aatcacagaa caccccagct ggtgtctgaa |
| 721 | attccggaga aggtgatcca agtagcctgt ggtggagagc atactgtggt tctcacggag |
| 781 | aatgctgtgt atacctttgg gctgggacaa tttggtcagc tgggtcttgg cacttttctt |
| 841 | tttgaaactt cagaacccaa agtcattgag aatattaggg atcaaacaat aagttatatt |
| 901 | tcttgtggag aaaatcacac agctttgata acagatatcg gccttatgta tacttttgga |
| 961 | gatggtcgcc acggaaaatt aggacttgga ctggagaatt ttaccaatca cttcattcct |
| 1021 | actttgtgct ctaatttttt gaggtttata gttaaattgg ttgcttgtgg tggatgtcac |
| 1081 | atggtagttt ttgctgctcc tcatcgtggt gtggcaaaag aaattgaatt cgatgaaata |
| 1141 | aatgatactt gcttatctgt ggcgactttt ctgccgtata gcagtttaac ctcaggaaat |
| 1201 | gtactgcaga ggactctatc agcacgtatg cggcgaagag agagggagag gtctccagat |
| 1261 | tctttttcaa tgaggagaac actacctcca atagaaggga ctcttggcct ttctgcttgt |
| 1321 | tttctcccca attcagtctt tccacgatgt tctgagagaa acctccaaga gagtgtctta |
| 1381 | tctgaacagg acctcatgca gccagaggaa ccagattatt tgctagatga aatgaccaaa |
| 1441 | gaagcagaga tagataattc ttcaactgta gaaagccttg gagaaactac tgatatctta |
| 1501 | aacatgacac acatcatgag cctgaattcc aatgaaaagt cattaaaatt atcaccagtt |
| 1561 | cagaaacaaa agaaacaaca aacaattggg gaactgacgc aggatacagc tcttactgaa |
| 1621 | aacgatgata gtgatgaata tgaagaaatg tcagaaatga aagaagggaa agcatgtaaa |
| 1681 | caacatgtgt cacaagggat tttcatgacg cagccagcta cgactatcga agcattttca |
| 1741 | gatgaggaag tagagatccc agaggagaag gaaggagcag aggattcaaa aggaaatgga |
| 1801 | atagaggagc aagaggtaga agcaaatgag gaaaatgtga aggtgcatgg aggaagaaag |
| 1861 | gagaaaacag agatcctatc agatgacctt acagacaaag cagaggtgag tgaaggcaag |
| 1921 | gcaaaatcag tgggagaagc agaggatggg cctgaaggta gaggggatgg aacctgtgag |
| 1981 | gaaggtagtt caggagcaga acactggcaa gatgaggaga gggagaaggg ggagaaagac |
| 2041 | aagggtagag gagaaatgga gaggccagga gagggagaga aggaactagc agagaaggaa |
| 2101 | gaatggaaga agagggatgg ggaagagcag gagcaaaagg agagggagca gggccatcag |
| 2161 | aaggaaagaa accaagagat ggaggaggga ggggaggagg agcatggaga aggagaagaa |
| 2221 | gaggagggag acagagaaga ggaagaagag aaggagggag aagggaaaga ggaaggagaa |
| 2281 | ggggaagaag tggagggaga acgtgaaaag gaggaaggag agaggaaaaa ggaggaaaga |
| 2341 | gcggggaagg aggagaaagg agaggaagaa ggagaccaag gagaggggga agaggaggaa |
| 2401 | acagagggga gaggggagga aaaagaggag ggaggggaag tagagggagg ggaagtagag |
| 2461 | gaggggaaag gagagaggga agaggaagag gaggagggtg agggggaaga ggaggaaggg |
| 2521 | gagggggaag aggaggaagg ggagggggaa gaggaggaag gagaagggaa aggggaggaa |
| 2581 | gaaggggaag aaggagaagg ggaggaagaa ggggaggaag gagaagggga gggggaagag |
| 2641 | gaggaaggag aaggggaggg agaagaggaa ggagaagggg agggagaaga ggaggaagga |
| 2701 | gaaggggagg gagaagagga aggagaaggg gagggagaag aggaggaagg agaagggaaa |
| 2761 | ggggaggagg aaggagagga aggagaaggg gagggggaag aggaggaagg agaaggggaa |
| 2821 | ggggaggatg gagaagggga gggggaagag gaggaaggag aatgggaggg ggaagaggag |
| 2881 | gaaggagaag gggaggggga agaggaagga gaaggggaag gggaggaagg agaaggggag |
| 2941 | ggggaagagg aggaaggaga aggggagggg gaagaggagg aaggggaaga agaaggggag |
| 3001 | gaagaaggag agggagagga agaaggggag ggagaagggg aggaagaaga ggaaggggaa |
| 3061 | gtggaagggg aggtggaagg ggaggaagga gagggggaag gagaggaaga ggaaggagag |
| 3121 | gaggaaggag aagaaaggga aaaggagggg gaaggagaag aaaacaggag gaacagagaa |
| 3181 | gaggaggagg aagaagaggg gaagtatcag gagacaggcg aagaagagaa tgaaaggcag |
| 3241 | gatggagagg agtacaaaaa agtgagcaaa ataaaaggat ctgtgaaata tggcaaacat |
| 3301 | aaaacatatc aaaaaaagtc agttactaac acacagggaa atgggaaaga gcagaggtcc |
| 3361 | aaaatgccag tccagtcaaa acgactttta aaaaacgggc catcaggttc caaaaagttc |
| 3421 | tggaataatg tattaccaca ttacttggaa ttgaagtaa. |

In some embodiments, the sequence encoding RPGR^{ORF15} comprises a codon optimized nucleotide sequence. RPGR^{ORF15} contains a highly repetitive purine-rich region at the 3′-end and a splice site immediately upstream, which can create significant challenges in cloning an AAV.RPGR vector. In some embodiments, codon optimization can be used to disable the endogenous splice site and stabilize the purine-rich sequence in the RPGR^{ORF15} transcript without altering the amino acid sequence of the RPGR^{ORF15} protein. In some embodiments, post-translation modifications such as glutamylation of RPGR protein are preserved following codon-optimization. In some embodiments, the RPGR^{ORF15} nucleotide sequence is codon optimized for expression in a mammal. In some embodiments, the RPGR^{ORF15} nucleotide sequence is codon optimized for expression in a human.

In some embodiments, the codon optimized 3459 bp human RPGR^{ORF15} cDNA comprises a nucleotide sequence that has at least 70% identity, at least 75% identity, at least 80% identity, at least 85% identity, at least 90% identity, at least 95% identity, at least 97% identity, at least 99% identity or any percentage in between of identity to the nucleotide sequence of:

| (SEQ ID NO: 3) | |
|---|---|
| 1 | atgagagagc cagaggagct gatgccagac agtggagcag tgtttacatt cggaaaatct |
| 61 | aagttcgctg aaaataaccc aggaaagttc tggtttaaaa acgacgtgcc cgtccacctg |
| 121 | tcttgtggcg atgagcatag tgccgtggtc actgggaaca ataagctgta catgttcggg |
| 181 | tccaacaact ggggacagct ggggctggga tccaaatctg ctatctctaa gccaacctgc |
| 241 | gtgaaggcac tgaaacccga gaaggtcaaa ctggccgctt gtggcagaaa ccacactctg |
| 301 | gtgagcaccg agggcgggaa tgtctatgcc accggaggca acaatgaggg acagctggga |
| 361 | ctgggggaca ctgaggaaag gaataccttt cacgtgatct ccttctttac atctgagcat |
| 421 | aagatcaagc agctgagcgc tggctccaac acatctgcag ccctgactga ggacgggcgc |
| 481 | ctgttcatgt ggggagataa ttcagagggc cagattgggc tgaaaaacgt gagcaatgtg |
| 541 | tgcgtccctc agcaggtgac catcggaaag ccagtcagtt ggatttcatg tggctactat |
| 601 | catagcgcct tcgtgaccac agatggcgag ctgtacgtct ttggggagcc cgaaaacgga |
| 661 | aaactgggcc tgcctaacca gctgctgggc aatcaccgga caccccagct ggtgtccgag |
| 721 | atccctgaaa aagtgatcca ggtcgcctgc gggggagagc atacagtggt cctgactgag |
| 781 | aatgctgtgt ataccttcgg actgggccag tttggccagc tggggctggg aaccttcctg |
| 841 | tttgagacat ccgaaccaaa agtgatcgag aacattcgcg accagactat cagctacatt |
| 901 | tcctgcggag agaatcacac cgcactgatc acagacattg gcctgatgta tacctttggc |
| 961 | gatggacgac acgggaagct gggactggga ctggagaact tcactaatca ttttatcccc |
| 1021 | accctgtgtt ctaacttcct gcggttcatc gtgaaactgg tcgcttgcgg cgggtgtcac |
| 1081 | atggtggtct tcgctgcacc tcataggggc gtggctaagg agatcgaatt tgacgagatt |
| 1141 | aacgatacat gcctgagcgt ggcaactttc ctgccataca gctccctgac ttctggcaat |
| 1201 | gtgctgcaga gaaccctgag tgcaaggatg cggagaaggg agagggaacg ctctcctgac |
| 1261 | agtttctcaa tgcgacgaac cctgccacct atcgagggaa cactgggact gagtgcctgc |
| 1321 | ttcctgccta actcagtgtt tccacgatgt agcgagcgga atctgcagga gtctgtcctg |
| 1381 | agtgagcagg atctgatgca gccagaggaa cccgactacc tgctggatga gatgaccaag |
| 1441 | gaggccgaaa tcgacaactc tagtacagtg gagtccctgg gcgagactac cgatatcctg |
| 1501 | aatatgacac acattatgtc actgaacagc aatgagaaga gtctgaaact gtcaccagtg |
| 1561 | cagaagcaga agaaacagca gactattggc gagctgactc aggacaccgc cctgacagag |
| 1621 | aacgacgata gcgatgagta tgaggaaatg tccgagatga aggaaggcaa agcttgtaag |
| 1681 | cagcatgtca gtcaggggat cttcatgaca cagccagcca caactattga ggctttttca |
| 1741 | gacgaggaag tggagatccc cgaggaaaaa gagggcgcag aagattccaa ggggaatgga |
| 1801 | attgaggaac aggaggtgga agccaacgag gaaaatgtga aagtccacgg aggcaggaag |
| 1861 | gagaaaacag aaatcctgtc tgacgatctg actgacaagg ccgaggtgtc cgaaggcaag |
| 1921 | gcaaaatctg tcggagaggc agaagacgga ccagagggac gaggggatgg aacctgcgag |
| 1981 | gaaggctcaa gcggggctga gcattggcag gacgaggaac gagagaaggg cgaaaaggat |
| 2041 | aaaggccgcg gggagatgga acgacctgga gagggcgaaa aagagctggc agagaaggag |
| 2101 | gaatggaaga aaagggacgg cgaggaacag gagcagaaag aaagggagca gggccaccag |
| 2161 | aaggagcgca accaggagat ggaagagggc ggcgaggaag agcatggcga gggagaagag |
| 2221 | gaagagggcg atagagaaga ggaagaggaa aaagaaggcg aagggaagga ggaaggagag |
| 2281 | ggcgaggaag tggaaggcga gagggaaaag gaggaaggag aacggaagaa agaggaaaga |
| 2341 | gccggcaaag aggaaaaggg cgaggaagag ggcgatcagg gcgaaggcga ggaggaagag |
| 2401 | accgagggcc gcggggaaga gaaagaggag ggaggagagg tggagggcgg agaggtcgaa |
| 2461 | gagggaaagg gcgagcgcga agaggaagag gaagagggcg agggcgagga agaagagggc |
| 2521 | gagggggaag aagaggaggg agagggcgaa gaggaagagg gggagggaaa gggcgaagag |
| 2581 | gaaggagagg aaggggaggg agaggaagag ggggaggagg gcgaggggga aggcgaggag |
| 2641 | gaagaaggag agggggaagg cgaagaggaa ggcgaggggg aaggagagga ggaagaaggg |
| 2701 | gaaggcgaag gcgaagagga gggagaagga gagggggagg aagaggaagg agaagggaag |
| 2761 | ggcgaggagg aaggcgaaga gggagagggg gaaggcgagg aagaggaagg cgagggcgaa |
| 2821 | ggagaggacg gcgagggcga gggagaagag gaggaagggg aatgggaagg cgaagaagag |
| 2881 | gaaggcgaag gcgaaggcga agaagagggc gaaggggagg gcgaggaggg cgaaggcgaa |
| 2941 | ggggaggaag aggaaggcga aggagaaggc gaggaagaag agggagagga ggaaggcgag |
| 3001 | gaggaaggag agggggagga ggagggagaa ggcgagggcg aagaagaaga agagggagaa |
| 3061 | gtggagggcg aagtcgaggg ggaggaggga gaaggggaag gggaggaaga agagggcgaa |
| 3121 | gaagaaggcg aggaaagaga aaaagaggga gaaggcgagg aaaaccggag aaatagggaa |
| 3181 | gaggaggaag aggaagaggg aaagtaccag gagacaggcg aagaggaaaa cgagcggcag |
| 3241 | gatggcgagg aatataagaa agtgagcaag atcaaaggat ccgtcaagta cggcaagcac |
| 3301 | aaaacctatc agaagaaaag cgtgaccaac acacagggga atggaaaaga gcagaggagt |
| 3361 | aagatgcctg tgcagtcaaa acggctgctg aagaatggcc catctggaag taaaaaattc |
| 3421 | tggaacaatg tgctgcccca ctatctggaa ctgaaataa. |

In some embodiments, the codon optimized 3459 bp human RPGR^{ORF15} cDNA comprises or consists of the nucleotide sequence of:

| (SEQ ID NO: 3) | |
|---|---|
| 1 | atgagagagc cagaggagct gatgccagac agtggagcag tgtttacatt cggaaaatct |
| 61 | aagttcgctg aaaataaccc aggaaagttc tggtttaaaa acgacgtgcc cgtccacctg |
| 121 | tcttgtggcg atgagcatag tgccgtggtc actgggaaca ataagctgta catgttcggg |
| 181 | tccaacaact ggggacagct ggggctggga tccaaatctg ctatctctaa gccaacctgc |
| 241 | gtgaaggcac tgaaacccga gaaggtcaaa ctggccgctt gtggcagaaa ccacactctg |
| 301 | gtgagcaccg agggcgggaa tgtctatgcc accggaggca acaatgaggg acagctggga |
| 361 | ctgggggaca ctgaggaaag gaataccttt cacgtgatct ccttctttac atctgagcat |
| 421 | aagatcaagc agctgagcgc tggctccaac acatctgcag ccctgactga ggacgggcgc |
| 481 | ctgttcatgt ggggagataa ttcagagggc cagattgggc tgaaaaacgt gagcaatgtg |
| 541 | tgcgtccctc agcaggtgac catcggaaag ccagtcagtt ggatttcatg tggctactat |
| 601 | catagcgcct tcgtgaccac agatggcgag ctgtacgtct ttggggagcc cgaaaacgga |
| 661 | aaactgggcc tgcctaacca gctgctgggc aatcaccgga caccccagct ggtgtccgag |
| 721 | atccctgaaa aagtgatcca ggtcgcctgc gggggagagc atacagtggt cctgactgag |
| 781 | aatgctgtgt ataccttcgg actgggccag tttggccagc tggggctggg aaccttcctg |
| 841 | tttgagacat ccgaaccaaa agtgatcgag aacattcgcg accagactat cagctacatt |
| 901 | tcctgcggag agaatcacac cgcactgatc acagacattg gcctgatgta tacctttggc |
| 961 | gatggacgac acgggaagct gggactggga ctggagaact tcactaatca ttttatcccc |
| 1021 | accctgtgtt ctaacttcct gcggttcatc gtgaaactgg tcgcttgcgg cgggtgtcac |
| 1081 | atggtggtct tcgctgcacc tcataggggc gtggctaagg agatcgaatt tgacgagatt |
| 1141 | aacgatacat gcctgagcgt ggcaactttc ctgccataca gctccctgac ttctggcaat |
| 1201 | gtgctgcaga gaaccctgag tgcaaggatg cggagaaggg agagggaacg ctctcctgac |
| 1261 | agtttctcaa tgcgacgaac cctgccacct atcgagggaa cactgggact gagtgcctgc |
| 1321 | ttcctgccta actcagtgtt tccacgatgt agcgagcgga atctgcagga gtctgtcctg |
| 1381 | agtgagcagg atctgatgca gccagaggaa cccgactacc tgctggatga gatgaccaag |
| 1441 | gaggccgaaa tcgacaactc tagtacagtg gagtccctgg gcgagactac cgatatcctg |
| 1501 | aatatgacac acattatgtc actgaacagc aatgagaaga gtctgaaact gtcaccagtg |
| 1561 | cagaagcaga agaaacagca gactattggc gagctgactc aggacaccgc cctgacagag |
| 1621 | aacgacgata gcgatgagta tgaggaaatg tccgagatga aggaaggcaa agcttgtaag |
| 1681 | cagcatgtca gtcaggggat cttcatgaca cagccagcca caactattga ggctttttca |
| 1741 | gacgaggaag tggagatccc cgaggaaaaa gagggcgcag aagattccaa ggggaatgga |
| 1801 | attgaggaac aggaggtgga agccaacgag gaaaatgtga aagtccacgg aggcaggaag |
| 1861 | gagaaaacag aaatcctgtc tgacgatctg actgacaagg ccgaggtgtc cgaaggcaag |
| 1921 | gcaaaatctg tcggagaggc agaagacgga ccagagggac gaggggatgg aacctgcgag |
| 1981 | gaaggctcaa gcggggctga gcattggcag gacgaggaac gagagaaggg cgaaaaggat |
| 2041 | aaaggccgcg gggagatgga acgacctgga gagggcgaaa aagagctggc agagaaggag |
| 2101 | gaatggaaga aaagggacgg cgaggaacag gagcagaaag aaagggagca gggccaccag |
| 2161 | aaggagcgca accaggagat ggaagagggc ggcgaggaag agcatggcga gggagaagag |
| 2221 | gaagagggcg atagagaaga ggaagaggaa aaagaaggcg aagggaagga ggaaggagag |
| 2281 | ggcgaggaag tggaaggcga gagggaaaag gaggaaggag aacggaagaa agaggaaaga |
| 2341 | gccggcaaag aggaaaaggg cgaggaagag ggcgatcagg gcgaaggcga ggaggaagag |
| 2401 | accgagggcc gcggggaaga gaaagaggag ggaggagagg tggagggcgg agaggtcgaa |
| 2461 | gagggaaagg gcgagcgcga agaggaagag gaagagggcg agggcgagga agaagagggc |
| 2521 | gagggggaag aagaggaggg agagggcgaa gaggaagagg gggagggaaa gggcgaagag |
| 2581 | gaaggagagg aaggggaggg agaggaagag ggggaggagg gcgaggggga aggcgaggag |
| 2641 | gaagaaggag agggggaagg cgaagaggaa ggcgaggggg aaggagagga ggaagaaggg |
| 2701 | gaaggcgaag gcgaagagga gggagaagga gagggggagg aagaggaagg agaagggaag |
| 2761 | ggcgaggagg aaggcgaaga gggagagggg gaaggcgagg aagaggaagg cgagggcgaa |
| 2821 | ggagaggacg gcgagggcga gggagaagag gaggaagggg aatgggaagg cgaagaagag |
| 2881 | gaaggcgaag gcgaaggcga agaagagggc gaaggggagg gcgaggaggg cgaaggcgaa |
| 2941 | ggggaggaag aggaaggcga aggagaaggc gaggaagaag agggagagga ggaaggcgag |
| 3001 | gaggaaggag agggggagga ggagggagaa ggcgagggcg aagaagaaga agagggagaa |
| 3061 | gtggagggcg aagtcgaggg ggaggaggga gaaggggaag gggaggaaga agagggcgaa |
| 3121 | gaagaaggcg aggaaagaga aaaagaggga gaaggcgagg aaaaccggag aaatagggaa |
| 3181 | gaggaggaag aggaagaggg aaagtaccag gagacaggcg aagaggaaaa cgagcggcag |
| 3241 | gatggcgagg aatataagaa agtgagcaag atcaaaggat ccgtcaagta cggcaagcac |
| 3301 | aaaacctatc agaagaaaag cgtgaccaac acacagggga atggaaaaga gcagaggagt |
| 3361 | aagatgcctg tgcagtcaaa acggctgctg aagaatggcc catctggaag taaaaaattc |
| 3421 | tggaacaatg tgctgcccca ctatctggaa ctgaaataa. |

In some embodiments of the compositions of the disclosure, the RPGR^{ORF15} polynucleotide comprises a promoter. In some embodiments, the promoter comprises a rhodopsin kinase promoter. In some embodiments, the rhodopsin kinase promoter is isolated or derived from the promoter of the G protein-coupled receptor kinase 1 (GRK1) gene. In some embodiments, the promoter is a GRK1 promoter. In some embodiments, the sequence encoding the GRK1 promoter comprises a sequence having at least 80% identity, at least 90% identity, at least 95% identity, at least 97% identity or at least 99% identity to:

| (SEQ ID NO: 1) | |
|---|---|
| 1 | gggccccaga agcctggtgg ttgtttgtcc ttctcagggg aaaagtgagg cggccccttg |
| 61 | gaggaagggg ccgggcagaa tgatctaatc ggattccaag cagctcaggg gattgtcttt |
| 121 | ttctagcacc ttcttgccac tcctaagcgt cctccgtgac cccggctggg atttagcctg |
| 181 | gtgctgtgtc agccccggg. |

In some embodiments, the GRK1 promoter comprises or consists of:

| (SEQ ID NO: 1) | |
|---|---|
| 1 | gggccccaga agcctggtgg ttgtttgtcc ttctcagggg aaaagtgagg cggccccttg |
| 61 | gaggaagggg ccgggcagaa tgatctaatc ggattccaag cagctcaggg gattgtcttt |
| 121 | ttctagcacc ttcttgccac tcctaagcgt cctccgtgac cccggctggg atttagcctg |
| 181 | gtgctgtgtc agccccggg. |

In some embodiments of the compositions of the disclosure, the RPGR^{ORF15} polynucleotide comprises a polyadenylation signal. In some embodiments, the sequence encoding the polyA signal comprises a polyA signal isolated or derived from a bovine growth hormone (BGH) polyA signal. In some embodiments, the BGH polyA signal comprises a nucleotide sequence that has at least 80% identity, at least 97% identity or 100% identity to the nucleotide sequence of:

(SEQ ID NO: 4)

| | |
|---|---|
| 1 | tcgctgatca gcctcgactg tgccttctag ttgccagcca |
| | tctgttgttt gcccctcccc |
| 61 | cgtgccttcc ttgaccctgg aaggtgccac tcccactgtc |
| | ctttcctaat aaaatgagga |
| 121 | aattgcatcg cattgtctga gtaggtgtca ttctattctg |
| | gggggtgggg tggggcagga |
| 181 | cagcaagggg gaggattggg aagacaatag caggcatgct |
| | ggggatgcgg tgggctctat |
| 241 | ggcttctgag gcggaaagaa ccagctgggg. |

In some embodiments, the sequence encoding the BGH polyA comprises or consists of the nucleotide sequence of:

(SEQ ID NO: 4)

| | |
|---|---|
| 1 | tcgctgatca gcctcgactg tgccttctag ttgccagcca |
| | tctgttgttt gcccctcccc |
| 61 | cgtgccttcc ttgaccctgg aaggtgccac tcccactgtc |
| | ctttcctaat aaaatgagga |
| 121 | aattgcatcg cattgtctga gtaggtgtca ttctattctg |
| | gggggtgggg tggggcagga |
| 181 | cagcaagggg gaggattggg aagacaatag caggcatgct |
| | ggggatgcgg tgggctctat |
| 241 | ggcttctgag gcggaaagaa ccagctgggg. |

In some embodiments of the compositions of the disclosure, the RPGR^{ORF15} polynucleotide further comprises a Kozak sequence. In some embodiments, the Kozak sequence comprises or consists of the nucleotide sequence of GGCCACCATG (SEQ ID NO:7).

In some embodiments of the compositions of the disclosure, the RPGR^{ORF15} polynucleotide further consists of a purified recombinant serotype 2 (rAAV) encoding the cDNA of RPGR^{ORF15}. In some embodiments, each 20 nm AAV virion contains a single stranded DNA insert sequence comprising: a 119 bp AAV2 5′ inverted terminal repeat (ITR), a 199 bp G protein-coupled rhodopsin kinase 1 (GRK1) promoter, a 10 bp Kozak sequence, a 3459 bp human RPGR^{ORF15} cDNA, a 270 bp Bovine growth hormone polyadenylation sequence (BGH-polyA), and a130 bp AAV2 3′ ITR, as well a short cloning sequences flanking the elements. The Kozak sequence may overlap the start of the RPGR^{ORF15} sequence, for example by 3 bp.

In some embodiments, the RPGR^{ORF15} polynucleotide comprises or consists of the sequence of:

(SEQ ID NO: 8)

| | |
|---|---|
| 1 | CTGCGCGCTC GCTCGCTCAC TGAGGCCGCC CGGGCGTCGG |
| | GCGACCTTTG GTCGCCCGGC |
| 61 | CTCAGTGAGC GAGCGAGCGC GCAGAGAGGG AGTGGCCAAC |
| | TCCATCACTA GGGGTTCCTG |
| 121 | CGGCAATTCA GTCGATAACT ATAACGGTCC TAAGGTAGCG |
| | ATTTAAATAC GCGCTCTCTT |
| 181 | AAGGTAGCCC CGGGACGCGT CAATTGGGGC CCCAGAAGCC |
| | TGGTGGTTGT TTGTCCTTCT |
| 241 | CAGGGGAAAA GTGAGGCGGC CCCTTGGAGG AAGGGGCCGG |
| | GCAGAATGAT CTAATCGGAT |
| 301 | TCCAAGCAGC TCAGGGGATT GTCTTTTTCT AGCACCTTCT |
| | TGCCACTCCT AAGCGTCCTC |
| 361 | CGTGACCCCG GCTGGGATTT AGCCTGGTGCTGTGTCAGCC |
| | CCGGGGCCAC CATGAGAGAG |
| 421 | CCAGAGGAGC TGATGCCAGA CAGTGGAGCA GTGTTTACAT |
| | TCGGAAAATC TAAGTTCGCT |
| 481 | GAAAATAACC CAGGAAAGTT CTGGTTTAAA AACGACGTGC |
| | CCGTCCACCT GTCTTGTGGC |
| 541 | GATGAGCATA GTGCCGTGGT CACTGGGAAC AATAAGCTGT |
| | ACATGTTCGG GTCCAACAAC |
| 601 | TGGGGACAGC TGGGGCTGGG ATCCAAATCT GCTATCTCTA |
| | AGCCAACCTG CGTGAAGGCA |
| 661 | CTGAAACCCG AGAAGGTCAA ACTGGCCGCT TGTGGCAGAA |
| | ACCACACTCT GGTGAGCACC |
| 721 | GAGGGCGGGA ATGTCTATGC CACCGGAGGC AACAATGAGG |
| | GACAGCTGGG ACTGGGGGAC |
| 781 | ACTGAGGAAA GGAATACCTT TCACGTGATC TCCTTCTTTA |
| | CATCTGAGCA TAAGATCAAG |
| 841 | CAGCTGAGCG CTGGCTCCAA CACATCTGCA GCCCTGACTG |
| | AGGACGGGCGCCTGTTCATG |
| 901 | TGGGGAGATA ATTCAGAGGG CCAGATTGGG CTGAAAAACG |
| | TGAGCAATGT GTGCGTCCCT |
| 961 | CAGCAGGTGA CCATCGGAAA GCCAGTCAGT TGGATTTCAT |
| | GTGGCTACTA TCATAGCGCC |
| 1021 | TTCGTGACCA CAGATGGCGA GCTGTACGTC TTTGGGGAGC |
| | CCGAAAACGG AAAACTGGGC |
| 1081 | CTGCCTAACC AGCTGCTGGG CAATCACCGG ACACCCCAGC |
| | TGGTGTCCGA GATCCCTGAA |
| 1141 | AAAGTGATCC AGGTCGCCTG CGGGGGAGAG CATACAGTGG |
| | TCCTGACTGA GAATGCTGTG |
| 1201 | TATACCTTCG GACTGGGCCA GTTTGGCCAG CTGGGGCTGG |
| | GAACCTTCCT GTTTGAGACA |
| 1261 | TCCGAACCAA AAGTGATCGA GAACATTCGC GACCAGACTA |
| | TCAGCTACAT TTCCTGCGGA |
| 1321 | GAGAATCACA CCGCACTGAT CACAGACATT GGCCTGATGT |
| | ATACCTTTGG CGATGGACGA |
| 1381 | CACGGGAAGC TGGGACTGGG ACTGGAGAAC TTCACTAATC |
| | ATTTTATCCC CACCCTGTGT |
| 1441 | TCTAACTTCC TGCGGTTCAT CGTGAAACTG GTCGCTTGCG |
| | GCGGGTGTCA CATGGTGGTC |
| 1501 | TTCGCTGCAC CTCATAGGGG CGTGGCTAAG GAGATCGAAT |
| | TTGACGAGAT TAACGATACA |
| 1561 | TGCCTGAGCG TGGCAACTTT CCTGCCATAC AGCTCCCTGA |
| | CTTCTGGCAA TGTGCTGCAG |
| 1621 | AGAACCCTGA GTGCAAGGAT GCGGAGAAGG GAGAGGGAAC |
| | GCTCTCCTGA CAGTTTCTCA |
| 1681 | ATGCGACGAA CCCTGCCACC TATCGAGGGA ACACTGGGAC |
| | TGAGTGCCTG CTTCCTGCCT |
| 1741 | AACTCAGTGT TTCCACGATG TAGCGAGCGG AATCTGCAGG |
| | AGTCTGTCCT GAGTGAGCAG |
| 1801 | GATCTGATGC AGCCAGAGGA ACCCGACTAC CTGCTGGATG |
| | AGATGACCAA GGAGGCCGAA |
| 1861 | ATCGACAACT CTAGTACAGT GGAGTCCCTG GGCGAGACTA |
| | CCGATATCCT GAATATGACA |
| 1921 | CACATTATGT CACTGAACAG CAATGAGAAG AGTCTGAAAC |
| | TGTCACCAGT GCAGAAGCAG |
| 1981 | AAGAAACAGC AGACTATTGG CGAGCTGACT CAGGACACCG |
| | CCCTGACAGA GAACGACGAT |
| 2041 | AGCGATGAGT ATGAGGAAAT GTCCGAGATG AAGGAAGGCA |
| | AAGCTTGTAA GCAGCATGTC |
| 2101 | AGTCAGGGGA TCTTCATGAC ACAGCCAGCC ACAACTATTG |
| | AGGCTTTTTC AGACGAGGAA |
| 2161 | GTGGAGATCC CCGAGGAAAA AGAGGGCGCA GAAGATTCCA |
| | AGGGGAATGG AATTGAGGAA |
| 2221 | CAGGAGGTGG AAGCCAACGA GGAAAATGTG AAAGTCCACG |
| | GAGGCAGGAA GGAGAAAACA |
| 2281 | GAAATCCTGT CTGACGATCT GACTGACAAG GCCGAGGTGT |
| | CCGAAGGCAA GGCAAAATCT |
| 2341 | GTCGGAGAGG CAGAAGACGG ACCAGAGGGA CGAGGGGATG |
| | GAACCTGCGA GGAAGGCTCA |
| 2401 | AGCGGGGCTG AGCATTGGCA GGACGAGGAA CGAGAGAAGG |
| | GCGAAAAGGA TAAAGGCCGC |
| 2461 | GGGGAGATGG AACGACCTGG AGAGGGCGAA AAAGAGCTGG |
| | CAGAGAAGGA GGAATGGAAG |
| 2521 | AAAAGGGACG GCGAGGAACA GGAGCAGAAA GAAAGGGAGC |
| | AGGGCCACCA GAAGGAGCGC |
| 2581 | AACCAGGAGA TGGAAGAGGG CGGCGAGGAA GAGCATGGCG |
| | AGGGAGAAGA GGAAGAGGGC |
| 2641 | GATAGAGAAG AGGAAGAGGA AAAAGAAGGC GAAGGGAAGG |
| | AGGAAGGAGA GGGCGAGGAA |
| 2701 | GTGGAAGGCG AGAGGGAAAA GGAGGAAGGA GAACGGAAGA |
| | AAGAGGAAAG AGCCGGCAAA |
| 2761 | GAGGAAAAGG GCGAGGAAGA GGGCGATCAG GGCGAAGGCG |
| | AGGAGGAAGA GACCGAGGGC |
| 2821 | CGCGGGGAAG AGAAAGAGGA GGGAGGAGAG GTGGAGGGCG |
| | GAGAGGTCGA AGAGGGAAAG |
| 2881 | GGCGAGCGCG AAGAGGAAGA GGAAGAGGGC GAGGGCGAGG |
| | AAGAAGAGGG CGAGGGGGAA |
| 2941 | GAAGAGGAGG GAGAGGGCGA AGAGGAAGAG GGGGAGGGAA |
| | AGGGCGAAGA GGAAGGAGAG |
| 3001 | GAAGGGGAGG GAGAGGAAGA GGGGGAGGAG GGCGAGGGGG |
| | AAGGCGAGGA GGAAGAAGGA |
| 3061 | GAGGGGGAAG GCGAAGAGGA AGGCGAGGGG GAAGGAGAGG |
| | AGGAAGAAGG GGAAGGCGAA |
| 3121 | GGCGAAGAGG AGGGAGAAGG AGAGGGGGAG GAAGAGGAAG |
| | GAGAAGGGAA GGGCGAGGAG |
| 3181 | GAAGGCGAAG AGGGAGAGGG GGAAGGCGAG GAAGAGGAAG |
| | GCGAGGGCGA AGGAGAGGAC |
| 3241 | GGCGAGGGCG AGGGAGAAGA GGAGGAAGGG GAATGGGAAG |
| | GCGAAGAAGA GGAAGGCGAA |
| 3301 | GGCGAAGGCG AAGAAGAGGG CGAAGGGGAG GGCGAGGAGG |
| | GCGAAGGCGA AGGGGAGGAA |
| 3361 | GAGGAAGGCG AAGGAGAAGG CGAGGAAGAA GAGGGAGAGG |
| | AGGAAGGCGA GGAGGAAGGA |
| 3421 | GAGGGGGAGG AGGAGGGAGA AGGCGAGGGC GAAGAAGAAG |
| | AAGAGGGAGA AGTGGAGGGC |
| 3481 | GAAGTCGAGG GGGAGGAGGG AGAAGGGGAA GGGGAGGAAG |
| | AAGAGGGCGA AGAAGAAGGC |
| 3541 | GAGGAAAGAG AAAAAGAGGG AGAAGGCGAG GAAAACCGGA |
| | GAAATAGGGA AGAGGAGGAA |
| 3601 | GAGGAAGAGG GAAAGTACCA GGAGACAGGC GAAGAGGAAA |
| | ACGAGCGGCA GGATGGCGAG |
| 3661 | GAATATAAGA AAGTGAGCAA GATCAAAGGA TCCGTCAAGT |
| | ACGGCAAGCA CAAAACCTAT |
| 3721 | CAGAAGAAAA GCGTGACCAA CACACAGGGG AATGGAAAAG |
| | AGCAGAGGAG TAAGATGCCT |
| 3781 | GTGCAGTCAA AACGGCTGCT GAAGAATGGC CCATCTGGAA |
| | GTAAAAAATT CTGGAACAAT |
| 3841 | GTGCTGCCCC ACTATCTGGA ACTGAAATAA GAGCTCCTCG |
| | AGGCGGCCCG CTCGAGTCTA |
| 3901 | GAGGGCCCTT CGAAGGTAAG CCTATCCCTA ACCCTCTCCT |
| | CGGTCTCGAT TCTACGCGTA |
| 3961 | CCGGTCATCA TCACCATCAC CATTGAGTTT AAACCCGCTG |
| | ATCAGCCTCG ACTGTGCCTT |
| 4021 | CTAGTTGCCA GCCATCTGTT GTTTGCCCCT CCCCCGTGCC |
| | TTCCTTGACC CTGGAAGGTG |
| 4081 | CCACTCCCAC TGTCCTTTCC TAATAAAATG AGGAAATTGC |
| | ATCGCATTGT CTGAGTAGGT |
| 4141 | GTCATTCTAT TCTGGGGGGT GGGGTGGGGC AGGACAGCAA |
| | GGGGGAGGAT TGGGAAGACA |
| 4201 | ATAGCAGGCA TGCTGGGGAT GCGGTGGGCT CTATGGCTTC |
| | TGAGGCGGAA AGAACCAGAT |
| 4261 | CCTCTCTTAA GGTAGCATCG AGATTTAAAT TAGGGATAAC |
| | AGGGTAATGG CGCGGGCCGC |
| 4321 | AGGAACCCCT AGTGATGGAG TTGGCCACTC CCTCTCTGCG |
| | CGCTCGCTCG CTCACTGAGG |
| 4381 | CCGGGCGACC AAAGGTCGCC CGACGCCCGG GCTTTGCCCG |
| | GGCGGCCTCA GTGAGCGAGC |
| 4441 | GAGCGCGCAG. |

In some embodiments of the compositions of the disclosure, the RPGR^{ORF15} polynucleotide further comprises a woodchuck hepatitis posttranscriptional regulatory element. In some embodiments, the RPGR^{ORF15} polynucleotide consists of a purified recombinant serotype 2 (rAAV) encoding the cDNA of RPGR^{ORF15}. In some embodiments, each 20 nm AAV virion contains a single stranded DNA insert sequence comprising: a 119 bp AAV2 5′ inverted terminal repeat (ITR), a 199 bp G protein-coupled rhodopsin kinase 1 (GRK1) promoter, a 10 bp Kozak sequence, a 3459 bp human RPGR^{ORF15} cDNA, a 588 bp WPRE, a 270 bp Bovine growth hormone polyadenylation sequence (BGH-polyA), and a 130 bp AAV2 3′ ITR, as well a short cloning sequences flanking the elements. In some embodiments, the sequence encoding the WPRE comprises a nucleotide sequence that has at least 80% identity, at least 97% identity or 100% identity to the nucleotide sequence of:

| | |
|---|---|
| 1 | atcaacctct ggattacaaa atttgtgaaa gattgactgg |
| | tattcttaac tatgttgctc |
| 61 | cttttacgct atgtggatac gctgctttaa tgcctttgta |
| | tcatgctatt gcttcccgta |
| 121 | tggctttcat tttctcctcc ttgtataaat cctggttgct |
| | gtctctttat gaggagttgt |
| 181 | ggcccgttgt caggcaacgt ggcgtggtgt gcactgtgtt |
| | tgctgacgca acccccactg |
| 241 | gttggggcat tgccaccacc tgtcagctcc tttccgggac |
| | tttcgctttc cccctcccta |
| 301 | ttgccacggc ggaactcatc gccgcctgcc ttgcccgctg |
| | ctggacaggg gctcggctgt |
| 361 | tgggcactga caattccgtg gtgttgtcgg ggaaatcatc |
| | gtcctttcct tggctgctcg |
| 421 | cctgtgttgc cacctggatt ctgcgcggga cgtccttctg |
| | ctacgtccct tcggccctca |
| 481 | atccagcgga ccttccttcc cgcggcctgc tgccggctct |
| | gcggcctctt ccgcgtcttc |
| 541 | gccttcgccc tcagacgagt cggatctccc tttgggccgc |
| | ctccccgc. |

In some embodiments, the sequence encoding the WPRE comprises or consists of the nucleotide sequence of:

(SEQ ID NO: 9)

| | |
|---|---|
| 1 | atcaacctct ggattacaaa atttgtgaaa gattgactgg |
| | tattcttaac tatgttgctc |
| 61 | cttttacgct atgtggatac gctgctttaa tgcctttgta |
| | tcatgctatt gcttcccgta |
| 121 | tggctttcat tttctcctcc ttgtataaat cctggttgct |
| | gtctctttat gaggagttgt |
| 181 | ggcccgttgt caggcaacgt ggcgtggtgt gcactgtgtt |
| | tgctgacgca acccccactg |
| 241 | gttggggcat tgccaccacc tgtcagctcc tttccgggac |
| | tttcgctttc cccctcccta |
| 301 | ttgccacggc ggaactcatc gccgcctgcc ttgcccgctg |
| | ctggacaggg gctcggctgt |
| 361 | tgggcactga caattccgtg gtgttgtcgg ggaaatcatc |
| | gtcctttcct tggctgctcg |
| 421 | cctgtgttgc cacctggatt ctgcgcggga cgtccttctg |
| | ctacgtccct tcggccctca |
| 481 | atccagcgga ccttccttcc cgcggcctgc tgccggctct |
| | gcggcctctt ccgcgtcttc |
| 541 | gccttcgccc tcagacgagt cggatctccc tttgggccgc |
| | ctccccgc. |

In some embodiments of the compositions of the disclosure, the RPGR^{ORF15} polynucleotide further comprises a sequence corresponding to a 5′ inverted terminal repeat (ITR) and a sequence corresponding to a 3′ inverted terminal repeat (ITR). In some embodiments, the sequence encoding the 5′ ITR and the sequence encoding the 3′ITR are identical. In some embodiments, the sequence encoding the 5′ ITR and the sequence encoding the 3′ITR are not identical. In some embodiments, the sequence encoding the 5′ ITR and the sequence encoding the 3′ITR are isolated or derived from an adeno-associated viral vector of serotype 2 (AAV2). In some embodiments, the sequence encoding the 5′ ITR and the sequence encoding the 3′ITR comprise a wild type sequence. In some embodiments, the sequence encoding the 5′ ITR and the sequence encoding the 3′ITR comprise a truncated wild type AAV2 sequence. In some embodiments, the sequence encoding the 5′ ITR and the sequence encoding the 3′ITR comprise a variation when compared to a wild type AAV2 sequence. In some embodiments, the variation comprises a substitution, an insertion, a deletion, an inversion, or a transposition. In some embodiments, the variation comprises a truncation or an elongation of a wild type or a variant sequence.

In some embodiments of the compositions of the disclosure, an AAV comprises a sequence corresponding to a 5′ inverted terminal repeat (ITR) and a sequence corresponding to a 3′ inverted terminal repeat (ITR). In some embodiments, the sequence encoding the 5′ ITR and the sequence encoding the 3′ITR are identical. In some embodiments, the sequence encoding the 5′ ITR and the sequence encoding the 3′ITR are not identical. In some embodiments, the sequence encoding the 5′ ITR and the sequence encoding the 3′ITR are isolated or derived from an adeno-associated viral vector of serotype 2 (AAV2). In some embodiments, the sequence encoding the 5′ ITR and the sequence encoding the 3′ITR comprise a wild type sequence. In some embodiments, the sequence encoding the 5′ ITR and the sequence encoding the 3′ITR comprise a truncated wild type AAV2 sequence. In some embodiments, the sequence encoding the 5′ ITR and the sequence encoding the 3′ITR comprise a variation when compared to a wild type AAV2 sequence. In some embodiments, the variation comprises a substitution, an insertion, a deletion, an inversion, or a transposition. In some embodiments, the variation comprises a truncation or an elongation of a wild type or a variant sequence.

In some embodiments of the compositions of the disclosure, an AAV comprises a viral sequence essential for formation of a replication-deficient AAV. In some embodiments, the viral sequence is isolated or derived from an AAV of the same serotype as one or both of the sequence encoding the 5′ITR or the sequence encoding the 3′ITR. In some embodiments, the viral sequence, the sequence encoding the 5′ITR or the sequence encoding the 3′ITR are isolated or derived from an AAV2.

In some embodiments of the compositions of the disclosure, an AAV comprises a viral sequence essential for formation of a replication-deficient AAV, a sequence encoding the 5′ITR and a sequence encoding the 3′ITR, but does not comprise any other sequence isolated or derived from an AAV. In some embodiments, the AAV is a recombinant AAV (rAAV), comprising a viral sequence essential for formation of a replication-deficient AAV, a sequence encoding the 5′ITR, a sequence encoding the 3′ITR, and a sequence encoding an RPGR^{ORF15} polynucleotide of the disclosure.

In some embodiments, a plasmid DNA used to create the rAAV in a host cell comprises a selection marker. Illustrative selection markers include, but are not limited to, antibiotic resistance genes. Illustrative antibiotic resistance genes include, but are not limited to, ampicillin and kanamycin. Illustrative selection markers include, but are not limited to, drug or small molecule resistance genes. Illustrative selection markers include, but are not limited to, dapD and a repressible operator including but not limited to a lacO/P construct controlling or suppressing dapD expression, wherein plasmid selection is performed by administering or contacting a transformed cell with a plasmid capable of operator repressor titration (ORT). Illustrative selection markers include, but are not limited to, a ccd selection gene. In some embodiments, the ccd selection gene comprises a sequence encoding a ccdA selection gene that rescues a host cell line engineered to express a toxic ccdB gene. Illustrative selection markers include, but are not limited to, sacB, wherein an RNA is administered or contacted to a host cell to suppress expression of the sacB gene in sucrose media. Illustrative selection markers include, but are not limited to, a segregational killing mechanism such as the parAB+locus composed of Hok (a host killing gene) and Sok (suppression of killing).

### AAV-RPGR^{ORF15} Structure

AAV-RPGR^{ORF15} consists of a purified recombinant serotype 2 adeno-associated viral vector (rAAV) encoding the RPGR^{ORF15}cDNA.

In some embodiments, AAV-RPGR^{ORF15} comprises one or more of a sequence encoding a 5′ ITR, a sequence encoding a 3′ ITR and a sequence encoding a capsid protein that is isolated and/or derived from a serotype 8 adeno-associated viral vector (AAV8). In some embodiments, the AAV-RPGR^{ORF15} comprises a truncated sequence encoding a 5′ ITR and a sequence encoding a 3′ ITR that is isolated and/or derived from a serotype 2 adeno-associated viral vector (AAV2) and a sequence encoding a capsid protein that is isolated and/or derived from a serotype 8 adeno-associated viral vector (AAV8). In some embodiments, the AAV-RPGR^{ORF15} comprises wild type AAV2 ITRs (a wild type 5′ ITR and a wild type 3′ ITR).

In some embodiments, each 20 nm AAV virion contains a single stranded DNA insert sequence (plus short cloning sites flanking each element) comprising: (a) a 5′ inverted terminal repeat (ITR), (b) a promoter suitable for expression in mammalian cells, (c) a cDNA encoding RPGR^{ORF15}, and (d) a 3′ ITR.

In some embodiments, each 20 nm AAV virion contains a single stranded DNA insert sequence (plus short cloning sites flanking each element) comprising: (a) a 5′ inverted terminal repeat (ITR), (b) a promoter suitable for expression in mammalian cells, (c) a cDNA encoding RPGR^{ORF15}, (c) a polyadenylation signal, and (d) a bp 3′ ITR.

In some embodiments, each 20 nm AAV virion contains a single stranded DNA insert sequence (plus short cloning sites flanking each element) comprising: (a) a 5′ inverted terminal repeat (ITR), (b) a promoter suitable for expression in mammalian cells, (c) a Kozak sequence, (d) a cDNA encoding RPGR^{ORF15}, (e) a polyadenylation signal, and (f) a bp 3′ ITR.

In some embodiments, each 20 nm AAV virion contains a single stranded DNA insert sequence (plus short cloning sites flanking each element) comprising: (a) a 5′ inverted terminal repeat (ITR), (b) a promoter suitable for expression in mammalian cells, (c) a cDNA encoding RPGR^{ORF15}, (d) a post-transcriptional regulatory element (PRE), (e) a polyadenylation sequence (polyA), and (f) a 3′ ITR.

In some embodiments, each 20 nm AAV virion contains a single stranded DNA insert sequence (plus short cloning sites flanking each element) comprising: (a) a 119 bp 5′ inverted terminal repeat (ITR), (b) a promoter, optionally, a 199 bp GRK1 promoter, (c) a cDNA encoding RPGR^{ORF15}, (d) a 270 bp Bovine growth hormone polyadenylation sequence (BGH-polyA), and (e) a 130 bp 3′ ITR.

In some embodiments, each 20 nm AAV virion contains a single stranded DNA insert sequence (plus short cloning sites flanking each element) comprising: (a) a 119 5′ inverted terminal repeat (ITR), (b) a promoter, optionally, a 199 bp GRK1 promoter, (c) a Kozak sequence, (d) a cDNA encoding RPGR^{ORF15}, (e) a 270 bp Bovine growth hormone polyadenylation sequence (BGH-polyA), and (f) a 130 3′ ITR.

In some embodiments, each 20 nm AAV virion contains a single stranded DNA insert sequence (plus short cloning sites flanking each element) comprising: (a) a 119 5′ inverted terminal repeat (ITR), (b) a promoter, optionally, a 199 bp GRK1 promoter, (c) a cDNA encoding RPGR^{ORF15}, (d) a 588 bp Woodchuck hepatitis virus post-transcriptional regulatory element (WPRE), (e) a 270 bp Bovine growth hormone polyadenylation sequence (BGH-polyA), and (f) a 130 3′ ITR.

In some embodiments, each 20 nm AAV virion contains a single stranded DNA insert sequence (plus short cloning sites flanking each element) comprising: (a) a 119 bp 5′ inverted terminal repeat (ITR), (b) a promoter, optionally, a 199 bp GRK1 promoter, (c) a 10 bp Kozak sequence, (d) a cDNA encoding RPGR^{ORF15}, (e) a 588 bp Woodchuck hepatitis virus post-transcriptional regulatory element (WPRE), (f) a 270 bp Bovine growth hormone polyadenylation sequence (BGH-polyA), and (g) a 130 bp 3′ ITR.

AAV-RPGR^{ORF15} of the disclosure may comprise a sequence encoding a promoter capable of expression in a mammalian cell. Preferably, AAVs or AAV-RPGR^{ORF15} constructs of the disclosure may comprise a sequence encoding a promoter capable of expression in a human cell. Illustrative promoters of the disclosure include, but are not limited to, constitutively active promoters, cell-type specific promoters, viral promoters, mammalian promoters, and hybrid or recombinant promoters. In some embodiments of the compositions of the disclosure, the RPGR^{ORF15} cDNA is under the control of a G protein-coupled receptor kinase 1 (GRK1) promoter.

AAV-RPGR^{ORF15} of the disclosure may comprise a sequence encoding a post-transcriptional regulatory element (PRE). Illustrative PREs of the disclosure include, but are not limited to, a Woodchuck hepatitis virus post-transcriptional regulatory element (WPRE). In some embodiments of the compositions of the disclosure, the AAV comprises a 588 bp WPRE, originating from the 3′ region of the viral S transcript, directly downstream of the cDNA encoding a therapeutic RPGR^{ORF15} of the disclosure. This WPRE is important for high-level expression of native mRNA transcripts, acting to enhance mRNA processing and transport of intronless genes. In some embodiments of the compositions of the disclosure, the WPRE has been modified to prevent expression of the viral X antigen by ablation of the translation initiation site. This has been achieved by deleting the We2 promoter/enhancer and mutating the We1 promoter.

AAV-RPGR^{ORF15} of the disclosure may comprise a polyadenosine (polyA) sequence. Illustrative polyA sequences of the disclosure include, but are not limited to, a bovine growth hormone polyadenylation (BGH-polyA) sequence. The BGH-polyA sequence is used to enhance gene expression and has been shown to yield three times higher expression levels than other polyA sequences such as SV40 and human collagen polyA. This increased expression is largely independent of the type of upstream promoter or transgene. Increasing expression levels using both BGH-polyA and WPRE sequences allows a lower overall dose of AAV or plasmid vector to be injected, which is less likely to generate a host immune response.

### Dosage Form

AAV-RPGR^{ORF15} compositions of the disclosure may be formulated for systemic or local administration. Preferably, AAV-RPGR^{ORF15} compositions of the disclosure may be formulated for local administration.

AAV-RPGR^{ORF15} compositions of the disclosure may be formulated as a Suspension for Injection or Infusion.

AAV-RPGR^{ORF15} compositions of the disclosure may be formulated for injection or infusion by any route, including but not limited to, an intravitreous injection or infusion, a subretinal injection or infusion, or a suprachoroidal injection or infusion.

In any of the compositions described herein, the amount of AAV-RPGR^{ORF15} in a composition may be expressed as an absolute amount (genome particles (gp or pg)) or a concentration (vector genomes (vg) per milliliter (mL)). The value for “genome particles” is equivalent to the value for “vector genomes”.

AAV-RPGR^{ORF15} compositions of the disclosure may be formulated at a concentration of between 0.5×10¹⁰ vector genomes (vg) per milliliter (mL) and 1×10¹³ vg/mL, e.g., 0.5×10¹⁰ vg/mL and 1×10¹³ vg/mL, 0.5×10¹¹ vg/mL and 1×10¹³ vg/mL, 0.5×10¹² vg/mL and 1×10¹³ vg/mL, 1×10¹² vg/mL and 1×10¹³ vg/mL, 2×10¹² vg/mL and 1×10¹³ vg/mL, inclusive of the endpoints. As used herein, vg/mL refers to the number of rAAV vector genomes per mL of solution, as measured by a quantitative assay such as qPCR or ddPCR. In some embodiments, compositions of the disclosure may be formulated at a concentration of 0.5×10¹¹ vg/mL or 1×10¹² vg/ml. In some embodiments, compositions of the disclosure may be formulated at a concentration of about 0.5×10¹¹ vg/mL. In some embodiments, compositions of the disclosure may be formulated at a concentration of about 1×10¹² vg/mL. In some embodiments, compositions of the disclosure may be formulated at a concentration of about 5×10¹² vg/mL. In some embodiments, compositions of the disclosure may be formulated at a concentration of about 1×10¹³ vg/mL. In some embodiments, the compositions of the disclosure may be formulated at a concentration of about 5×10⁹ gp/mL and 1×10¹³ gp/mL, e.g., 0.5×10¹⁰ gp/mL and 1×10¹³ gp/mL, 0.5×10¹¹ gp/mL and 1×10¹³ gp/mL, 0.5×10¹² gp/mL and 1×10¹³ gp/mL, 1×10¹² gp/mL and 1×10¹³ gp/mL, 2×10¹² gp/mL and 1×10¹³ gp/mL. In some embodiments, the compositions of the disclosure may be formulated at a concentration of about 1×10¹⁰ gp/ml. In some embodiments, the compositions of the disclosure may be formulated at a concentration of about 5×10¹⁰ gp/mL. In some embodiments, the compositions of the disclosure may be formulated at a concentration of about 1×10¹¹ gp/mL. In some embodiments, the compositions of the disclosure may be formulated at a concentration of about 2.5×10¹¹ gp/mL. In some embodiments, the compositions of the disclosure may be formulated at a concentration of about 5×10¹¹ gp/mL. In some embodiments, the vector genomes (vg) is determined by a quantitative assay such as qPCR or ddPCR after treatment of the particles with a DNase, i.e. as DNase Resistant Particles (DRP).

AAV-RPGR^{ORF15} compositions of the disclosure may be formulated at a concentration of between 0.5×10¹⁰ DNase Resistant Particles (DRP) per milliliter (mL) and 1×10¹³ DRP/mL, e.g., 0.5×10¹⁰ DRP/mL and 1×10¹³ DRP/mL, 0.5×10¹¹ DRP/mL and 1×10¹³ DRP/mL, 0.5×10¹² DRP/mL and 1×10¹³ DRP/mL, 1×10¹² DRP/mL and 1×10¹³ DRP/mL, 2×10¹² DRP/mL and 1×10¹³ DRP/mL, inclusive of the endpoints. As used herein, DRP/mL refers to the number of rAAV DNase resistant particles per mL of solution, as measured by methods disclosed herein. In some embodiments, compositions of the disclosure may be formulated at a concentration of 0.5×10¹¹ DRP/mL or 1×10¹² DRP/mL. In some embodiments, compositions of the disclosure may be formulated at a concentration of about 0.5×10¹¹ DRP/mL. In some embodiments, compositions of the disclosure may be formulated at a concentration of about 1×10¹² DRP/mL. In some embodiments, compositions of the disclosure may be formulated at a concentration of about 5×10¹² DRP/mL. In some embodiments, compositions of the disclosure may be formulated at a concentration of about 1×10¹³ DRP/mL.

In some embodiments, the compositions of the disclosure may be formulated at a concentration of about 5×10⁹ DRP/mL and 1×10¹³ DRP/mL, e.g., 0.5×10¹⁰ DRP/mL and 1×10¹³ DRP/mL, 0.5×10¹¹ DRP/mL and 1×10¹³ DRP/mL, 0.5×10¹² DRP/mL and 1×10¹³ DRP/mL, 1×10¹² DRP/mL and 1×10¹³ DRP/mL, 2×10¹² DRP/mL and 1×10¹³ DRP/mL. In some embodiments, the compositions of the disclosure may be formulated at a concentration of about 1×10¹⁰ DRP/mL. In some embodiments, the compositions of the disclosure may be formulated at a concentration of about 5×10¹⁰ DRP/mL. In some embodiments, the compositions of the disclosure may be formulated at a concentration of about 1×10¹¹ DRP/mL. In some embodiments, the compositions of the disclosure may be formulated at a concentration of about 2.5×10¹¹ DRP/mL. In some embodiments, the compositions of the disclosure may be formulated at a concentration of about 5×10¹¹ DRP/mL.

In some embodiments, the compositions of the disclosure comprises between 1.25×10¹² DRP/mL and 1.0×10¹³ DRP/mL, e.g. 1.25×10¹² DRP/mL, 1.5×10¹² DRP/mL, 1.75×10¹² DRP/mL, 2.0×10¹² DRP/mL, 2.5×10¹² DRP/mL, 3.0×10¹² DRP/mL, 3.5×10¹² DRP/mL, 4.0×10¹² DRP/mL, 4.5×10¹² DRP/mL, 5.0×10¹² DRP/mL, 5.5×10¹² DRP/mL, 6.0×10¹² DRP/mL, 6.5×10¹² DRP/mL, 7.0×10¹² DRP/mL, 7.5×10¹² DRP/mL, 8.0×10¹² DRP/mL, 8.5×10¹² DRP/mL, 9.0×10¹² DRP/mL, 9.5×10¹² DRP/mL, or 1.0×10¹³ DRP/mL.

Compositions of the disclosure may be diluted prior to administration using a diluent of the disclosure. In some embodiments, the diluent is identical to a formulation buffer used for preparation of the AAV-RPGR^{ORF15} composition. In some embodiments, the diluent is not identical to a formulation buffer used for preparation of the AAV-RPGR^{ORF15} composition.

Compositions of the disclosure may comprise full and empty AAV particles. In some embodiments, a full AAV particle comprises a single stranded DNA encoding a AAV-RPGR^{ORF15} of the disclosure. The ordinarily skilled artisan can determine whether an AAV particle is full or empty through, for example, transmission electron microscopy analysis, qPCR or ddPCR. In some embodiments of the composition of the disclosure, the composition comprises at least 30%, at least 35%, at least 40%, at least 45%, at least 50%, at least 55%, at least 60%, 65%, at least 67%, at least 69%, at least 70%, at least 71%, at least 72%, at least 73%, at least 76%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% full AAV particles. In some embodiments, the composition comprises at least 70% full AAV particles.

### Administration

AAV-RPGR^{ORF15} compositions of the disclosure may be administered to the eye of a subject by subretinal, direct retinal, suprachoroidal or intravitreal delivery.

### Subretinal Administration

Subretinal delivery may comprise an injection or infusion into a subretinal space. In some embodiments of the disclosure, the subretinal delivery comprises an injection or infusion into a subretinal space. In some embodiments, the subretinal delivery comprises one or more injection(s) or infusion(s) into a subretinal space. In some embodiments, the subretinal delivery comprises at least one injection or infusion into a subretinal space. In some embodiments, the subretinal delivery comprises a plurality of injections or infusions into a subretinal space.

Subretinal delivery may comprise an injection or infusion into a fluid-filled bleb in a subretinal space. In some embodiments of the disclosure, the subretinal delivery comprises an injection or infusion into a subretinal space. In some embodiments, the subretinal delivery comprises one or more injection(s) or infusion(s) into a fluid-filled bleb in a subretinal space. In some embodiments, the subretinal delivery comprises at least one injection or infusion into a fluid-filled bleb in a subretinal space. In some embodiments, the subretinal delivery comprises a plurality of injections or infusions into a fluid-filled bleb in a subretinal space.

The subretinal space is the space underneath the neurosensory retina. During a subretinal injection, material is injected into and creates a space between the photoreceptor cell and retinal pigment epithelial (RPE) layers. When the injection is carried out through a small retinotomy, a retinal detachment may be created. The detached, raised layer of the retina that is generated by the injected material is referred to as a “bleb”. In some embodiments, the hole created by the subretinal injection is sufficiently small that the injected solution does not significantly reflux back into the vitreous cavity after administration. Preferably, the injection creates a self-sealing entry point in the neurosensory retina, i.e. once the injection needle is removed, the hole created by the needle reseals such that very little or substantially no injected material is released through the hole.

In some embodiments, the device used for subretinal injection comprises a microdelivery device. In some embodiments, the microdelivery device comprises a microneedle suitable for subretinal injection. Suitable microneedles are commercially available. In some embodiments, the microneedle comprises a DORC 41G Teflon subretinal injection needle (Dutch Ophthalmic Research Center International BV, Zuidland, The Netherlands). In some embodiments, the device comprises a volume of at least 50 μL. In some embodiments, the device comprises a volume of at least 100 μL or up to 100 μL (e.g., 25-100 μL, 50-100 μL, 75-100 μL). In some embodiments, the device comprises a volume of at least 200 μL. In some embodiments, the device comprises 80-110 μL of dead volume in addition to the volume of AAV-RPGR^{ORF15} that will be administered to the subject (i.e., volume of the composition that is used to prime the device, but cannot be injected or recovered).

In some embodiments, subretinal injections can be performed by delivering the composition comprising AAV particles under direct visual guidance using an operating microscope (Leica Microsystems, Germany). One illustrative approach is that of using a scleral tunnel approach through the posterior pole to the superior retina with a Hamilton syringe and 34-gauge needle (ESS labs, UK). Alternatively, sub-retinal injections can be performed using an anterior chamber paracentesis with a 33G needle prior to the subretinal injection using a WPI syringe and a beveled 35G-needle system (World Precision Instruments, UK). An additional alternative is a WPI Nanofil Syringe (WPI, part #NANOFIL) and a 34 gauge WBI Nanofil needle (WPI, part # NF34BL-2).

In some embodiments, the subretinal injection comprises two-step subretinal injection. In some embodiments, the two-step subretinal injection comprises: (a) inserting a subretinal injection needle between a photoreceptor cell layer and a retinal pigment epithelial layer in an eye of the subject; (b) injecting a solution between the photoreceptor cell layer and a retinal pigment epithelial layer in the eye of the subject in an amount sufficient to partially detach the retina from the RPE and form a bleb; and (c) injecting the composition into the bleb. In some embodiments, the solution comprises a balanced salt solution.

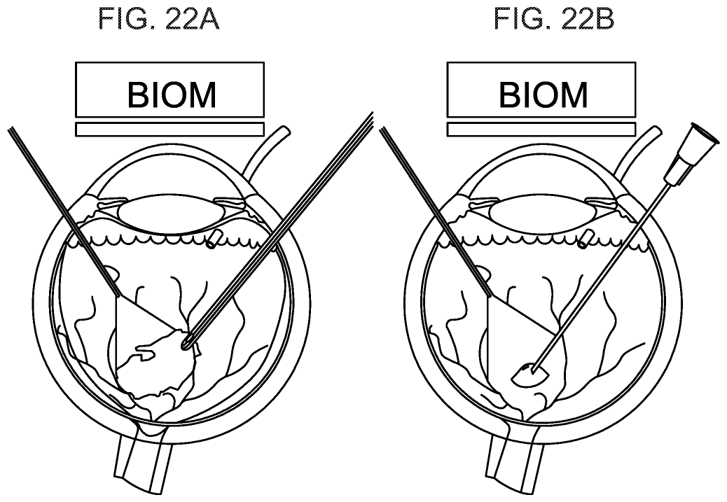

In some embodiments, subretinal delivery comprises a vitrectomy and an injection into the subretinal space. In some embodiments, the surgery may be conducted with the BIOM® (binocular indirect ophthalmomicroscope) vitrectomy system. For example, a subject may undergo a vitrectomy and detachment of the posterior hyaloid (FIG. 22A). In some embodiments, prior to sub-retinal injection, the retina may be detached with up to 0.5 mL of balanced salt solution (BSS). In some embodiments, prior to sub-retinal injection, the retina may be detached with 0.05-0.5 mL of BSS. In some embodiments, prior to sub-retinal injection, the retina may be detached with 0.1-0.5 mL of BSS. In some embodiments, prior to sub-retinal injection, the retina may be detached with 0.1-0.5 mL of balanced salt solution (BSS) injected through a 41-gauge sub-retinal cannula connected to a vitreous injection set (FIG. 22B). In some embodiments, prior to sub-retinal injection, the retina may be detached with 0.01-1.0 mL, 0.05-1.0 mL, 0.1-1 mL, 0.01-0.5 mL, 0.05-0.5 mL, or 0.1-0.5 mL of BSS. In some embodiments, prior to sub-retinal injection, the retina may be detached with about 0.05 mL, about 0.1 mL, about 0.2 mL, about 0.3 mL, about 0.4 mL, about 0.5 mL, or about 0.6 mL of BSS. A single dose of the viral vector may then be injected into the sub-retinal fluid through the same entry site. If detachment of the macula occurs with a smaller volume of fluid, then additional subretinal sites in the posterior globe (e.g. nasal to the disc) may also be chosen to deliver up to the entire dose (e.g., 0.1 mL) of vector. This avoids excessive foveal stretch. If unexpected complications of retinal detachment are encountered (e.g., a macular hole created requiring treatment with gas), the injection of vector may be deferred until a later date.

In some embodiments, subretinal delivery comprises more than one subretinal injection. In some embodiments, subretinal delivery comprises multiple subretinal injections administered at different locations in the eye. In some embodiments, subretinal delivery comprises multiple subretinal injections administered to the same location in the eye at different times. In some embodiments, an additional subretinal injection occurs at at least 1 week, 2 weeks, 3 weeks, 1 month, 2 months, 3 months, 4 months, 5 months 6 months, 12 months, 18 months, 24 months or 3 years after the previous subretinal injection. In some embodiments, subretinal delivery comprises multiple subretinal injections administered both to different locations of the eye and at different times.

### Suprachoroidal Administration

Suprachoroidal delivery may comprise an injection or infusion into a suprachoroidal space. In some embodiments of the disclosure, the suprachoroidal delivery comprises an injection or infusion into a suprachoroidal space. In some embodiments, the suprachoroidal delivery comprises one or more injection(s) or infusion(s) into a suprachoroidal space. In some embodiments, the suprachoroidal delivery comprises at least one injection or infusion into a suprachoroidal space. In some embodiments, the suprachoroidal delivery comprises a plurality of injections or infusions into a suprachoroidal space.

Suprachoroidal delivery may comprise an injection or infusion into a fluid-filled bleb in a suprachoroidal space. In some embodiments of the disclosure, the suprachoroidal delivery comprises an injection or infusion into a suprachoroidal space. In some embodiments, the suprachoroidal delivery comprises one or more injection(s) or infusion(s) into a fluid-filled bleb in a suprachoroidal space. In some embodiments, the suprachoroidal delivery comprises at least one injection or infusion into a fluid-filled bleb in a suprachoroidal space. In some embodiments, the suprachoroidal delivery comprises a plurality of injections or infusions into a fluid-filled bleb in a suprachoroidal space.

The suprachoroidal space is the space between the sclera and the choroid of the retina. During a suprachoroidal injection, material is injected into this space. The suprachoroidal space traverses the circumference of the posterior segment of the eye. By delivering a composition to the suprachoroidal space, the composition may be delivered directly to the choroid, retinal pigment epithelium, and retina (including the photoreceptor cells) at a high concentration (and without dilution in the space), preserving or maintaining bioavailability of the composition at the site of injection or infusion.

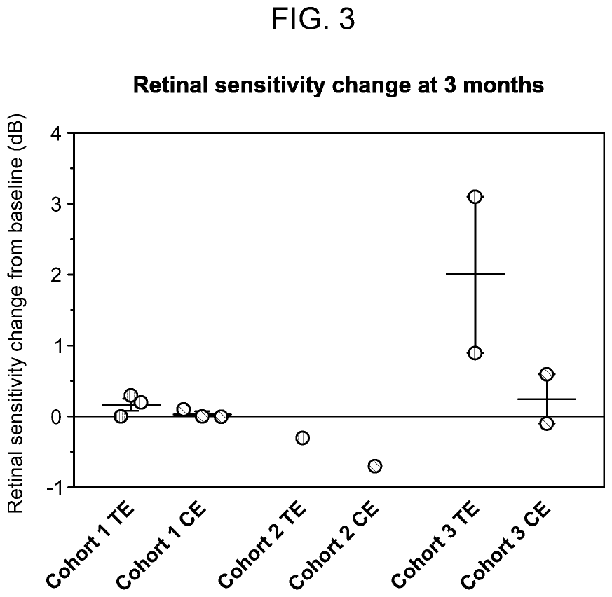

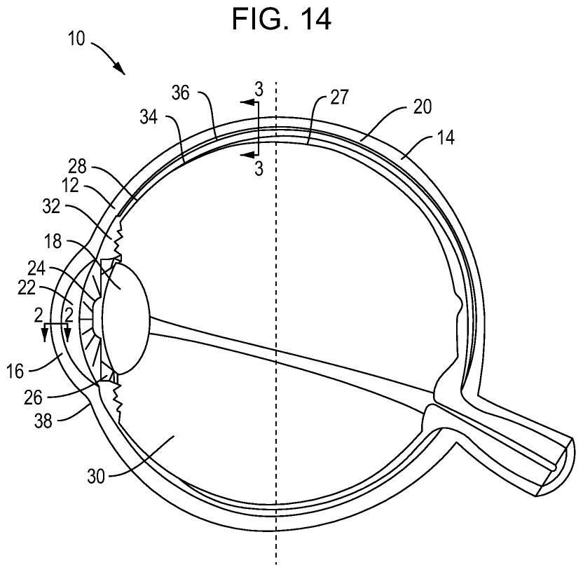

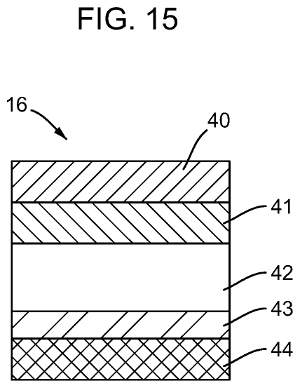

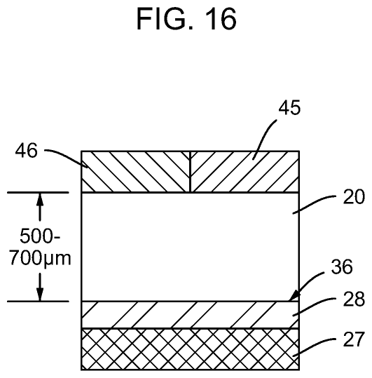

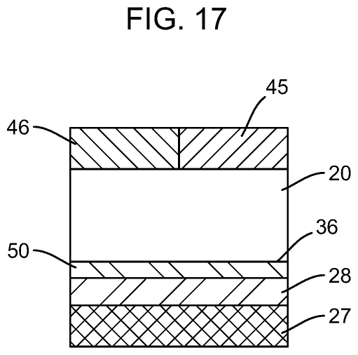

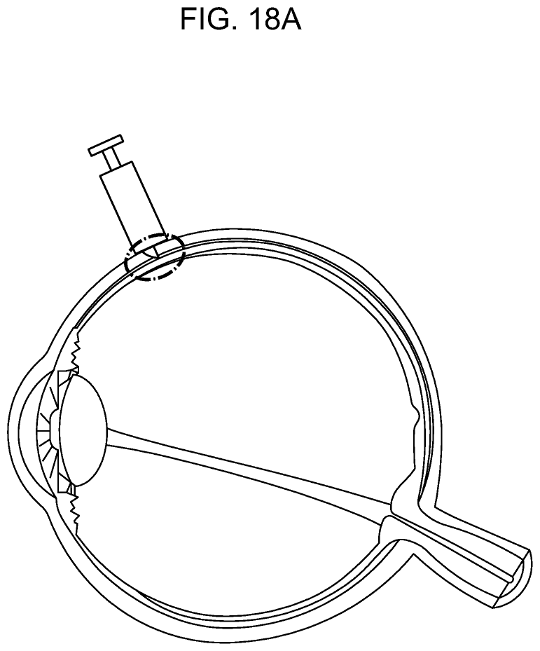

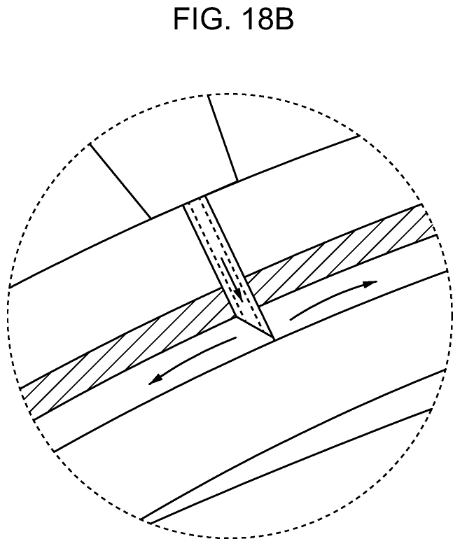

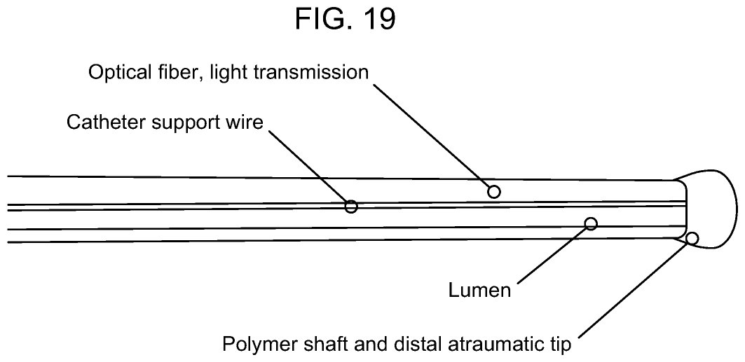

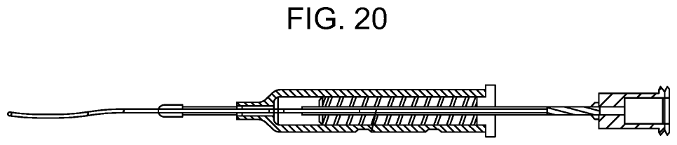

FIGS. 14-17 are various views of a human eye 10 (with FIGS. 15-17 being cross-sectional views). While specific regions are identified, those skilled in the art will recognize that the proceeding identified regions do not constitute the entirety of the eye 10, rather the identified regions are presented as a simplified example suitable for the discussion of the embodiments herein. The eye 10 includes both an anterior segment 12 (the portion of the eye in front of and including the lens) and a posterior segment 14 (the portion of the eye behind the lens). The anterior segment 12 is bounded by the cornea 16 and the lens 18, while the posterior segment 14 is bounded by the sclera 20 and the lens 18. The anterior segment 12 is further subdivided into the anterior chamber 22, between the iris 24 and the cornea 16, and the posterior chamber 26, between the lens 18 and the iris 24. The cornea 16 and the sclera 20 collectively form a limbus 38 at the point at which they meet. The exposed portion of the sclera 20 on the anterior segment 12 of the eye is protected by a clear membrane referred to as the conjunctiva 45 (see e.g., FIGS. 15 and 16). Underlying the sclera 20 is the choroid 28 and the retina 27, collectively referred to as retinachoroidal tissue. A vitreous humor 30 (also referred to as the “vitreous”) is disposed between a ciliary body 32 (including a ciliary muscle and a ciliary process) and the retina 27. The anterior portion of the retina 27 forms an or a serrata 34. The loose connective tissue, or potential space, between the choroid 28 and the sclera 20 is referred to as the suprachoroid. FIG. 15 illustrates the cornea 16, which is composed of the epithelium 40, the Bowman's layer 41, the stroma 42, the Descemet's membrane 43, and the endothelium 44. FIG. 16 illustrates the sclera 20 with surrounding Tenon's Capsule 46 or conjunctiva 45, suprachoroidal space 36, choroid 28, and retina 27, substantially without fluid and/or tissue separation in the suprachoroidal space 36 (i.e., the in this configuration, the space is “potential” suprachoroidal space). As shown in FIG. 3, the sclera 20 has a thickness between about 500 μm and 700 μm. FIG. 17 illustrates the sclera 20 with the surrounding Tenon's Capsule 46 or the conjunctiva 45, suprachoroidal space 36, choroid 28, and retina 27, with fluid 50 in the suprachoroidal space 36.

As used herein, the term “suprachoroidal space,” describes the space (or volume) and/or potential space (or potential volume) in the region of the eye 10 disposed between the sclera 20 and choroid 28. This region is composed of closely packed layers of long pigmented processes derived from each of the two adjacent tissues; however, a space can develop in this region because of fluid or other material buildup in the suprachoroidal space and the adjacent tissues. The suprachoroidal space can be expanded by fluid buildup because of some disease state in the eye or because of some trauma or surgical intervention. In some embodiments, the fluid buildup is intentionally created by the delivery, injection and/or infusion of a drug formulation into the suprachoroid to create and/or expand further the suprachoroidal space 36 (i.e., by disposing a gene therapy composition of the disclosure therein). This volume may serve as a pathway for uveoscleral outflow (i.e., a natural process where fluid exits the eye through a pressure-independent process) and may become a space in instances of choroidal detachment from the sclera.

The dashed line in FIG. 14 represents the equator of the eye 10. In some embodiments, the contacting step may comprise piercing an outer surface of the sclera at position between the equator and the limbus 38 (i.e., in the anterior portion 12 of the eye 10) For example, in some embodiments, the position is between about two millimeters and 10 millimeters (mm) posterior to the limbus 38. In other embodiments, the position is at about the equator of the eye 10. In still other embodiments, the position is posterior the equator of the eye 10. In this manner, a gene therapy composition of the disclosure can be introduced (e.g., via the needle, a microneedle, a catheter, or a microcatheter) into the suprachoroidal space 36 through at least one channel in the sclera and can flow through the suprachoroidal space 36 away from the at least one channel during an infusion event (e.g., during injection).

### Suprachoroidal Route

Compositions of the disclosure provide a therapeutic benefit when they are administered by a subretinal route, however, in a subject with a retinal disease or disorder (particularly when the retinal damage is severe and the tissue is weakened), it may be difficult to administer by a subretinal route without causing additional damage to the disease-weakened retina. Moreover, even when a subretinal injection would not cause permanent damage the retina, due to the physical constraints of the injection, the maximal volume that may be administered per injection is limited.

Suprachoroidal injections or infusions overcome many of the challenges faced by using an intravitreal or subretinal route. Suprachoroidal injections or infusions may be used to treat retinal disease and provide access to cells of the retinal pigment epithelium (RPE) without contacting the retina or RPE itself with any medical device. Injections or infusions made by a suprachoroidal route are may be targeted to a region of the RPE and retina. Depending, in part, upon the formulation of the gene therapy composition and the dispersion methods used (passive v. active), the composition can be spread evenly over a larger surface of the retina or RPE than the targeted injection site. Within a single procedure or over the course of multiple procedures, suprachoroidal administration permits multiple injections or infusions at multiple positions across the outer surface of the retina.

The suprachoroidal space may hold up to 1 mL of an injected or infused composition. Moreover, composition injected or infused into the suprachoroidal space may rapidly diffuse into the posterior segment of the eye. However, diffusion of compositions from suprachoroidal space into the vitreous decreases as the lipophilicity and molecular weight of the composition increases. In preferred embodiments of the compositions of the disclosure, the compositions comprise a viral vector, and, therefore, these compositions do not diffuse past the RPE to reach the vitreous.

The disclosure provides methods of administering an AAV-RPGR^{ORF15} composition of the disclosure by a suprachoroidal route to multiple focal areas of the retina for the purpose of improving the ellipsoid zone (EZ), retinal sensitivity, visual acuity, retinal thickness or ONL thickness, or a combination thereof. Retinal neurons form a spatial map of the entire visual field in each eye. With respect to the each human eye, left and right, and from the perspective of the subject, the left half of the visual field is perceived by neurons on the right half of the retina. Conversely, with respect to the each human eye, left and right, and from the perspective of the subject, the right half of the visual field is perceived by neurons on the left half of the retina.

In some embodiments, the device used for suprachoroidal injection comprises a microdelivery device. In some embodiments, the microdelivery device comprises a microcatheter suitable for suprachoroidal injection. Suitable microcatheters are commercially available. In some embodiments, the device comprises a volume of at least 50 μL. In some embodiments, the device comprises a volume of at least 100 μL or up to 100 μL (e.g., 25-100 μL, 50-100 μL, 75-100 μL). In some embodiments, the device comprises a volume of at least 200 μL. In some embodiments, the device comprises 50-200 μL of dead volume in addition to the volume of AAV-RPGR^{ORF15} that will be administered to the subject (i.e., volume of the composition that is used to prime the device, but cannot be injected or recovered).

To improve the EZ, retinal sensitivity, visual acuity, retinal thickness or ONL thickness, or a combination thereof across the left-right axis of the visual field, according to some embodiments of the methods of the disclosure, an AAV-RPGR^{ORF15} composition of the disclosure may be administered by a suprachoroidal route to at least one focal position on the left half of the retina and to at least one focal position on the right half of the retina of the eye to improve the retina's ability, and, consequently, the subject's visual system to use the improved visual acuity in these two areas to comparatively differentiate light sources, and therefore, improve vision. This principle applies to any axis of the visual field, including, generally top versus bottom halves of the visual field and left versus right halves of the visual field.

With greater precision, should the retina be partitioned into at least two parts, in some embodiments of the methods of the disclosure, an AAV-RPGR^{ORF15} composition of the disclosure may be administered by a suprachoroidal route to at least one focal position in a first part of the retina and to at least one focal position in a second part of the retina. Preferably, the at least one focal position in a first part of the retina and the at least one focal position in a second part of the retina lie on opposite sides of the retina, which could be connected by a theoretical line that bisects a center of the retina. In some embodiments, the center of the retina is the center of a circle overlaid upon an image of the retina wherein the circle comprises 360 degrees. In some embodiments, the center of the retina is the fovea of the retina, wherein the retina is either physically flattened or theoretically flattened by merging one or more photographs. In some embodiments, including those wherein the center of the retina is the center of a circle overlaid upon an image of the retina wherein the circle comprises 360 degrees, the retina may be partitioned into between 1 and 360 parts, inclusive of the endpoints, the AAV-RPGR^{ORF15} composition may be administered by a suprachoroidal route to at least one focal position in a first part of the retina and to at least one focal position in a second part of the retina, and the first and second parts of the retina are directly opposite of one another on the circle (e.g., 0° and 180° or 90° and 270°). In some embodiments, including those wherein the center of the retina is the center of a circle overlaid upon an image of the retina wherein the circle comprises 360 degrees, the retina may be partitioned into between 1 and 360 parts, inclusive of the endpoints, the AAV-RPGR^{ORF15} composition may be administered by a suprachoroidal route to at least one focal position in a first part of the retina and to at least one focal position in a second part of the retina, and the first and second parts of the retina are opposite of one another on the circle within a range of positions (e.g., 0-30° and 180-210° or 90-120° and 270-300°).

In some embodiments of the methods of the disclosure, the AAV-RPGR^{ORF15} composition of the disclosure may be administered by a suprachoroidal route to at least one pair of opposed positions of the retina. In some embodiments, the gene therapy vector of the disclosure may be administered by a suprachoroidal route to at least 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 120, 140, 140, 160, 180 or any number in between of pairs of opposed positions of the retina.

In some embodiments of the methods of the disclosure, including those wherein the AAV-RPGR^{ORF15} composition of the disclosure may be administered by a suprachoroidal route to at least one pair of opposed positions of the retina, the dose provided at the first position and the dose provided at the second position of the pair are identical.

In some embodiments of the methods of the disclosure, including those wherein the AAV-RPGR^{ORF15} composition of the disclosure may be administered by a suprachoroidal route to at least one pair of opposed positions of the retina, the dose provided at the first position and the dose provided at the second position of the pair are not identical. In some embodiments, the dose provided at the first position and the dose provided at the second position of the pair comprises varying injection or infusion volumes. In some embodiments, the dose provided at the first position comprises a greater volume that the dose provided at the second position of the pair. In some embodiments, the dose provided at the second position comprises a greater volume that the dose provided at the first position of the pair. In some embodiments, the dose provided at the first position and the dose provided at the second position of the pair comprises varying concentrations of the AAV-RPGR^{ORF15} composition. In some embodiments, the dose provided at the first position comprises a greater concentration that the dose provided at the second position of the pair. In some embodiments, the dose provided at the first position comprises a greater concentration that the dose provided at the second position of the pair. In some embodiments, the dose provided at the second position comprises a greater concentration that the dose provided at the first position of the pair.

In some embodiments of the methods of the disclosure, including those wherein the AAV-RPGR^{ORF15} composition of the disclosure may be administered by a suprachoroidal route to at least two pairs of opposed positions of the retina, the doses provided to the first pair of opposed positions and the dose provided to the second pair of opposed positions are identical.

In some embodiments of the methods of the disclosure, including those wherein the AAV-RPGR^{ORF15} composition of the disclosure may be administered by a suprachoroidal route to at least two pairs of opposed positions of the retina, the doses provided to the first pair of opposed positions and the dose provided to the second pair of opposed positions are not identical. In some embodiments, the doses provided to the first pair of opposed positions and the dose provided to the second pair of opposed positions comprise varying injection or infusion volumes. In some embodiments, the dose provided to the first pair of opposed positions comprises a greater volume that the dose provided to the second pair of opposed positions. In some embodiments, the dose provided to the second pair of opposed positions comprises a greater volume that the dose provided to the first pair of opposed positions. In some embodiments, the doses provided to the first pair of opposed positions and the dose provided to the second pair of opposed positions comprise varying concentrations of the gene therapy concentrations. In some embodiments, the dose provided to the first pair of opposed positions comprises a greater concentration than the dose provided to the second pair of opposed positions. In some embodiments, the dose provided to the second pair of opposed positions comprises a greater concentration than the dose provided to the first pair of opposed positions.

### Suprachoroidal Devices

Suprachoroidal administration may be performed using a standard small gauge needle. However, specialized devices for suprachoroidal administration are also contemplated.

Microneedles

Microneedles may be used for administration to subjects of any age, however, microneedles may be particularly useful for the delivery of a composition of the disclosure to a child (a pediatric patient) due to the smaller dimensions of the anatomy.

Microneedles of the disclosure may include a bevel, which allows for ease of penetration into the sclera and/or suprachoroidal space with minimal collateral damage. The beveled surface of the microneedle defines a tip angle of less than about 20 degrees and a ratio of a bevel height to a bevel width of less than about 2.5. The beveled microneedle, in one embodiment, allows for accurate and reproducible drug delivery to the suprachoroidal space of the eye.

In some embodiments, a microneedle has a first end and a second end, the space between which defines a lumen. The first end of the microneedle may include a beveled surface. The beveled surface defines a first bevel angle and a second bevel angle different from the first bevel angle. In some embodiments, the first bevel angle is less than the second bevel angle. In some embodiments, the first bevel angle is less than about 20 degrees and the second bevel angle is less than about 30 degrees.

In some embodiments, the microneedles of the disclosure can define a narrow lumen (e.g., gauge size greater than or equal to 30 gauge, 32 gauge, 34 gauge, 36 gauge, etc.) to allow for suprachoroidal drug delivery while minimizing the diameter of the channel formed by the piercing of the sclera by the microneedle. In some embodiments, the lumen and bevel aspect ratio of the microneedles of the disclosure are distinct from standard small gauge needles (e.g., 27 gauge and 30 gauge needles) used for other routes of intraocular injection. For example, the microneedles included in the embodiments described herein can be any of those described in International Patent Application Publication No. WO2014/036009, U.S. Pat. Nos. 9,636,253, 9,788,995, 8,808,225, and 8,197,435 (the contents of which are each herein incorporated by reference in their entirety).

Cannula

In some embodiments, the microdelivery device comprises or consists of a cannula, and the hollow first end of the microdelivery device comprises or consists of a needle. The cannula may comprise an elongated tubular lumen. The elongated tubular lumen may further comprise a force element such as a spring or gas reservoir that provides a force to advance or deploy the cannula through the lumen and out from a hollow first end of the needle. Alternatively or in addition, the force element may provide a force to flow the gene therapy composition through the hollow first end of the needle and/or the cannula.

The force element may be mechanically coupled to the cannula by a push rod or plunger between the push rod and the cannula. Alternatively, the end of the force element may be directly mated to a section of the cannula. The force element, force element plunger or force element push rod may be connected to the cannula by an interfacing sleeve or other forms of attachment.

Prior to use, the first end of the cannula is within the needle and body of the microdelivery device. The cannula is configured to extend from the hollow first end of the needle once deployed by the force element. The cannula has a length to allow extension of the distal end of the cannula from the distal tip of the needle when deployed. The cannula is configured with a deployed length from the hollow first end of the needle to the intended site of delivery of the gene therapy composition. In one embodiment, the length of the cannula from the hollow first end of the needle in the deployed state ranges from 2 to 15 mm. A very short length deployed cannula is useful for directing the material for administration in a preferred direction from the needle penetration site. In particular, a deployed length from the distal tip of the needle in the range of 6 to 12 mm allows the cannula to be introduced in the eye at the pars plana to avoid potential damage to the retina and place the distal tip of the cannula near the posterior retina to deliver a material for administration to the most visually important portion of the eye.

The cannula is sized with a diameter less than or equal to the inner diameter of the needle lumen and is slidably disposed in the needle lumen. The cannula has a second end to receive the gene therapy composition and a first end to deliver the gene therapy composition. In one embodiment, the first end of the cannula is configured with a rounded profile to provide for an atraumatic tip for entering a tissue (e.g., an outer and/or inner surface of a sclera of an eye).

The size of the reservoir may be configured appropriately for the volume of composition to be delivered. The reservoir may be sized for delivery volumes ranging from, for example, 0.1 microliters to 1000 microliters. The compositions of the disclosure may be delivered manually by a plunger or by actuation of a force element acting on a plunger to move the plunger in the reservoir and provide a delivery force on the material for administration. For small volumes of administration, the lumen of the cannula may also act as a reservoir for the gene therapy composition. For small volumes of administration, the lumen of the cannula may also act as a reservoir for the gene therapy composition and a plunger may be configured to move distally in the lumen of the cannula to provide a delivery force on the material for administration.

In one embodiment, the deployment force is activated immediately after or simultaneous with advancement of the first end of the needle into a tissue (piercing of an outer surface of the sclera). The activation may be performed by release of the force element by the user or by a mechanism at the first end of the device.

In one embodiment, the microdelivery device also comprises a tissue interface with a seal secured to the first end of the microdelivery device thereby sealing the needle lumen during application of the deployment force. The distal seal is penetrable by the first end of the needle by the application of pressure on the tissue surface with the first end of the cannulation device and the penetrated tissue interface becomes slidable on the needle to allow advancement of the needle into tissue. Penetration of the seal opens a path for delivery of the cannula from the first end of the needle. The cannulation device with a force element is activated prior to or simultaneous with penetration of the seal by the needle and advancement of the first end of the needle into an outer surface of the sclera. The resulting self-actuating deployment mechanism ensures opening of the delivery path for the cannula immediately when the needle is placed on or in a tissue, regardless of the orientation and speed of needle insertion (e.g., piercing). The self-actuation mechanism enables simple one-handed operation of the cannulation device to administer the cannula to the suprachoroidal space of an eye.

In one embodiment, the tissue interface and seal are mounted on a tubular housing. The tubular housing is fit to the exterior of the needle and may be sealed to the surface of the needle at some point along its length. In one embodiment the housing may be sealed by means of an elastomeric element which is compressed between the housing and the needle. The elastomeric element may therefore be annular. In one embodiment, the elastomeric element may be compressed between the housing and the body of the device. The elastomeric element may reside at or near the proximal end of the housing. In one embodiment the elastomeric element serves as a seal between the housing and the needle. In one embodiment the elastomeric element serves as a frictional element or component which limits the housing travel in the proximal direction to thereby apply a force against the tissue surface by the tissue interface as the needle penetrates the tissues. In some embodiments, the distal element comprises a tissue interface and a distal seal and is slidably attached to the exterior of the needle without a distal housing.

Once the path from the first end the needle lumen is opened by needle penetration of the seal and insertion into the eye, the cannula cannot extend or deploy from the first end of the needle until a space to accept the cannula is reached by the distal end of the needle. Scleral tissue in particular is very resilient and effectively seals the needle tip during passage of the needle tip to the suprachoroidal space, hence the unique properties of the sclera do not allow for the cannula to enter the sclera. Once an underlying space such as the suprachoroidal space is reached by the first end of the needle, the cannula is able to advance out of the needle and be deployed into the space. By this mechanism the cannula is directed to a location that can accept the cannula at the first end of the needle. Subsequent to the deployment of the cannula, a composition of the disclosure may be delivered through the lumen of the cannula to the eye.

The flexible cannula of the cannulation device is designed with the appropriate mechanical properties with suitable flexural modulus to allow the cannula to bend to advance into the suprachoroidal space and with a suitable axial compressive stiffness to allow advancement of the cannula into the space by the deployment force on a proximal segment of the cannula. The mechanical properties can be suitably tailored by the selection of the cannula material and the cannula dimensions. In addition, the cannula may have features to tailor the mechanical properties. A stiffening element such as a wire may be placed in the lumen or wall of the cannula to increase axial buckling strength. The first tip of the cannula may also be reinforced for example with a coil or coating to tailor both the buckling strength and flexibility of the distal portion of the cannula. The coil can be fabricated from metal or high modulus polymers and placed on the outer surface of the cannula, the inner surface of the cannula or within the wall of the cannula. The cannula may be fabricated from polymers such as polyether block amide (PEBA), polyamide, perfluoroalkoxy polymer, fluorinated ethylenepropylene polymer, ethylenetetrafluoroethylene copolymer, ethylene chlorotrifluoroethylene copolymer polystyrene, polytetrafluoroethylene, polyvinylidene, polyethylene, polypropylene, polyethylene-propylene block copolymers, polyurethane, polyethylene terephthalate, polydimethylsiloxane, polyvinylchloride, polyetherimide and polyimide. For some applications, the cannula may be fabricated from a flexible metal such as a nickel titanium super elastic alloy (nitinol).

The delivery of the compositions of the disclosure may be aided by the tissue interface. The tissue interface may optionally apply a force to the surface of the eye to aid sealing of the at least one channel at the outer surface of the sclera to prevent reflux of the gene therapy composition. With an appropriate needle length and orientation, the microdelivery device may be used to deploy a cannula and deliver compositions of the disclosure into the suprachoroidal space.

In some embodiments of the disclosure, the needle comprises a stiff material, with a diameter to allow the cannula to pass through the lumen of the needle, typically in the range of 20 gauge to 40 gauge (for example, less than 0.91 mm outer diameter/0.6 mm inner diameter), where the length of the needle is suitable to reach the outer surface of the sclera of the eye. The needle is fixed to the body or barrel of the device and generally does not slide or move in relation to the body to provide precise control of needle depth during penetration of tissues.

The hollow first end of the needle may be beveled or sharpened to aid penetration. The bevel angle may be designed to facilitate entry into a specific target. For example, a short bevel of 18 degree bevel angle may be used to cannulate into narrower spaces. A medium bevel needle of 15 degree bevel angle may be used to cannulate into spaces such as the suprachoroidal space. Longer bevels, such as 12 degree bevel angle may be used to cannulate into the anterior or posterior chambers of the eye.

The needle may be constructed from a metal, ceramic, high modulus polymer or glass. The length of the needle in tissue is selected to match the target location for the cannulation and the variation in target location due to anatomical variability. The effective full length of the needle is the length of the first end of the needle the surface of the tissue interface. The tissue interface moves slidably on the needle during needle advancement into tissue, allowing for progressive increase in the length of needle protruding through the tissue interface and seal during advancement into tissue. The cannula is deployed automatically once the needle reaches the appropriate location which may be less than the effective full length of the needle. The release of force and resultant time for deployment occurs quickly, in approximately 0.1 to 3 seconds depending on the deployed length of the cannula and the amount of force from the force element. The time for deployment may also be controlled by a damping or frictional mechanism coupled to advancement of the cannula to limit the speed of cannula advancement or deployment. The release of force from the force element communicates to the physician with both visible and tactile feedback that there is no need for additional advancement of the needle. The rapid deployment event gives the physician sufficient time to halt needle advancement, resulting in an effective variable needle length to accommodate patient to patient differences in tissue thickness. The variable needle length and self-actuation of deployment is especially useful for cannulation into spaces that are not normally open, such as the suprachoroidal space. For the suprachoroidal space, the needle effective full length is in the range of 1 mm to 4 mm depending on the angle of insertion. The effective full needle length may, for example, be 0.3 mm to 3 mm, 0.35 to 2 mm, 1 mm to 4 mm, 10 to 15 mm.

In some embodiments of the disclosure, the micodelivery device comprises a means for providing a deployment force to the cannula. In some embodiments of the disclosure, the device comprises a means for providing a force to deliver gene therapy composition from a reservoir within the device. The means as described herein could be, for example, a compressible reservoir or levers that can be “squeezed” or compressed by a user (directly or indirectly) to effect deployment of the cannula or delivery of the material for administration. Alternatively, in one embodiment, the means is a mechanism with a biasing means or force element (such as a compression spring or a pressurized gas).

The device may be disposable and/or for single use. Alternatively, the device may be reusable.

Additional cannulation devices contemplated for use by the methods of the disclosure are described in, for example WO 2017/158366 (the contents of which are incorporated by reference herein in their entirety).

Microcatheters

In some embodiments of the disclosure, the microdelivery devices comprise a microcatheter. Microcatheters of the disclosure are similar to microcannulae of the disclosure, however, the microcatheter may pierce the outer surface of the sclera and contact the suprachoroidal space prior to extending an inner tip further into the suprachoroidal space to deliver a gene therapy composition to a target location.

Illustrative microcatheters of the disclosure include, but are not limited to, an iTrack™ 250A microcatheter (iScience Interventional, Menlo Park, Calif.) optionally connected to the iLumin™ laser-diode based micro-illumination system (iScience Interventional, Menlo Park, Calif.) (see, for example, Peden et al. (2011) PLoS One 6(2): e17140).

### Two-Step Injection

An AAV-RPGR^{ORF15} composition of the disclosure may be administered by a two-step procedure. Injection of the AAV-RPGR^{ORF15} composition is performed by an appropriately qualified and experienced retinal surgeon. For example, for injection of the composition into a subretinal space via a suprachoroidal route, the retina may first be detached from the choroid (which can be extremely thin and fused in places). This involves performing the composition delivery in 2 steps. An advantage of a 2-step procedure is that any unexpected complications of retinal detachment can be managed conservatively, minimizing concerns about the composition escaping into the vitreous. Since the volume of fluid required to detach the fovea is variable, by removing the vector from the first step, a precise consistent dose in terms of genome particles can still be applied into the sub-retinal space.

Initially, subjects undergo a detachment of the posterior hyaloid in the respective study eye. The retina may be detached with, for example, 0.1-0.5 mL of balanced salt solution (BSS) injected into the subretinal space (forming a “bleb”). At least one dose of the AAV-RPGR^{ORF15} composition may be injected into the sub-retinal fluid through the same entry site.

In the second step of the procedure, the AAV-RPGR^{ORF15} composition is prepared for injection. At least one dose of the AAV-RPGR^{ORF15} composition is injected into the sub-retinal space through the same entry site and into the bleb. Delivery to the subretinal space can targets any area of the macula (including multiple areas of the macula) but also include the fovea if possible. In each case, the vector is injected so that the sub-retinal fluid overlies all edge boundaries of the central region that has yet to undergo chorioretinal degeneration, as identified by fundus autofluorescence.

In other embodiments, the two step procedure is used to deliver a AAV-RPGR^{ORF15} composition to a suprachoroidal space by first injecting a sufficient amount of a buffer or other liquid to generate a “bleb” or to expand a compact space, and in step 2, to inject the gene therapy composition into the bleb or into the expanded space created by the introduction of additional liquid.

For delivery to any portion of the eye via a suprachoroidal approach, the AAV-RPGR^{ORF15} composition may be delivered by, for example, a microneedle, a microcannula, or a microcatheter. In some embodiments, the gene therapy composition may be delivered by a microcatheter.

### Corticosteroids

In some embodiments of the methods of the disclosure, a course of corticosteroid (e.g., oral corticosteroid) can be administered to a subject before, during and/or after administration of a AAV-RPGR^{ORF15} composition. For example, a 21-day course of corticosteroid may be started 2 days, or 3 days, before the date of administration of the AAV-RPGR^{ORF15} composition. In some embodiments, oral corticosteroid is administered for about 9 weeks (e.g., 21 days at 60 mg, followed by six weeks of tapering doses). In some embodiments, the corticosteroid is tetriamcinolone, prednisolone and/or prednisone. The corticosteroid may reduce inflammation resulting from the surgery and/or the vector/transgene. Alternatively, or in addition to this corticosteroid, a subject can be administered triamcinolone at or about the time of surgery, e.g., via a deep sub-Tenon approach. In some embodiments, up to about 1 mL of triamcinolone is administered at or about the time of surgery. In some embodiments, the concentration of the administered triamcinolone is 10 mg/mL to 200 mg/mL, 20 mg/mL to 100 mg/mL, or about 30 mg/mL, about 40 mg/mL, or about 50 mg/mL. In one embodiment, up to or about 1 mL of triamcinolone at a concentration of about 40 mg/mL is administered to the subject at or about the time of surgery.

### The Ellipsoid Zone

The ellipsoid zone (EZ) is a structure at the photoreceptor inner segment/outer segment (IS/OS) boundary in the retina. In subjects with Retinitis Pigmentosa, the EZ degenerates and decreases in width when measured along the anterior to posterior axis of the eye. In subjects with Retinitis Pigmentosa, the EZ is a marker of the usable visual field of the retina, as its disappearance marks the border between healthy and diseased retina as Retinitis Pigmentosa progresses. Without wishing to be bound by theory, the degradation of the EZ in subjects with Retinitis Pigmentosa may arise as a result of decreasing numbers of photoreceptors, decreasing numbers of cilia in the photoreceptors, or a combination thereof. Mutations in the RPGR gene account for 70-90% of the X-linked form of RP (XLRP), with the ORF15 isoform of RPGR expressed in the photoreceptors. The outer segments of the photoreceptors, whose junction with the photoreceptor inner segment is the EZ, contain specialized sensory cilia. These sensory cilia are critical for photoreceptor function, and therefore vision. RPGR^{ORF15} localizes to photoreceptor receptor cilia, and the retinal degeneration observed in subjects with Retinitis Pigmentosa includes ciliary defects. In addition, RPGR is also implicated in protein trafficking at the photoreceptor outer segment, which is important for photoreceptor viability. EZ width or EZ area is thus a valuable objective, clinical measurement that can be used to assess the efficacy of therapies for the treatment of Retinitis Pigmentosa.

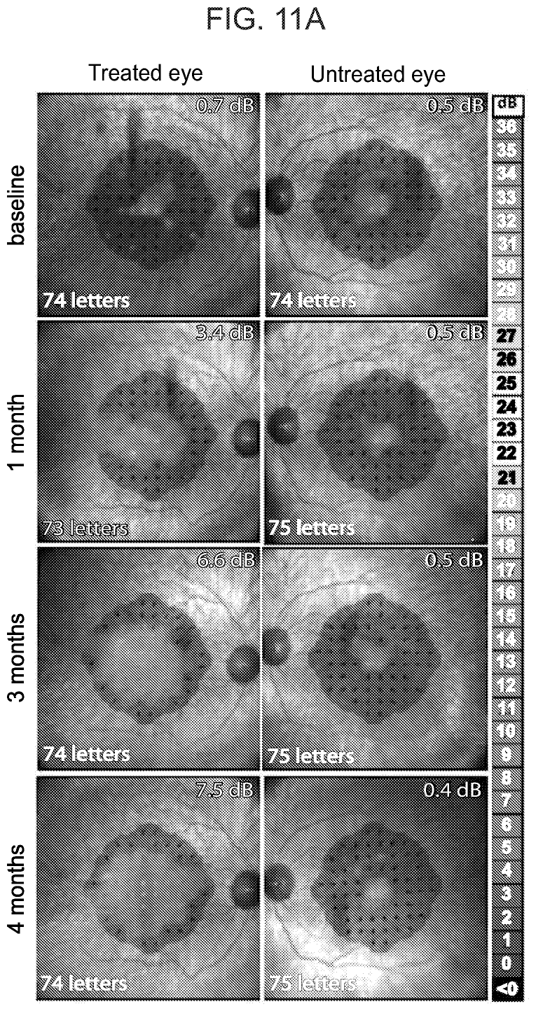

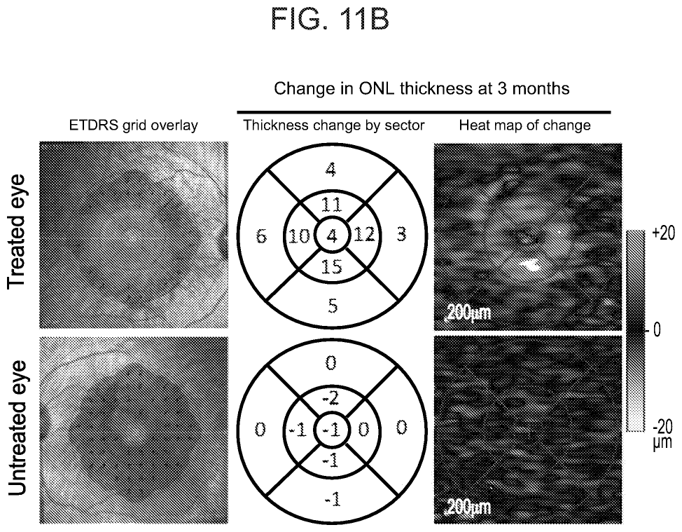

The disclosure provides a method of treating Retinitis Pigmentosa in a subject in need thereof, comprising administering to the subject a therapeutically effective amount of an AAV-RPGR^{ORF15} composition of the disclosure. In some embodiments, administering to the subject the therapeutically effective amount of the AAV-RPGR^{ORF15} composition improves a sign or a symptom of Retinitis Pigmentosa. In some embodiments, the sign of Retinitis Pigmentosa comprises a degeneration of the ellipsoid zone (EZ). In some embodiments, the degeneration of the EZ comprises a reduction in photoreceptor cell density, a reduction in number of photoreceptor cilia, or a combination thereof. In some embodiments, degeneration of the EZ can be measured as a reduction of the width of the EZ along the anterior to posterior (A/P) axis in a transverse view of an OCT z-stack centered on the fovea of the eye. In some embodiments, degeneration of the EZ comprises degeneration in one or more sectors of the eye along the dorsoventral and mediolateral axes. An example of a sectored eye can be seen in FIG. 11B.

In some embodiments of the methods of the disclosure, the subject has detectable degeneration of the EZ when compared to a control EZ. In some embodiments, the control EZ comprises an EZ from a healthy individual, who is age and gender matched to the subject, as the thickness of the EZ can vary with age and gender in healthy subjects. In some embodiments, the control EZ comprises an average of measurements of multiple EZs from individuals who are age and gender matched to the subject. In some embodiments, the subject's EZ on SD-OCT before administration of a therapeutically effective amount of the AAV-RPGR^{ORF15} composition is within the nasal and temporal border of any B-scan and is not visible on the most inferior and superior B-scan.

In some embodiments of the methods of the disclosure, administering a therapeutically effective amount of the AAV-RPGR^{ORF15} composition restores the EZ of the subject who has detectable degeneration of the EZ. In some embodiments, restoring the EZ comprises increasing the number of photoreceptors, the numbers of cilia, or a combination thereof. In some embodiments, restoring the EZ comprises increasing the width of the EZ after administration of an AAV-RPGR^{ORF15} composition. In some embodiments, this increase in width is an increase to the width of a normal EZ zone (i.e. to fully healthy EZ from a control subject). In some embodiments, the width of the EZ zone is partially restored. In some embodiments, the increase in the width of the EZ comprises an increase in width to at least 10%, 20%, 30%, 40%, 50%, 60%, 70%, 80% or 90% of the width of a healthy EZ. In some embodiments, restoring the EZ comprises increasing the area of the EZ after administration of an AAV-RPGR^{ORF15} composition. In some embodiments, this increase in area is an increase to the area of a normal EZ zone (i.e. to fully healthy EZ from a control subject). In some embodiments, the area of the EZ zone is partially restored. In some embodiments, the increase in the area of the EZ comprises an increase in area to at least 10%, 20%, 30%, 40%, 50%, 60%, 70%, 80% or 90% of the area of a healthy EZ.

In some embodiments of the methods of the disclosure, administering a therapeutically effective amount of the AAV-RPGR^{ORF15} composition induces regeneration of photoreceptor outer segments. Without wishing to be bound by theory, regeneration of photoreceptor outer segments may be linked to genetic restoration of ciliary trafficking. In some embodiments, re-emergence of the EZ over areas of previously degenerate macula on OCT after administration of a therapeutically effective amount of the AAV-RPGR^{ORF15} composition may be linked to regeneration of photoreceptor outer segments. In some embodiments, administering a therapeutically effective amount of the AAV-RPGR^{ORF15} composition induces retinal thickening and/or ONL thickening as visualized by OCT.

Increases in width can be measured by comparing the width of the EZ prior to administration of an AAV-RPGR^{ORF15} composition of the disclosure (a ‘baseline’ measurement) to the width of the EZ after administration of an AAV-RPGR^{ORF15} composition. In some embodiments, the width of the EZ is measured at baseline, and at least at one of 1 week, 1 month, 2 months, 3 months, 4 months, 6 months, 9 months or 12 months after administration of an AAV-RPGR^{ORF15} composition. In some embodiments, the width of the EZ is measured at baseline, and at 1 month after administration of an AAV-RPGR^{ORF15} composition. In some embodiments, the width of the EZ is measured at baseline, and at 3 months after administration of an AAV-RPGR^{ORF15} composition. In some embodiments, the width of the EZ is measured at baseline, and at 1 month, at 3 months and at 4 months after administration of an AAV-RPGR^{ORF15} composition of the disclosure.

In some embodiments, restoring the EZ comprises increasing the width of the EZ when the width of the EZ after administration of an AAV-RPGR^{ORF15} composition is compared to the EZ at baseline. In some embodiments, increasing the width of the EZ comprises an increase in width along the A/P axis of 1 to 20 μm, inclusive of the endpoints. In some embodiments, increasing the width of the EZ comprises an increase in width along the A/P axis of 3-15 μm, inclusive of the endpoints. In some embodiments, increasing the width of the EZ comprises an increase in width along the A/P axis of at least 1 μm.

In some embodiments, restoring the EZ comprises increasing the width of the EZ, when the width of the EZ after administration of an AAV-RPGR^{ORF15} composition is compared to the width of the EZ at baseline. In some embodiments, the increase in width of the EZ along the A/P axis is uniform across more than one sector of the eye. In some embodiments, the increase in width of the EZ along the A/P axis is non-uniform across more than one sector of the eye.

In some embodiments, restoring the EZ comprises increasing the area of the EZ when the area of the EZ after administration of an AAV-RPGR^{ORF15} composition is compared to the EZ at baseline. In some embodiments, increasing the area of the EZ comprises an increase in area of 0.8 to 324 μm², inclusive of the endpoints. In some embodiments, increasing the area of the EZ comprises an increase in area of 7-180 μm², inclusive of the endpoints. In some embodiments, increasing the area of the EZ comprises an increase of at least 0.8 μm².

In some embodiments, restoring the EZ comprises increasing the area of the EZ, when the area of the EZ after administration of an AAV-RPGR^{ORF15} composition is compared to the area of the EZ at baseline. In some embodiments, the increase in area of the EZ is uniform across more than one sector of the eye. In some embodiments, the increase in area of the EZ is non-uniform across more than one sector of the eye.

In some embodiments, administering the therapeutically effective amount of an AAV-RPGR^{ORF15} composition inhibits further degeneration of the EZ when the EZ after administration of the composition is compared to the EZ at baseline. In those embodiments wherein administering the therapeutically effective amount of an AAV-RPGR^{ORF15} composition inhibits further degeneration of the EZ, there is no change in the width of the EZ when measurements at baseline and after administration of the AAV-RPGR^{ORF15} composition are compared.

In some embodiments, changes in the thickness of the EZ correlate with changes in retinal sensitivity. For example, increases in the width of the EZ in subjects with Retinitis Pigmentosa are positively correlated with increases in retinal sensitivity.

### Optical Coherence Tomography

In some embodiments of the methods of the disclosure, the EZ, retinal thickness and/or ONL thickness is imaged using optical coherence tomography (OCT). OCT is an imaging technique that uses coherent light to capture micrometer resolution, two and three dimension images of the eye. In some embodiments, OCT imaging captures z-stack of images that comprises an area of the eye centered on the fovea. The x-y plane of the images are along the dorventral and mediolateral axes of the eye. The z-stack of images are then imported into processing software (for example Heidelberg Eye Explorer, version 1.9.10.0; Heidelberg Engineering) to generate 3-dimensional and transverse views. In some embodiments, the boundaries of the EZ are manually delineated in the transverse view of the retina. In some embodiments, the maximal width of the EZ in the transverse view is measured. In some embodiments, the maximal width of the EZ in the transverse view is measured manually. In some embodiments, EZ area is measured from a series of B scans (the number depends on how many are taken) and then the area is calculated. In some embodiments, EZ area is measured by an en face methodology.

In some embodiments, OCT (e.g. spectral domain OCT or SD-OCT) can be performed prior to administration of the AAV-RPGR^{ORF15} composition (at “baseline”), and at about 3 months, at about 6 months, at about 12 months, at about 18 months and/or at about 24 months after administration of the AAV-RPGR^{ORF15} composition. The measurements after administration can be compared to the baseline measurement to see if the EZ measurement, retinal thickness and/or ONL thickness via OCT imaging improves following administration of the AAV-RPGR^{ORF15} composition.

### Perimetry

Microperimetry combines fundus imaging, retinal sensitivity mapping and fixation analysis. Retinal images are acquired by scanning laser ophthalmoscopy (SLO) and an eye tracker compensates for eye movements in real time. Illustrative microperimetry systems include MAIA (CenterVue SpA, Padova, Italy). Illustrative automated static perimetry systems include Octopus 900 (Haag-Streit Diagnostics, Bern, Switzerland).

In some embodiments, microperimetry can be measured prior to administration of the AAV-RPGR^{ORF15} composition (at “baseline”), and at about 3 months, at about 6 months, at about 12 months, at about 18 months and/or at about 24 months after administration of the AAV-RPGR^{ORF15} composition. The measurements after administration can be compared to the baseline measurement to see if microperimetry improves following administration of the AAV-RPGR^{ORF15} composition.

### Retinal Sensitivity

Retinal sensitivity is the minimum light level perceptible to a subject. Retinal sensitivity across areas of the retina is measured using perimetry (e.g., microperimetry and/or automated static perimetry). In some embodiments, a scanning laser ophthalmoscope (SLO) is used to create a high resolution image of the retina. A grid of point stimuli is then projected onto a region of the retina in the SLO image, and the patient's response to each stimulus at each point of the grid is measured to determine the minimum perceptible stimulus at that position.

In some embodiments of the compositions and methods of the disclosure for performing microperimetry, including those wherein the microperimetry is performed using a MAIA device, the grid comprises at least 30 points. In some embodiments, the grid is a 37 point grid. In some embodiments, the grid is a 68 point grid. In some embodiments, the size of the stimulus is Goldmann III (a diameter of 0.43° of the visual range). In some embodiments, the background luminance is 4 apostilb (asb). In some embodiments, the maximum luminance applied as a stimulus is about 1000 asb. In some embodiments, the region of the eye assayed comprises all or a part of the macula. In some embodiments, the region of the eye assayed is the macula. In some embodiments, the region assayed is a 10° diameter area of the eye within the macula. In some embodiments, the region assayed is a 10° diameter area of the eye centered on the fovea.

In some embodiments of the compositions and methods of the disclosure for performing perimetry, including those wherein the automated static perimetry is performed using an Octopus 900 device, the grid comprises at least 30 points. In some embodiments, the grid is a 37 point grid. In some embodiments, the grid is a 68 point grid. In some embodiments, the size of the stimulus is Goldmann III (a diameter of 0.43° of the visual range). In some embodiments, the background luminance is 4 apostilb (asb). In some embodiments, the maximum luminance applied as a stimulus is about 1000 asb. In some embodiments, the region of the eye assayed comprises all or a part of the macula. In some embodiments, the region of the eye assayed is the macula. In some embodiments, the region assayed is a 10° diameter area of the eye within the macula. In some embodiments, the region assayed is a 10° diameter area of the eye centered on the fovea.

In some embodiments of the compositions and methods of the disclosure for performing microperimetry, including those wherein the microperimetry is performed using a MAIA device, stimulus luminance is measured in apostilbs (asb). Asbs are absolute units of luminance, and each asb is equal to 0.3183 candela/m². The decibel (dB) scale is a log 10 based scale used to report the dynamic range of the stimuli used in a retinal sensitivity assessment. In some embodiments, the minimum and maximum stimulus intensities delivered by a microperimetry instrument are set to 36 dB and 0 dB, respectively, and the dB scale between these values is calculated. In some embodiments, dB reporting is color coded, and black represents no response (scotoma), red is abnormal, yellow is suspect, and green is normal.

In some embodiments of the compositions and methods of the disclosure for performing perimetry, including those wherein the perimetry is performed using an Octopus 900 device, stimulus luminance is measured in apostilbs (asb). Asbs are absolute units of luminance, and each asb is equal to 0.3183 candela/m². The decibel (dB) scale is a log 10 based scale used to report the dynamic range of the stimuli used in a retinal sensitivity assessment. In some embodiments, the minimum and maximum stimulus intensities delivered by a perimetry instrument are set to 47 dB and 0 dB, respectively, and the dB scale between these values is calculated. In some embodiments, dB reporting is color coded, and black represents no response (scotoma), red is abnormal, yellow is suspect, and green is normal.

In order to measure retinal sensitivity, various stimulus projection strategies can be used. In some embodiments, each stimulus at each point is delivered repeatedly in 4 dB increasing steps until there is a change in response (e.g., from not seen to seen). In some embodiments, the stimulus then changes to 2 dB steps until there is another change in response (i.e. from seen to not seen). The threshold value for retinal sensitivity is the minimum value, in dB, at which a stimulus is seen by the subject when that stimulus is projected at increasing intensity onto a single point of the retina.

In some embodiments, the mean retinal sensitivity is the average of the threshold values in dB across all the points in the grid of point stimuli. In some embodiments, improvement in retinal sensitivity is observed in at least 3, 4, 5, 6, 7, 8, or 9 or the 16 central loci. In some embodiments, improvement in retinal sensitivity is observed in at least 5 of the 16 central loci.

The disclosure provides a method of treating Retinitis Pigmentosa in a subject in need thereof, comprising administering to the subject a therapeutically effective amount of an AAV-RPGR^{ORF15} composition of the disclosure. In some embodiments, administering to the subject the therapeutically effective amount of the AAV-RPGR^{ORF15} composition improves a sign or a symptom of Retinitis Pigmentosa. In some embodiments, the sign of Retinitis Pigmentosa comprises a loss of retinal sensitivity. In some embodiments, retinal sensitivity is measured with microperimetry. In some embodiments, measuring retinal sensitivity with microperimetry comprises (a) imaging the fundus of an eye of the subject; (b) projecting a grid of points onto the image the fundus of the eye of the subject; (c) repeatedly stimulating the eye at each point on the grid with a light stimulus, wherein each progressive stimulus is a greater intensity than the previous stimulus, and wherein the stimuli range from approximately 4 to 1000 apostilb (asb); (d) determining for each point on the grid a minimum threshold value, wherein the minimum threshold value is the intensity of light stimulus at which the subject can first perceive the stimulus; and (e) converting the minimum threshold value from asb to decibels (dB) on a dB scale, wherein a maximum stimulus is set to 0 dB and a minimum stimulus is set to the maximum dB value of the scale. In some embodiments, the maximum stimulus is about 1000 asb and is set to 0 dB, and the minimum stimulus is about 4 asb and is set to 36 dB. In some embodiments, the grid comprises or consists of 68 points. In some embodiments, the points are evenly spaced over a circle with a diameter that covers 10° of the eye. In some embodiments, the circle is centered on the macula. In some embodiments, the circle is centered on the fovea. In some embodiments, the microperimetry measurement of retinal sensitivity further comprises averaging the minimum threshold value measured at each point in the grid to produce a mean retinal sensitivity.

In some embodiments, the subject has a detectable loss of retinal sensitivity when compared to retinal sensitivity in a control subject. Control subjects are, for example, healthy subjects without Retinitis Pigmentosa who are age and gender matched to the subject.

In some embodiments of the methods of the disclosure, administering a therapeutically effective amount of an AAV-RPGR^{ORF15} composition restores the retinal sensitivity of the subject. Retinal sensitivity can be measured prior to administration of the AAV-RPGR^{ORF15} composition (at “baseline”), and after administration of the AAV-RPGR^{ORF15} composition, and the two measurements compared to see if retinal sensitivity improves following administration of the AAV-RPGR^{ORF15} composition. In some embodiments, restoring the loss of retinal sensitivity comprises an increase in mean retinal sensitivity when retinal sensitivity following administration of an AAV-RPGR^{ORF15} composition is compared to baseline retinal sensitivity. In some embodiments, the increase in mean retinal sensitivity comprises an increase of 1 to 30 decibels (dB), inclusive of the endpoints. In some embodiments, increase in mean retinal sensitivity comprises an increase of 1 to 15 dB, inclusive of the endpoints. In some embodiments, increase in mean retinal sensitivity comprises an increase of 2 to 10 dB, inclusive of the endpoints.

In some embodiments of the methods of the disclosure, restoring retinal sensitivity comprises an increase in threshold sensitivity at at least one point of the grid when retinal sensitivity after administration of an AAV-RPGR^{ORF15} composition is compared to retinal sensitivity at baseline. In some embodiments, the increase in threshold sensitivity at at least one point comprises an increase of between 1 to 36 decibels (dB), inclusive of the endpoints. In some embodiments, the increase in threshold sensitivity at at least one point comprises an increase of 1 to 15 decibels (dB), inclusive of the endpoints. In some embodiments, the increase in threshold sensitivity at at least one point comprises an increase of 2 to 10 decibels (dB), inclusive of the endpoints. In some embodiments, the increase in threshold sensitivity of at least 1 dB comprises an increase of at least 1 dB in between 1-68 points, inclusive of the endpoints. In some embodiments, the increase in threshold sensitivity of at least 1 dB comprises an increase of at least 1 dB in at least 2, at least 3, at least 4, at least 5, at least 10, at least 15, at least 20, at least at least 25, at least 30, at least 35, at least 40, at least 45, at least 50, at least 55, at least 60 or at least 65 points.

In some embodiments of the methods of the disclosure, restoring retinal sensitivity comprises an increase in the number of points with a threshold retinal sensitivity of at least 1 Db when retinal sensitivity after administration of an RPGR^{ORF15} composition of the disclosure is compared to retinal sensitivity at baseline. In some embodiments, the number of points with a threshold sensitivity greater than 1 dB increases by between 1 to 68 points, inclusive of the endpoints, after administration of AAV-RPGR^{ORF15}. In some embodiments, the number of points with a threshold sensitivity greater than 1 dB increases by at least 1 point, after administration of AAV-RPGR^{ORF15}. In some embodiments, the number of points with a threshold sensitivity greater than 1 dB increases by at least 15 points after administration of AAV-RPGR^{ORF15}. In some embodiments, the number of points with a threshold sensitivity greater than 1 dB increases by at least 20 points, after administration of AAV-RPGR^{ORF15}. In some embodiments, the number of points with a threshold sensitivity greater than 1 dB increases by at least 25 points, after administration of AAV-RPGR^{ORF15}. In some embodiments, an increase of at least 5 db at at least 5 loci in the central 16 loci is observed after administration of AAV-RPGR^{ORF15}. In some embodiments, an increase of at least 6 db at at least 5 loci in the central 16 loci is observed after administration of AAV-RPGR^{ORF15}. In some embodiments, an increase of at least 7 db at at least 5 loci in the central 16 loci is observed after administration of AAV-RPGR^{ORF15}. In some embodiments, an increase of at least 8 db at at least 5 loci in the central 16 loci is observed after administration of AAV-RPGR^{ORF15}.

In some embodiments of the methods of the disclosure, administering the therapeutically effective amount of an AAV-RPGR^{ORF15} composition inhibits any further loss of retinal sensitivity of the subject when retinal sensitivity after administration of the AAV-RPGR^{ORF15} composition is compared to retinal sensitivity at baseline.

Increases in retinal sensitivity can be measured by comparing retinal sensitivity prior to administration of an AAV-RPGR^{ORF15} composition of the disclosure (a ‘baseline’ measurement) to retinal sensitivity after administration of an AAV-RPGR^{ORF15} composition using microperimetry. In some embodiments, retinal sensitivity is measured at baseline, and at least at one of 1 week, 1 month, 2 months, 3 months, 4 months, 6 months, 9 months, 12 months, 18 months, 24 months or 3 years after administration of an AAV-RPGR^{ORF15} composition of the disclosure. In some embodiments, retinal sensitivity is measured at baseline, and at 1 month after administration of an AAV-RPGR^{ORF15} composition. In some embodiments, retinal sensitivity is measured at baseline, and at 3 months after administration of an AAV-RPGR^{ORF15} composition of the disclosure. In some embodiments, retinal sensitivity is measured at baseline, and at 1 month, at 3 months and at 4 months after administration of an AAV-RPGR^{ORF15} composition.

### Visual Field

The visual field is the total area of the eye in which objects can be seen when the eye is focused on a central point. The extent of the visual field can be determined through retinal sensitivity analysis. In some embodiments, the visual field is the portion of the area of the retina, as measured by perimetry, in which a response to a stimulus of at least 1 dB is measured.

### Fixation

Microperimetry can also measure fixation, or the process of attempting to look at a selected visual target, sometimes called a preferred retinal locus (PRL). In normal subjects, the fovea is the preferred area of the retina for fixation. When the fovea is affected, fixation degrades and subjects use extra-foveal regions. Fixation can be assessed by tracking eye movements, for example, 25 times a second and plotting the resulting distribution over the SLO image. The overall cloud of points describes the PRL.

### Fixation Stability

Microperimetry can also be used to measure fixation stability. Fixation stability can be measured two ways. First, fixation stability is measured by calculating the percentage of fixation points located within a distance of 1° or 2° respectively (P1 and P2) durating a fixation attempt. If more than 75% of the fixation points are located within P1, the fixation is classified as stable. If less than 75% of fixation points are located within P1, but more than 75% of fixation points are located within P2, the fixation is classified as relatively unstable. If less than 75% of fixation points are located within P2, the fixation is unstable. Second, an area of an ellipse which encompasses the cloud of fixation points for a given proportion based on standard divisions of the horizontal and vertical eye positions during the fixation attempt is calculated (the bivariate contour ellipse area).

### Visual Acuity

Visual acuity refers to sharpness of vision, and is measured by the ability to discern letters or numbers at a given distance according to a fixed standard. In some embodiments, visual acuity is measured while fixating, and is a measure of central, or foveal, visual acuity. Best-corrected visual acuity (BCVA) can be measured using the Early Treatment Diabetic Retinopathy Study (ETDRS) chart. EDTRS charts are charts with 5 letters per row of equal difficulty, whose spacing between and within rows decreases on a log scale. In some embodiments, BCVA testing comprises having the subject read down the chart (from largest to smallest letters) until reaching a row where a minimum of three letters cannot be read. In some embodiments, BCVA testing comprises having the subject read the smallest row of letters where all letters are discernable, and then continue until down the chart until reaching a row where a minimum of three letters cannot be read. In some embodiments, the BCVA score is calculated by determining the last row where the patient can correctly identify all 5 letters on the row, determine the log score for that row from the ETDRS chart, and subtracting 0.02 log units for every letter that is correctly identified beyond the last row where all of the letters are correctly identified.

In some embodiments, BCVA can be measured prior to administration of the AAV-RPGR^{ORF15} composition (at “baseline”), and at about 3 months, at about 6 months, at about 12 months, at about 18 months and/or at about 24 months after administration of the AAV-RPGR^{ORF15} composition. The measurements after administration can be compared to the baseline measurement to see if BCVA improves following administration of the AAV-RPGR^{ORF15} composition.

### Autofluorescence

To assess changes in the area of viable retinal tissue, fundus autofluorescence can be measured. In some embodiments, fundus autofluorescence can be recorded using a confocal scanning laser ophthalmoscope. In some embodiments, fundus autofluorescence can be measured prior to administration of the AAV-RPGR^{ORF15} composition (at “baseline”), and at about 3 months, at about 6 months, at about 12 months, at about 18 months and/or at about 24 months after administration of the AAV-RPGR^{ORF15} composition. The measurements after administration can be compared to the baseline measurement to see if fundus autofluorescence improves following administration of the AAV-RPGR^{ORF15} composition.

### Risk Factors

The disclosure provides a method of preventing Retinitis Pigmentosa in a subject at risk of developing Retinitis Pigmentosa, comprising administering to the subject a prophylactically effective amount of an AAV-RPGR^{ORF15} composition of the disclosure.

In some embodiments, the subject has one or more risk factors for Retinitis Pigmentosa. In some embodiments, the one or more risk factors comprise a genetic risk factor, a family history of Retinitis Pigmentosa or a symptom of Retinitis Pigmentosa.

Retinitis Pigmentosa is an inherited genetic disease. In X-linked Retinitis Pigmentosa (XLRP), the genetic mutations leading to the development of Retinitis Pigmentosa is on the X chromosome. XLRP is estimated to occur in approximately 1 in 15,000 people. Because XLRP is X-linked, a man whose grandfather had XLRP a 50% chance of inheriting a mutation associated with X-linked Retinitis Pigmentosa. Thus, in some embodiments, a risk factor for the development of Retinitis Pigmentosa is a family history of Retinitis Pigmentosa. A subject who has family history of Retinitis Pigmentosa can prevent the onset of XLRP through the administration of a prophylactically effective amount of an AAV-RPGR^{ORF15} composition of the disclosure.

In some embodiments, a risk factor for the development of Retinitis Pigmentosa comprises a genetic risk factor. Exemplary genetic risk factors for the development of Retinitis Pigmentosa include, but are not limited to mutations that cause XLRP (e.g., mutations in RPGR). Thus, in some embodiments of the methods of the disclosure, the development of Retinitis Pigmentosa may be prevented in a subject who has a mutation known to cause Retinitis Pigmentosa, such as a mutation in RPGR, through the administration of a prophylactically effective amount of an AAV-RPGR^{ORF15} composition of the disclosure.

In some embodiments, a risk factor for the development of Retinitis Pigmentosa comprises a symptom of Retinitis Pigmentosa. In some embodiments, the symptom of Retinitis Pigmentosa comprises loss of night vision, loss of peripheral vision, loss of visual acuity, loss of color vision or a combination thereof. Mild symptoms of Retinitis Pigmentosa may occur early on in the course of the disease, and occur prior to a diagnosis of Retinitis Pigmentosa. Thus, in some embodiments of the methods of the disclosure, the development of Retinitis Pigmentosa may be prevented in a subject who has a symptom associated with Retinitis Pigmentosa, such as a mild loss of night vision or peripheral vision, can prevent Retinitis Pigmentosa through the administration of a prophylactically effective amount of an AAV-RPGR^{ORF15} composition of the disclosure.

### Near Darkness Agility Maze

The baseline or improved visual acuity of a subject of the disclosure may be measured by having the subject navigate through an enclosure characterized by low light or dark conditions and including one or more obstacles for the subject to avoid. The subject may be in need of a composition of the disclosure, optionally, provided by a method of treating of the disclosure. The subject may have received a composition of the disclosure, optionally, provided by a method of treating of the disclosure in one or both eyes and in one or more doses and/or procedures/injections. The enclosure may be indoors or outdoors. The enclosure is characterized by a controlled light level ranging from a level that recapitulates daylight to a level that simulates complete darkness. Within this range, the controlled light level of the enclosure may be preferably set to recapitulate natural dusk or evening light levels at which a subject of the disclosure prior to receiving a composition of the disclosure may have decreased visual acuity. Following administration of a composition of the disclosure, the subject may have improved visual acuity and/or functional vision at all light levels, but the improvement is preferably measured at lower light levels, including those that recapitulate natural dusk or evening light levels (indoors or outdoors). Functional vision may be assessed, e.g., using a multi-luminance mobility test (MLMT), such as the described in Chung et al. Clin. Exp. Opthalmol. 46:247-59 (2018).

In some embodiments of the enclosure, the one or more obstacles are aligned with one or more designated paths and/or courses within the enclosure. A successful passage through the enclosure by a subject may include traversing a designated path and avoiding traversal of a non-designated path. A successful passage through the enclosure by a subject may include traversing any path, including a designated path, while avoiding contact with one or more obstacles positioned either within a path or in proximity to a path. A successful or improved passage through the enclosure by a subject may include traversing any path, including a designated path, while avoiding contact with one or more obstacles positioned either within a path or in proximity to a path with a decreased time required to traverse the path from a designated start position to a designated end position (e.g. when compared to a healthy individual with normal visual acuity or when compared to a prior traversal by the subject). In some embodiments, an enclosure may comprise at least 1, 2, 3, 4, 5, 6, 7, 8, 9 or 10 paths or designated paths. A designated path may differ from anon-designated path by the identification of the designated path by the experimenter as containing an intended start position and an intended end position.

In some embodiments of the enclosure, the one or more obstacles are not fixed to a surface of the disclosure. In some embodiments, the one or more obstacles are fixed to a surface of the disclosure. In some embodiments, the one or more obstacles are fixed to an internal surface of the enclosure, including, but not limited to, a floor, a wall and a ceiling of the enclosure. In some embodiments, the one or more obstacles comprise a solid object. In some embodiments, the one or more obstacles comprise a liquid object (e.g. a “water hazard”). In some embodiments, the one or more obstacles comprise in any combination or sequence along at least one path or in close proximity to a path, an object to be circumvented by a subject; an object to be stepped over by a subject; an object to be balanced upon by walking or standing; an object having an incline, a decline or a combination thereof; an object to be touched (for example, to determine a subject's ability to see and/or judge depth perception); and an object to be traversed by walking or standing beneath it (e.g., including bending one or more directions to avoid the object). In some embodiments of the enclosure, the one or more obstacles must be encountered by the subject in a designated order.

In certain embodiments, baseline or improved visual acuity and/or functional vision of a subject may be measured by having the subject navigate through a course or enclosure characterized by low light or dark conditions and including one or more obstacles for the subject to avoid, wherein the course or enclosure is present in an installation. In particular embodiments, the installation includes a modular lighting system and a series of different mobility course floor layouts. In certain embodiments, one room houses all mobility courses with one set of lighting rigs. For example, a single course may be set up at a time during mobility testing, and the same room/lighting rigs may be used for mobility testing independent of the course (floor layout) in use. In particular embodiments, the different mobility courses provided for testing are designed to vary in difficulty, with harder courses featuring low contrast pathways and hard to see obstacles, and easier courses featuring high contrast pathways and easy to see obstacles.

In some embodiments of the enclosure, the subject may be tested prior to administration of a composition of the disclosure to establish, for example, a baseline measurement of accuracy and/or speed or to diagnose a subject as having a retinal disease or at risk of developing a retinal disease. In some embodiments, the subject may be tested following administration of a composition of the disclosure to determine a change from a baseline measurement or a comparison to a score from a healthy individual (e.g. for monitoring/testing the efficacy of the composition to improve visual acuity).

### Adaptive Optics and Scanning Laser Ophthalmoscopy (AOSLO)

The baseline or improved measurement of retinal cell viability of a subject of the disclosure may be measured by one or more AOSLO techniques. Scanning Laser Ophthalmoscopy (SLO) may be used to view a distinct layer of a retina of an eye of a subject. Preferably, adaptive optics (AO) are incorporated in SLO (AOSLO), to correct for artifacts in images from SLO alone typically caused by structure of the anterior eye, including, but not limited to the cornea and the lens of the eye. Artifacts produced by using SLO alone decrease resolution of the resultant image. Adaptive optics allow for the resolution of a single cell of a layer of the retina and detect directionally backscattered light (waveguided light) from normal or intact retinal cells (e.g. normal or intact photoreceptor cells).

In some embodiments of the disclosure, using an AOSLO technique, an intact cell produce a waveguided and/or detectable signal. In some embodiments a non-intact cell does not produce a waveguided and/or detectable signal.

AOSLO may be used to image and, preferably, evaluate the retina or a portion thereof in a subject. In some embodiments, the subject has one or both retinas imaged using an AOSLO technique. In some embodiments, the subject has one or both retinas imaged using an AOSLO technique prior to administration of a composition of the disclosure (e.g. to determine a baseline measurement for subsequent comparison following treatment and/or to determine the presence and/or the severity of retinal disease). In some embodiments, the subject has one or both retinas imaged using an AOSLO technique following an administration of a composition of the disclosure (e.g. to determine an efficacy of the composition and/or to monitor the subject following administration for improvement resulting from treatment).

In some embodiments of the disclosure, the retina is imaged by either confocal or non-confocal (split-detector) AOSLO to evaluate a density of one or more retinal cells. In some embodiments, the one or more retinal cells include, but are not limited to a photoreceptor cell. In some embodiments, the one or more retinal cells include, but are not limited to a cone photoreceptor cell. In some embodiments, the one or more retinal cells include, but are not limited to a rod photoreceptor cell. In some embodiments, the density is measured as number of cells per millimeter. In some embodiments, the density is measured as number of live or viable cells per millimeter. In some embodiments, the density is measured as number of intact cells per millimeter (cells comprising an AAV particle or a transgene sequence of the disclosure). In some embodiments, the density is measured as number of responsive cells per millimeter. In some embodiments, a responsive cell is a functional cell.

In some embodiments, AOSLO may be used to capture an image of a mosaic of photoreceptor cells within a retina of the subject. In some embodiments, the mosaic includes intact cells, non-intact cells or a combination thereof. In some embodiments, an image of a mosaic comprises an image of an entire retina, an inner segment, an outer segment or a portion thereof. In some embodiments, the image of a mosaic comprises a portion of a retina comprising or contacting a composition of the disclosure. In some embodiments, the image of a mosaic comprises a portion of a retina juxtaposed to a portion of the retina comprising or contacting a composition of the disclosure. In some embodiments, the image of a mosaic comprises a treated area and an untreated area, wherein the treated area comprises or contacts a composition of the disclosure and the untreated area does not comprise or contact a composition of the disclosure.

In some embodiments, AOSLO may be used alone or in combination with optical coherence tomography (OCT) to visualize directly a retinal, a portion of a retinal or a retinal cell of a subject. In some embodiments, adaptive optics may be used in combination with OCT (AO-OCT) to visualize directly a retinal, a portion of a retinal or a retinal cell of a subject.

In some embodiments of the disclosure, the outer or inner segment is imaged by either confocal or non-confocal (split-detector) AOSLO to evaluate a density of cells therein or a level of integrity of the outer segment, the inner segment or a combination thereof. In some embodiments, AOSLO may be sued to detect a diameter of an inner segment, an outer segment or a combination thereof.

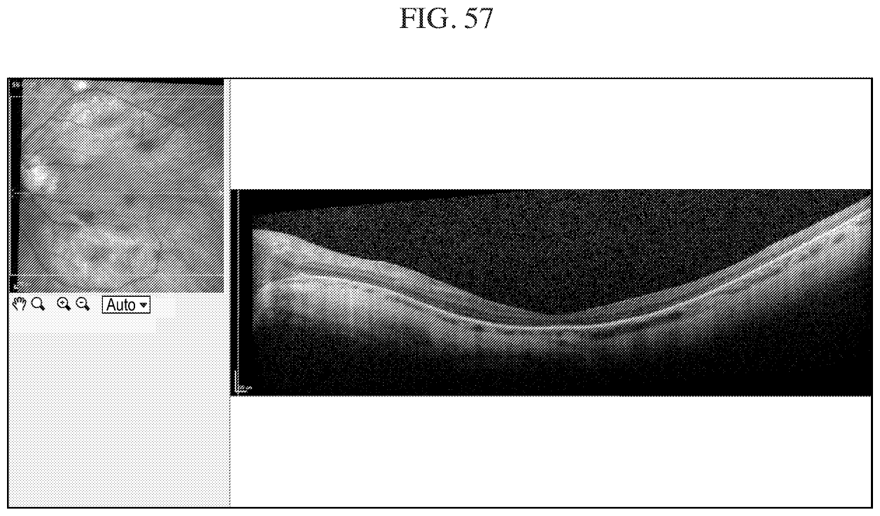

An illustrative AOSLO system is shown in FIG. 57.

Additional description of AOSLO and various techniques may be described at least in Georgiou et al. Br J Opthalmol 2017; 0:1-8; Scoles et al. Invest Opthalmol Vis Sci. 2014; 55:4244-4251; and Tanna et al. Invest Opthalmol Vis Sci. 2017; 58:3608-3615.

### Pharmaceutical Formulations

Compositions of the disclosure may comprise a Drug Substance. In some embodiments, the Drug Substance comprises or consists of AAV-RPGR^{ORF15}. In some embodiments, the Drug Substance comprises or consists of an AAV-RPGR^{ORF15} and a formulation buffer. In some embodiments, the formulation buffer comprises 20 mM Tris, 1 mM MgCl₂, and 200 mM NaCl at pH 8. In some embodiments, the formulation buffer comprises 20 mM Tris, 1 mM MgCl₂, and 200 mM NaCl at pH 8 with poloxamer 188 at 0.001%.

### Excipients

Compositions of the disclosure may comprise a AAV-RPGR^{ORF15} Drug Product. In some embodiments, the Drug Product comprises or consists of a Drug Substance and a formulation buffer. In some embodiments, the Drug Product comprises or consists of a Drug Substance diluted in a formulation buffer. In some embodiments, the Drug Product comprises or consists of an AAV2-RPGR^{ORF15} Drug Substance diluted to a final Drug Product AAV-RPGR^{ORF15} vector genome (vg) concentration in a formulation buffer.

### Ocular Formulations

Compositions of the disclosure may be formulated to comprise, consist essentially of or consist of an AAV-RPGR^{ORF15} Drug Substance at an optimal concentration for ocular injection or infusion.

Compositions of the disclosure may comprise one or more buffers that increase or enhance the stability of an AAV of the disclosure. In some embodiments, compositions of the disclosure may comprise one or more buffers that ensure or enhance the stability of an AAV of the disclosure. Alternatively, or in addition, compositions of the disclosure may comprise one or more buffers that prevent, decrease, or minimize AAV particle aggregation. In some embodiments, compositions of the disclosure may comprise one or more buffers that prevent, decrease, or minimize AAV particle aggregation.

Compositions of the disclosure may comprise one or more components that induce or maintain a neutral or slightly basic pH. In some embodiments, compositions of the disclosure comprise one or more components that induce or maintain a neutral or slightly basic pH of between 7 and 9, inclusive of the endpoints. In some embodiments, compositions of the disclosure comprise one or more components that induce or maintain a pH of about 8. In some embodiments, compositions of the disclosure comprise one or more components that induce or maintain a pH of between 7.5 and 8.5. In some embodiments, compositions of the disclosure comprise one or more components that induce or maintain a pH of between 7.7 and 8.3. In some embodiments, compositions of the disclosure comprise one or more components that induce or maintain a pH of between 7.9 and 8.1. In some embodiments, compositions of the disclosure comprise one or more components that induce or maintain a pH of 8.

Following contact of a composition of the disclosure and a cell, the AAV-RPGR^{ORF15} expresses a gene or a portion thereof, resulting in the production of a product encoded by the gene or a portion thereof. In some embodiments, the cell is a target cell. In some embodiments, the target cell is a retinal cell. In some embodiments, the retinal cell is a neuron. In some embodiments, the neuron is a photoreceptor. In some embodiments, the cell is in vivo, in vitro, ex vivo or in situ. In some embodiments, including those wherein the cell is in vivo, the contacting occurs following administration of the composition to a subject. In some embodiments, the AAV-RPGR^{ORF15} expresses a RPGR^{ORF15} or a portion thereof, results in the production of a product encoded by the gene or a portion thereof at a therapeutically-effective level of expression of the RPGR^{ORF15} protein.

Physical Titre: Genomic titre is determined using qPCR. This method allows quantification of genomic copy number. Samples of the vector stock are diluted in buffer. The samples are DNase treated and the viral capsids lysed with proteinase K to release the genomic DNA. A dilution series is then made. Replicates of each sample are subjected to qPCR using a Taqman based Primer/Probe Set specific for the CAG sequence. A standard curve is produced by taking the average for each point in the linear range of the standard plasmid dilution series and plotting the log copy number against the average CT value for each point. In some embodiments, the plasmid DNA used in the standard curve is in the supercoiled conformation. In some embodiments, the plasmid DNA used in the standard curve is in the linear conformation. Linearized plasmid can be prepared, for example by digestion with HindIII restriction enzyme, visualized by agarose gel electrophoresis and purified using the QIAquick Gel Extraction Kit (Qiagen) following manufacturer's instructions. Other restriction enzymes that cut within the plasmid used to generate the standard curve may also be appropriate. In some embodiments, the use of supercoiled plasmid as the standard increased the titre of the AAV vector compared to the use of linearized plasmid. The titre of the rAAV vector can be calculated from the standard curve and is expressed as DNase Resistant Particles (DRP)/mL.

Droplet Digital PCR (ddPCR): ddPCR can be used as an alternative to, or in addition to qPCR to measure genomic titre. ddPCR uses Taq polymerase in a standard PCR reaction to amplify a target DNA fragment from a complex sample using a pre-validated primer or primer/probe assay. The PCR reaction is partitioned into thousands of individual reaction vessels prior to amplification, and the data is acquired at the reaction end point. ddPCR offers direct and independent quantification of DNA without standard curves, and can give a precise and reproducible data. End point measurement enables nucleic acid quantitation independent of reaction efficiency. ddPCR can be used for extremely low target quantitation from variably contaminated samples.

Full: empty Ratio (Analytical Ultracentrifugation): The full: empty ratio of AAV8 particles may be determined using analytical ultracentrifugation (AUC). AUC has an advantage over other methods of being non-destructive, meaning that samples may be recovered following AUC for additional testing. Samples comprising empty and full AAV8 particles are applied to a liquid composition through which the AAV8 move during an ultracentrifugation. A measurement of sedimentation velocity of one or more AAV8 particles provides hydrodynamic information about the size and shape of the AAV particles. A measure of sedimentation equilibrium provides thermodynamic information about the solution molar masses, stoichiometries, association constants, and solution nonideality of the AAV8 particles. Illustrative measurements acquired during AUC are radial concentration distributions, or “scans”. In some embodiments, scans are acquired at intervals ranging from minutes (for velocity sedimentation) to hours (for equilibrium sedimentation). The scans of the methods of the disclosure may contain optical measurements (e.g., light absorbance, interference and/or fluorescence). Ultracentrifugation speeds may range from between 10,000 rotations per minute (rpm) and 75,000 rpm, inclusive of the endpoints. As full AAV8 particles and empty AAV8 particles demonstrate distinct measurements by AUC, the full/empty ratio of a sample may be determined using this method.

Vector Identity (DNA): This assay provides a confirmation of the viral DNA sequence. The assay is performed by digesting the viral capsid and purifying the viral DNA. The DNA is sequenced with a minimum of 2 fold coverage both forward and reverse where possible (some regions, e.g., ITRs are problematic to sequence). The DNA sequencing contig is compared to the expected sequences to confirm identity.

Replication Competent AAV: Test article is used to transduce HEK293 cells in the presence or the absence of wild type adenovirus. Three successive rounds of cell amplification will be conducted and total genomic DNA is extracted at each amplification step.

The rcAAV8 are detected by real-time quantitative PCR. Two sequences are isolated genomic DNA; one specific to the AAV2 Rep gene and one specific to an endogenous gene of the HEK293 cells (human albumin). The relative copy number of the Rep gene per cell is determined. The positive control is the wild type AAV virus serotype 8 tested alone or in the presence of the rAAV vector preparation.

The limit of detection of the assay is challenged for each tested batch. The limit of detection is 10 rcAAV per 1×10\hat{ }8, or 1×10\hat{ }10, genome copies of test sample. If a test sample is negative for Rep sequence, the result for this sample will be reported as: NO REPLICATION, <10 rcAAV per 1×10\hat{ }8 (or 1×10\hat{ }10) genome copies of test sample. If a test sample is positive for Rep sequence, the result for this sample will be reported as: REPLICATION.

Total DNA: Picogreen reagent is an ultra-sensitive fluorescent nucleic acid stain that binds double-stranded DNA and forms a highly luminescent complex (λexcitation=480 nm−λemission=520 nm). This fluorescence emission intensity is proportional to dsDNA quantity in solution. Using a DNA standard curve with known concentrations, DNA content in test samples is obtained by converting measured fluorescence.

### Stability of AAV Compositions

Compositions of the disclosure maintain long term stability when stored at <−60° C. For example, compositions of the disclosure maintain long term stability when stored at temperature between −80° C. and 40° C. (approximately human body temperature), inclusive of the endpoints. For example, compositions of the disclosure maintain long term stability when stored at temperature between −80° C. and 5° C., inclusive of the endpoints. For example, compositions of the disclosure maintain long term stability when stored at −80° C., −20° C. or 5° C. In some embodiments, compositions of the disclosure are formulated as liquids or suspensions, aliquotted into one or more containers (e.g., vials), and stored at <−60° C. In some embodiments, compositions of the disclosure are formulated as liquids or suspensions, aliquotted into one or more containers (e.g., vials), and stored at −80° C., −20° C. or 5° C.

Compositions of the disclosure may be provided in a container with an optimal surface area to volume ratio for maintaining long term stability when stored at <−60° C. Compositions of the disclosure may be provided in a container with an optimal surface area to volume ratio for maintaining long term stability when stored at −80° C., −20° C. or 5° C. In some embodiments, compositions of the disclosure are formulated as liquids or suspensions, aliquotted into one or more containers (e.g., vials), and stored in one or more containers with a surface area to volume ratio as large as possible when all storage requirements are considered.

Compositions of the disclosure maintain long term stability when stored at ambient relative humidity.

### EXAMPLES

### Example 1: Gene Therapy for Retinitis Pigmentosa in Human Subjects

Male subjects 18 years and older with a genetically confirmed diagnosis of Retinitis Pigmentosa (RP) were injected subretinally with a single dose of an AAV RPGR^{ORF15} gene therapy vector. The study involved 6 dose cohorts, with AAV8-RPGR doses of 5×10⁹ gp (Cohort 1), 1×10¹⁰ gp (Cohort 2), 5×10¹⁰ gp (Cohort 3), and 1×10¹¹ gp (Cohort 4), 2.5×10¹¹ gp (Cohort 5), and 5×10¹¹ gp (Cohort 6). Subjects were subsequently followed for 12 months and evaluated for best corrected visual acuity (BCVA), retinal sensitivity and fixation via microperimetry and retinal thickness via optical coherence tomography (OCT). The methods for subject treatment and analysis are provided in Example 3.

### Gene Therapy Surgery

The AAV8.RK.coRPGR vector was delivered into the sub-macula space via a two-step subretinal injection. Briefly, a standard 23-gauge three-port pars plana vitrectomy was performed using the Alcon Constellation Vision System (Alcon Inc, Fort Worth, USA). Posterior vitreous detachment was induced followed by core and peripheral vitrectomy. A small subretinal fluid bleb was first initiated by subretinal injection of balanced salt solution using a 41G subretinal cannula (Dutch Ophthalmic Research Center BV, Zuidland, Netherlands) connected to a vitreous injection set. The bleb was then enlarged by further subretinal injection of 0.1 ml of viral vector at the appropriate concentration through the same entry site, leading to iatrogenic detachment of the macula. All sclerostomies were secured with absorbable polyglactin sutures and the vitreous cavity was left fluid filled at the end of the procedure. As part of standard protocol, subjects received a 21-day course of oral prednisone/prednisolone starting from 2 days prior to gene therapy: at 1 mg/kg/day for 10 days, followed by 0.5 mg/kg for 7 days, 0.25 mg/kg for 3 days, and 0.125 mg/kg for 3 days.

### Visual Function Testing

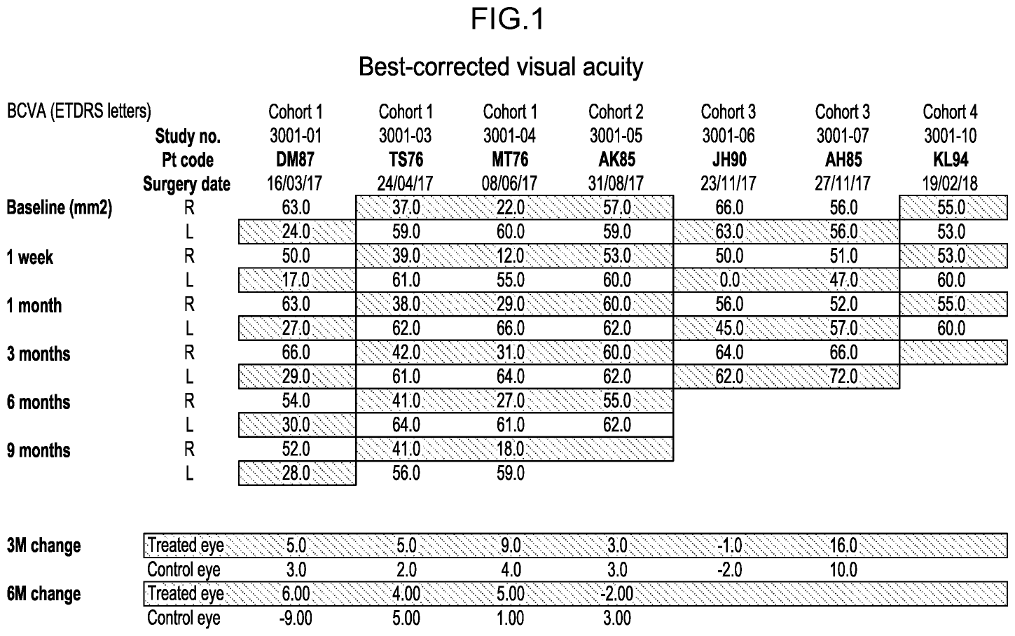

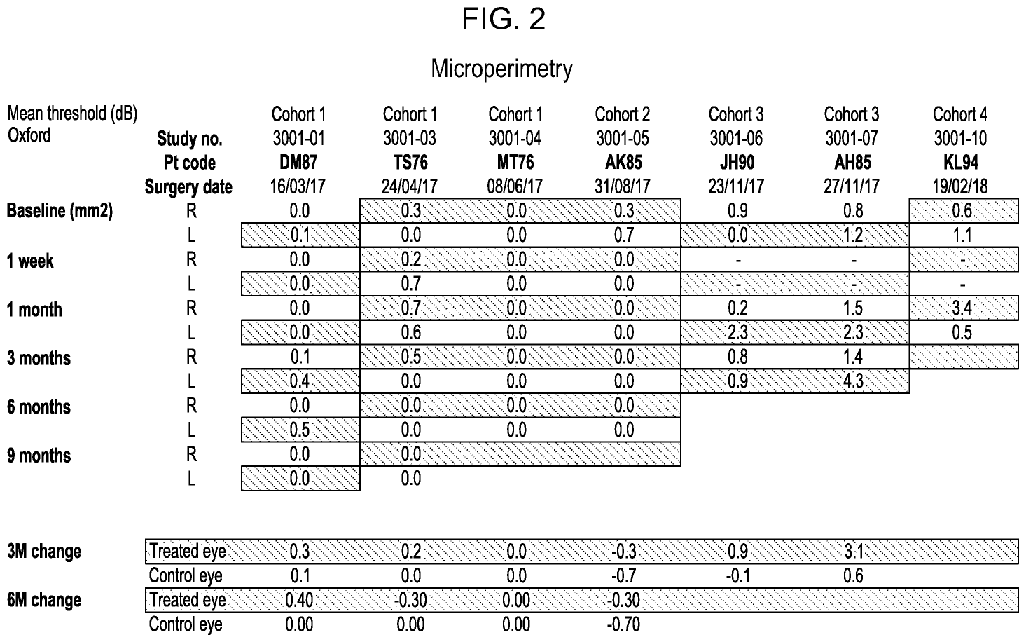

The best-corrected visual acuity (BCVA) was measured at each scheduled visit using the Early Treatment Diabetic Retinopathy Study (ETDRS) chart (FIG. 1).

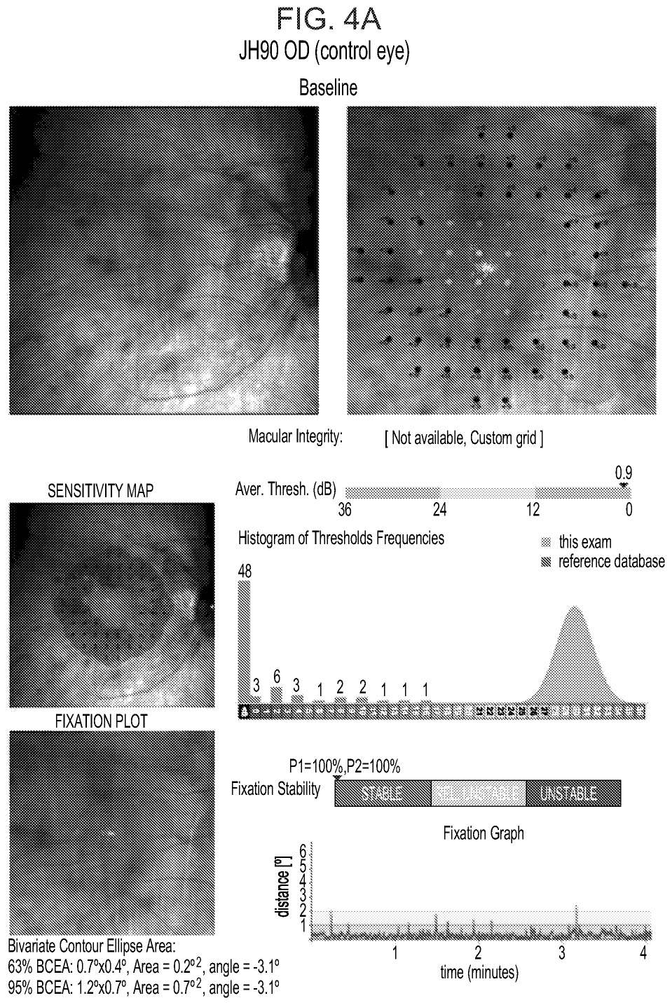

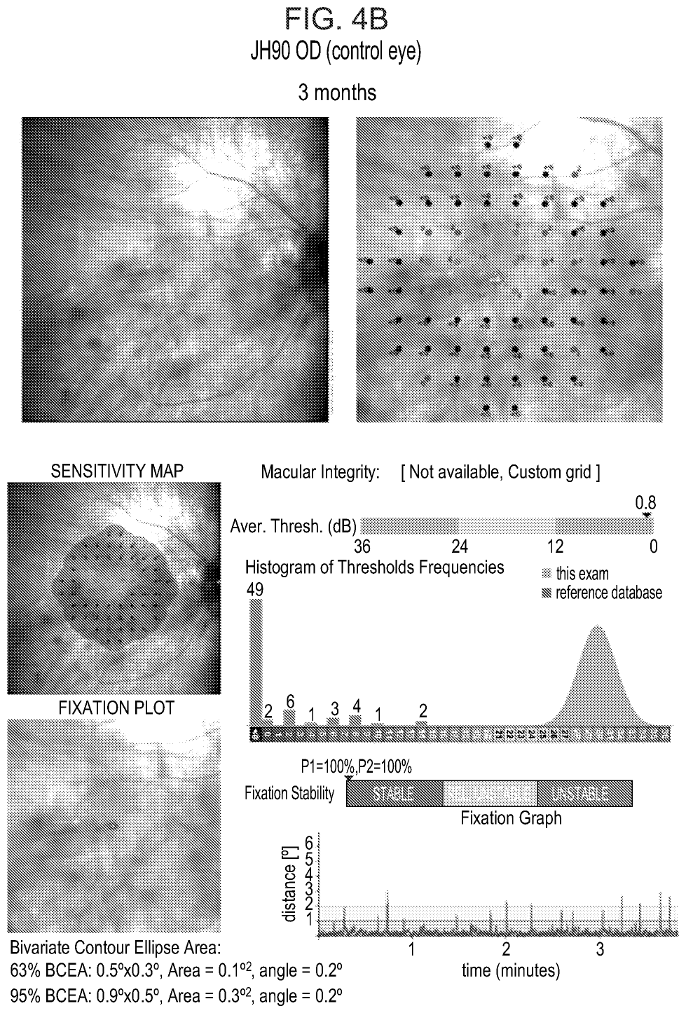

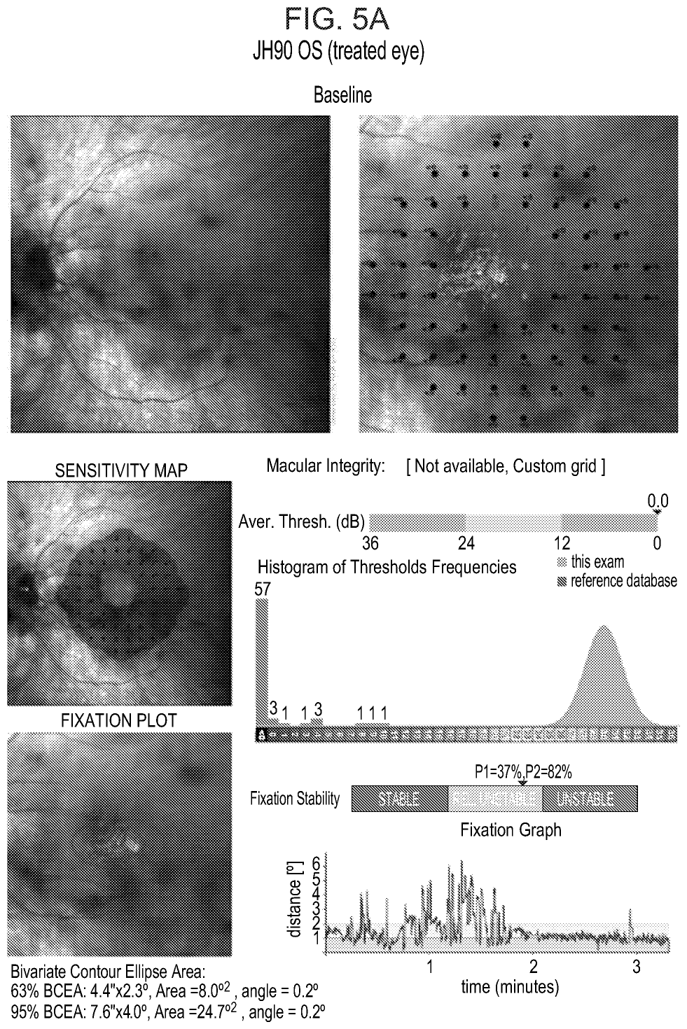

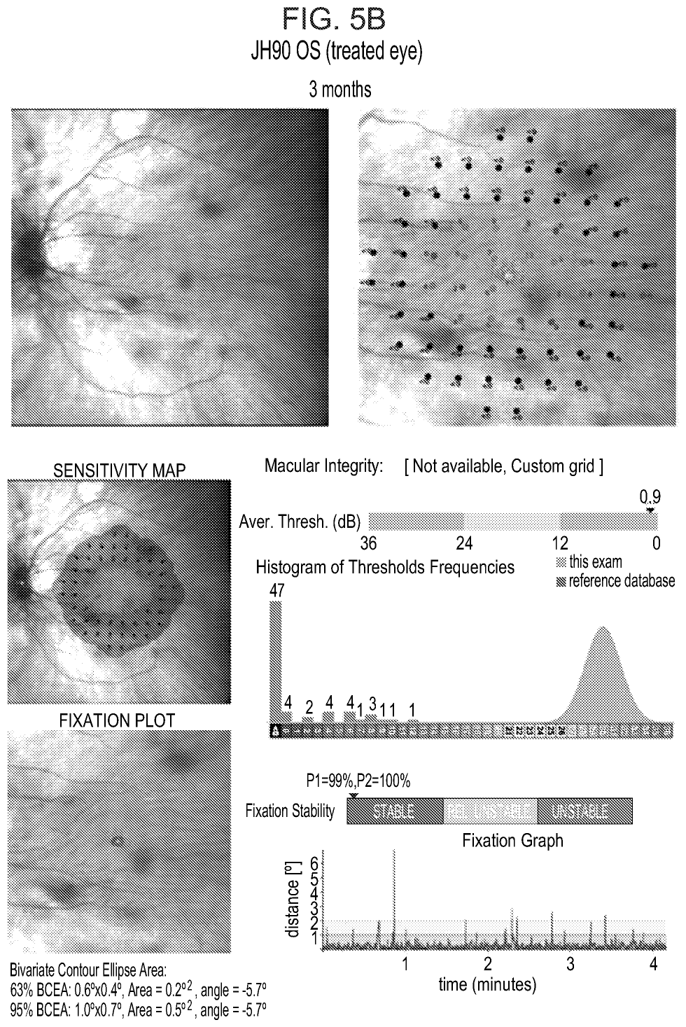

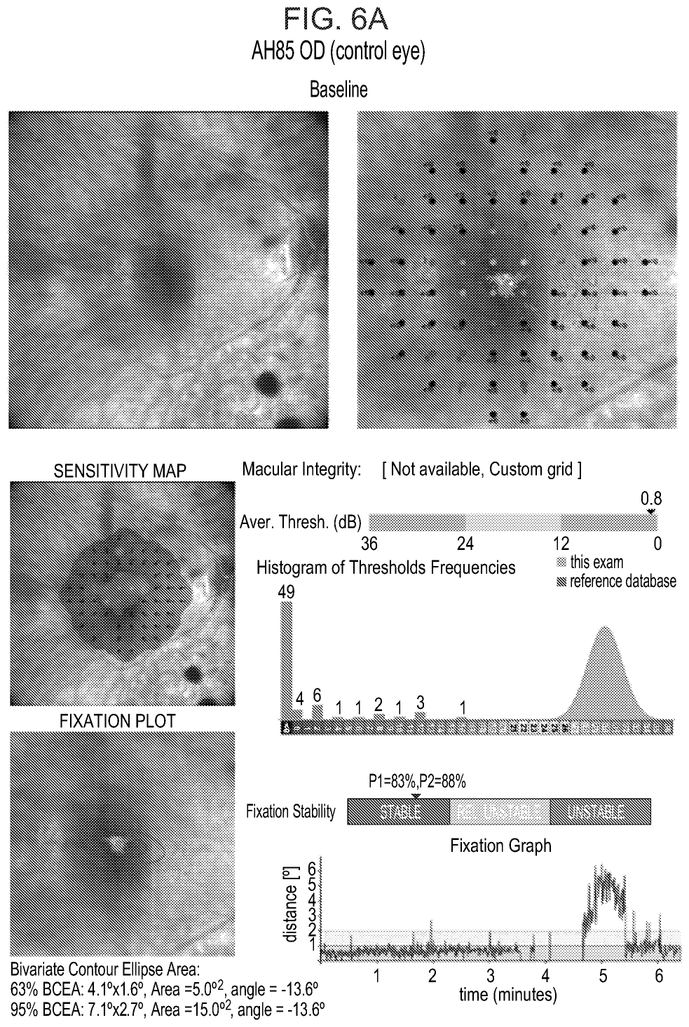

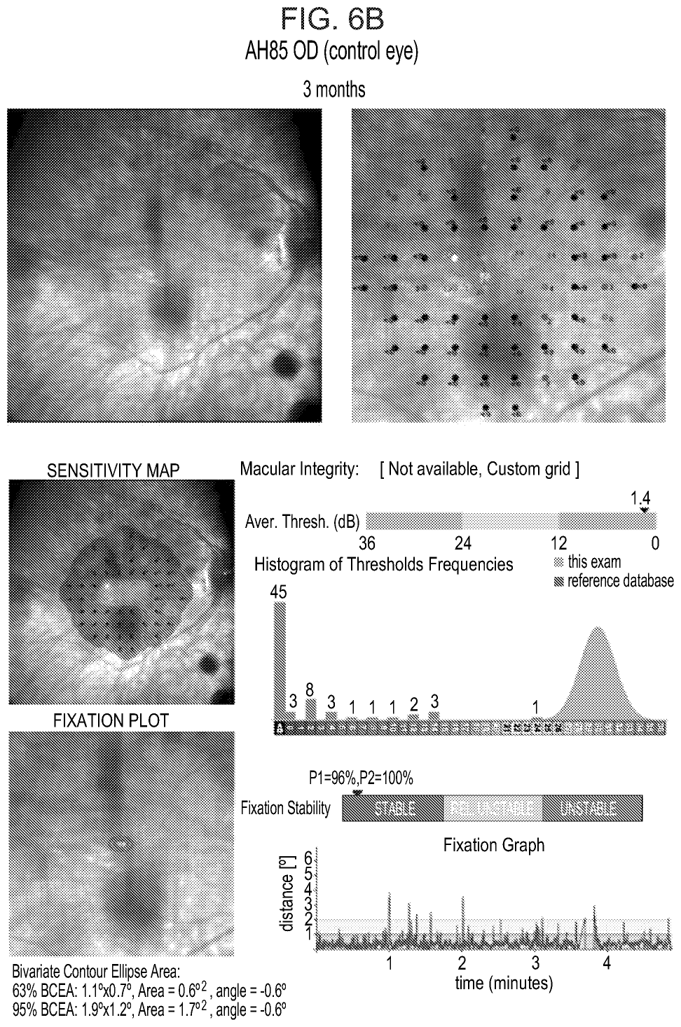

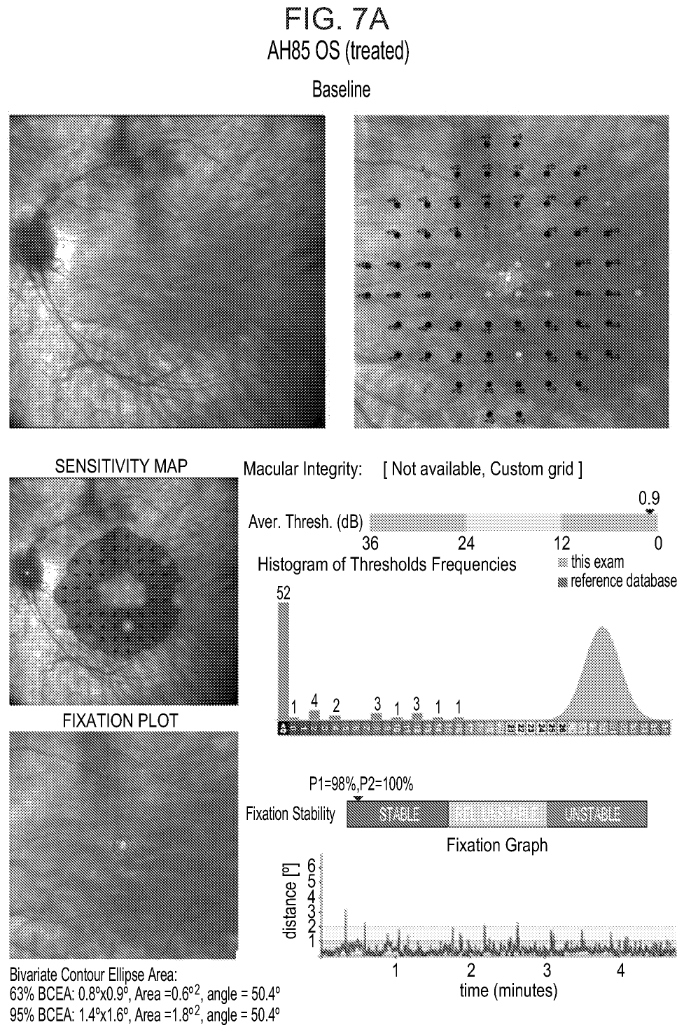

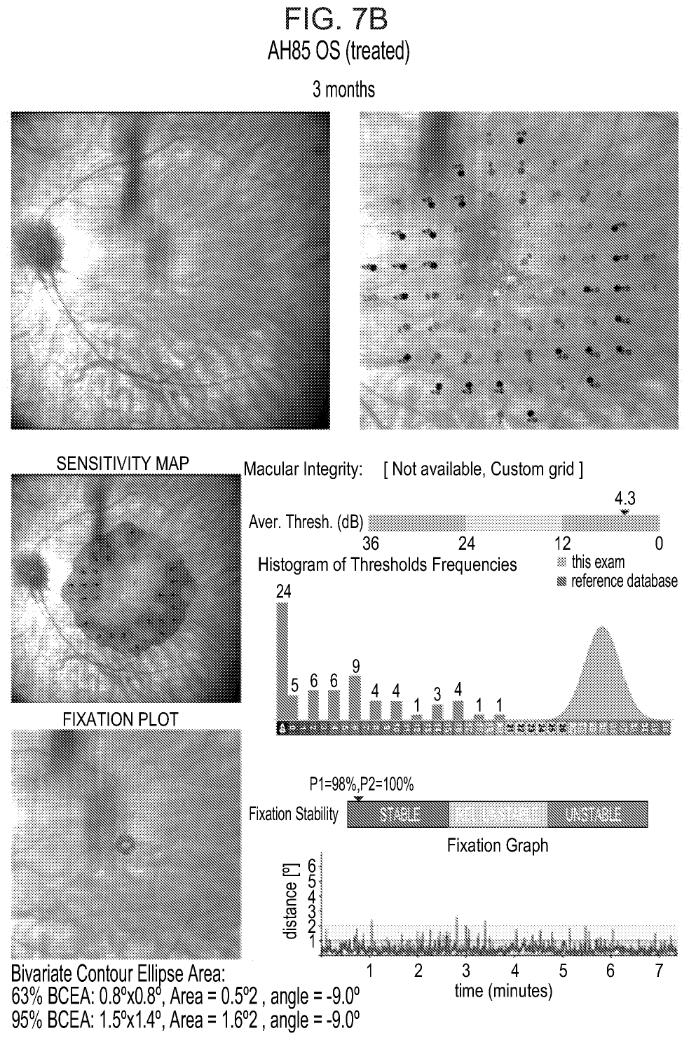

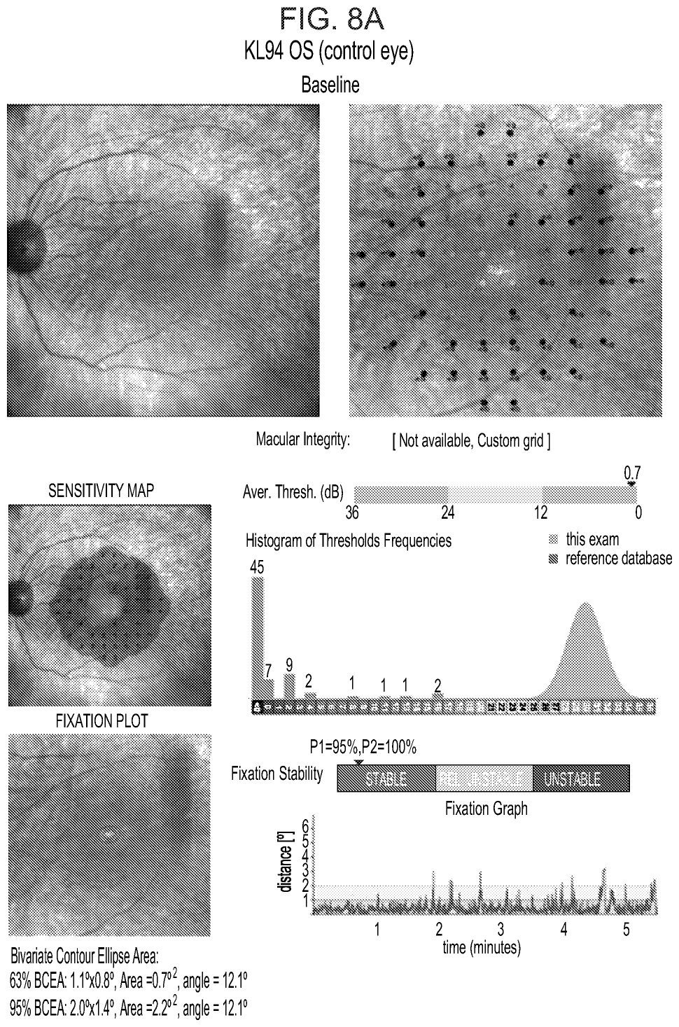

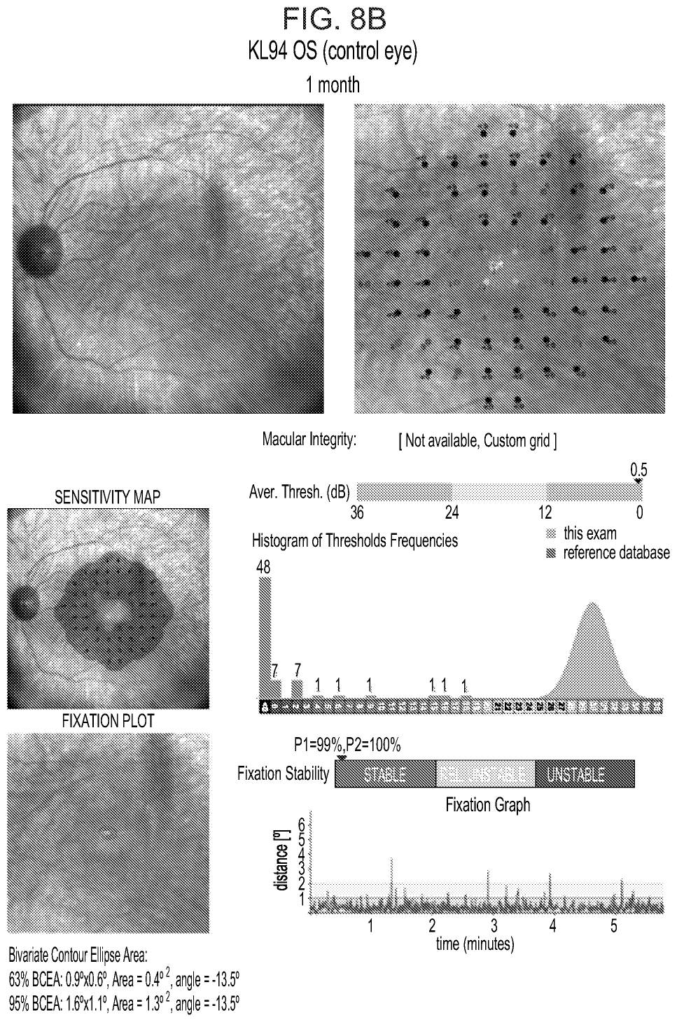

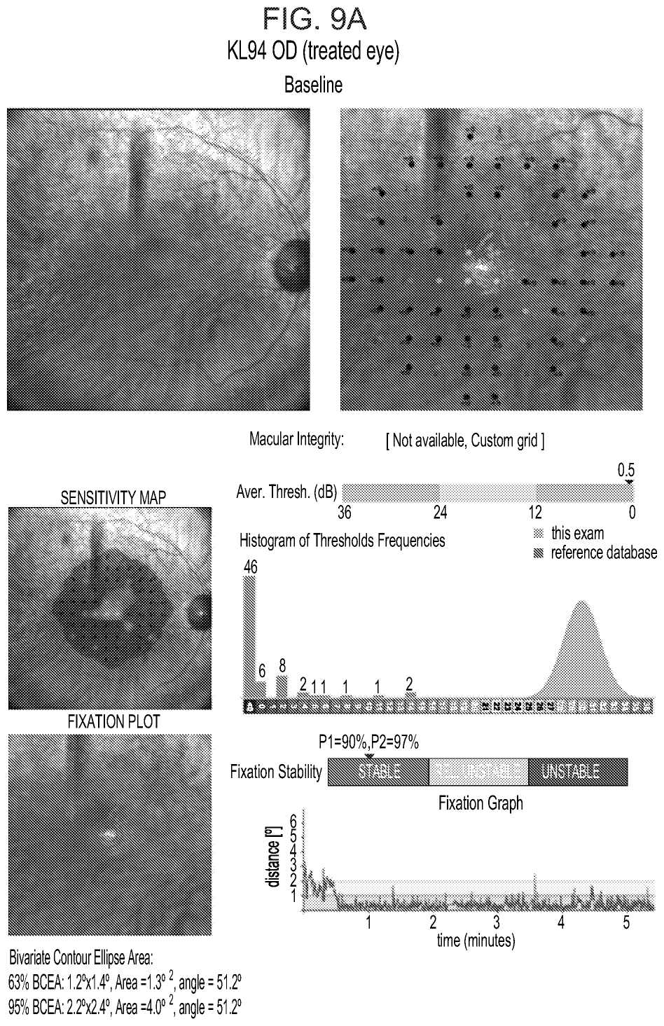

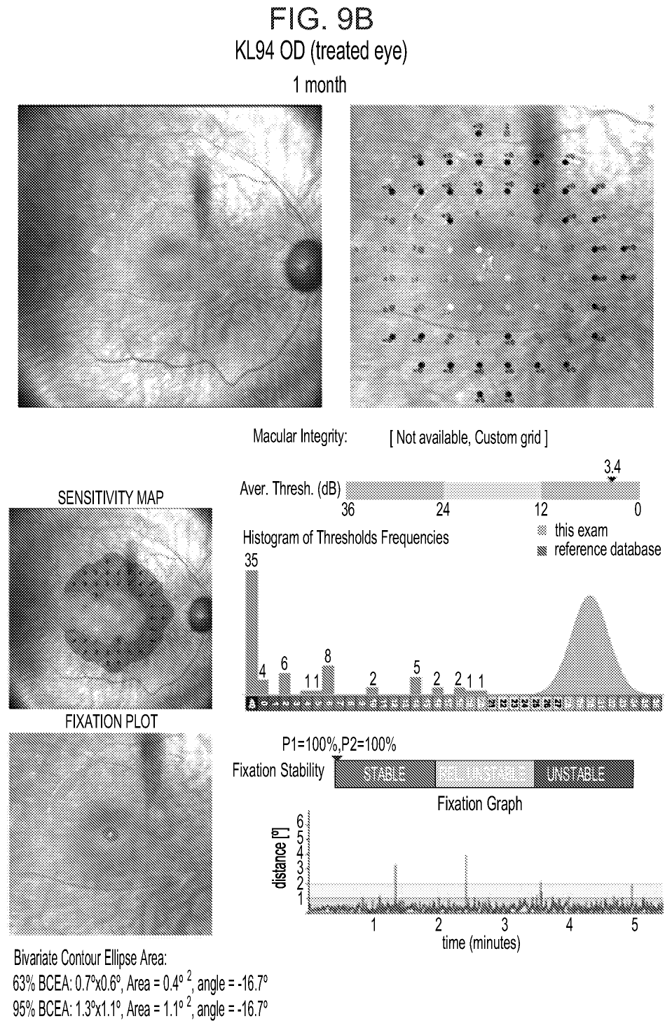

Retinal sensitivity was measured by mesopic microperimetry (MAIA, CenterVue SpA, Padova, Italy) using a standard 68-stimuli (10-2) grid covering the central 10 degrees of the macula. Raw microperimetry data is disclosed in FIGS. 4-9. In each panel, clockwise from upper left, are shown: a scanning laser ophthalmoscopy (SLO) image of the fundus; a map of sensitivity values (36 dB scale, color coded from purple=0 to green=36) and preferred retinal location (PRL) over the zoomed SLO image; a bar showing the average threshold (dB) on a scale from 36 (left) to 0 (right); a histogram of threshold values, with the exam shown in grey and a normal distribution shown in green; a bar showing fixation stability from stable (green), relatively unstable (yellow) and unstable (red) and disclosing the percentage of fixation points in P1 and P2; a fixation graph showing the amplitude of eye movements as distance in degrees (y-axis) versus time in minutes (x-axis); the calculated bivariate contour ellipse area corresponding to the point cloud in the fixation plot above; a fixation plot over the zoomed SLO image showing PRL area, P1 and P2; an interpolated sensitivity plot over the full SLO image, with a heat map of sensitivity from 0 dB (purple) to 36 dB (green) overlaid.

### Results

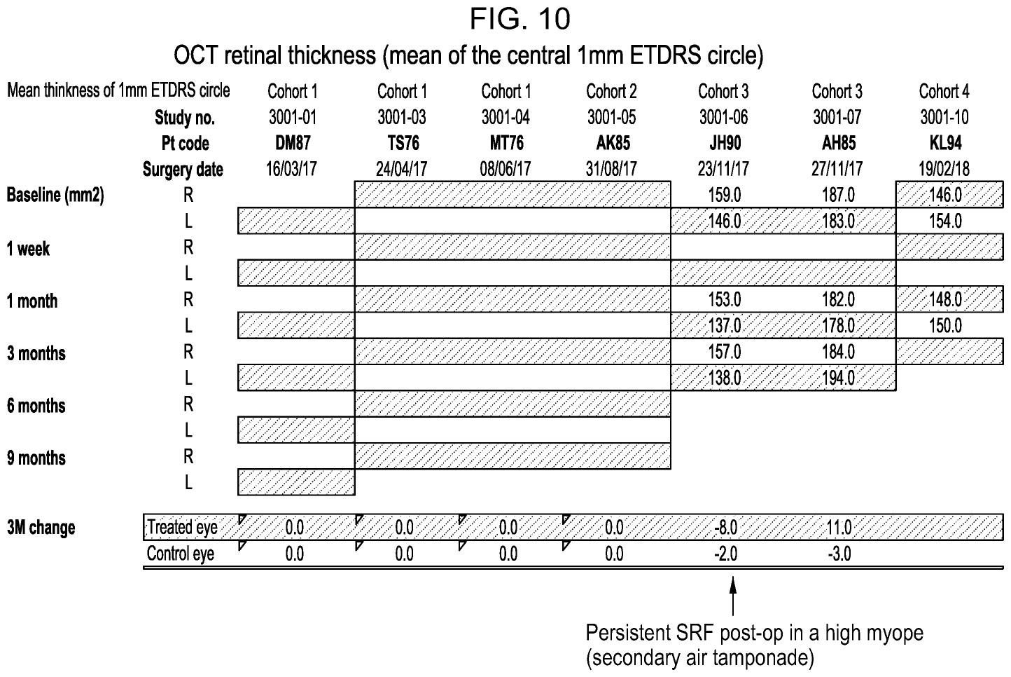

Significant gains were seen in mean retinal sensitivity, sensitivity histogram and visual field (see heat map) in the treated eyes of the cohort 3 and 4 patients. An 11 μm increase in retinal thickness was seen in the treated eye of AH85 (cohort 3) at 3 months (FIG. 10). Visual acuity returned to baseline or minimally improved in all treated eyes. There were no adverse events, except for 1 case of persistent subretinal fluid post-op in a high myope (JH90), which resolved after air tamponade (indicated in FIG. 10). One subject showed an increase in mean retinal thickness of 11 mm in the centra 1 mm EDTRS circle at 3M after treatment with AAV-RPGR^{ORF15} (FIG. 10).

### Example 2: Reversal of Visual Field Loss Ina Subject Following Gene Therapy for Retinitis Pigmentosa

Retinitis pigmentosa (RP) is a neurodegenerative disorder affecting photoreceptors in the retina. It causes progressive visual field constriction and eventual blindness. Loss-of-function mutations in the Retinitis Pigmentosa GTPase Regulator (RPGR) gene account for 15-20% of all RP. Although RPGR is within the coding capacity of the adeno-associated viral (AAV) vector, a highly repetitive purine-rich region at the 3′-end and a splice site immediately upstream of this have created significant challenges in cloning an AAV.RPGR vector, with several groups reporting miss-spliced or truncated variants during preclinical testing. Codon optimization can be used to disable the endogenous splice site and stabilize the purine-rich sequence in the photoreceptor-specific RPGR transcript without altering the amino acid sequence. Glutamylation of RPGR protein, a key post-translational modification was also preserved following codon-optimization and more importantly, functional effects were seen when delivered using an AAV8 vector in two mouse models of human RPGR disease.

### Validation of AAV Vector for RPGR Gene Therapy

The retinal spliceoform of RPGR, RPGR^{ORF15}, contains the highly repetitive purine-rich exon (or open-reading frame) 15, which is prone to mutations as well as errors during viral vector cloning. To create a stable vector for human gene therapy, the AAV serotype 8 vector construct contains a codon-optimized version of human RPGR^{ORF15} (coRPGR) driven by the human photoreceptor-specific rhodopsin kinase promoter (RK). The vector was tested in Rpgr−/− mice and shown to generate full length RPGR protein with identical glutamylation pattern as wild-type RPGR^{ORF15}, and rescue retinal function as measured by electroretinography (ERG) amplitudes up to 6 months. The clinical grade AAV8.RK.coRPGR vector was validated in Rpgr−/− mice through subretinal injections. Immunostaining showed co-localization of human RPGR with its known interaction partner, RPGR-interacting protein 1 (RPGRIP1), in the region of the photoreceptor connecting cilia.

### Gene Therapy Surgery

The AAV8.RK.coRPGR vector was delivered into the sub-macula space via a two-step subretinal injection. Briefly, a standard 23-gauge three-port pars plana vitrectomy was performed using the Alcon Constellation Vision System (Alcon Inc, Fort Worth, USA). Posterior vitreous detachment was induced followed by core and peripheral vitrectomy. A small subretinal fluid bleb was first initiated by subretinal injection of balanced salt solution using a 41G subretinal cannula (Dutch Ophthalmic Research Center BV, Zuidland, Netherlands) connected to a vitreous injection set. The bleb was then enlarged by further subretinal injection of 0.1 ml of viral vector at the appropriate concentration through the same entry site, leading to iatrogenic detachment of the macula. All sclerostomies were secured with absorbable polyglactin sutures and the vitreous cavity was left fluid filled at the end of the procedure. As part of standard protocol, the patient received a 21-day course of oral prednisone/prednisolone starting from 2 days prior to gene therapy: at 1 mg/kg/day for 10 days, followed by 0.5 mg/kg for 7 days, 0.25 mg/kg for 3 days, and 0.125 mg/kg for 3 days.

### Visual Function Testing

The best-corrected visual acuity (BCVA) was measured at each scheduled visit using the Early Treatment Diabetic Retinopathy Study (ETDRS) chart. Retinal sensitivity was measured by mesopic microperimetry (MAIA, CenterVue SpA, Padova, Italy) using a standard 68-stimuli (10-2) grid covering the central 10 degrees of the macula. To minimize learning effect, three microperimetry tests were performed in each eye over two days at baseline with the result of the third attempt taken forward for data analysis.

### Results

Previous natural history study showed that the retinal degeneration in RPGR-related retinitis pigmentosa is characterized by photoreceptor outer segment shortening seen as outer nuclear layer (ONL) thinning on OCT, eventually leading to loss of the ellipsoid zone (EZ) and visual field.

Subretinal injection of AAV RPGR^{ORF15} reversed retinal degeneration in a patient undergoing retinal gene therapy for RPGR-associated RP (Clinicaltrials.gov: NCT03116113). The novelty of this observation has implications for other clinical studies. While long term preservation of the visual field following retinal gene therapy was predicted, an unexpected reversal of visual field loss over a period of three months was observed in a 24-year-old patient who received 1×10¹¹ gp of AAV8.RPGR. The patient described subjective improvement in visual clarity and field in the treated eye at 2 weeks. Functional assessment showed the visual acuity to be unchanged from baseline, however retinal sensitivity improved progressively from 0.7 to 7.5 dB in the treated eye over 4 months (FIG. 11). Full segmentation of the macular OCT revealed thickening of the outer nuclear layer with geographic correspondence to areas of sensitivity gain and magnitudes (˜20 μm) compatible with the length of photoreceptor outer segments. This thickening was not seen in the treated eye of a patient who received the lowest dose (0.5×10¹⁰ gp) who did not have any observed improvement in visual function.

Until now the concept of improving vision in RP was generally thought be in the realm of stem cell treatments, however, these early observations raise the possibility that gene therapy can not only slow down the rate of degeneration, but also reverse some functional and anatomical deficits by rescuing ‘dormant’ (dysfunctional) photoreceptors.

Table 1 shows the demographics and confirmed pathogenic RPGR mutations of the patient in whom retinal sensitivity gain was observed following high dose gene therapy and the control participant who received the lowest dose.

TABLE 1
The trial participants are Caucasian males with clinically confirmed X-linked
retinitis pigmentosa and genetically confirmed mutations within RPGR.

| | | | Predicted protein | Vector dose |
|---|---|---|---|---|
| | Age (yr) | RPGR mutation | sequence | (gp) |
| Patient | 24 | c.2993_2997delAAGGG | p.(Glu998GlyfsTer79) | 1.0 × 10¹¹ |
| Control | 41 | c.1571delA | p.(Lys524fs) | 0.5 × 10¹⁰ |

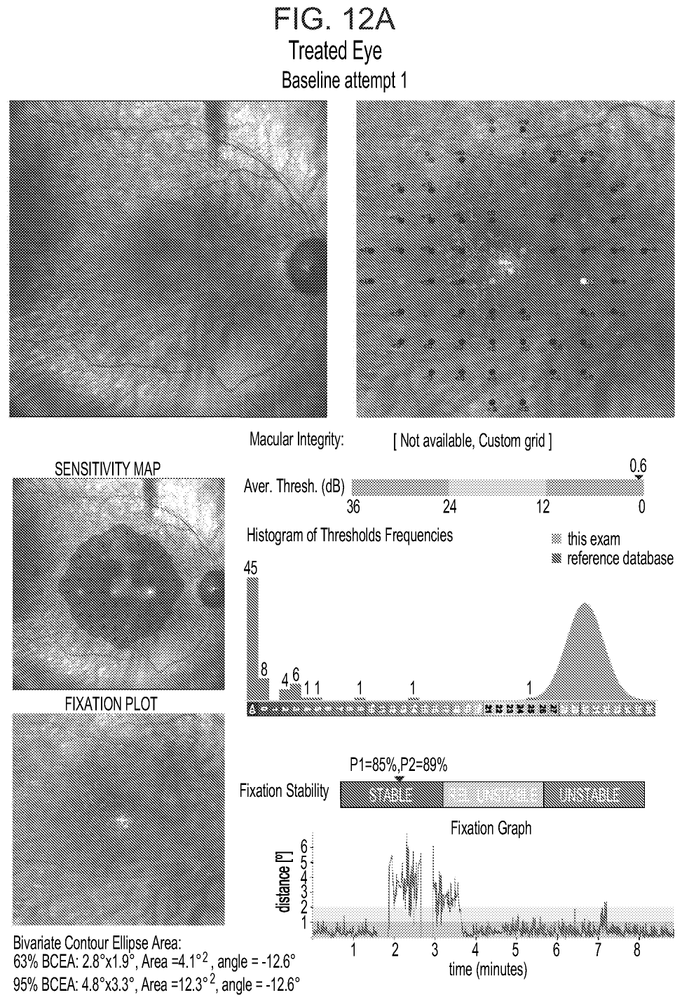

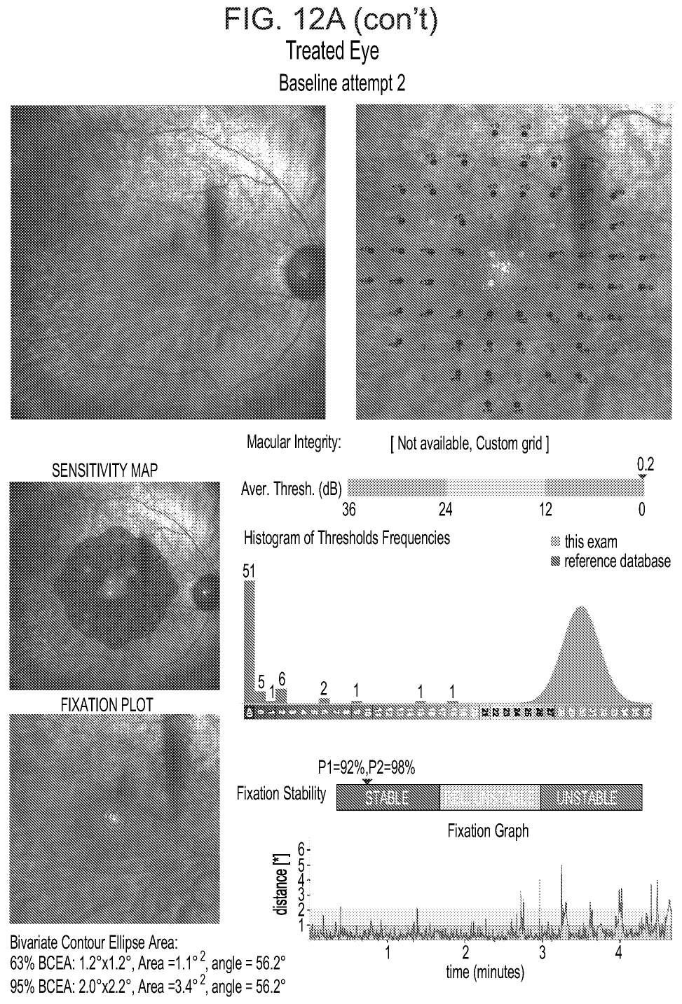

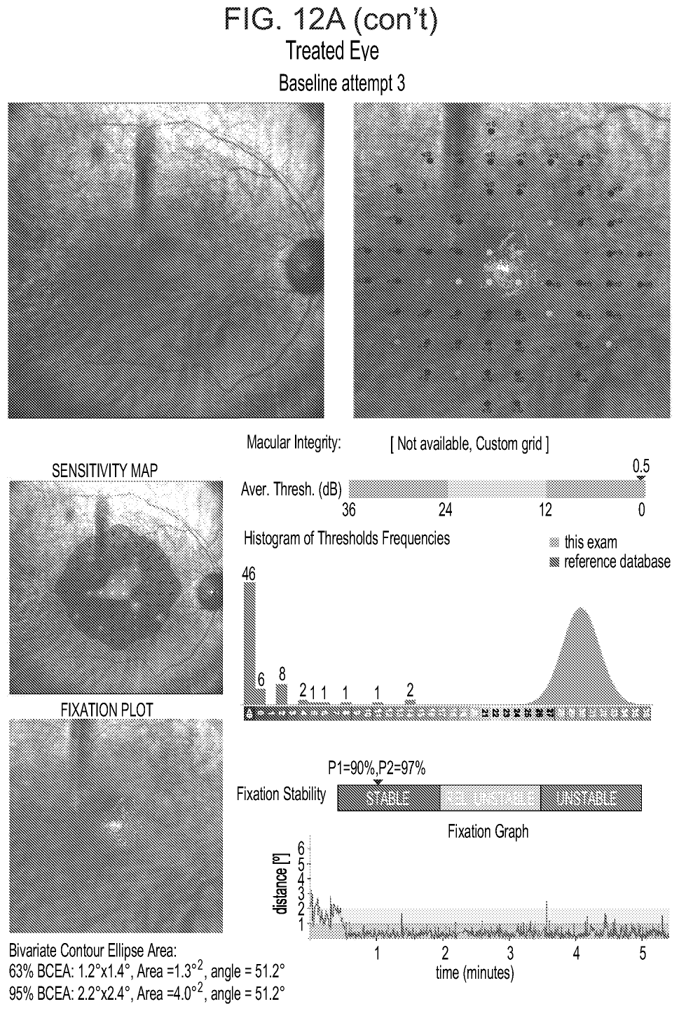

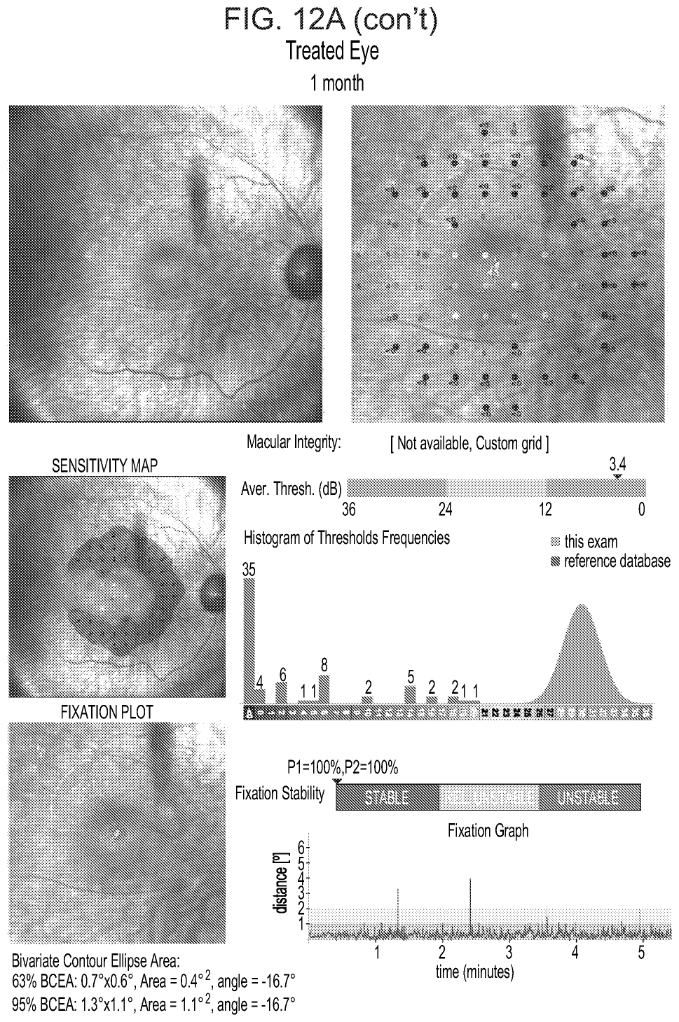

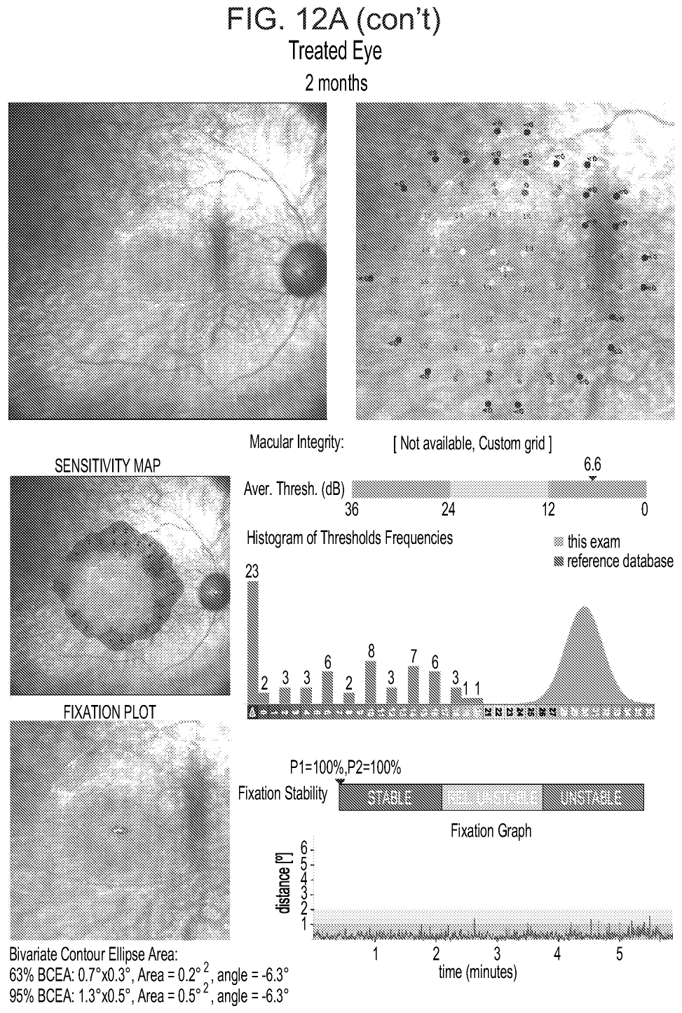

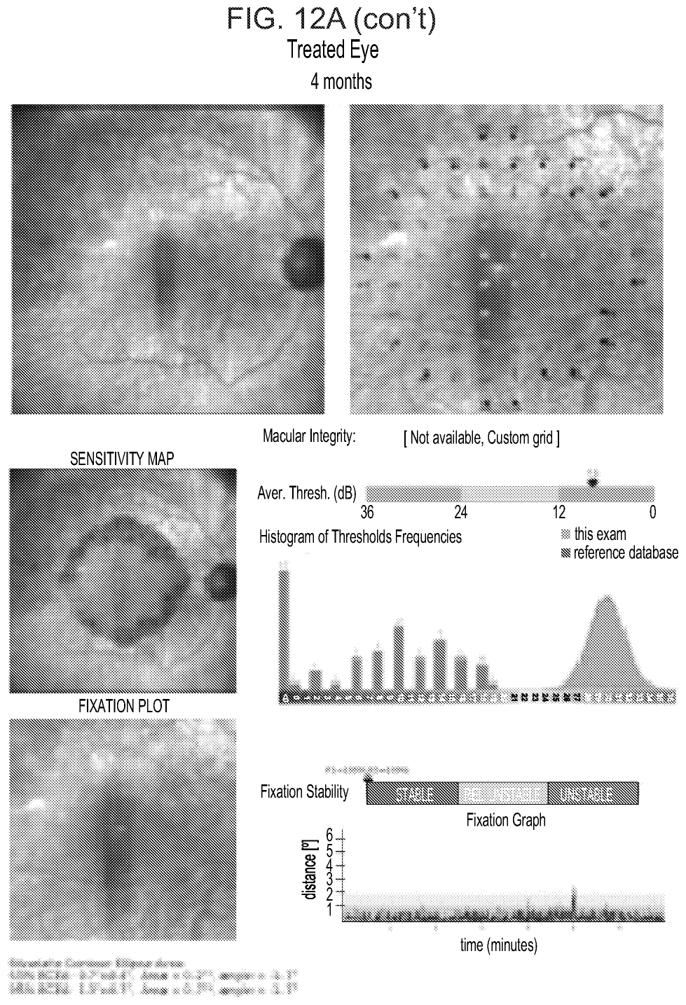

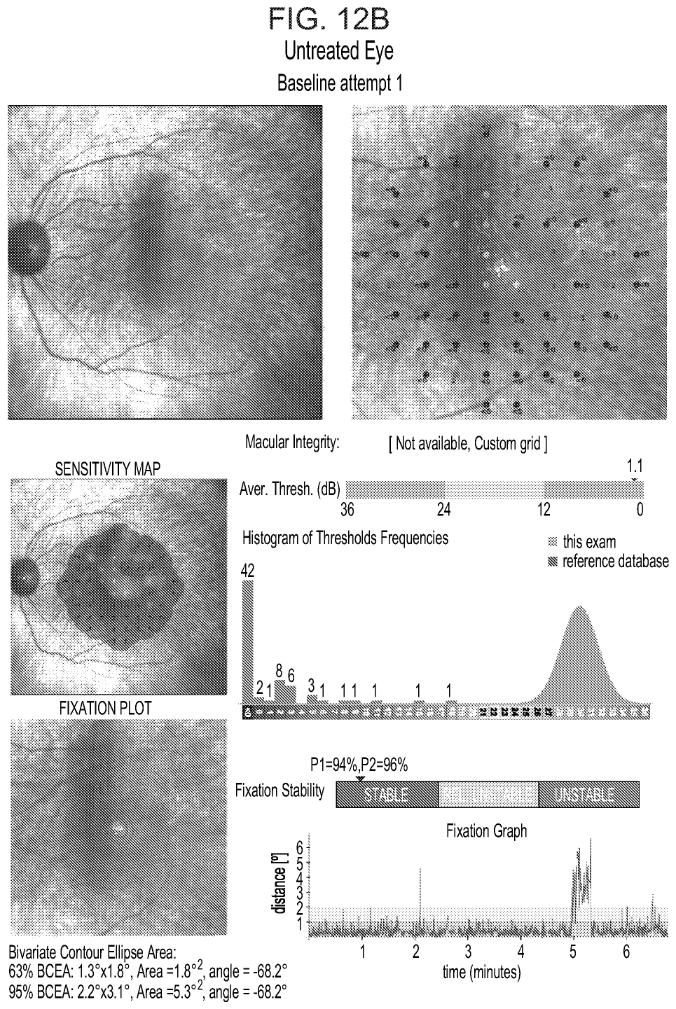

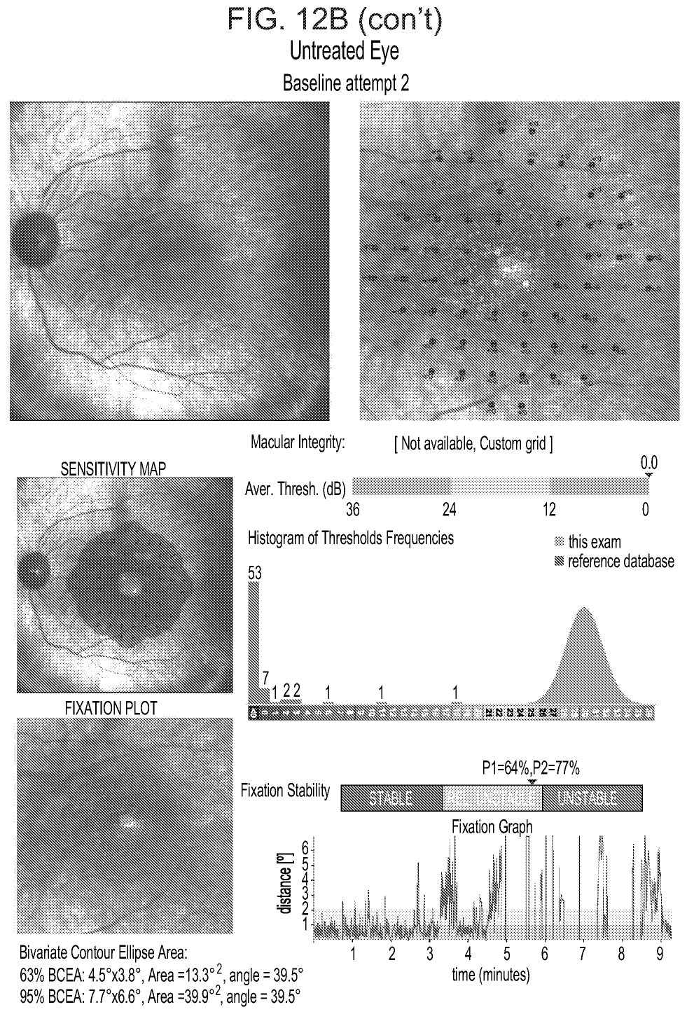

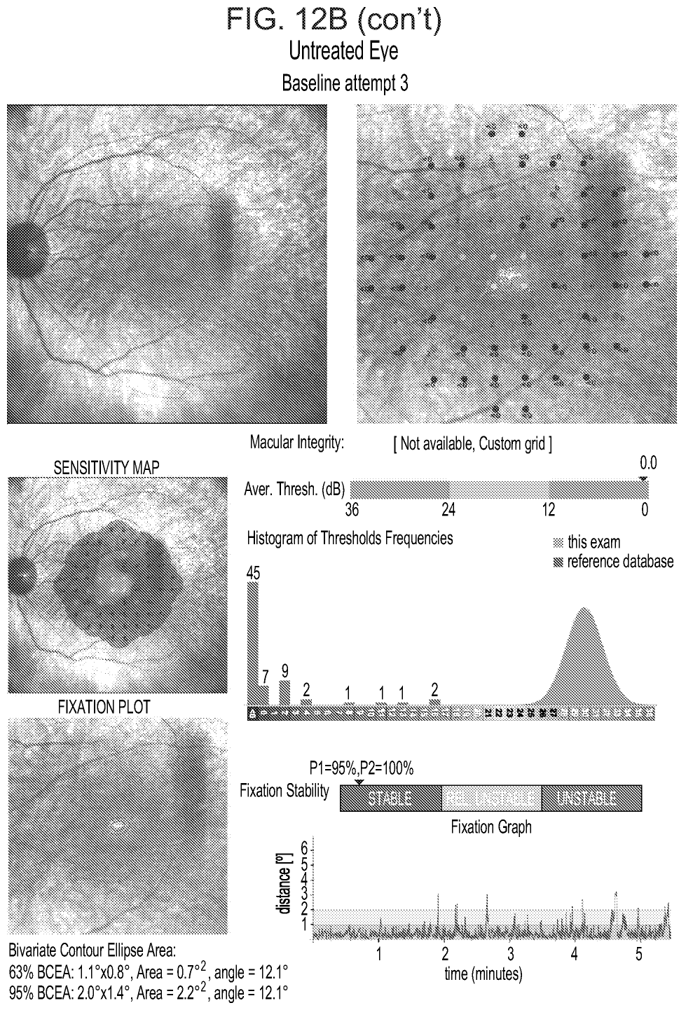

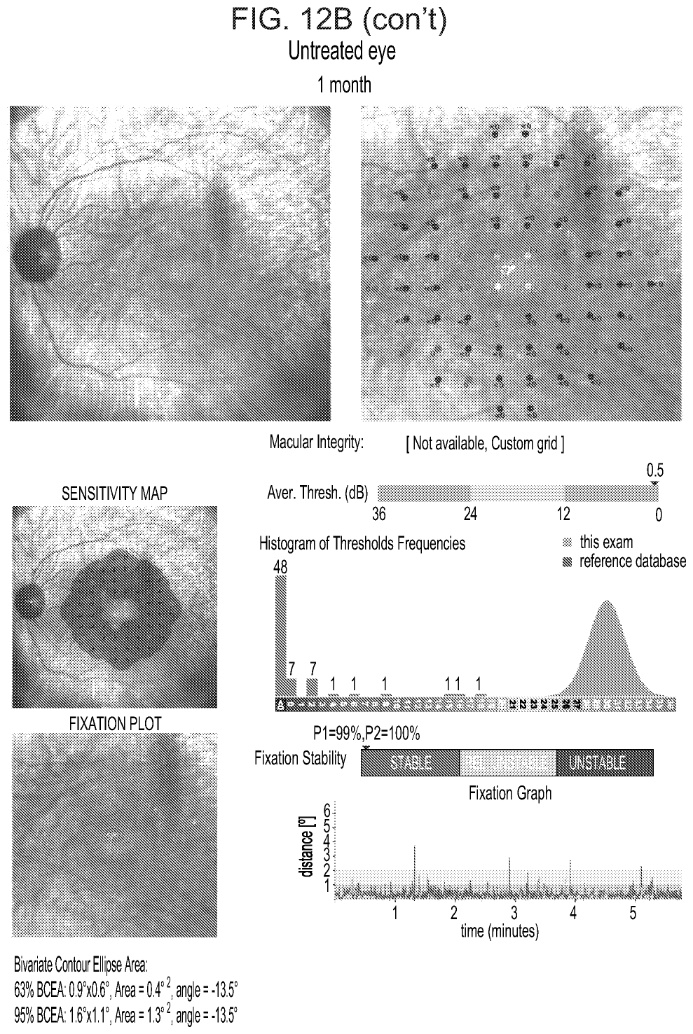

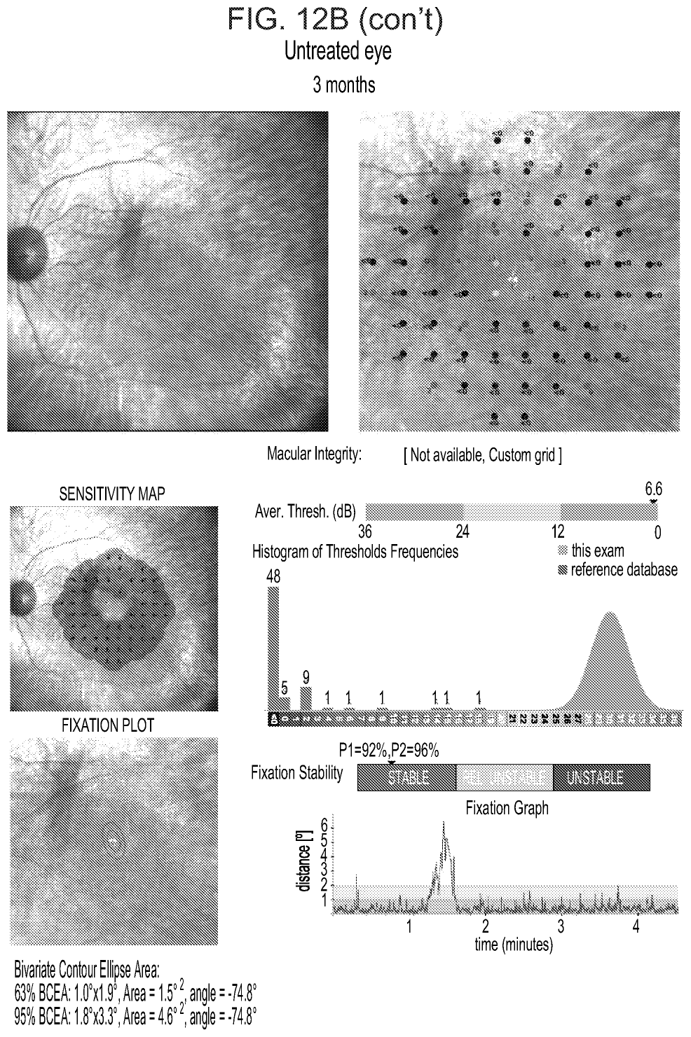

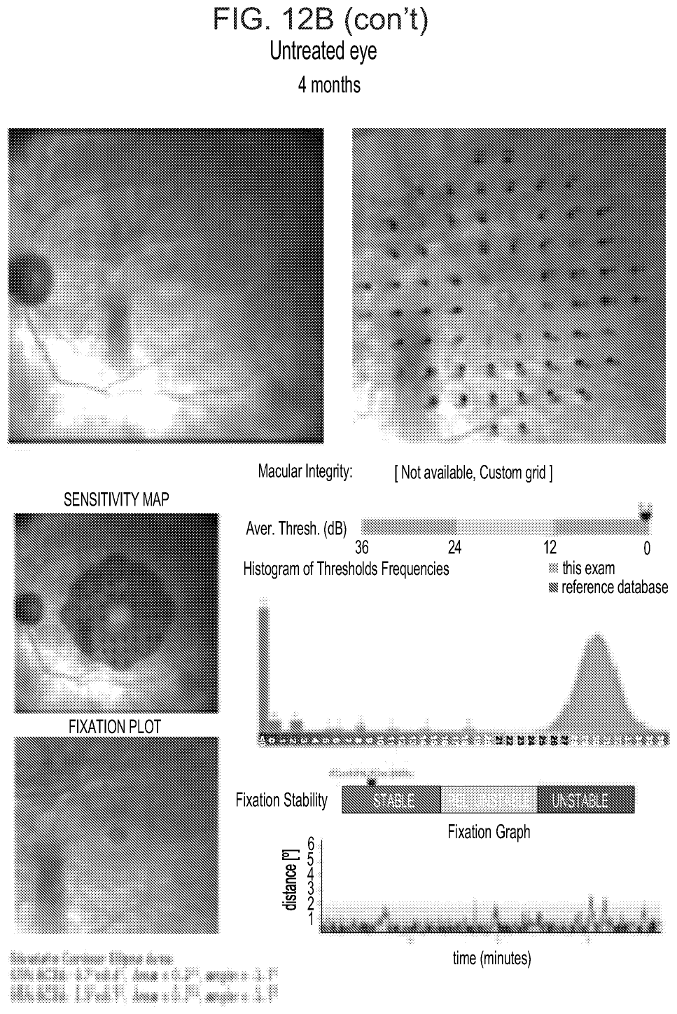

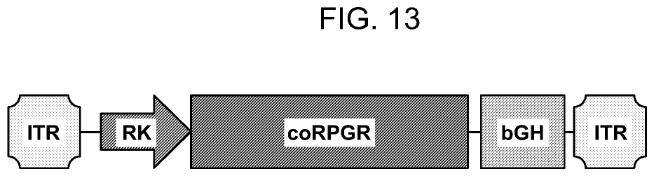

The patient underwent uneventful RPGR-gene therapy at a high dose (1.0×10¹¹ gp) to one eye with resolution of subretinal fluid by day 1 post-operatively. The methods for subject treatment and analysis are provided in Example 3. Two weeks after treatment, the patient described subjective improvement in visual clarity and visual field in the treated eye, which was corroborated by microperimetry testing of retinal sensitivity at 1 month follow-up (FIG. 11 and raw data in FIG. 12). At 5 weeks, the patient noticed partial visual regression in the treated eye with subjective paracentral scotomas. Microperimetry testing confirmed a reduction in retinal sensitivity in the treated eye (mean threshold sensitivity=0.0 dB).

### Example 3: Clinical Trial of Gene Therapy for Retinitis Pigmentosa

### 1.0: Investigational Plan

### 1.1: Overall Study Design

The safety, tolerability and efficacy of a single sub-retinal injection of an Adeno-Associated Viral Vector encoding Retinitis Pigmentosa GTPase Regulator (AAV8-RPGR) was evaluated in subjects with X-Linked Retinitis Pigmentosa (XLRP). A Phase 1/2, first-in-human, multi-center, dose-escalation interventional study of AAV8-RPGR in male subjects with genetically confirmed XLRP was conducted. The study was conducted in two parts: Part I was a dose escalation study, Part II was a Maximum Tolerated Dose (MTD) expansion study (as determined in Part I).

The study consists of 11 visits over a 24-month evaluation period. At the Screening/Baseline Visit, each subject was assessed for eligibility of both eyes. Only one eye received treatment (the “study eye”), and the untreated eye was designated as the “fellow eye.” Selection of the “study eye” was made on clinical grounds and was generally the worse eye affected. This was discussed in detail and agreed with each subject as part of the informed consent process.

At the Injection Day Visit (Visit 2, Day 0), subjects underwent vitrectomy and iatrogenic retinal detachment as part of a sub-retinal injection procedure for administration of AAV8-RPGR in their study eye. To minimize inflammation resulting from surgery and/or vector/transgene, all subjects were given a 21-day course of oral corticosteroid (e.g., prednisolone/prednisone) that started 2 days before the planned date of surgery (see Section 3.8 for details).

Subjects were assessed for safety and efficacy throughout the study as indicated in the Schedule of Study Procedures (see Table 2). The safety evaluation was based on the occurrence of adverse event (AE) reporting (including dose-limiting toxicity (DLTs)); full ophthalmic examination (including indirect ophthalmoscopy, slit-lamp examination, intraocular pressure [IOP], anterior chamber and vitreous inflammation grading and lens opacities classification system III [LOCS III] cataract grading); fundus photography; vital signs; and laboratory assessments (including laboratory safety parameters, viral shedding and immunogenicity). The efficacy evaluation was based on BCVA, SD-OCT, fundus autofluorescence, microperimetry, visual fields, contrast sensitivity, low luminance visual acuity (LLVA), full-field stimulus threshold test (FST), color vision, and reading test. Any safety information collected as a result of the efficacy assessments (e.g., BCVA) was also used in the overall safety evaluation, as applicable.

Subjects who develop cataracts may undergo cataract surgery if deemed clinically necessary; if surgery is performed, it should be carried out at least 4 weeks before Visit 9 (Year 1) or Visit 11 (Year 2).

TABLE 2
Schedule of Study Procedures

| | Study Visit | | | | | |
|---|---|---|---|---|---|---|
| | Screening/Baseline^{a} | Day 0 | Day 1 | Day 7 | Month 1 | Month 3 |
| Assessments/Procedures | Visit Window | | | | | |
| (All Subjects/ Both Eyes, | | | | (±3 d) | (±7 d) | (±7 d) |
| Unless Otherwise | Visit Number | | | | | |
| Specified) | Visit 1 | Visit 2 | Visit 3 | Visit 4 | Visit 5 | Visit 6 |
| Informed consent/assent | X | | | | | |
| Demography | X | | | | | |
| Medical history, incl ocular | X | | | | | |
| history and prior meds | | | | | | |
| Blood pressure | X | X | X | X | | |
| Pulse | X | X | X | X | | |
| Safety blood samples^{d} | X | | | X | X | X |
| RPGR mutation screen^{e} | X | | | | | |
| Full ophthalmic | X | | X | X | X | X |
| examination^{f} | | | | | | |
| Surgical procedure/dosing^{g} | | X | | | | |
| Dosing with oral steroids^{h} | X | | | | | |
| ETDRS BCVA | X^{m} | | X | X | X | X |
| SD-OCT | X | | X | X | X | X |
| LLVA | X^{m} | | | | X | X |
| Fundus autofluorescence | X | | | | X | X |
| Microperimetry | X^{m} | | | | X | X |
| Fundus photography | X | | | | | |
| FST | X | | | | | |
| Visual fields | X^{m} | | | | | |
| Contrast sensitivityⁱ | X | | | | | X |
| Color vision test | X | | | | | X |
| Speed reading test | X | | | | | |
| Viral shedding^{j} | X | | X | X | X | |
| Immunogenicity sampling^{k} | X | | X | X | X | X |
| AE, SAE monitoring^{l} | X | X | X | X | X | X |
| Concomitant medication | X | X | X | X | X | X |
| review | | | | | | |
| Corticosteroid compliance | | X | X | X | X | |
| review | | | | | | |
| Randomisationn | X | | | | | |

| | Study Visit | | | | | | |
|---|---|---|---|---|---|---|---|
| | Month 6 | Month 9 | Year 1 | Month 18 | Year 2 | ET Visit^{b} | Uns. Visit^{c} |
| Assessments/Procedures | Visit Window | | | | | | |
| (All Subjects/ Both Eyes, | (±14 d) | (±14 d) | (±14 d) | (±14 d) | (±14 d) | | |
| Unless Otherwise | Visit Number | | | | | | |
| Specified) | Visit 7 | Visit 8 | Visit 9 | Visit 10 | Visit 11 | | |
| Informed consent/assent | | | | | | | |
| Demography | | | | | | | |
| Medical history, incl ocular | | | | | | | |
| history and prior meds | | | | | | | |
| Blood pressure | | | | | | | |
| Pulse | | | | | | | |
| Safety blood samples^{d} | | | X | | X | X | |
| RPGR mutation screen^{e} | | | | | | | |
| Full ophthalmic | X | X | X | X | X | X | X |
| examination^{f} | | | | | | | |
| Surgical procedure/dosing^{g} | | | | | | | |
| Dosing with oral steroids^{h} | | | | | | | |
| ETDRS BCVA | X | X | X^{m} | X | X^{m} | X^{m} | X |
| SD-OCT | X | X | X | X | X | X | X |
| LLVA | X | X | X^{m} | X | X^{m} | X^{m} | |
| Fundus autofluorescence | X | X | X | X | X | X | |
| Microperimetry | X | X | X | X | X | X | |
| Fundus photography | X | | X | X | X | X | |
| FST | X | | X | | X | X | |
| Visual fields | X | | X | | X | X | |
| Contrast sensitivityⁱ | X | | X | | X | X | |
| Color vision test | X | | X | | X | X | |
| Speed reading test | X | | X | | X | X | |
| Viral shedding^{j} | | | | | | | |
| Immunogenicity sampling^{k} | X | | X | | | X | |
| AE, SAE monitoring^{l} | X | X | X | X | X | X | X |
| Concomitant medication | X | X | X | X | X | X | X |
| review | | | | | | | |
| Corticosteroid compliance | | | | | | | |
| review | | | | | | | |
| Randomisationn | | | | | | | |

Abbreviations:
AE = adverse event;
BCVA = best-corrected visual acuity;
ET = early termination;
ETDRS = Early Treatment of Diabetic Retinopathy Study;
IOP = intraocular pressure;
LOCS III = Lens Opacities Classification System III;
FST = Full field stimulus threshold test;
LLVA = Low luminance visual acuity;
SAE = serious adverse event;
SD-OCT = spectral domain optical coherence tomography

All procedures will be performed for both eyes, unless otherwise specified.
a The Screening/Baseline Visit must be performed within 8 weeks of Visit 2 (±2 weeks).
b An early termination (ET) visit is to be performed if a subject discontinues at any time.
c If clinically indicated, subjects may need to return to the site for an unscheduled visit. As a minimum, the following assessments will be performed: full ophthalmic examination, BCVA, SD-OCT, fundus autofluorescence, AE/SAE monitoring, and concomitant medication review.
d Includes haematology and clinical chemistry.
e To be conducted only if unavailable at Visit 1.
f Includes indirect ophthalmoscopy, slit lamp examination, IOP, anterior chamber and vitreous inflammation grading and LOCS III cataract grading.
g Study eye only.
h Subjects will be given a 21-day course of oral prednisone/prednisolone and instructed to start taking the drug 2 days before Visit 2. Subjects will take 1 mg/kg/day prednisone/prednisolone for a total of 10 days (beginning 2 days before the vector injection, on the day of injection, and then for 7 days); followed by 0.5 mg/kg/day for 7 days; 0.25 mg/kg/day for 2 days; and 0.125 mg/kg/day for 2 days (21 days in total).
i Pelli Robson chart will be used for contrast sensitivity.
j Blood, tears (both eyes), saliva, and urine samples will be collected for the viral shedding assay.
k Immunogenicity sampling at the ET Visit is to be conducted only if visit occurs prior to Year 1 Visit.
l SAEs will be collected from the time the subject provides written informed consent/assent through Visit 11 (or ET Visit if applicable). Non-serious AEs will be collected from Visit 2 through Visit 11 (or ET Visit if applicable)
m To be performed in triplicate
n Part II only

A subject was considered to have completed the study if he completed the Year 2 assessments. The end of the trial is the date the last subject completes his Year 2 assessments (or early termination [ET] assessments in the event of premature discontinuation) or the date of last data collection if the last subject is lost to follow-up.

### 1.2: Dose-Limiting Toxicity

DLTs were defined as any of the following events considered to be related to AAV8-RPGR:
Sustained decrease in BCVA of ≥30 letters on the Early Treatment of Diabetic Retinopathy Study (ETDRS) chart compared to baseline; sustained is defined as lasting 48 hours or more until recovery, with recovery defined as visual acuity (VA) returning to within 10 letters of baseline VA. An exception is made for surgery-related events occurring in close temporal association (within <24 hours) of the surgery.
Vitreous inflammation, vitritis (>Grade 3 using standardised Nussenblatt vitreous inflammation scale grading) (Nussenblatt et al., Ophthalmology. 1985; 92(4):467-471).
Any clinically significant retinal damage observed (e.g., retinal atrophy) that is not directly attributed to complications of surgery.
Any clinically relevant suspected unexpected serious adverse reaction, with the exception of vision loss or vision-threatening events (as defined in Section 6.2.1.2)

When triplicate BCVA assessments were performed at screening, the median BCVA result was used for change-from-baseline BCVA computation.

### 1.3: Part I: Dose-Escalation Study

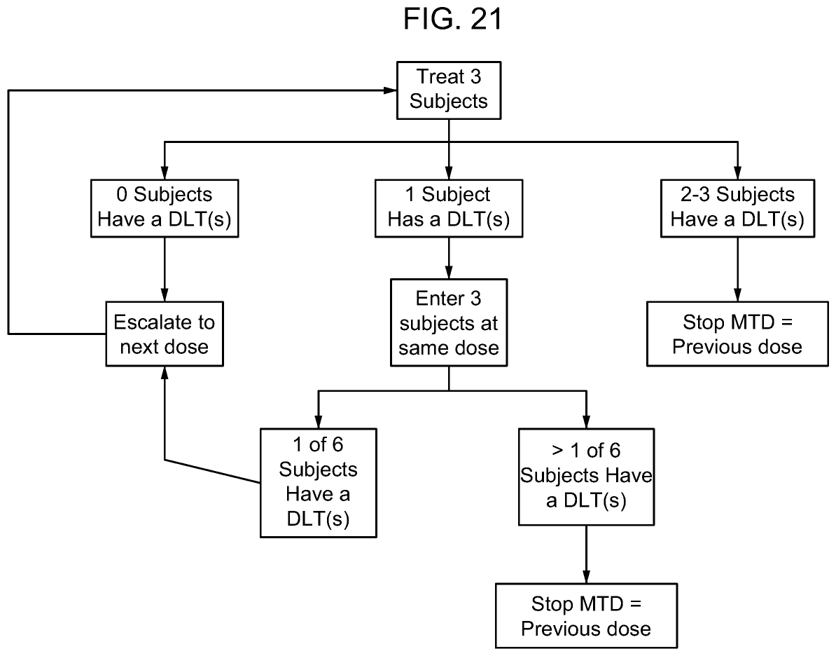

The study used a 3+3 escalation scheme (Storer, Biometrics. 1989; 45(3):925-937) for administration of AAV8-RPGR; a schematic diagram of the escalation scheme is displayed in FIG. 21.

The study involved up to 6 dose cohorts, with AAV8-RPGR doses of 5×10⁹ gp (Cohort 1), 1×10¹⁰ gp (Cohort 2), 5×10¹⁰ gp (Cohort 3), and 1×10¹¹ gp (Cohort 4), 2.5×10¹¹ gp (Cohort 5), and 5×10¹¹ gp (Cohort 6). Each eligible subject received AAV8-RPGR in their study eye and was monitored for DLTs.

An independent Data Monitoring Committee (DMC) was used to review safety data before confirming whether escalation to a higher dose level can occur. There is a potential for surgical complications resulting in safety events that meet the criteria for a DLT. In such cases, the DMC would the final adjudication as to whether the event is a DLT.

The DMC reviews safety data for each cohort when at least 3 subjects have been dosed at a particular level. However, if 2 subjects within a cohort have a DLT(s), dosing will not proceed to subsequent subjects until safety data are reviewed by the DMC.

For the purpose of making decisions regarding dose escalation, the DMC reviewed safety data collected for at least 4 weeks from each subject in the last dosed cohort. In addition, the DMC reviewed cumulative safety data collected from all previously-dosed cohorts and take these findings into consideration when making decisions on dose escalation.

There was a minimum of 4 weeks between each subject dosed in Cohort 1. Unless otherwise specified by the DMC, there are no restrictions on the interval between subjects being dosed in Cohort 2 onwards.

Three to 6 subjects are planned per dose cohort; however, the actual number of subjects enrolled into each cohort depends on the toxicity observed. If no DLTs are observed in the first 3 subjects treated within a cohort, then escalation to the next dose cohort can proceed. If one DLT is reported within a 3-subject cohort, an additional 3 subjects will be treated at the same dose. If there are no further DLTs reported in the additional 3 subjects, then escalation to the next dose cohort can proceed. If ≥2 subjects within a cohort (3 or 6 subjects) have a DLT(s), then the maximum tolerated dose (MTD) will be identified as the previous (lower) dose. If ≥2 subjects with a DLT are reported within Cohort 1 (3 or 6 subjects), then dosing will cease under this protocol and further investigation may occur following a protocol amendment.

### 1.4: Part II: Maximum Tolerated Dose Expansion Study

Once the MTD was identified, up to 45 additional subjects were randomized, in a 2:1 allocation ratio. Subjects received AAV8-RPGR either at the MTD (MTD cohort), or at a low dose (active-control cohort), three dose-levels below the MTD (e.g., low dose=5×10¹⁰ gp if MTD=5×10¹¹ gp). Part II of the study was randomized and double-masked to the assigned dose, and open-label to the treatment administration.

### 1.5: Number of Subjects

Overall, the study was expected to enroll approximately 63 subjects: 18 in Part I and 45 in Part II.

### 1.6: Discussion of Study Design and Dose Selection

Guidelines published by the European Medicines Agency (EMA) and Food and Drug Administration (FDA) on mitigating risks in first-in-human studies and use of gene therapy in clinical trials were used in the design of this study (ICH-E4, Guideline for Industry. Dose-Response Information to Support Drug Registration. November 1994; EMA Committee for Medicinal Products for Human Use, Guideline on strategies to identify and mitigate risks for first-in-human clinical trials with investigational medicinal products. July 2007, Concept paper on the revision of the ‘Guideline on 4 strategies to identify and mitigate risks for first-in-human 5 clinical trials with investigational medicinal products’. September 2016; EMA Committee for Advanced Therapies. Guideline on the quality, non-clinical and clinical aspects of gene therapy medicinal products. March 2015; FDA Guidance for Industry. Considerations for the design of early-phase clinical trials of cellular and gene therapy products. June 2015). An independent DMC is used to review safety data before any dose escalation decisions are made.

The subjects included in the study are representative of active XLRP disease and are selected to optimize observance of meaningful change in the outcome measures. The planned sample size is consistent with a 3+3 escalation scheme. A prospective trial period of 24 months is considered to be a sufficient period of time to monitor for any AEs related to the vector and/or transgene/administration procedure.

The starting dose used in this clinical study was 5×10⁹ gp AAV8-RPGR. This dose was primarily based on human equivalent doses (calculated on the basis of vitreous volume) from the AAV8-RPGR 26-week single-dose toxicity and biodistribution studies conducted by the sponsor of this study (NightstaRx) and the mouse studies conducted at the University of Oxford (Fischer et al., Mol Ther. 2017; 25(8):1854-1865). In the Fischer studies, treatment with 1.5×10⁹ gp AAV8-RPGR did not lead to toxic ocular effects in C57BL/6JWT. Results from the sponsor's toxicity and biodistribution studies indicated that AAV8-RPGR was well tolerated in male C57BL/6J mice at dose levels of 1×10⁹ and 3.54×10⁹ gp/eye. The NOAEL (no-observed-adverse-effect level) was determined to be greater than 3.54×10⁹ gp/eye in mice, providing a 700-fold safety margin compared to the starting dose.

The second and third dose levels in this study were 1×10¹⁰ and 5×10¹⁰ gp. These dose increments are less than a 1-log increase from the previous dose levels (i.e. 5×10⁹ and 1×10¹⁰ gp, respectively), considering the possibility of a narrow safe range for RPGR expression. Smaller dose increments were not expected to add meaningful information. Further, in a monkey study, dose thresholds of AAV8-GFP (an AAV8 virus particle encoding green fluorescence protein) were identified to effectively deliver gene product to target cells without toxicity, with the highest safe dose identified as 1×10¹⁰ gp (Vandenberghe et al., Sci Transl Med. 2011; 3(88):88ra54). In an ongoing Phase 1/2 clinical trial evaluating the safety and tolerability of sub-retinal AAV-CNGA3 vector (rAAV8.hCNGA3) in patients with CNGA3-linked achromatopsia, patients receive vector at doses between 1×10¹⁰ and 1×10¹¹ gp (ClinicalTrials.gov Identifier: NCT02610582). Preliminary results from this clinical study in subjects dosed with 1×10¹⁰ gp demonstrate acceptable safety (Fischer et al., Abstract 5207. 2016 Annual Meeting of the Association for Research in Vision and Ophthalmology), as do higher doses of up to 1×10¹¹ gp

The fourth (1×10¹¹ gp), fifth and sixth (2.5×10¹¹ and 5×10¹¹ gp) dose levels were less than a 0.5-log increase from the previous dose levels, ensuring a more conservative approach at the upper end of the dose-exploration range. The NOAEL in mice provides a 7-fold safety margin compared to the clinical maximum dose (5×10¹¹ gp).

A summary of the AAV8-RPGR doses in the toxicology species is presented in Table 3. The safety and efficacy findings from other pre-clinical and clinical studies with AAV8 vector for subretinal delivery are also included for comparison.

TABLE 3
Toxicology Safety Margin for Clinical Trials

| | | | | Safety Margin in | Safety Margin in |
|---|---|---|---|---|---|
| | | | | Comparison to | Comparison to |
| | | Vector/Dose | | Clinical | Clinical |
| | | Administered | HED* | Starting Dose | Maximum Dose |
| Reference | Species | (gp/eye) | (gp/eye) | 5 × 10⁹ gp/eye | 5 × 10¹¹ gp/eye |
| Fischer et al., | Mouse | AAV8- | 1.5 × 10¹² | 300 | 3.3 |
| 2017 | | RPGR: | | | |
| | | 1.5 × 10⁹ | | | |
| NightstaRx | Mouse | AAV8- | 1 × 10¹² | 200 | 2 |
| Toxicity and | | RPGR: | | | |
| biodistribution | | 1 × 10⁹ per eye | | | |
| studies - low | | (both eyes | | | |
| dose (Study | | were treated) | | | |
| LF66QG) | | | | | |
| NightstaRx | Mouse | AAV8- | 3.54 × 10¹² | 708 | 7.1 |
| Toxicity and | | RPGR: | | | |
| biodistribution | | 3.54 × 10⁹ per | | | |
| studies - high | | eye (both | | | |
| dose (Study | | eyes were | | | |
| LF66QG) | | treated) | | | |
| Vandenberghe et | Monkey | AAV8-GFP: | 2.4 × 10¹⁰ | 4.8 | — |
| al., 2011 | | 1 × 10¹⁰ | | | |
| Fischer et al., | Human | AAV- | 1 × 10¹⁰ | 2 | — |
| 2016 - low dose | | CNGA3: | | | |
| | | 1 × 10¹⁰ | | | |
| Fischer et al., | Human | AAV- | 1 × 10¹¹ | 20 | — |
| 2016 - high dose | | CNGA3: | | | |
| | | 1 × 10¹¹ | | | |

*Vitreal volumes are used for calculation of safety margins to correct for species differences after sub-retinal injection of vector. Ratio of vitreal volumes for human:mouse is 1:1000, and for human:monkey is 1:2.4 (Atsumi et al., 2013).

According to vitreous volume criteria used for calculation of HED in ophthalmic indications (1000-fold difference in the vitreous volume between mouse and human) and knowledge of safe, higher dose administration with subretinal injection of AAV8 vector (Vandenberghe et al., 2011), the higher doses with AAV8-RPGR may be possible if the safe RPGR expression through transgene does not exhibit a narrow range at lower end of doses.

Application of AAV8-RPGR to the under-surface of the retina requires retinal detachment following vitrectomy. As such, sub-retinal injection of AAV8-RPGR carries the risks associated with vitrectomy and retinal detachment, which include intra-operative and post-operative complications: infection (most notably infectious endophthalmitis); low and elevated IOP; choroidal detachment; macular oedema; vitreous haemorrhage; visual impairment; metamorphopsia; and photopsia (Park et al., Ophthalmology. 1995; 102:775-781; Thompson et al., Am J Ophthalmol. 1996; 121(6):615-622; Banker et al., Ophthalmology 1997; 104 (9):1442-1452; discussion 1452-1453; Cheng et al., Am J Ophthalmol. 2001; 132(6):881-887; Anderson et al., Ophthalmology. 2006; 113(1):42-47. Epub 2005 Dec. 19; Stein et al., Arch Ophthalmol. 2009; 127(12):1656-1663; Recchia et al., Ophthalmology 2010; 117(9):1851-1857). Post-operative intraocular inflammation caused by vitrectomy is often associated with transient visual impairment. A long-term complication of vitrectomy is cataract formation, which may require an additional surgical procedure (cataract extraction) (Park et al., 1995; Cheng et al., 2001; Recchia et al., 2010). To minimise inflammation resulting from potential immune responses to vector, subjects receiving AAV8-RPGR will be given a course of oral corticosteroid.

Once the MTD was identified and the safety and tolerability of AAV8-RPGR was demonstrated in adults, subjects ≥10 years of age were enrolled in Part II of the study. The 10-years age cut-off safeguards that participating pediatric subjects will be able to comply, adequately perform study assessments, and have sufficiently advanced disease that is encroaching on the macula (i.e. the AAV8-RPGR treatment administration area).

In Part II, subjects were randomized to the “MTD cohort,” the “active-control cohort,” or untreated control. This allowed for a parallel, active-control group and masking of the treatment dose, which enhanced the robustness of the efficacy and safety outcomes. The active-control cohort is three dose-levels below the MTD. This assures a 1-1.5-log difference in dose between these two cohorts, and allows for identifying a dose response while mitigating the possibility of a subtherapeutic low dose.

### 1.7: Endpoints

Primary Endpoint. The primary safety endpoint was incidence of dose-limiting toxicities (DLTs) and treatment-emergent adverse events (TEAEs) over a 24-month period.

Secondary and Exploratory Endpoints. Secondary endpoints of the study included:
Changes from baseline in microperimetry at 3, 6, 12, 18, and 24 months.
Changes from baseline in best-corrected visual acuity (BCVA) at 3, 6, 12, 18, and 24 months.
Changes from baseline in spectral domain optical coherence tomography (SD-OCT) at 3, 6, 12, 18, and 24 months.
Changes from baseline in autofluorescence at 3, 6, 12, 18, and 24 months.

Exploratory endpoints of the study included:
Changes from baseline in other anatomic and functional outcomes at 3, 6, 12, 18 and 24 months.

### 2.0: Selection and Withdrawal of Subjects

### 2.1: Inclusion Criteria

Subjects were eligible for study participation if they met all the following inclusion criteria.

1. Subject/parent (if applicable) is willing and able to provide informed consent for participation in the study
2. Are male and able to comply and adequately perform all study assessments
Part I: ≥18 years of age
Part II: ≥10 years of age
   3. Have a genetically confirmed diagnosis of XLRP (with RPGR mutation)
   4. Have active disease clinically visible within the macular region in both eyes and defined as follows:
   ellipsoid zone (EZ) on SD-OCT measured at screening, must be within the nasal and temporal border of any B-scan, and not be visible on the most inferior and superior B-scan
   5. Have a BCVA in both eyes that meets the following criteria, based on the cohort level
Cohort 1: better than or equal to light perception
Cohorts 2-3: BCVA of 34-73 ETDRS letters (equivalent to worse than or equal to 6/12 or 20/40 Snellen acuity, but better than or equal to 6/60 or 20/200 Snellen acuity).
Cohort 4-6 and Part II: better than or equal to BCVA of 34 ETDRS letters (equivalent to better than or equal to 6/60 or 20/200 Snellen acuity).

### 2.2: Exclusion Criteria

Subjects were not eligible for study participation if they met any of the following exclusion criteria:

1. Have a history of amblyopia in either eye
2. Are unwilling to use barrier contraception methods (if applicable), for a period of 3 months following treatment with AAV8-RPGR
3. Have any other significant ocular or non-ocular disease/disorder which, in the opinion of the investigator, may put the subjects at risk because of participation in the study, may influence the results of the study, may influence the subject's ability to perform study diagnostic tests, or impact the subject's ability to participate in the study. This would include, but is not limited to, the following:
clinically significant cataract
contraindication to oral corticosteroid
   4. Have participated in another research study involving an investigational product in the past 12 weeks or received a gene/cell-based therapy at any time previously (including, but not limited to, Intelligent Retinal Implant System implantation, ciliary neurotrophic factor therapy, nerve growth factor therapy).

### 2.3: Subject Withdrawal Criteria

Each subject has the right to withdraw from the study at any time without prejudice. In addition, the investigator may discontinue a subject from the study at any time if the investigator considers it necessary for any reason, including:
Significant protocol deviation
Significant non-compliance with study requirements
AE which results in an inability to continue to comply with study assessments
Lost to follow up
Death
Other (to be specified on the electronic case report form [eCRF]).

In the event that a subject discontinues the study, the reason for withdrawal is to be recorded in the eCRF. In the event that a subject discontinues the study early, the site should use every reasonable effort to ensure that an ET Visit is conducted as outlined in the Schedule of Study Procedures (see Table 2). If the subject is withdrawn due to an AE, the investigator will arrange for follow-up until the event has resolved or stabilised. For subjects who withdraw consent/assent, data will be collected through their last available study visit. Subjects withdrawn from the MTD cohort may possibly be replaced.

Withdrawal from the study will not result in the exclusion of a subject's data acquired up to the point of withdrawal.

### 3.0: Study Treatment

### 3.1: Treatments Administered

At the Injection Day Visit (Visit 2, Day 0), subjects underwent vitrectomy and retinal detachment in their study eye and then received a single, sub-retinal injection of AAV8-RPGR (See Section 3.4 for details). Subjects received an AAV8-RPGR dose of 5×10⁹ gp (Cohort 1), 1×10¹⁰ gp (Cohort 2), 5×10¹⁰ gp (Cohort 3), 1×10¹¹ gp (Cohort 4), 2.5×10¹¹ gp (Cohort 5), or 5×10¹¹ gp (Cohort 6). (see Section 1.3 for details).

### 3.2: Description of Study Drug

The drug substance was the AAV8 vector containing recombinant human complementary deoxyribonucleic acid (cDNA) encoding RPGR (AAV8-RPGR). The vector genome (AAV8-coRPGR-BGH, known as AAV8-RPGR) is comprised of a strong constitutive expression cassette, a rhodopsin kinase promoter, the codon-optimised human cDNA encoding RPGR (coRPGR), and a bovine growth hormone (BGH)-polyA sequence flanked by AAV2 inverted terminal repeats. The codon-optimized human coding sequence of the retina-specific isoform RPGR^{ORF15} was synthesised; the WT sequence of RPGR^{ORF15} was also synthesised and provided in a pCMV6-XL vector backbone or in a pUC57 vector backbone for cloning.

The AAV8-RPGR drug product was formulated in a sterile, 20 mM Tris-buffered solution, pH 8.0, and contains 1 mM MgCl₂, 200 mM NaCl, and 0.001% PF68. The drug product was a clear to slightly opalescent, colorless, sterile-filtered suspension with a target concentration of 5×10¹² gp/mL.

### 3.3: Packaging, Labeling, Preparation and Storage

AAV8-RPGR was supplied in labelled sterile polypropylene tubes, with each tube containing 0.3 mL vector suspension. Thus, each tube contained 1.5×10¹² gp in total.

AAV8-RPGR was delivered in a total volume of up to 0.1 mL. Instructions for preparation and dilution of drug product to deliver the desired dose of AAV8-RPGR were provided in the study procedure manual.

Prior to shipment, each vial was placed in a labelled secondary container. The drug product was to be stored at <−60° C. (<−76° F.) in a controlled access, temperature monitored freezer.

The Investigational Medicinal Product was labelled in compliance with regulatory standards (on either the primary or secondary container) and included the protocol study number, Sponsor's name, product name, titer, vial and lot number, expiration date, storage conditions and caution statement.

### 3.4: Vitrectomy Procedure and Injection of AAV8-RPGR

The subretinal injection technique to be used in this study was similar to that developed in the sponsor's Choroideremia programme in Oxford and other international investigator-sponsored trials in the United States, Canada and Germany. To date, over 185 subjects have been injected by four retinal surgeons using the technique described below.

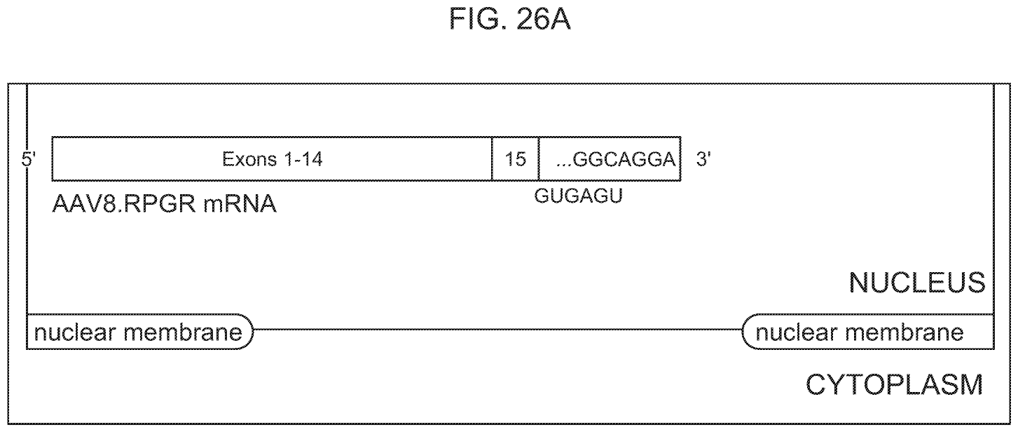

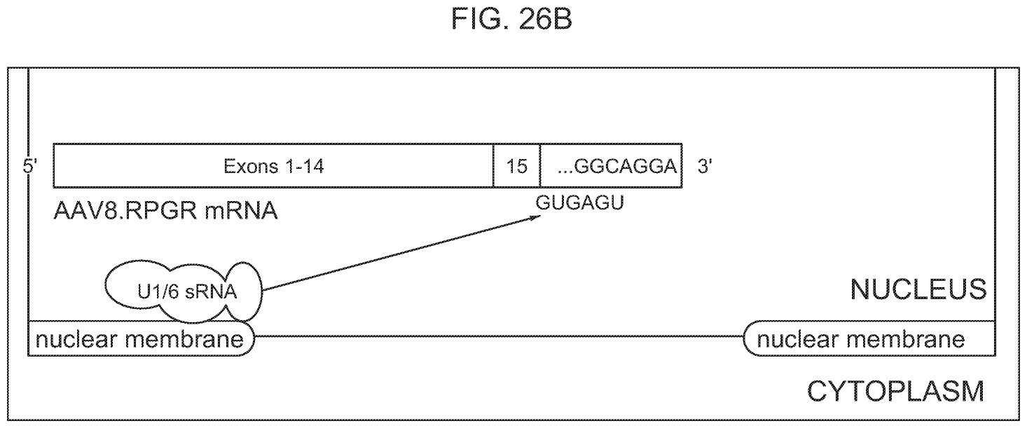

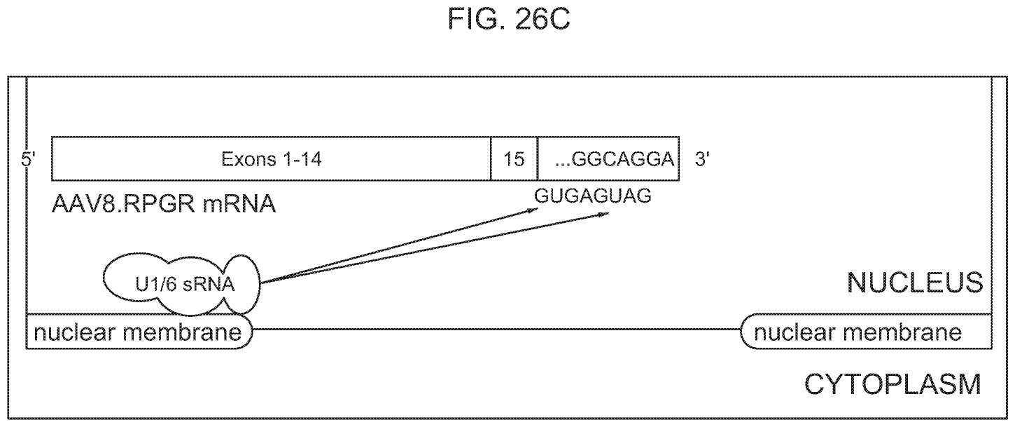

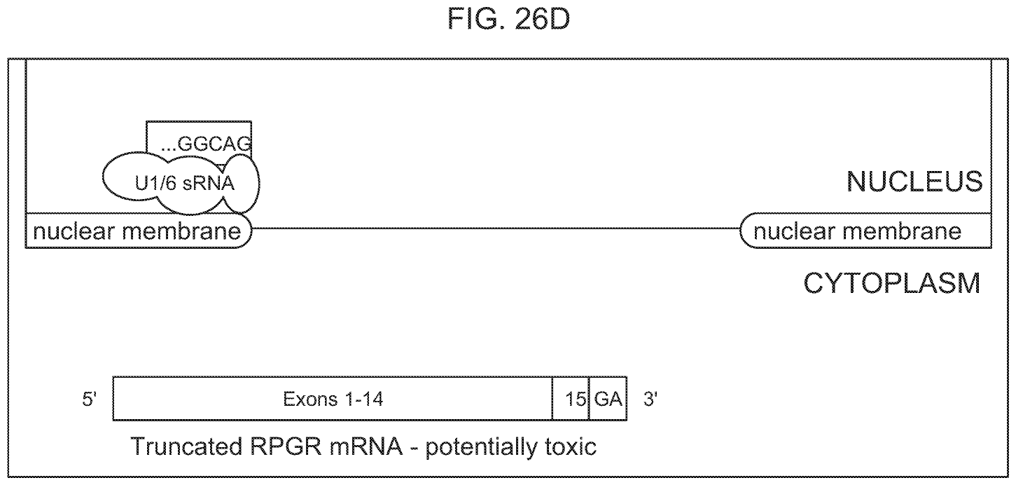

Injection of AAV8-RPGR was to be performed by an appropriately qualified and experienced retinal surgeon. Initially, subjects underwent a standard vitrectomy and detachment of the posterior hyaloid (FIGS. 26A-26B). All surgery was conducted using the standard BIOM® (binocular indirect ophthalmomicroscope) (OCULUS Surgical, Inc.) vitrectomy system. A 23-gauge sutured approach was usually favored to avoid any potential risks of wound leakage. If deemed easier, prior to sub-retinal injection of AAV8-RPGR, the retina was detached with 0.1-0.5 mL of balanced salt solution (BSS) injected through a 41-gauge sub-retinal cannula connected to a vitreous injection set. A single dose of AAV8-RPGR was injected into the sub-retinal fluid through the same entry site. If detachment of the macula occurred with a smaller volume of fluid, then additional subretinal sites in the posterior globe (e.g., nasal to the disc) may also be chosen to deliver up to the entire 0.1 ml of vector. This avoids excessive foveal stretch.

If unexpected complications of retinal detachment were encountered (e.g., macular hole created requiring treatment with gas), the injection of vector could deferred until a later date.

Subjects were monitored for the occurrence of AEs peri- and post-operatively. All AEs, irrespective of relationship to the study drug and/or the surgical procedure were captured in the subject's medical record and reported in the eCRF.

### 3.5: Randomization

The dose-escalation portion of this study was not randomized.

In Part II, after the study eye was assigned, subjects were randomized in a 2:1 ratio to receive either AAV8-RPGR MTD or a lower dose of AAV8-RPGR, three dose-levels from MTD (e.g., low dose=5×10¹⁰ gp if MTD=5×10¹¹ gp) for the active-control cohort.

Randomization was generated using a validated system that automates the random assignment of treatment groups to randomization numbers. Once a subject is deemed eligible, the investigative site (or authorized designee) accessed the system, and the subject was randomized using a standard blocked randomization. The randomization number included the center number and subject number.

### 3.6: Study Masking

Part I of the study was open-label.

Part II was double-masked (subject, surgeon, investigator/site team, sponsor were masked to the assigned dose, and open-label with respect to the treatment administration).

### 3.7: Study Drug Accountability

Records of the receipt and dispensing of study drug were kept by each study center until the end of the study to provide complete accounting of all used and unused study drug. Dispensation logs were checked by the sponsor (or its designee). Study centers destroyed all used vials in accordance with local procedures and returned all unused study drug to the sponsor (or its designee) at the end of the study. Final drug accountability was verified by the sponsor (or its designee).

### 3.8: Concomitant Therapy

Subjects cannot have participated in another research study involving an investigational product in the past 12 weeks or received a gene/cell-based therapy at any time previously (including, but not limited to, IRIS implantation, ciliary neurotrophic factor therapy, nerve growth factor therapy).

Throughout the study, investigators prescribed any concomitant medications or treatments deemed necessary to provide adequate supportive care. Details of concomitant medications were collected at the Screening/Baseline Visit and updated at every study visit (including the ET Visit, if applicable). Concomitant medications (including prednisone/prednisolone) taken during the study were to be recorded in the subject's medical records and eCRF; an exception to this is any medication used in the course of conducting a study procedure (e.g., anaesthesia, dilating eye drops).

To minimize inflammation resulting from surgery and potential or unexpected immune responses to vector/transgene, adult subjects are given a 9-week course of oral corticosteroid starting 3 days before surgery: 21 days at 60 mg, followed by 6 weeks of tapering doses. The dose regimen is adjusted for pediatric subjects treated in Part II (see Section 9.8) Subjects may also be treated at the time of surgery with up to 1 mL of triamcinolone (40 mg/mL), administered via a deep sub-Tenon approach.

### 3.9: Treatment Compliance

This study involved a single sub-retinal injection of up to 0.1 mL AAV8-RPGR. Measure of treatment compliance with AAV8-RPGR was therefore not necessary. Compliance with the use of prednisone/prednisolone was captured in the eCRF.

### 4.0: Study Visits and Procedures

The schedule of study procedures is presented in Table 2. Visits are described in more detail below.

### 4.1: Visit 1 (Screening/Baseline Visit)

The investigator explained the study purpose, procedures and subject responsibilities to each potential study subject. The subject's willingness and ability to meet the protocol requirements was determined.

Prior to any study-specific procedure, written informed consent was obtained. The subject or parent signed and dated one copy of the consent form in the presence of the investigator or his/her designee. The original signed form was retained at the study site and an additional copy remained in the subject's medical records; a copy was given to the subject or parent. Where applicable, an assent form was completed by the subject.

After informed consent/assent had been obtained, the subject was evaluated to determine eligibility. Screening assessments were considered baseline measurements and consisted of the following:
Demography
Medical history, including ocular history and prior medications
Blood pressure and pulse
Collection of safety blood samples (haematology and clinical chemistry)
RPGR mutation screen (only if not conducted previously)
Full ophthalmic examination, including indirect ophthalmoscopy, a slit-lamp examination, IOP, anterior chamber and vitreous inflammation grading and lens LOCS III cataract grading
ETDRS BCVA*
SD-OCT
LLVA*
Fundus autofluorescence
Microperimetry*
Fundus photography
Visual fields*
Contrast sensitivity test
Color vision test
Speed reading test
FST
Viral shedding
Immunogenicity sampling
SAE monitoring
Concomitant medication review
Randomization**
   *Assessments collected in triplicate. To facilitate triplicate testing, the visit was conducted over 2 days. It was recommended to measure BCVA and LLVA twice on the first day and once on the second day (prior to pupil dilation). All 3 BCVA and all 3 LLVA values must be recorded in the eCRF. The highest BCVA score was used to define subject eligibility. LLVA was conducted immediately after each BCVA assessment. Visual field and microperimetry outputs were sent to a CRC for review. Data was generated and collated within the CRC and exported to the Sponsor or designee for inclusion in the study database.
   **Randomisation for Part II only.

Subjects who met all of the inclusion criteria and none of the exclusion criteria had a study eye assigned and were enrolled into the study. In Part II, subjects were then randomized to the AAV8-RPGR treatment groups (MTD cohort, active-control cohort, or untreated control), and remained masked to the treatment dose. See Section 3.5 for details on randomization and assignment of subject numbers.

The next study visit (Visit 2) was to be scheduled within 8 weeks of the Screening/Baseline Visit (±2 weeks). Subjects were given a 21-day course of oral prednisone/prednisolone and instructed to start taking the drug 2 days before their next study visit (Visit 2). Where applicable, subjects were also instructed to use barrier contraception for a period of 3 months from the time they are treated.

### 4.2: Visit 2 (Day 0, Surgery/Injection Day Visit)

At Visit 2, the following assessments were performed prior to surgery:
Blood pressure and pulse
AE/SAE monitoring
Concomitant medication review
Corticosteroid compliance review

Subjects then underwent vitrectomy and receives a sub-retinal injection of AAV8-RPGR (see Section 3.4 for details). Subjects were carefully monitored for the occurrence of AEs during the procedure. Subjects could stay overnight or return to the site 1 day and then 7 days after surgery for post-operative follow-up (Visits 3 [Day 1] and 4 [Day 7], respectively).

### 4.3: Visit 3 (Day 1 Post-Operative Visit)

At Visit 3, the first post-operative visit, the following assessments were performed:
Blood pressure and pulse
Full ophthalmic examination, including indirect ophthalmoscopy, a slit-lamp examination, IOP, anterior chamber and vitreous inflammation grading and LOCS III cataract grading
ETDRS BCVA
SD-OCT
Viral shedding
Immunogenicity sampling
AE/SAE monitoring
Concomitant medication review
Corticosteroid compliance review

Where applicable, subjects were reminded of the requirement to use barrier contraception for a period of 3 months from the time of treatment.

4.4: Visit 4 (Day 7 Post-Operative visit ±3 Days)

At Visit 4, the second post-operative visit, the following assessments were performed:
Blood pressure and pulse
Collection of safety blood samples (haematology and clinical chemistry)
Full ophthalmic examination, including indirect ophthalmoscopy, a slit-lamp examination, IOP, anterior chamber and vitreous inflammation grading and LOCS III cataract grading
ETDRS BCVA
SD-OCT
Viral shedding
Immunogenicity sampling
AE/SAE monitoring
Concomitant medication review
Corticosteroid compliance review

### 4.5: Visit 5 (Month 1±7 Days)

At Visit 5, the following assessments were performed:
Collection of safety blood samples (haematology and clinical chemistry)
Full ophthalmic examination, including indirect ophthalmoscopy, a slit-lamp examination, IOP, anterior chamber and vitreous inflammation grading and LOCS III cataract grading
ETDRS BCVA
SD-OCT
LLVA
Fundus autofluorescence
Microperimetry
Viral shedding
Immunogenicity sampling
AE/SAE monitoring
Concomitant medication review
Corticosteroid compliance review

### 4.6: Visit 6 (Month 3±7 Days)

At Visit 6, the following assessments were performed:
Collection of safety blood samples (haematology and clinical chemistry)
Full ophthalmic examination, including indirect ophthalmoscopy, a slit-lamp examination, IOP, anterior chamber and vitreous inflammation grading and LOCS III cataract grading
ETDRS BCVA
SD-OCT
LLVA
Fundus autofluorescence
Microperimetry
Contrast sensitivity test
Color vision test
Immunogenicity sampling
AE/SAE monitoring
Concomitant medication review

### 4.7: Visit 7 (Month 6±14 Days)

At Visit 7, the following assessments were performed:
Full ophthalmic examination, including indirect ophthalmoscopy, a slit-lamp examination, IOP, anterior chamber and vitreous inflammation grading and LOCS III cataract grading
ETDRS BCVA
SD-OCT
LLVA
Fundus autofluorescence
Microperimetry
Fundus photography
Visual fields
Contrast sensitivity test
Color vision test
Speed reading test
FST
Immunogenicity sampling
AE/SAE monitoring
Concomitant medication review

### 4.8: Visit 8 (Month 9±14 Days)

At Visit 8, the following assessments were performed:
Full ophthalmic examination, including indirect ophthalmoscopy, a slit-lamp examination, IOP, anterior chamber and vitreous inflammation grading and LOCS III cataract grading
ETDRS BCVA
SD-OCT
LLVA
Fundus autofluorescence
Microperimetry
AE/SAE monitoring
Concomitant medication review

### 4.9: Visit 9 (Year 1±14 Days)

At Visit 9, the following assessments were performed:
Collection of safety blood samples (haematology and clinical chemistry)
Full ophthalmic examination, including indirect ophthalmoscopy, a slit-lamp examination, IOP, anterior chamber and vitreous inflammation grading and LOCS III cataract grading
ETDRS BCVA*
SD-OCT
LLVA*
Fundus autofluorescence
Microperimetry
Fundus photography
Visual Fields
Contrast sensitivity test
Color vision test
Speed reading test
FST
Immunogenicity sampling
AE/SAE monitoring
Concomitant medication review
   *Assessments collected in triplicate. To facilitate triplicate testing, the visit was conducted over 2 days. It was recommended to measure BCVA and LLVA twice on the first day and once on the second day (prior to pupil dilation). All 3 BCVA and all 3 LLVA values were recorded in the eCRF. LLVA should be conducted immediately after each BCVA assessment.

Subjects who develop cataracts may undergo cataract surgery if deemed clinically necessary; if surgery is performed, it should be carried out at least 4 weeks before the Visit 9 (Year 1) or Visit 11 (Year 2).

### 4.10: Visit 10 (Month 18±14 Days)

At Visit 10 the following ocular assessments were performed:
Full ophthalmic examination, including indirect ophthalmoscopy, a slit-lamp examination, TOP, anterior chamber and vitreous inflammation grading and LOCS III cataract grading
ETDRS BCVA
SD-OCT
LLVA
Fundus autofluorescence
Microperimetry
Fundus photography
AE/SAE monitoring
Concomitant medication review

### 4.11: Visit 11 (Year 2±14 Days, End of Study Visit)

At Visit 11 the following ocular assessments were performed:
Full ophthalmic examination, including indirect ophthalmoscopy, a slit-lamp examination, IOP, anterior chamber and vitreous inflammation grading and LOCS III cataract grading
ETDRS BCVA*
SD-OCT
LLVA*
Fundus autofluorescence
Microperimetry
Fundus photography
Visual Fields
Contrast sensitivity test
Color vision test
Speed reading test
FST
AE/SAE monitoring
Concomitant medication review
   * Assessments collected in triplicate. To facilitate triplicate testing, the visit was conducted over 2 days. It was recommended to measure BCVA and LLVA twice on the first day and once on the second day (prior to pupil dilation). All 3 BCVA and all 3 LLVA values were recorded in the eCRF. LLVA was conducted immediately after each BCVA assessment.

### 4.12: Early Termination (ET) Visit

In the event that a subject discontinues the study at any time, the site should use every reasonable effort to ensure that an ET Visit is conducted. The following assessments should be performed:
Full ophthalmic examination, including indirect ophthalmoscopy, a slit-lamp examination, IOP, anterior chamber and vitreous inflammation grading and LOCS III cataract grading
ETDRS BCVA*
SD-OCT
LLVA*
Fundus autofluorescence
Microperimetry
Fundus photography
Visual Fields
Contrast sensitivity test
Color vision test
Speed reading test
FST
Immunogenicity sampling
AE/SAE monitoring
Concomitant medication review
   * Assessments collected in triplicate. To facilitate triplicate testing, the visit should be conducted over 2 days. It is recommended to measure BCVA and LLVA twice on the first day and once on the second day (prior to pupil dilation). All 3 BCVA and all 3 LLVA values must be recorded in the eCRF. LLVA should be conducted immediately after each BCVA assessment.

### 4.13: Unscheduled Visits

If clinically indicated, subjects may need to return to the site for an unscheduled visit. At a minimum, the following assessments are to be performed.
Full ophthalmic examination, including indirect ophthalmoscopy, a slit-lamp examination, IOP, anterior chamber and vitreous inflammation grading and LOCS III cataract grading
ETDRS BCVA
SD-OCT
AE/SAE monitoring
Concomitant medication review

### 5.0: Assessment of Efficacy

### 5.1: Best-Corrected Visual Acuity (BCVA)

To evaluate changes in VA over the study period, BCVA were assessed for both eyes using the ETDRS VA chart at the times indicated in Table 2.

The BCVA test was performed prior to pupil dilation, and distance refraction was carried out before BCVA was measured. Initially, letters were read at a distance of 4 meters from the chart. If <20 letters were read at 4 meters, testing at 1 meter should be performed. BCVA was reported as number of letters read correctly by the subject. At the Screening/Baseline Visit, eyes were eligible for the study if they:
Have better than or equal to light perception (Cohort 1 only), or
Have a BCVA of 34-73 ETDRS letters (equivalent to worse than or equal to 6/12 or 20/40 Snellen acuity, but better than or equal to 6/60 or 20/200 Snellen acuity) (Cohorts 2-3)
Have a BCVA better than or equal to 34 ETDRS letters (equivalent to better than or equal to 6/60 or 20/200 Snellen acuity) (Cohort 4 [5 and 6, if applicable] and MTD cohort)

For BCVA, assessors were appropriately qualified for conducting the assessment. BCVA was performed in triplicate over a 2-day period at Visits 1, 9 and 11 (or ET Visit) for all subjects. It was recommended that BCVA be conducted twice on the first day and once on the second day. All values were entered in the eCRF.

### 5.2: Spectral Domain Optical Coherence Tomography (SD-OCT)

SD-OCT was performed for both eyes at the times indicated in Table 2. SD-OCT measurements were taken by certified technicians at the site after dilation of the subject's pupil. All OCT scans were submitted by the sites to a Central Reading Center (CRC) where the scans were evaluated; the CRC will enter the data into the Electronic Data Capture (EDC) system. SD-OCT was used to quantify integrity of the ellipsoid zone and reduction in the signal from the outer nuclear layer and choroid. In addition, foveal changes were assessed.

### 5.3: Fundus Autofluorescence

To assess changes in the area of viable retinal tissue, fundus autofluorescence was performed for both eyes at the times indicated in Table 2. All fundus autofluorescence images were performed by certified technicians at the site after dilation of the subject's pupil and sent to a CRC for review; the CRC entered the data into the EDC system.

### 5.4: Microperimetry

Microperimetry was conducted for both eyes at the times indicated in Table 2. Microperimetry was performed in triplicate over a 2-day period at Visit 1 for all subjects. Microperimetry was conducted by certified technicians to assess changes in retinal sensitivity within the macula. All microperimetry images were sent by the sites to a CRC for review; the CRC entered the data into the EDC system.

### 5.5: Visual Fields

Visual fields were assessed in both eyes at the times indicated in Table 2 only at sites where the required perimetry equipment was available. Visual fields were assessed in triplicate over a 2-day period at Visit 1 for all subjects. Visual field outputs were sent to a CRC for review. Data was generated and collated within the CRC and exported to the sponsor or designee for inclusion in the study database.

### 5.6: Contrast Sensitivity

Contrast sensitivity was measured for both eyes at the times indicated in Table 2. Contrast sensitivity was measured prior to pupil dilation using a Pelli Robson chart. For contrast sensitivity, assessors were appropriately qualified for conducting the assessment.

### 5.7: Low Luminance Visual Acuity (LLVA)

LLVA was measured for both eyes at the times indicated in Table 2. The test was performed after BCVA testing and prior to pupil dilation. LLVA was measured by placing a 2.0-log-unit neutral density filter over the front of each eye and having the subject read the normally illuminated ETDRS chart. Initially, letters were read at a distance of 4 meters from the chart. If <20 letters are read at 4 meters, testing at 1 meter should be performed. LLVA was reported as number of letters read correctly by the subject. LLVA was performed in triplicate over a 2-day period at Visit 1 and Visit 9 and 11 (or ET Visit) for all subjects. It was recommended that LLVA be conducted twice on the first day and once on the second day. All values were entered into the eCRF.

### 5.8: Full Field Stimulus Threshold Test (FST)

FST was measured for both eyes after a period of dark adaptation and at the times indicated in Table 2 only at sites where the required FST equipment was available. FST measurements were taken by appropriately qualified technicians.

### 5.9: Color Vision

Color vision was tested for both eyes prior to pupil dilation, at the times indicated in Table 2. Eyes were tested separately and in the same order at each assessment. For color vision testing, assessors were appropriately qualified for conducting the assessment.

### 5.10: Reading Test

Reading performance was evaluated prior to pupil dilation for both eyes at the times indicated in Table 2. The reading test was provided to each site by the sponsor. For the reading test, assessors were appropriately qualified for conducting the assessment.

### 6.0: Assessment of Safety

### 6.1: Dose Limiting Toxicity

See Section 1.2 for definitions of DLTs.

### 6.2: Evaluation, Recording, and Reporting Adverse Events

### 6.2.1 Definitions

### 6.2.1.1 Adverse Event

An AE is any untoward medical occurrence in a clinical investigation subject, which does not necessarily have a causal relationship with the study medication/surgical procedure. An AE can therefore be any unfavourable and unintended sign (including an abnormal laboratory finding), symptom, or disease temporally associated with the use of the study medication/surgical procedure, whether or not related to the investigational product or with the surgical procedure described in this protocol.

AEs are to also include any pre-existing condition (other than XLRP) or illness that worsens during the study (i.e., increases in frequency or intensity).

### 6.2.1.2 Serious Adverse Event

An SAE is defined as any untoward medical occurrence that:
Results in death
Is life-threatening
Requires inpatient hospitalization or prolongation of existing hospitalization
Results in persistent or significant disability/incapacity
Is a congenital anomaly/birth defect
Results in vision loss or is vision threatening
Is another important medical event(s).

The term ‘life-threatening’ in the definition of ‘serious’ refers to an event in which the subject is at risk of death at the time of the event. It does not refer to an event that hypothetically might cause death if it were more severe.

Hospitalization for a pre-existing condition, including elective procedures, which has not worsened, does not constitute an SAE.

Other events that may not result in death, are not life threatening or do not require hospitalization, may be considered an SAE when, based upon appropriate medical judgment, the event may jeopardize the subject and may require medical or surgical intervention to prevent one of the outcomes listed above.

The following vision loss or vision-threatening events were to be reported as SAEs:
sustained decrease in VA of ≥15 letters on ETDRS chart compared to baseline, except for surgery-related events. Sustained is defined as lasting 48 hours or more until recovery; recovery defined as VA returned to within 10 letters of baseline VA.
Surgery-related events of VA decrease are defined as VA decreases occurring in close temporal association (within <24 hours) of the surgery. These events are not to be reported as an SAE, however, they should be reported as an AE if in the investigator's opinion, their evolution in terms of duration or severity is atypical for the surgical procedure. This would include, but not be limited to, instances where the abnormal course of post-surgery VA decrease is associated with another complication attributable to the surgery or the study medication, or where the abnormal course of post-surgery VA decrease can be attributed to another identifiable cause.
AEs that in the opinion of the investigator, actually or potentially require any surgical or medical intervention to prevent permanent loss of sight.

### 6.2.2 Recording of Adverse Event

SAEs were to be collected from the time the subject or parent (where applicable) provides written informed consent through Visit 11 (or ET Visit or Unscheduled Visits, if applicable). Non-serious AEs were to be collected from Visit 2 through Visit 11 (or ET Visit or Unscheduled Visits, if applicable). Subjects were questioned on the occurrence of an AE at every visit including any unscheduled visit, by using non-leading questioning such as ‘How have you been since the last visit?’

All AEs occurring during the study observed by the investigator or reported by the subject, whether or not attributed to study medication or the surgical procedure, were to be recorded in the subject's medical records and in the eCRF. Any clinically significant changes in laboratory results or vital sign measurements (as determined by the investigator) were to be recorded as an AE.

The following information was to be recorded in the eCRF for each AE: description, date of onset and end date, outcome, severity, assessment of relatedness to study medication/study procedure, the action taken and confirmation of whether the event is considered serious (see Section 6.2.1.2 for the definition of seriousness). Follow-up information should be provided as necessary (see Section 6.2.3 for specifics on follow-up procedures). The severity of events was to be assessed on the following scale: 1.=mild (awareness of sign or symptom, but easily tolerated) 2.=moderate (discomfort sufficient to cause interference with normal activities) 3.=severe (incapacitating, with inability to perform normal activities). When assigning relatedness of the AE, consideration will be given to whether there is a plausible relationship to either the study medication or the surgical procedure.

The following are definitions of relatedness that were used in this study: Unrelated: is not reasonably related in time to the administration of the study medication/surgical procedure or exposure of the study medication/surgical procedure has not occurred Unlikely to be related: there are factors (evidence) explaining the occurrence of the event (e.g., progression of the underlying disease or concomitant medication more likely to be associated with the event) or a convincing alternative explanation for the event Possibly related: clinically or biologically reasonable relative to the administration of the study medication/surgical procedure, but the event could have been due to another equally likely cause Probably related: is clinically/biologically reasonable relative to the administration of the study medication/surgical procedure, and the event is more likely explained by exposure to/administration of the study medication/surgical procedure than by other factors and causes Definitely related: a causal relationship of the onset of the event, relative to administration of the study medication/surgical procedure and there is no other cause to explain the event.

AE severity and relationship to the study medication or the surgical procedure was to be assessed at the site by the investigator or a medically qualified designee.

### 6.2.3 Follow-up of Adverse Events

AEs were to be followed until the subject has recovered or the subject's participation in the study is complete.

Subjects who are withdrawn from the study as a result of a drug-related AE will be followed up until the event has resolved, subsided, stabilized or the subject or parent (where applicable) withdraws consent or is lost to follow-up.

All SAEs, regardless of attribution to study medication or the surgical procedure, should be followed-up until the event has resolved, subsided, stabilised or the subject or parent (where applicable) withdraws consent or is lost to follow-up. The Sponsor (or designee) will follow up SAE reports to completion. Investigators were expected to timely provide the requested additional information for a complete assessment and documentation of the SAE reports.

### 6.2.4 Reporting of Serious Adverse Events and DLTs

The investigator shall immediately (within 24 hours of learning of the event) report any SAE (and/or DLT) to the Sponsor (or its designee). The initial report shall be promptly followed up with a more detailed report providing specifics about the subject and the event. Copies of hospital reports, autopsy reports and other documents should be provided (if applicable).

The sponsor will report Suspected Unexpected Serious Adverse Reactions (SUSARs) to investigative sites, Institutional Review Boards/Independent Ethics Committees (IRBs/IECs) and regulatory authorities in compliance with current legislation. All cases that are fatal or life-threatening were to be reported no later than 7 days after the sponsor received the initial report from the investigator. All non-fatal or non-life-threatening cases were to be reported within a maximum of fifteen days after the initial investigator's report. The sponsor will also provide periodic safety reports to IRBs/IECs and regulatory authorities as applicable.

### 6.2.5 Data Monitoring Committee (DMC)

An independent DMC was used in this study to safeguard the safety and interests of study subjects and assess the safety and risk/benefit of the gene therapy intervention during the trial. At regular intervals during the study, the DMC reviewed the progress and accrued study data and provided advice to the Sponsor on the safety aspects of the study, including recommendations for dose escalation (see Section 1.3). The DMC was to inform the Sponsor if there is a consensus that the ongoing data show that the gene therapy, its method of administration, and/or the study design are no longer in the best interests of study subjects.

### 6.3: Pregnancy

Any pregnancy that occurs during the clinical study in a female partner of a study subject should be recorded on a Pregnancy Notification Form. The investigator shall immediately (within 24 hours of learning of the event) report the pregnancy to the Sponsor (or its designee). In addition, if possible, outcome of the pregnancy fathered by the subject should be recorded and followed up until delivery for congenital abnormality or birth defect.

### 6.4: Full Ophthalmic Examination

A full ophthalmic examination was conducted for both eyes at the times indicated in Table 2. The ophthalmic examination included indirect ophthalmoscopy, slit lamp examination, TOP, anterior chamber and vitreous inflammation grading and LOCS III cataract grading. The same slit lamp machine and lighting conditions should be used across study visits for any given subject.

Subjects who develop cataracts may undergo cataract surgery if deemed clinically necessary; if surgery is performed, it should be carried out at least 4 weeks before the Visit 9 (Year 1) or Visit 11 (Year 2).

### 6.5: Fundus Photography

To aid in the objective clinical assessment of progressive retinal changes in the periphery of the retina, fundus photography was performed for both eyes at the times indicated in Table 2. Fundus photography was performed by certified technicians following pupil dilation. All fundus photographs were sent by the sites to the CRC for review; the CRC entered the data into the EDC system.

### 6.6: Vital Signs

Vital signs (pulse and systolic and diastolic blood pressure) were taken at the times indicated in Table 2. Vital signs were taken after the subject is seated for at least 5 minutes.

### 6.7: Laboratory Assessments

### 6.7.1 Laboratory Safety Parameters

Blood samples were collected at the times indicated in Table 2 for measurement of hematology and clinical chemistry parameters. Samples were sent to a central laboratory for analysis.

The hematology and clinical chemistry parameters to be evaluated are outlined in Table 4.

TABLE 4
Laboratory Safety Parameters

| Hematology | Clinical Chemistry |
|---|---|
| Hematocrit | Albumin |
| Hemoglobin | Alkaline phosphatase |
| Platelet count | Aspartate transaminase |
| White blood cell count with differential | Alanine transaminase |
| | Bilirubin (total) |
| | Blood urea nitrogen |
| | Calcium |
| | Chloride |
| | Creatinine |
| | C-reactive protein |
| | Gamma glutamyl transferase |
| | Globulin |
| | Glucose (non-fasting) |
| | Lactate dehydrogenase |
| | Magnesium |
| | Phosphate |
| | Potassium |
| | Protein (total) |
| | Sodium |

### 6.7.2 Viral Shedding

Blood, tears (both eyes), saliva and urine samples were collected at the times indicated in Table 2 and tested by polymerase chain reaction amplification of vector genomes to assay for evidence of vector shedding and dispersion. Samples were sent to a central laboratory for analysis.

### 6.7.3 Immunogenicity

For the evaluation of immunogenicity, blood was collected at the times indicated in Table 2. Immunoassays were planned to assess antibody and cell based responses against AAV8-RPGR. Enzyme-linked immunospot assays were used for T-cell mediated immune responses to transgene, and antibody responses were assayed using enzyme-linked immunosorbent assay-based methods. All immunogenicity samples were sent to and stored at a central laboratory for future analyses.

### 7.0: Statistical Considerations

### 7.1: Sample Size

Due to the nature of the study design, no formal sample size computation was performed. A sample size of 30 subjects at the MTD dose ensures that events with an incidence ≥10% will be identified with a 95% probability.

### 7.2: Procedure for Accounting for Missing Data

All reasonable efforts will be made to obtain complete data for both eyes on all subjects. However, missing observations may occur. Management of dropouts and missing observations will depend on their nature and frequency. Safety and efficacy data will be analyzed on observed data only. Missing data will not be imputed.

### 7.3: Analysis Sets

### 7.3.1 Safety Analysis Set

The Safety Analysis Set consisted of all subjects who receive study treatment (vitrectomy/AAV8-RPGR). The Safety Analysis Set was the primary population for demographics, baseline characteristics and safety analyses.

### 7.3.2 Full Analysis Set

The Full Analysis Set included all subjects for whom data of at least 1 post-baseline efficacy assessment was available in at least one eye. The Full Analysis Set was used for efficacy analyses.

### 7.4: Descriptive Statistics

Summary statistics were presented for both eyes (Study Eyes versus Fellow Eyes). No formal statistical comparison was performed. For categorical/binary data, the number and proportion of subjects pertaining to each category was presented over time with its 95% confidence interval (CI). Continuous data was summarized over time using mean, and its 95% CI, standard deviation, median, minimum and maximum. 95% CIs were 2-sided. Summaries were generated by dose and overall, in Part I and, by group (MTD dose and low-dose) in Part II.

### 7.5: Demographics and Baseline Characteristics

Demographics and baseline ocular characteristics were summarized for the safety analysis set and the full analysis set.

### 7.6: Safety Analyses

Due to the potential systemic effect of study treatment (surgery/study medication) on the contralateral eye, ocular assessments and AEs were summarized by eye (Study Eye and Fellow Eye) while systemic assessments were analyzed at the subject level. No formal statistical testing was performed for safety analyses. Safety analyses were performed on the Safety Analysis Set.

### 7.6.1 Adverse Events

AEs were coded using the Medical Dictionary for Regulatory Activities. The version of the dictionary current at the time of the database lock was used. AEs were summarized by system organ class and preferred term. Both the number of eyes/subjects experiencing an AE and the number of events were summarized. Similar summaries were produced for study drug/procedure-related AEs, AEs leading to discontinuation and SAEs. AEs were also summarized by maximum severity, relationship to study drug/procedure and time to onset.

A by-subject listing of DLTs was prepared.

### 7.6.2 Ocular Safety Evaluations

TOP and changes from baseline in IOP, abnormal slit lamp examination findings and indirect ophthalmoscopy findings, and anterior chamber and vitreous inflammation grading were summarized by visit and eye.

Lens opacity categories and shifts from baseline were summarized by visit and eye.

Categories of fundus photography findings (none/mild/moderate/severe) were summarized by visit and eye.

The number of subjects with a 10- and 15-letter decrease from baseline in BCVA were tabulated by visit and by eye.

### 7.6.3 Laboratory Assessments and Vital Signs

Laboratory assessments and vital signs were summarized in a descriptive manner.

### 7.7: Efficacy Analyses

Efficacy assessments are ocular in nature and therefore were tabulated by eye (Study Eye and Fellow Eye). Efficacy data was summarized using descriptive statistics.

Change from baseline in BCVA were tabulated by visit and by eye.

### 7.7.1 Alpha Adjustment

Alpha adjustment was not applicable in this exploratory Phase 1/2 study.

### 7.8: Interim Analysis

In Part I, exploratory interim analysis were conducted after each dose cohort. In Part II, secondary endpoints were analyzed at 3, 6, 12, 18 and 24 months with masking to treatment dose maintained.

### Example 4: Preparation of RPGR^{ORF15} Transgene for Gene Therapy for Retinitis Pigmentosa

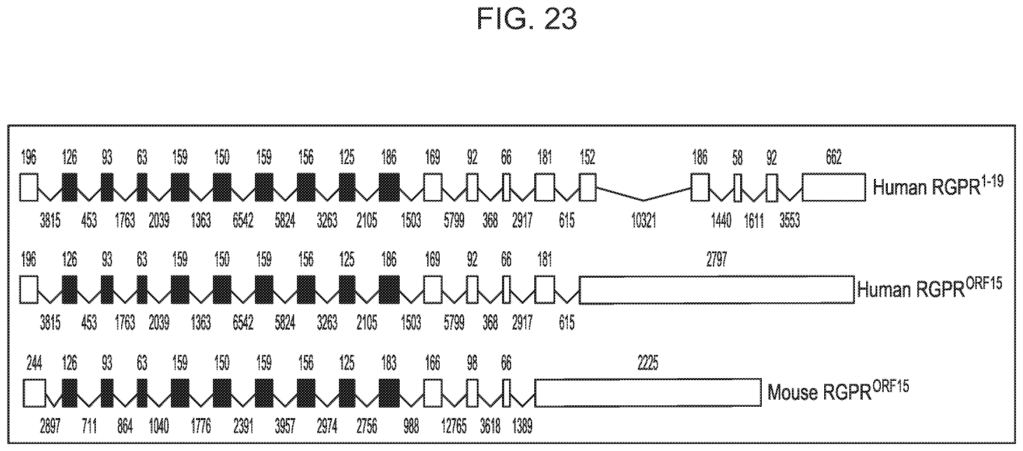

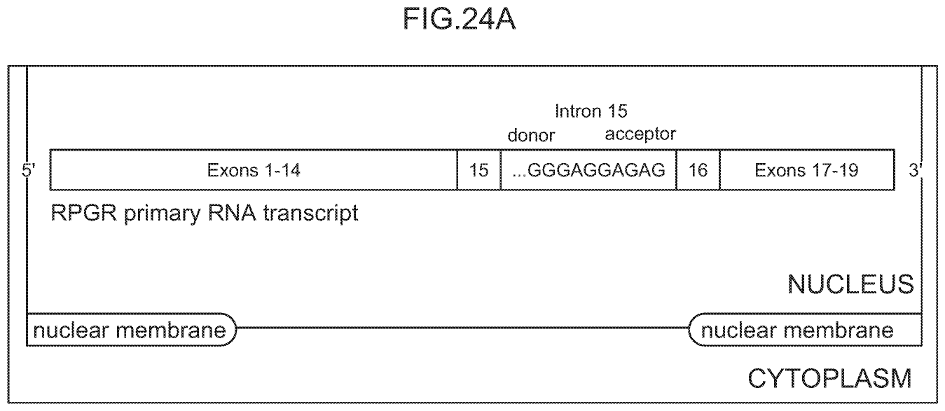

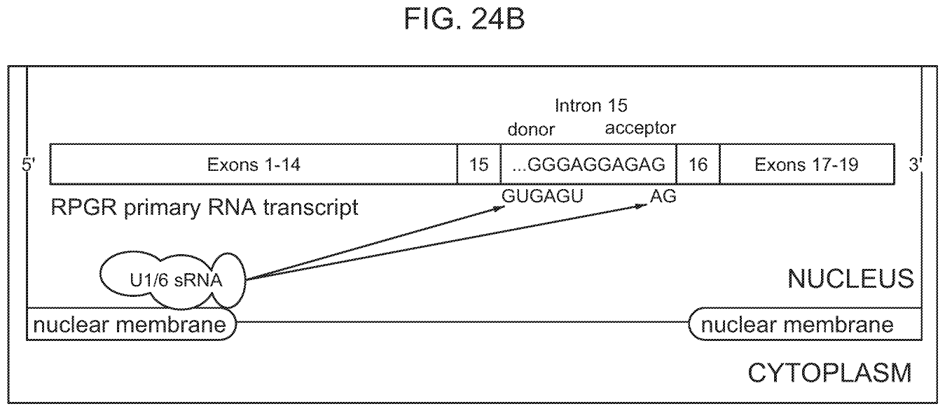

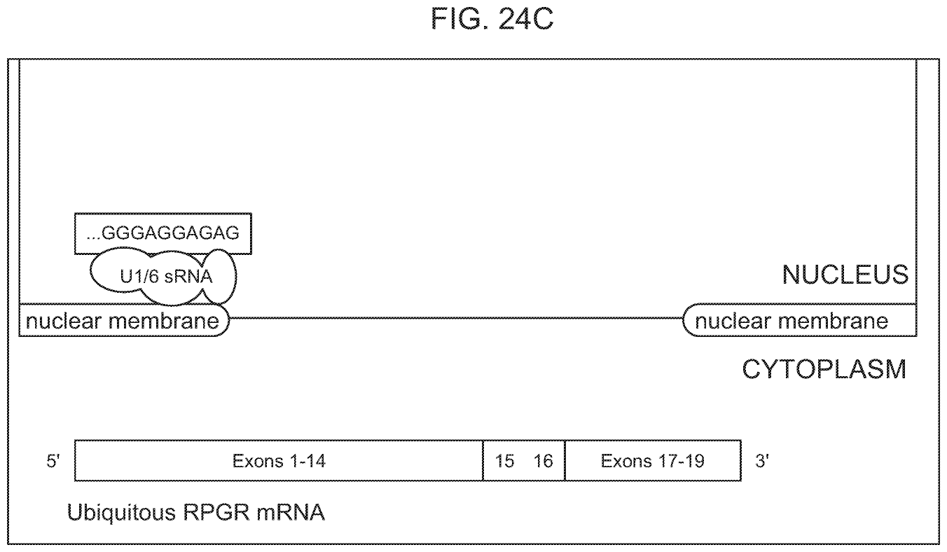

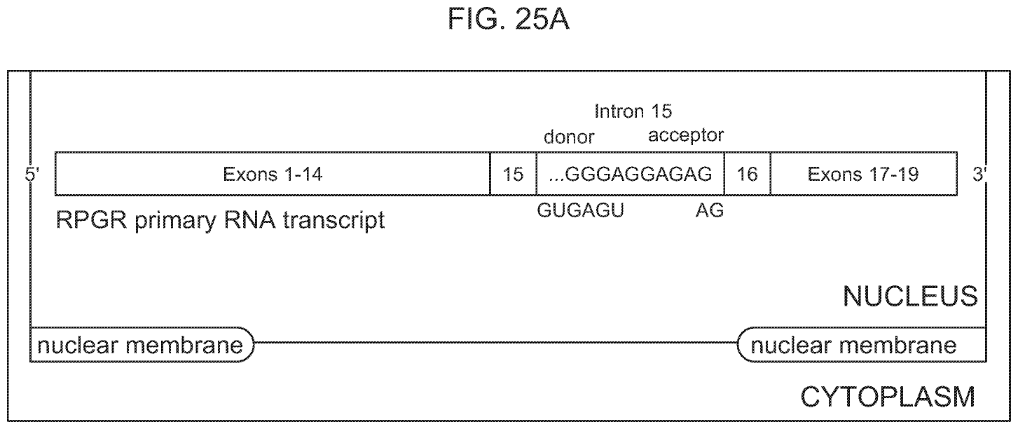

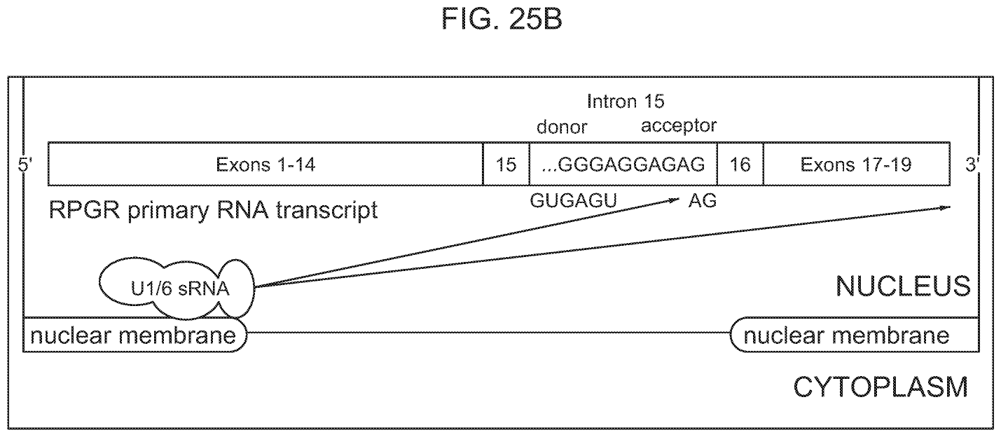

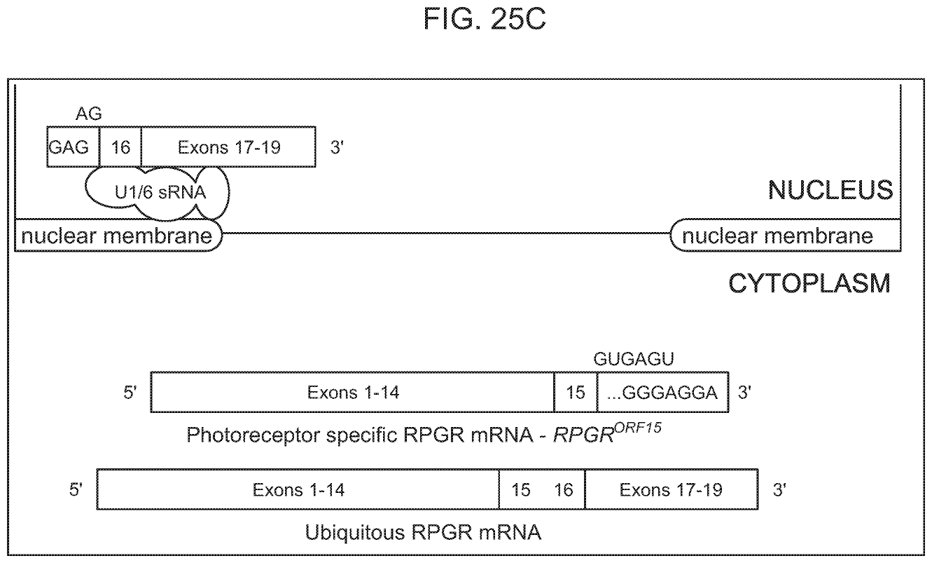

The RPGR gene is alternatively spliced (FIG. 23). The two major RPGR isoforms are the constitutive variant encoded by exons 1-19 (RPGR^{Ex1-19}) and the RPGR^{ORF15} isoform, which consists of exons 1-14 of RPGR^{Ex1-19} followed by a unique C-terminal exon called open reading frame 15. The splicing events that produce ubiquitous RPGR mRNA are shown in FIGS. 24A-24C. The splicing events that produce photoreceptor specific RPGR mRNA-RPGR^{ORF15} are shown in FIGS. 25A-25C. The RPGR^{ORF15} isoform is expressed in the photoreceptor cilium of vertebrates.

The RPGR^{ORF15} isoform contains the highly repetitive purine-rich exon (or open-reading frame) 15, which is prone to mutations as well as errors during viral vector cloning (FIGS. 26A-26D). Although RPGR is within the coding capacity of the adeno-associated viral (AAV) vector, the highly repetitive purine-rich region at the 3′-end and a splice site immediately upstream of this region have created significant challenges in cloning an AAV.RPGR vector, with several groups reporting miss-spliced or truncated variants during preclinical testing.

The sequence of codon-optimized RPGR^{ORF15} is provided below:

(SEQ ID NO: 3)
ATGAGAGAGCCAGAGGAGCTGATGCCAGACAGTGGAGCAGTGTTTACATT
CGGAAAATCTAAGTTCGCTGAAAATAACCCAGGAAAGTTCTGGTTTAAAA
ACGACGTGCCCGTCCACCTGTCTTGTGGCGATGAGCATAGTGCCGTGGTC
ACTGGGAACAATAAGCTGTACATGTTCGGGTCCAACAACTGGGGACAGCT
GGGGCTGGGATCCAAATCTGCTATCTCTAAGCCAACCTGCGTGAAGGCAC
TGAAACCCGAGAAGGTCAAACTGGCCGCTTGTGGCAGAAACCACACTCTG
GTGAGCACCGAGGGCGGGAATGTCTATGCCACCGGAGGCAACAATGAGGG
ACAGCTGGGACTGGGGGACACTGAGGAAAGGAATACCTTTCACGTGATCT
CCTTCTTTACATCTGAGCATAAGATCAAGCAGCTGAGCGCTGGCTCCAAC
ACATCTGCAGCCCTGACTGAGGACGGGCGCCTGTTCATGTGGGGAGATAA
TTCAGAGGGCCAGATTGGGCTGAAAAACGTGAGCAATGTGTGCGTCCCTC
AGCAGGTGACCATCGGAAAGCCAGTCAGTTGGATTTCATGTGGCTACTAT
CATAGCGCCTTCGTGACCACAGATGGCGAGCTGTACGTCTTTGGGGAGCC
CGAAAACGGAAAACTGGGCCTGCCTAACCAGCTGCTGGGCAATCACCGGA
CACCCCAGCTGGTGTCCGAGATCCCTGAAAAAGTGATCCAGGTCGCCTGC
GGGGGAGAGCATACAGTGGTCCTGACTGAGAATGCTGTGTATACCTTCGG
ACTGGGCCAGTTTGGCCAGCTGGGGCTGGGAACCTTCCTGTTTGAGACAT
CCGAACCAAAAGTGATCGAGAACATTCGCGACCAGACTATCAGCTACATT
TCCTGCGGAGAGAATCACACCGCACTGATCACAGACATTGGCCTGATGTA
TACCTTTGGCGATGGACGACACGGGAAGCTGGGACTGGGACTGGAGAACT
TCACTAATCATTTTATCCCCACCCTGTGTTCTAACTTCCTGCGGTTCATC
GTGAAACTGGTCGCTTGCGGCGGGTGTCACATGGTGGTCTTCGCTGCACC
TCATAGGGGCGTGGCTAAGGAGATCGAATTTGACGAGATTAACGATACAT
GCCTGAGCGTGGCAACTTTCCTGCCATACAGCTCCCTGACTTCTGGCAAT
GTGCTGCAGAGAACCCTGAGTGCAAGGATGCGGAGAAGGGAGAGGGAACG
CTCTCCTGACAGTTTCTCAATGCGACGAACCCTGCCACCTATCGAGGGAA
CACTGGGACTGAGTGCCTGCTTCCTGCCTAACTCAGTGTTTCCACGATGT
AGCGAGCGGAATCTGCAGGAGTCTGTCCTGAGTGAGCAGGATCTGATGCA
GCCAGAGGAACCCGACTACCTGCTGGATGAGATGACCAAGGAGGCCGAAA
TCGACAACTCTAGTACAGTGGAGTCCCTGGGCGAGACTACCGATATCCTG
AATATGACACACATTATGTCACTGAACAGCAATGAGAAGAGTCTGAAACT
GTCACCAGTGCAGAAGCAGAAGAAACAGCAGACTATTGGCGAGCTGACTC
AGGACACCGCCCTGACAGAGAACGACGATAGCGATGAGTATGAGGAAATG
TCCGAGATGAAGGAAGGCAAAGCTTGTAAGCAGCATGTCAGTCAGGGGAT
CTTCATGACACAGCCAGCCACAACTATTGAGGCTTTTTCAGACGAGGAAG
TGGAGATCCCCGAGGAAAAAGAGGGCGCAGAAGATTCCAAGGGGAATGGA
ATTGAGGAACAGGAGGTGGAAGCCAACGAGGAAAATGTGAAAGTCCACGG
AGGCAGGAAGGAGAAAACAGAAATCCTGTCTGACGATCTGACTGACAAGG
CCGAGGTGTCCGAAGGCAAGGCAAAATCTGTCGGAGAGGCAGAAGACGGA
CCAGAGGGACGAGGGGATGGAACCTGCGAGGAAGGCTCAAGCGGGGCTGA
GCATTGGCAGGACGAGGAACGAGAGAAGGGCGAAAAGGATAAAGGCCGCG
GGGAGATGGAACGACCTGGAGAGGGCGAAAAAGAGCTGGCAGAGAAGGAG
GAATGGAAGAAAAGGGACGGCGAGGAACAGGAGCAGAAAGAAAGGGAGCA
GGGCCACCAGAAGGAGCGCAACCAGGAGATGGAAGAGGGCGGCGAGGAAG
AGCATGGCGAGGGAGAAGAGGAAGAGGGCGATAGAGAAGAGGAAGAGGAA
AAAGAAGGCGAAGGGAAGGAGGAAGGAGAGGGCGAGGAAGTGGAAGGCGA
GAGGGAAAAGGAGGAAGGAGAACGGAAGAAAGAGGAAAGAGCCGGCAAAG
AGGAAAAGGGCGAGGAAGAGGGCGATCAGGGCGAAGGCGAGGAGGAAGAG
ACCGAGGGCCGCGGGGAAGAGAAAGAGGAGGGAGGAGAGGTGGAGGGCGG
AGAGGTCGAAGAGGGAAAGGGCGAGCGCGAAGAGGAAGAGGAAGAGGGCG
AGGGCGAGGAAGAAGAGGGCGAGGGGGAAGAAGAGGAGGGAGAGGGCGAA
GAGGAAGAGGGGGAGGGAAAGGGCGAAGAGGAAGGAGAGGAAGGGGAGGG
AGAGGAAGAGGGGGAGGAGGGCGAGGGGGAAGGCGAGGAGGAAGAAGGAG
AGGGGGAAGGCGAAGAGGAAGGCGAGGGGGAAGGAGAGGAGGAAGAAGGG
GAAGGCGAAGGCGAAGAGGAGGGAGAAGGAGAGGGGGAGGAAGAGGAAGG
AGAAGGGAAGGGCGAGGAGGAAGGCGAAGAGGGAGAGGGGGAAGGCGAGG
AAGAGGAAGGCGAGGGCGAAGGAGAGGACGGCGAGGGCGAGGGAGAAGAG
GAGGAAGGGGAATGGGAAGGCGAAGAAGAGGAAGGCGAAGGCGAAGGCGA
AGAAGAGGGCGAAGGGGAGGGCGAGGAGGGCGAAGGCGAAGGGGAGGAAG
AGGAAGGCGAAGGAGAAGGCGAGGAAGAAGAGGGAGAGGAGGAAGGCGAG
GAGGAAGGAGAGGGGGAGGAGGAGGGAGAAGGCGAGGGCGAAGAAGAAGA
AGAGGGAGAAGTGGAGGGCGAAGTCGAGGGGGAGGAGGGAGAAGGGGAAG
GGGAGGAAGAAGAGGGCGAAGAAGAAGGCGAGGAAAGAGAAAAAGAGGGA
GAAGGCGAGGAAAACCGGAGAAATAGGGAAGAGGAGGAAGAGGAAGAGGG
AAAGTACCAGGAGACAGGCGAAGAGGAAAACGAGCGGCAGGATGGCGAGG
AATATAAGAAAGTGAGCAAGATCAAAGGATCCGTCAAGTACGGCAAGCAC
AAAACCTATCAGAAGAAAAGCGTGACCAACACACAGGGGAATGGAAAAGA
GCAGAGGAGTAAGATGCCTGTGCAGTCAAAACGGCTGCTGAAGAATGGCC
CATCTGGAAGTAAAAAATTCTGGAACAATGTGCTGCCCCACTATCTGGAA
CTGAAATAA.

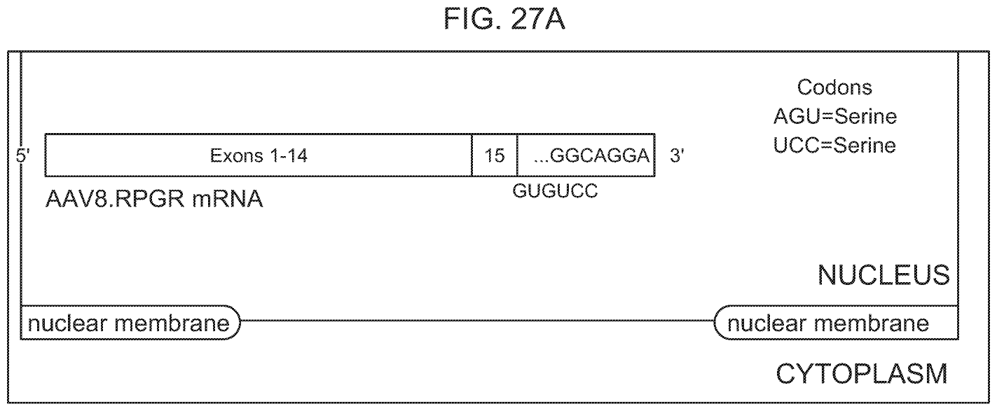

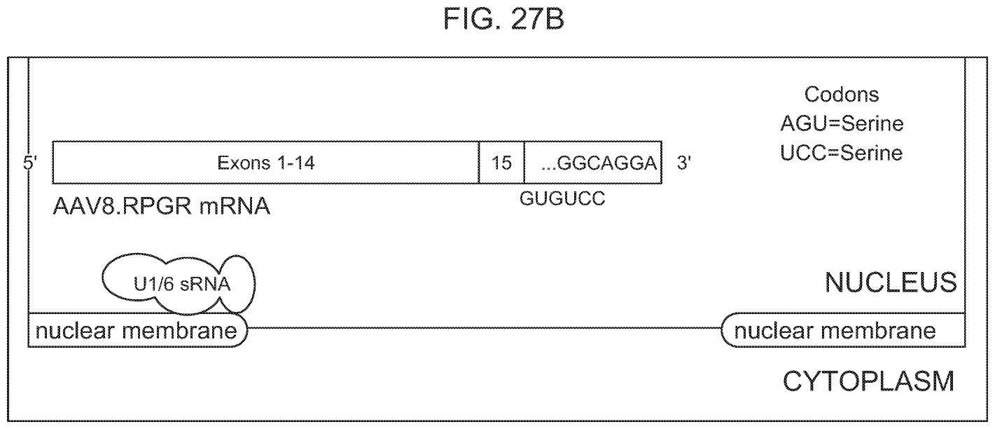

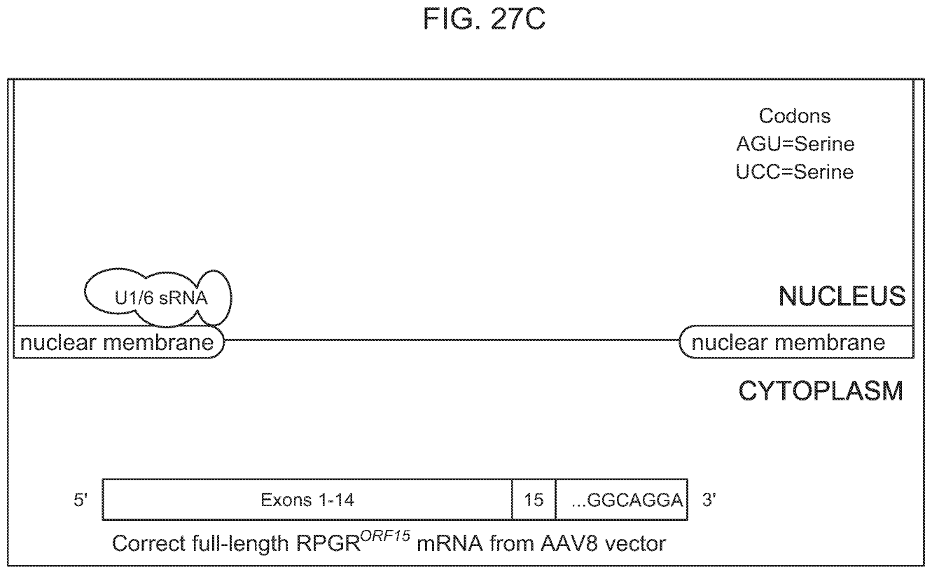

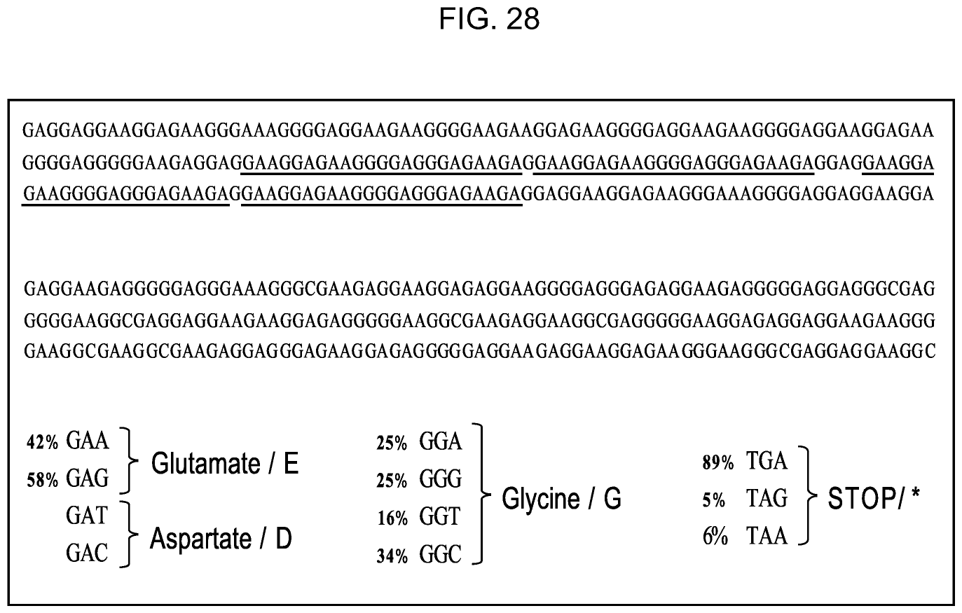

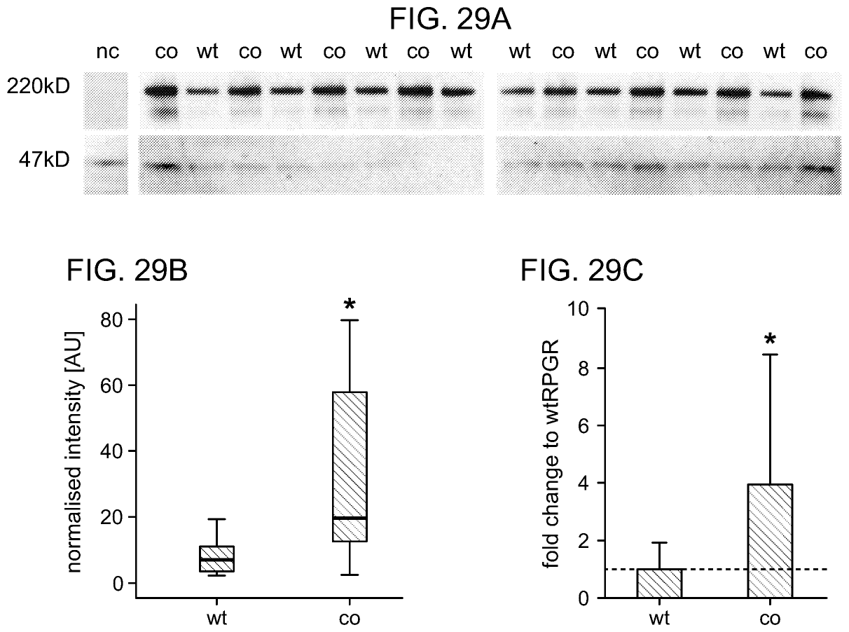

Codon optimization was used to disable the endogenous splice site and stabilize the purine-rich sequence in the photoreceptor-specific RPGR transcript without altering the amino acid sequence (FIGS. 27A-27C). Codon optimization was used to (1) remove repetitive purine sequences and cryptic splice sites; (2) remove polyA signals and reduce out of frame stop codons; and (3) consider optimal human tRNA codon bias with minimal CpG (FIG. 28). A codon-optimized version of human RPGR^{ORF15} (coRPGR) produced the correct-sized protein as shown via Western blot (FIG. 29A-29C). See, Fischer et al. Mol Ther. 2017; 25(8):1854-1865.

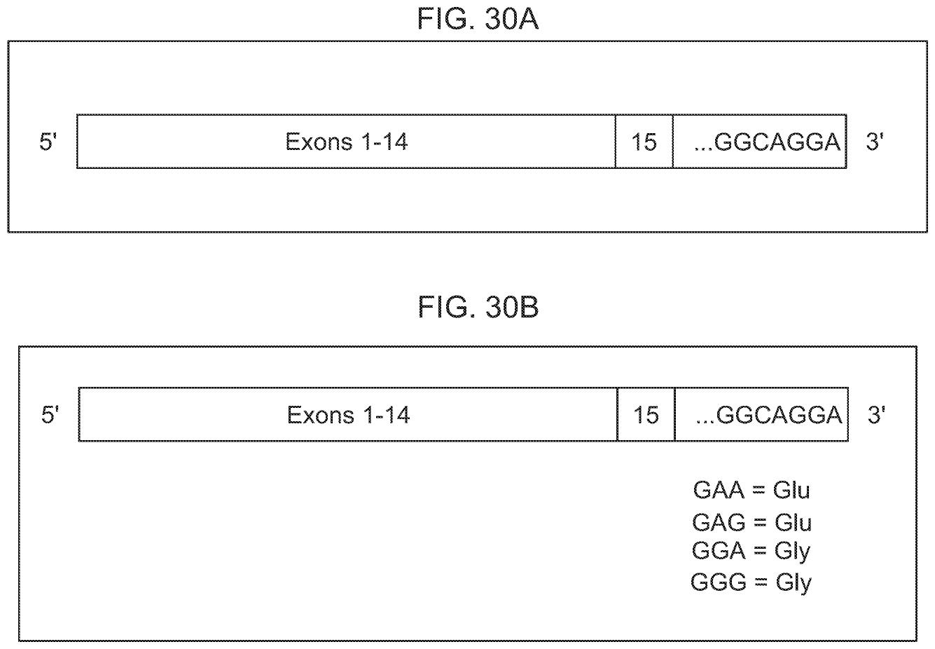

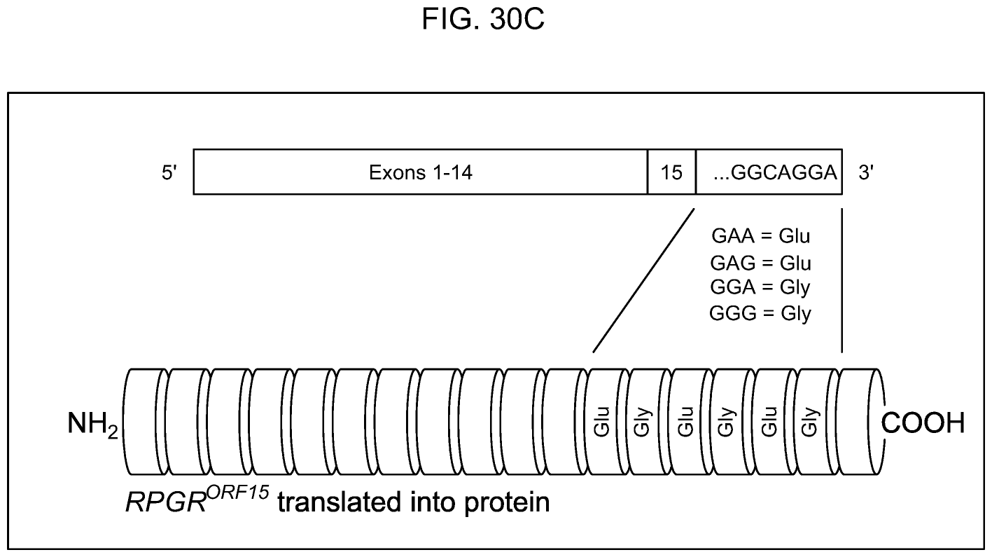

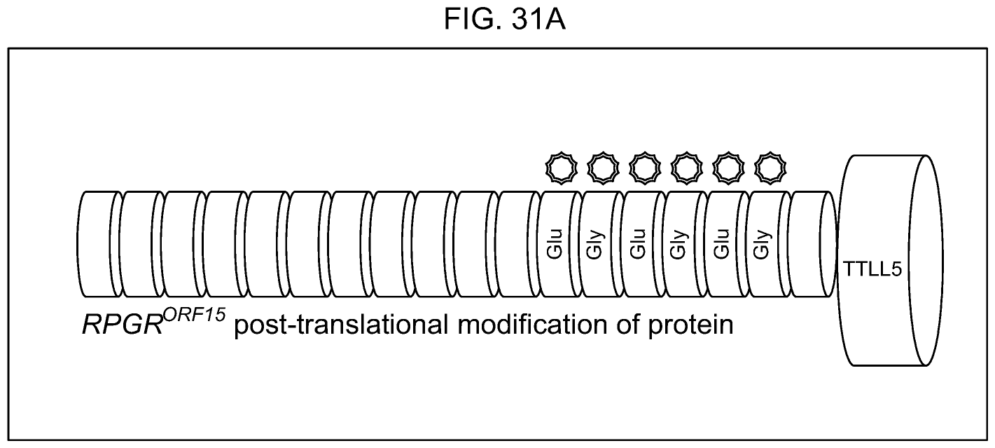

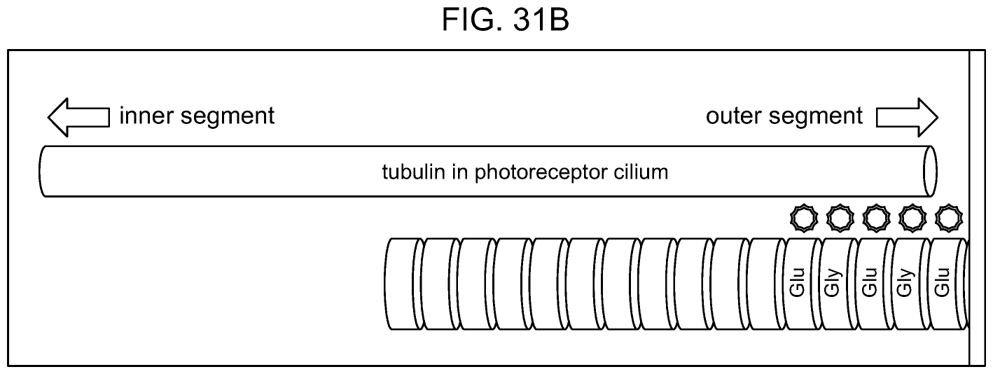

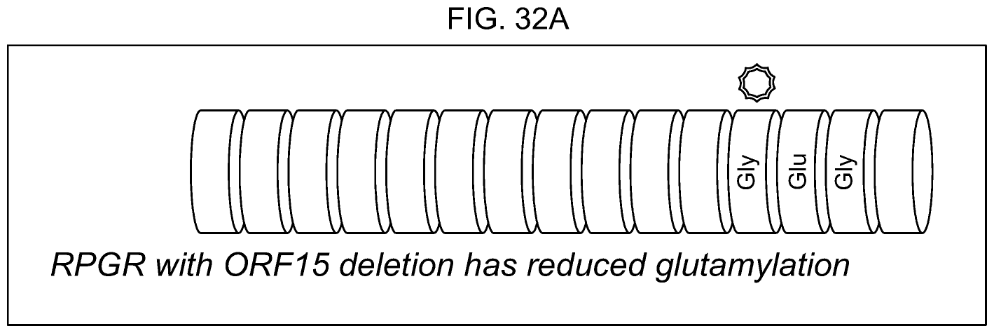

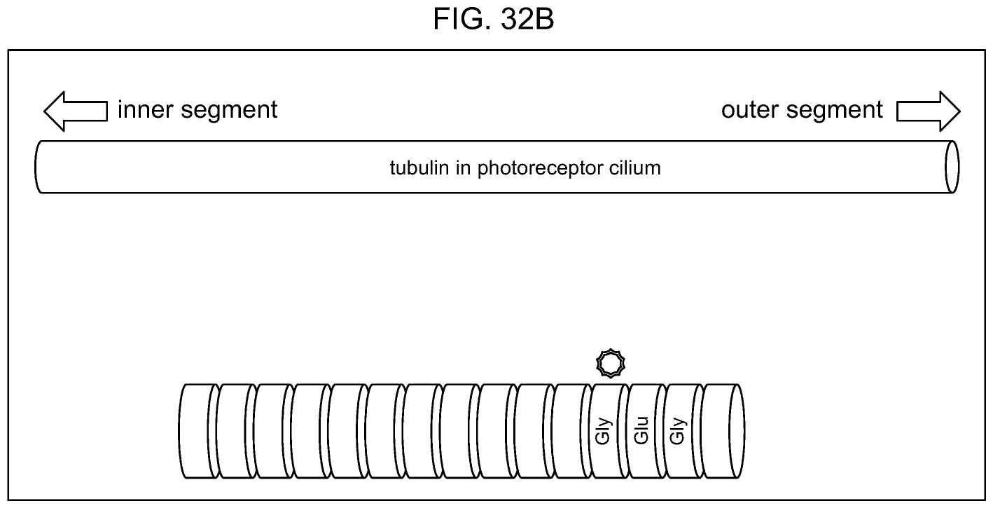

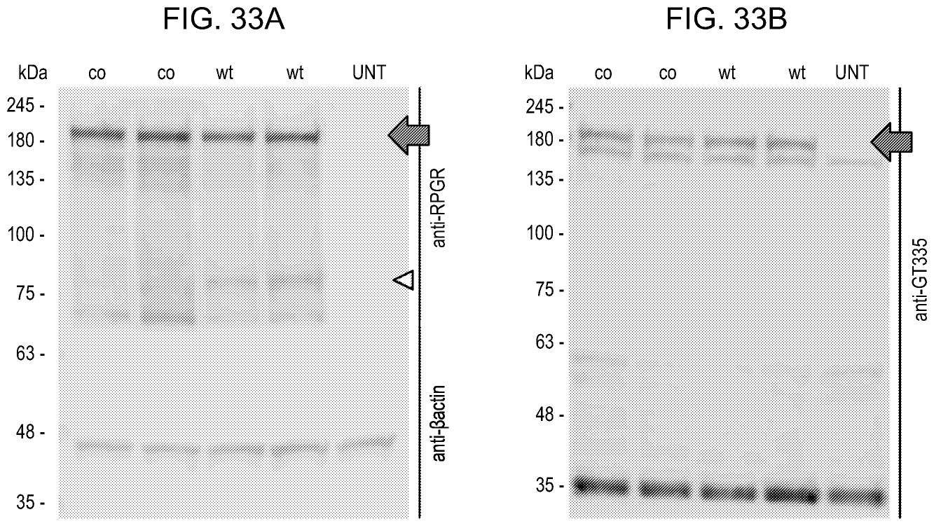

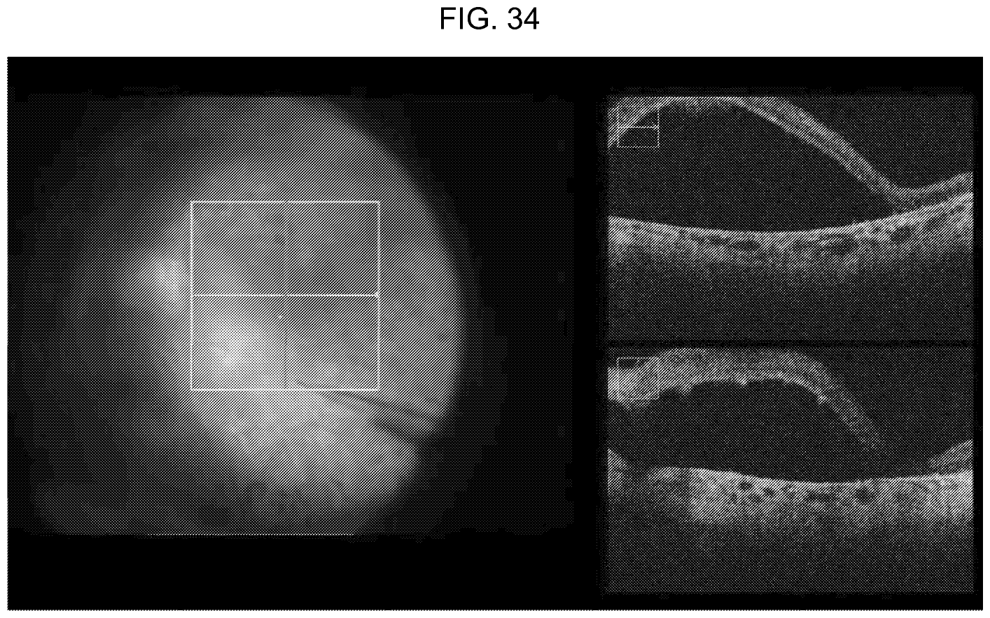

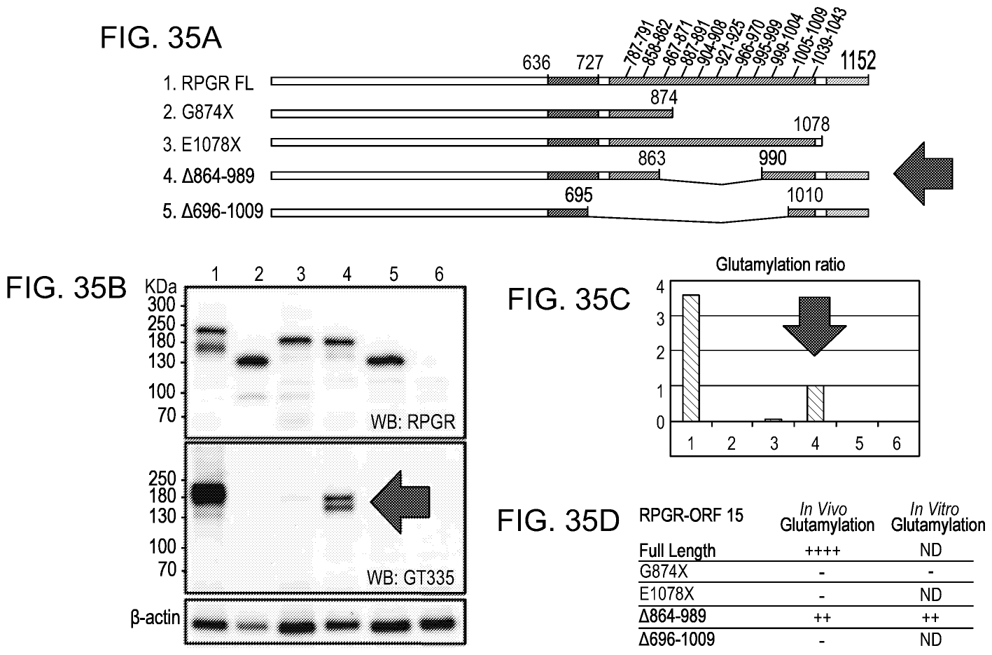

Glutamylation of RPGR protein, a key post-translational modification, was also preserved following codon optimization. RPGR glutamylation in vivo requires both the C-terminal basic domain and the Glu-Gly-rich region (FIGS. 35A-35D). See, Sun et al. PNAS, 2016, 113 (21) E2925-E2934. RPGR is glutamylated with TTLL5, and glutamylation moves RPGR along tubulin in photoreceptor cilia (FIGS. 30A-30C and FIGS. 31A-31B). RPGR with ORF15 deletion has reduced glutamylation; thus, deleted RPGR is defective (FIG. 32A-32B). Codon-optimized RPGR produced in vitro demonstrated correct splicing and correct glutamylation (FIGS. 33A-33B). See, Fischer et al. Mol Ther. 2017; 25(8):1854-1865. A codon-optimized version of human RPGR^{ORF15} (coRPGR) was used in human RPGR gene therapy (FIG. 34).

### Example 5: Optimization of AAV-RPGR Compositions

This study demonstrates the optimization of the codon usage of RPGR^{ORF15} coding sequence (cds). The most important advantage of optimising codons in difficult sequences such as RPGR^{ORF15} lies in the potential to improve sequence fidelity. Changing nucleotides without changing the resulting amino acid sequence carries the potential to make the sequence more stable and less prone to spontaneous mutations during the production of vectors for gene therapy. This optimization may further lead to higher transgene expression without the use of accessory regulatory elements in the transgene cassette. Once the codon sequence was established, additional in vitro investigations were conducted to develop and optimize a gene therapy strategy aimed to engineer a pseudotyped, recombinant adeno associated virus (AAV) vector with the capsid from the AAV8 serotype, while using the well-characterized, gutted genome from AAV2 for an optimized AAV vector for gene replacement therapy in patients with mutations in RPGR^{ORF15}.

The cds of a gene serves as template for translation of nucleic acid sequence into peptides. This process involves the cds contained in the messenger ribonucleic acid (mRNA) transcript, ribosomal complexes and amino acids, which are bound to transfer ribonucleic acid (tRNA) molecules. Three consecutive nucleotides in the cds (eg, UUA) constitute a codon. tRNA molecules have complementary anti-codon sequences (eg, AAU), and briefly bind to the codon sequence within the ribosomal complex and contribute a single amino acid (eg, Leucine) they are carrying to the growing chain of amino acids forming the growing peptide encoded by the cds. In the context of gene therapy using AAV as the vector system with its limited packaging capacity, codon optimization offers the potential to increase transgene expression without additional cis acting regulatory elements, such as woodchuck hepatitis virus post-transcriptional regulatory element (WPRE) in the expression cassette, leading to a cleaner design and higher efficiency in AAV production cycles. Moreover, the nucleotide sequence van be changed without altering the translated amino acid sequence (silent substitutions) of the transgene in order to improve cytosine/guanine content, to remove unwanted repeat sequences and/or restriction sites that may interfere with cloning. These often are the most important advantages of optimizing codons in difficult sequences such as RPGR^{ORF15}: the potential to improve sequence fidelity. Changing nucleotides without changing the resulting amino acid sequence carries the potential to make the sequence more stable and less prone to spontaneous mutations during the production of vectors for gene therapy.

Recombinant AAVs have become the gold standard of retinal gene therapy leading the way into multiple successful clinical trials over the last decade. The excellent safety profile in preclinical models, as well as human patients, and the versatility of its components to adapt to new target genes are important factors in selection of AAV as the vector system for RPGR^{ORF15} delivery.

Different AAV serotypes lead to distinct expression patterns due to specific interactions between AAV surface proteins and target cell receptors. The naturally occurring serotype AAV2, for example, is very efficient to transduce retinal pigment epithelium, but less effective in delivering the transgene into photoreceptor cells. In contrast, AAV8 capsid structures lead to rapid and efficient uptake of virions by mammalian photoreceptor cells.

Photoreceptors are expressing RPGR^{ORF15} and direct it to localize to the connecting cilium, where it organizes intracellular protein-transport along a bottleneck structure called the connecting cilium. Photoreceptors without functional RPGR^{ORF15} suffer from accumulation of highly expressed proteins such as opsins, which leads to photoreceptor dysfunction and ultimately cell death.

Photoreceptors are the target cell population for RPGR^{ORF15} gene delivery; therefore, AAV8 capsid proteins were selected as a candidate viral serotype for XLRP gene therapy. Due to the success of AAV2 based transgene cassettes in all retinal gene therapy trials, a pseudotyped construct, AAV2/8, which combines the AAV8 capsid proteins with the AAV2 based genome, was developed. Briefly, the therapeutic transgene cassette is flanked by AAV2 inverted terminal repeat (ITR) sequences, which coordinates the packaging of the genome during vector production and serves as starting point for second strand synthesis after successful delivery of the therapeutic transgene into the nucleus of the target cell.

### Materials and Methods

Table 5 provides a description of test and control articles used in the study.

| Full Name of Construct | Referred to as | Description |
|---|---|---|
| ITR.CAG.Kozak.wtRPGR^{ORF15}.bGHpA.ITR | CAG.wtRPGR | Plasmid DNA |
| ITR.CAG.Kozak.coRPGR^{ORF15}.bGHpA.ITR | CAG.coRPGR | Plasmid DNA |
| ITR.RK.Kozak.coRPGR^{ORF15}.bGHpA.ITR | RK.coRPGR | Plasmid DNA |
| | | Construct |
| | | Features no |
| | | restriction site |
| | | between RK |
| | | promoter and |
| | | Kozak or |
| | | coRPGR^{ORF15} cds, |
| | | but was ligated into |
| | | the vector |
| | | backbone between |
| | | the upstream ITR |
| | | (MfeI) and the |
| | | downstream MCS |
| | | (SacI) |
| ITR.RK.Kozak.wtRPGR^{ORF15}.bGHpA.ITR | RK.wtRPGR | Plasmid DNA |
| | | Construct |
| | | Features no |
| | | restriction site |
| | | between |
| | | RK promoter and |
| | | Kozak or |
| | | coRPGR^{ORF15} cds, |
| | | but was ligated |
| | | into the vector |
| | | backbone between |
| | | the upstream ITR |
| | | (MfeI) and the |
| | | downstream MCS |
| | | (SacI), but using |
| | | restriction sites |
| | | BglII after the |
| | | upstream ITR |
| | | (wtRPGR^{ORF15} cds |
| | | features a MfeI |
| | | restriction site at |
| | | position 1583- |
| | | 1588) |
| ITR.EF-1a.loxP.[EYFP].loxP.WPRE.bGHpA.ITR | Control | Plasmid DNA |
| | | Construct with the |
| | | identical backbone |
| | | and ITR sequences, |
| | | but with a stuffer |
| | | sequence (double- |
| | | floxed and |
| | | reversed |
| | | fluorescent protein |
| | | EYFP) |
| AAV2/8.RK.coRPGR vector | AAV2/8.RK.coRPGR | Codon optimized |
| | (AAV8-RPGR) | RPGR plasmid |
| | | construct in viral |
| | | vector |
| AAV2/8.RK.wtRPGR vector | AAV2/8.RKwtRPGR | Wild-type RPGR |
| | | plasmid construct |
| | | in viral vector |
| AAV2/8 vector | AAV2/8 | Viral vector with |
| | | no transgene |

RPGR = retinitis pigmentosa GTPase regulator protein.
All constructs were suspended in molecular biology grade water (DEPC-treated and sterile filtered, Sigma-Aldrich).

### Test System

Several cell cultures, including HEK293T, SH-SY5Y, and 661W cells, were used to:
Study levels of RPGR^{ORF15} expression from wild type or optimized codon sequences
Overexpress the RPGR protein for sequence analysis
Produce of recombinant AAV
Test AAV transduction efficiencies

All cell culture work was performed in regularly serviced class II cell culture hoods, and flasks were incubated at 37° C. and 5% CO2 in a Galaxy R incubator (Eppendorf AG, Hamburg, Germany), unless stated otherwise. All media were freshly prepared and pre-warmed in a water bath to 37° C., unless stated otherwise. The individual cell culture systems used are described below.

Human Embryonic Kidney 293T Cells (HEK293T): HEK293T is a human embryonic kidney cell line. Cells were obtained from European Collection of Authenticated Cell Cultures (ECACC), Public Health England, Porton Down, Salisbury, SP4 0JG, UK.

Cells were stored in aliquots of 2×10⁶ cells in 1.5 mL 90% FBS 10% dimethyl sulfoxide (DMSO) at −196° C. in liquid nitrogen. Aliquots were resuscitated when needed in 10 mL complete cell culture media (88% DMEM [Invitrogen, Carlsbad, Calif.], substituted with 2 mM L-glutamine, 100 IU/mL Penicillin, and 100 μg/mL Streptomycin, and 10% FBS [all from Sigma-Aldrich Company Ltd., Dorset, UK]) after quickly thawing and mixing them into a single cell suspension. The cells were then spun at 1200×g for 5 minutes at 4° C., re-suspended in 1 mL culture media, and pipetted to achieve single-cell suspension before seeding cells into T75 flasks (Sarstedt Inc., Newton N.C., USA) with the required volume of media. Cells were fed fresh media after 24 hours to remove damaged and non-adherent cells and monitored daily until normal proliferation rates were achieved (3 to 5 days).

Once stable proliferation had been established, HEK293T cells were cultured with freshly prepared media every 2 to 3 days and passaged at 75% to 80% confluence: old media were removed, and cells washed once with 5 mL pre-warmed 0.01 M phosphate-buffered saline (PBS; Invitrogen Life Technologies Ltd., Paisley, UK) before adding 0.25% trypsin (Sigma-Aldrich) in 2 mL of PBS for 2 minutes. Cells were brought into solution and 8 mL of complete cell culture media (see above) added. Two milliliters of this suspension were then transferred to a new T75 flask and 13 mL media added.

Human Neuroblastoma-derived Cells: SH-SY5Y cells are adherent, neuroblast-derived cells. They are subclones from the original SK-N-SH cells, which were isolated from a bone marrow biopsy of a female 4 years of age with neuroblastoma. SH-SY5Y cells had been originally obtained from the ECACC, Public Health England, Porton Down, Salisbury, SP4 0JG, UK.

Cells were stored in aliquots of 2×10⁶ cells in 1.5 mL 90% FBS 10% DMSO at −196° C. in liquid nitrogen. Resuscitation was performed as described for HEK293T cells, except the culture media composition was: 1 to 1 mixture of Ham's F12 and Eagle minimum essential media with Earle's Balanced Salt Solution (EMEM [EBSS]) with 2 mM Glutamine, 1% Non Essential Amino Acids, 15% FBS, 100 μg/mL Penicillin, and 100 μg/mL Streptomycin (all Sigma-Aldrich). Cells were maintained in T75 flasks and split as subconfluent cultures (70% to 80%) in a 1:50 ratio, ie, seeding at approximately 5×10⁴ cells/cm². The splitting was performed again as described for the HEK293T cells, except for the constitution of the cell culture medium. For induction of a neuron-specific differentiation, media was changed to that containing 1.6×10⁻⁸ M Tetradecanoylphorbol-13-acetate (TPA) and 10-5 M retinoic acid (RA, both Sigma-Aldrich) 24 hours after seeding.

Mouse Cone Photoreceptor-like Cells: The 661W cell line was originally cloned from retinal tumors of a transgenic mouse line expressing the Simian virus (SV) 40 T antigen under control of the human inter-photoreceptor retinol-binding protein (IRBP) promoter. It is described as ‘cone photoreceptor like cell line’, as it was reported to demonstrate cellular and biochemical characteristics of cone photoreceptor cells, such as expression of short- and medium-wavelength sensitive cone opsins.

The cell line was imported from Dr Muayyad R. Al-Ubaidi (Oklahoma, USA) under a material transfer agreement and cultured strictly according to his suggestions. Aliquots had been cryopreserved for long-term storage and resuscitated when needed as described for the HEK293T and SH-SY5Y cells, except for the culture medium composition: DMEM (Gibco, Thermo Fisher Scientific) with 40 μg/L Hydrocortisone, 40 μg/L Progesterone, 0.032 g/L Putrescine, 40 μL/L β-mercaptoethanol, 100 mg/L Penicillin, 100 mg/L Streptomycin (all Sigma-Aldrich), and 7.5% FBS (Gibco).

Cells were maintained in T75 flasks and split as subconfluent cultures (70% to 80%) at a 1:5 ratio performed again as described for the HEK293T cells, except for the constitution of the cell culture medium.

Rationale for test system: The cell lines used in these investigations, HEK293T, SH SY5Y, and 661W, are representative of normal human cells, human neural cells, and photoreceptor cells.

Human HEK293T are normal human embryonic kidney cells stably transformed with Adenovirus 5 and a single clone was isolated from the 293rd experiment (293T). The 293T cell line contains the SV40 Large T-Antigen, allowing for efficient plasmid replication. Adenovirus are known to transduce cells of neuronal lineage more efficiently than non-neuronal cells, and HEK293 cells have many properties of immature neurons. Through transcriptome analysis, these cells were found to most closely resemble adrenal cells (kidney-associated cells with some neuronal characteristics). Therefore, HEK293/HEK293T cells are embryonic adrenal precursor cells (with neuronal properties) that are efficiently transduced by adenovirus or AAV. Human SH-SY5Y were derived from a bone marrow-derived cell line (SK-N-SH) and are often used as a cell model of neuronal function. In addition, SH-SY5Y cells have the ability to differentiate along a neuronal lineage. Therefore, SH-SY5Y cells represent a model with a greater number of neuronal characteristics.

The murine 661W cell line was cloned from retinal tumors expressing the SV-40 T antigen under the control of the inter-photoreceptor retinal binding protein promoter (IRBP). Despite their highly transformed state, 661W cells have been shown to express several markers of photoreceptor cells. Therefore, these cells are useful for examining the expression of RPG-ORF15, a photoreceptor-specific protein isoform, and may provide a highly useful testing system before moving into animals.

### Experimental Methods

Transgene Detection: HEK293T cells were transfected with CAG.coRPGRORF15 and CAG.wtRPGRORF15 plasmid constructs in order to evaluate transgene expression levels by antibody-based detection method. All antibody-based detection methods made use of following primary and secondary antibodies at given dilutions unless otherwise stated. Antibodies were stored as aliquots according to the manufacturers' instruction to avoid freeze-thaw cycles. Antibodies used are described in Table 6 and Table 7.

TABLE 6
Primary Antibodies Used in Evaluation of Transgene Expression Levels.

| | Source/ | | | | |
|---|---|---|---|---|---|
| | Target/ | | Stock | Dilution | |
| Antibody | Clonality | Epitope | [mg/mL] | Used | Technique |
| C- | Rabbit/human/ | EKSLKLSPVQKQKKQQTIGE | 1.28 | 1:500 | WB, ICC, |
| RPGRA\hat{ }51 | polyclonal | (SEQ ID NO: 12) | | | IHC |
| 2-531 | | | | | |
| N- | Rabbit/human/ | KSKFAENNPGKFWFKND/ | 1.9 | 1:500 | WB, ICC |
| RPGRA\hat{ } | polyclonal | GNNEGQLGLGDTEERNT | | | |
| 19-35/ | | (SEQ ID NOS: 13 and 14) | | | |
| 113-129 | | | | | |
| Anti- | Rabbit/human/ | EINDTCLSVATFLPYSSLTSG | 0.2 | 1:500 | WB, ICC, |
| RPGR | polyclonal | NVLQRTLSARMRRRERERSP | | (WB) | IHC |
| (N term) | | DSFSMRRTLPPIEGTLGLSAC | | and | |
| antibody | | FLPNSVFPRCSERNLQESVLS | | 1:200 | |
| | | EQDLMQPEEPDYLLDEMTK | | (IHC) | |
| | | EAEIDNSSTVESLGETTDILN | | | |
| | | MTHIMSLN (SEQ ID NO: 15) | | | |
| Anti- | Rabbit/human/ | C-terminus | 0.25 | 1:500 | WB, ICC |
| RPGR | polyclonal | | | | |
| (C term) | | | | | |
| antibody | | | | | |
| produced | | | | | |
| in rabbit | | | | | |
| Rpgr | Goat/mouse/ | Mouse Rpgr (near C-terminus) | 0.2 | 1:200 | IHC |
| Antibody | polyclonal | | | | |
| (M-20) | | | | | |
| Rpgripl | Goat/mouse/ | Mouse Rpgrip1 | 0.2 | 1:200 | IHC |
| Antibody | polyclonal | | | | |
| (E14) | | | | | |
| Anti- | Mouse/human, | Full length human recombinant | 1 | 1:2,000 | WB |
| GAPDH | mouse, rat, | protein of human GAPDH | | | |
| | dog, | (NP_002037) | | | |
| | monkey/ | | | | |
| | monoclonal | | | | |
| Anti-beta | Mouse/human, | A slightly modified synthetic | | 1:1000 | WB, ICC |
| actin | mouse/ | beta-cytoplasmic actin | | | |
| | monoclonal | N-terminal peptide conjugated | | | |
| | | to KLH | | | |

GADPH = glyceraldehyde 3-phosphate dehydrogenase;
ICC = immunocytochemistry;
IHC = immunohistochemistry;
WB = Western blot.

TABLE 7
Secondary Antibodies Used in Evaluation of Transgene Expression Levels

| | Source/ | | | | |
|---|---|---|---|---|---|
| | Target/ | | Stock | Dilution | |
| Antibody | Clonality | Epitope | [mg/mL] | Used | Technique |
| IRDye ® | Donkey/mouse/ | Mouse IgG (H&L) | 1 | 1:10,000 | WB, |
| 680RD | polyclonal | | | | fluorescent |
| IRDye | Donkey/mouse/ | Mouse IgG (H&L) | 1 | 1:10,000 | WB, |
| 800CW | polyclonal | | | | fluorescent |
| IRDye | Donkey/rabbit/ | Rabbit IgG (H&L) | 1 | 1:10,000 | WB, |
| 800CW | polyclonal | | | | fluorescent |
| IRDye | Donkey/goat/ | Goat IgG (H&L) | 1 | 1:10,000 | WB, |
| 680RD | polyclonal | | | | fluorescent |
| Donkey | Donkey/rabbit/ | Rabbit IgG (H&L) | 0.5 | 1:10,000 | WB |
| Anti- | polyclonal | | | | |
| Rabbit | | | | | |
| HRP | | | | | |
| Donkey | Donkey/mouse/ | Mouse IgG (H&L) | 0.5 | 1:10,000 | WB |
| Anti- | polyclonal | | | | |
| Mouse | | | | | |
| HRP | | | | | |

WB = Western blot.

Immunocytochemistry and Flow Cytometry: HEK293T cells were used for expression of transgene (RPGRORF15) by transfection with respective expression-plasmids. Indirect labeling of the RPGRORF15 required 2 incubation steps, first with a primary antibody directed against RPGRORF15, then with a compatible secondary antibody, with conjugated fluorescent dye at the following concentrations (Table 8).

TABLE 8
Primary and Secondary Antibody Combinations
Used for Indirect Labeling of the RPGR^{ORF15}.

| Primary | | Primary | Secondary |
|---|---|---|---|
| Antibody Target | Species/Kind | Antibody/Concentration | Antibody/Concentration |
| RPGR (N- | Rabbit | 1:500 in PBS-T w/t 1% | Donkey anti-rabbit |
| terminal) | polyclonal | BSA | 1:5000 |
| Beta actin | Rabbit | 1:500 in PBS-T w/t 1% | Donkey anti-rabbit |
| | polyclonal | BSA | 1:5000 |
| GAPDH | Rabbit | 1:5000 in PBS-T w/t 1% | Donkey anti-rabbit |
| | polyclonal | BSA | 1:5000 |

GAPDH = glyceraldehyde 3-phosphate dehydrogenase; RPGR = retinitis pigmentosa GTPase regulator.

Forty-eight hours after transfection, cells were washed before resuspension to approximately 1 to 5×10⁶ cells/mL in ice cold 0.01 M PBS. After fixation in 1% (v/v) paraformaldehyde (PFA) for 10 minutes at 4° C., cells were gently pelleted down at 120×g for 5 minutes at 4° C. Aqueous solution was carefully aspirated and cells re-suspended in blocking solution (10% [w/v] donkey serum in PBS-T [0.1% Triton-X in 0.01 M PBS]). After 30 minutes, cells were spun again as above and supernatant removed. Primary antibody solution was added at the appropriate concentration and sample incubated at room temperature for 2 hours. After 3 wash steps (cells pelleted down at 120×g for 5 minutes at 4° C., supernatant removed, cells re-suspended in ice cold PBS-T), a fluorochrome-labeled secondary antibody (optionally, Hoechst 33342 dye was added to the secondary antibody solution at 1:5000) was added for 30 minutes in the dark at room temperature, followed by the same washing procedure. Cells were kept on ice until further processing on the same day.

Cell suspension was either added drop-wise on a poly-L-lysin coated glass slide (Gerhard Menzel GmbH, Braunschweig, Germany) or mounted in ProLong® Gold (Life Technologies) for fluorescence microscopy. Alternatively, cells were subjected to flow cytometry using a CyAn Advanced Digital Processing (ADP) LX High-Performance Research Flow Cytometer (DakoCytomation, Beckman Coulter Ltd, High Wycombe, UK) at the Flowcytometry Facility of the University of Oxford (The Jenner Institute, Nuffield Department of Medicine). This 9-color digital flow analyser features 3 solid-state lasers (488, 635, and 405 nm) and analyses up to 500,000 events per second. Gate settings were chosen based on data gained from the positive controls for a false discovery rate of <1 and their median fluorescence intensity.

Liquid Chromatography-Tandem Mass Spectrometry: Expression of transgene (RPGR^{ORF15}) was evaluated by liquid chromatography-tandem mass spectrometry (LC-MS/MS) following transfection of HEK293T cells with respective expression-plasmids, according to the method described below.

Forty-eight hours after transfection, cells were washed and brought into suspension with 0.01 M PBS before spinning at 120×g and 4° C. for 10 minutes. Centrifugation was repeated after re-suspending pellet in 500 μL of 0.01 M PBS. Supernatant was discarded and cell pellets subjected to a single freeze-thaw cycle before adding 200 μL ice-cold Radio-Immunoprecipitation Assay (RIPA) buffer with 1 dissolved complete mini EDTA-free protease inhibitor cocktail tablet (Roche Products Ltd., Welwyn Garden City, UK) per 10 mL of RIPA buffer. Cell pellets were mechanically disrupted with polypropylene pellet pestles on a motor-driven grinder (Sigma-Aldrich) and cell fragments spun down at 14,000 rpm and 4° C. for 30 minutes. Supernatant was quantified using the Pierce™ bicinchoninic acid (BCA) Protein Assay Kit (Thermo Scientific) according to the manufacturer's instructions. The microplate procedure was used for colorimetric quantitation of total protein: first, the working reagent and 9 BSA standards were prepared with final concentrations ranging from 25 to 2000 μg/mL. After 25 μL of each standard or unknown sample replicate was pipetted into a white 96 microplate well, 200 μL of the working reagent was added, and the plate mixed on a shaker for 30 seconds before incubating at 37° C. for 30 minutes. After the plate cooled to room temperature, the absorbance at 562 nm was assessed on a Biochrom EZ Read 400 plate reader.

Samples were diluted to 1 μg/μL total protein concentration and denatured in Laemmli buffer (Sigma-Aldrich) for 20 minutes at RT. 10 μg total protein was loaded per well using 7.5% sodium dodecyl sulfate polyacrylamide gels (Criterion™ TGX™ Precast Gels, Bio-Rad Laboratories Ltd., Hemel Hempstead, UK) for electrophoresis at 100 V for 2 hours (SDS-PAGE). EZBlue™ Gel Staining Reagent (SIGMA) was used to stain proteins according to the manufacturer's instructions: the SDS-PAGE Gel was rinsed 3 times for 5 minutes each in an excess of water to remove SDS before incubating the Gel in the EZBlue Gel Staining Reagent for 2 hours at room temperature on a shaker. The gel was then washed in excess water for 2 hours before an image was taken and the appropriate bands excised with a disposable scalpel. Bands were transferred to 1.5-mL Eppendorf tubes and stored at 4° C. until further processing at the Proteomics Centre of the University of Oxford (Dunn School of Pathology). Samples were digested using trypsin, lysine C, lysine N, pepsin, formic acid, elastase, and V8 protease followed by LC-MS/MS. Peptide fragments were recorded along their sequence identity and matched to the human proteome. All testing was conducted in accordance with the established procedures of the Proteomics Centre.

Western Blot: The expression level of RPGR in the transfected HEK293T cells was evaluated by Western blot analysis according to the following protocol. Protein samples from the plasmid transfection experiments were prepared and separated using SDS-PAGE.

Gels were carefully placed onto polyvinylidene difluoride (PVDF) membranes with 0.2 μM pore size (Trans-Blot® Turbo™ Midi PVDF, Bio-Rad) and proteins blotted using the Trans-Blot Turbo Transfer Starter System (Bio-Rad), according to the manufacturer's instructions, using the midi setting (7 minutes at 25 V). PVDF membranes were then cut into sections depending on size of target protein and loading control to stain independently with respective primary (Table 6) and/or secondary (Table 7) antibodies.

PVDF membranes were blocked, washed, and incubated with antibody solutions in the SNAP i.d.™ protein detection system (Millipore (U.K.) Ltd., Feltham, UK), according to instructions by the manufacturer. Briefly, membranes were placed in wells of appropriate size with the protein-loaded side facing up towards the open chamber of the well. 0.01 M PBS with 0.1% Triton-X (PBS-T) was combined with 1% BSA. To block unspecific binding, 10 mL PBS-T with 1% BSA was added to each well and vacuum applied to draw solution through PVDF membrane. Primary antibody solution (3 mL) was applied to the well and left to incubate for 10 minutes at RT before applying vacuum, followed by washing 3 times with approximately 30 mL PBS-T. Incubation with horseradish peroxidase (HRP)-linked secondary antibody followed the same steps as with the primary antibody solution. After the final washing step, membranes were removed from wells and incubated with Luminata forte ELISA HRP substrate to allow activation of chemiluminescence. Membrane sections were carefully re-assembled in a BAS cassette 2040 (FUJIFILM UK Ltd., Bedford, UK) for exposure on CL-Xposure™ film (Thermo Scientific) in a dark chamber. Films were developed in a Compact X4 Automatic X-ray Film Processor (Xograph Healthcare, Gloucestershire, UK), and resulting films scanned using an Epson Perfection V30 flatbed scanner (Epson (UK) Ltd., Hertfordshire, UK) in an uncompressed tagged image file format (TIFF) with 16-bit color depth and 1200-dpi resolution.

### Methods Used in Codon Optimisation of RPGR

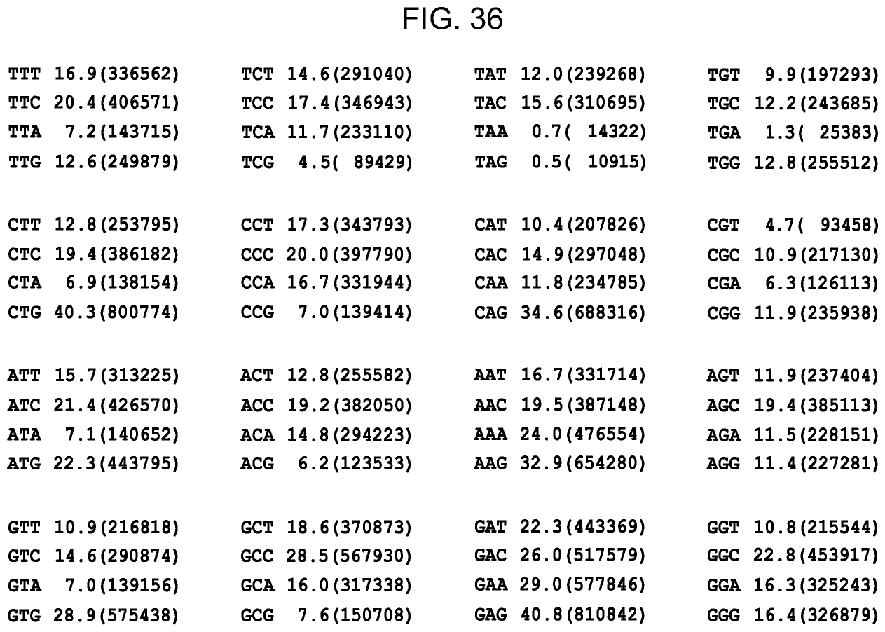

Geneious software (version 6.1.6 for Mac OS X 10.7.5; Biomatters Ltd, Auckland, New Zealand) was used to search the consensus cd database (CCDS) of the National Center for Biotechnology Information (NCBI) for the reference human RPGRORF15 nucleotide sequence. The complete cds was subjected to the OptimumGene™ algorithm (GenScript, Piscataway, USA) to optimize a variety of parameters that are critical to the efficiency of gene expression, including codon usage bias, GC content, CpG dinucleotides content, mRNA secondary structure, cryptic splicing sites, premature poly-A sites, internal chi sites and ribosomal binding sites, negative CpG islands, RNA instability motif (ARE), repeat sequences (direct repeat, reverse repeat, and Dyad repeat), and restriction sites that may interfere with cloning. The codon frequency table that was used is displayed in FIG. 36.

The codon-optimised human cds of the retina-specific isoform RPGR^{ORF15} was synthesised by GenScript. The wild type sequence of RPGR^{ORF15} was synthesised by OriGene and provided in the pCMV6-XL vector backbone and by GenScript in a pUC57 vector backbone for cloning.

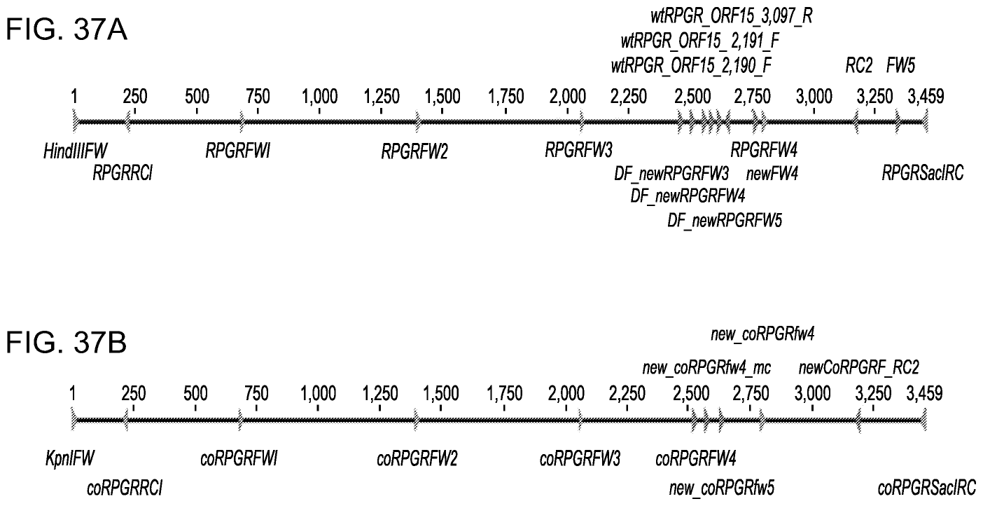

Sequences were confirmed by Sanger sequencing by Source BioScience services at the Department of Biochemistry, University of Oxford. For this, multiple samples were prepared at 100 ng/μL plasmid DNA and appropriate sequencing primers were added at 3.2 pmol/μL to initiate reads at various locations along the predicted sequence (FIG. 37). Samples were analyzed according to standard laboratory procedures.

### Codon Optimization

The cds of a gene serves as template for translation of nucleic acid sequence into peptides. This process involves the cds contained in the mRNA transcript, ribosomal complexes, and amino acids, which are bound to tRNA molecules. Three consecutive nucleotides in the cds (eg, UUA) constitute a codon. tRNA molecules have complementary anti-codon sequences (eg, AAU), briefly bind to the codon sequence within the ribosomal complex, and contribute a single amino acid (eg, Leucine) they are carrying to the growing chain of amino acids forming the growing peptide encoded by the cds.

With 4 nucleotides available to encode each of the 3 positions in a codon, 4³=64 codons can be formed. Because 3 combinations encode stop signals (UAA, UAG, UGA), 61 possible combinations are available for 20 amino acids. This redundancy results in multiple codons translating into the same amino acid: leucine, for example, is added at codon sequences UUA, UUG, CUU, CUC, CUA, or CUG. Highly expressed genes preferentially use so-called major codons.

Human RPGR^{ORF15} cds encodes an 1152 amino acid protein with a highly repetitive, purine-rich mutational hotspot as C-terminal exon. Cloning this isoform without random mutations being introduced is difficult, as is direct sequencing of the adenine/guanine rich regions, since polymerases have a tendency to stop at guanine repeats. The codon usage of RPGR^{ORF15} cds was optimized to increase sequence fidelity during the cloning process and provide a construct with the potential to sidestep previous problems in clinical vector design. Additionally, increasing the codon adaptation index (CAI) of the RPGR^{ORF15} cds through introducing synonymous major codons where possible might lead to higher transgene expression without the use of accessory regulatory elements in the transgene cassette. This is important as the cds of RPGR^{ORF15} even without promoter or polyadenylation site already fills more than 3 quarters of the available space between the inverted terminal repeats of the gutted AAV genome.

The result of the database query for human RPGR^{ORF15} was a 3459-bp long cds (CCDS 35229.1), known as X-linked retinitis pigmentosa GTPase regulator isoform C, transcribed and spliced from gene ID 6103 on the minus strand of the X chromosome at Xp21.1.

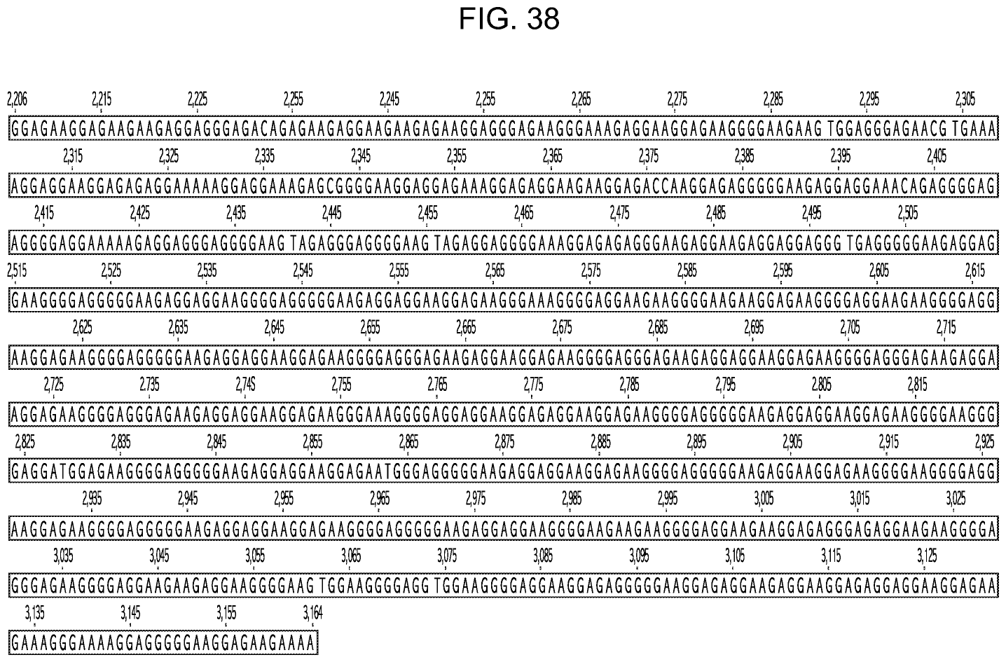

The sequence featured a well-balanced GC content of 47.2% and a Tm at 84.1° C., but an overabundance (72%) of purines versus pyrimidines with 36% adenine and 35.5% guanine. This imbalance was even most pronounced regionally within the cds. In one particular 959 base pair fragment (FIG. 38) of the central ORF15 region, 93% of nucleotides were purines (56% guanine >37% adenine >>6% cytosine >1% thymidine).

This limited variability leads to high rate of repetitions of 15 to 33 bp long nucleotide sequences in the region between 2458 and 2799 of the cds and multiple poly-guanine runs (5′-GGGGAGGGG-3′), which are notoriously difficult to sequence as the long run of G's inhibits the ability of the polymerase to unwind the template.

Another consequence of the repetitive, purine-rich nucleotide sequence is a skewing of the amino acid frequency towards glutamic acid (26.6%) and glycine (15.4%), with all 17 other amino acids (not counting methionine) featuring in only 0.7% to 6.6% of the cases. These particular characteristics of the wild type human RPGR^{ORF15} cds almost certainly contributes to the genetic instability of the gene, thereby leading to the high prevalence of mutations found in patient populations.

### An Optimised Coding Sequence for RPGR

Analysis of the CAI of wtRPGRORF15 showed a moderate CAI of 0.73 with 10% use of minor codons (low abundance codons), but only 32% use of major codons, ie, codons with the highest usage frequency for a given amino acid in Homo sapiens. This frequency of optimal codons (FOP) was changed in favour of higher codon quality groups during codon optimisation: only 1% minor codons were left unchanged and the frequency of major codons was increased to 56%. This improved the CAI to 0.87 for coRPGR^{ORF15}.

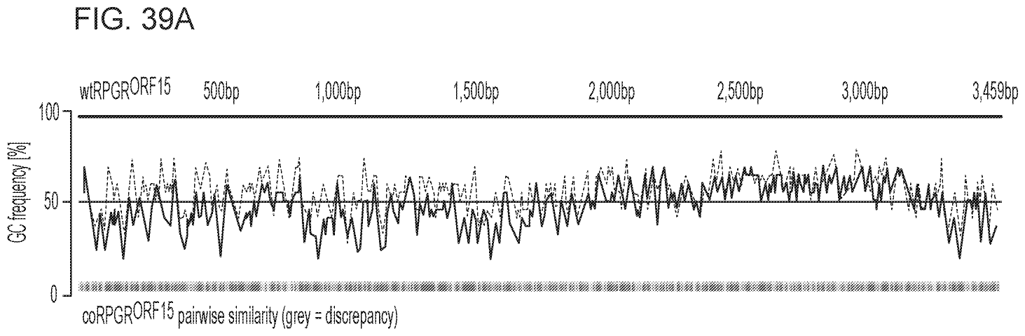

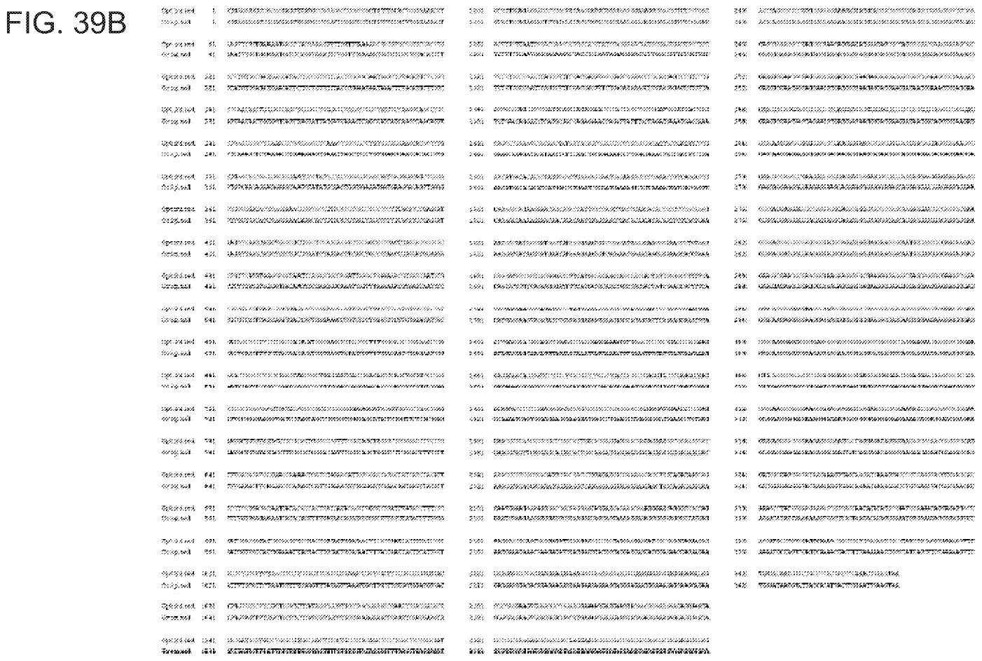

In addition to increasing the CAI, codon optimisation also removed an MfeI restriction site and several cis-acting elements, such as a potential splice site (GGTGAT), 4 polyadenylation signals (3 AATAAA and 1 ATTAAA), 2 polyT (TTTTTT), and 1 polyA (AAAAAAA) sites. GC content and unfavourable peaks were optimised to prolong the half-life of the mRNA. Secondary structure formations (stem-loops), which would reduce the chance of ribosomal binding and render mRNA less stable, were disabled. The pairwise % identity between wtRPGR^{ORF15} and coRPGR^{ORF15} was 77.2% with most changes occurring in the ORF15 region (FIG. 39).

Codon-optimised RPGR Shows Higher Sequence Fidelity than Wild Type RPGR

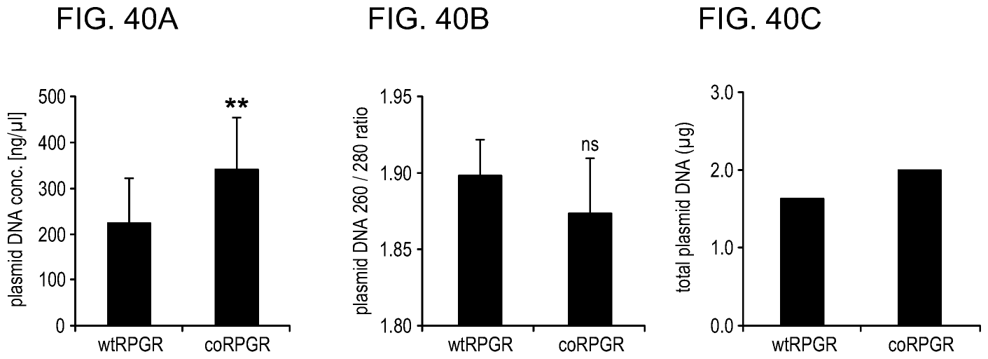

The synthesized sequence of coRPGR^{ORF15} showed no sequence deviation throughout the necessary steps towards successfully sub-cloning it into the Vector BioLabs pAAV2 plasmid for downstream AAV vector production. Synthesis of the original plasmid product containing coRPGR^{ORF15} at GenScript took approximately 6 weeks. Synthesis of wtRPGR^{ORF15} by GenScript took approximately double the time compared with the coRPGR^{ORF15} (approximately 12 weeks). All subsequent steps involving wtRPGR^{ORF15} en route to the sub-cloning into the pAAV2 plasmid showed lower numbers of clones with correct fragment size: out of 24 colonies of XL10-Gold bacteria following transformation with wtRPGR^{ORF15}, only 3 samples featured expected fragment sizes (FIG. 40). In contrast, cloning of coRPGR^{ORF15} resulted in 18 out of 24 positive clones. Furthermore, plasmid DNA concentration from coRPGR^{ORF15} mini-preparations were approximately 50% higher (n=24, unpaired, 2-tailed t-test: p=0.0004), while 260/280 ratio remained unchanged.

Sequencing the wtRPGR^{ORF15} construct at various stages of the sub-cloning posed a major challenge due to the repetitive nature and poly-G runs within the ORF15 region. Some regions required use of deoxyguanosine triphosphate (dGTP) sequencing to improve read-through in purine-rich regions (e.g., FIG. 38) with long guanine runs. While this technique provides better read-through, it is more likely to introduce band compressions (the reason why in standard Sanger reactions, the analogous molecule deoxyinosine triphosphate [dITP] is used instead of dGTP).

In 8 independent cloning experiments (n=4 for each construct), an average of 30 sequence runs were necessary to gain full coverage of wild type construct, while a mean of 8 sequence runs were sufficient for the coRPGR^{ORF15} sequence. Alignment of sequence data to the reference revealed numerous deletions, insertions, and point mutations of (mostly) single nucleotides in wtRPGR^{ORF15}, but none in coRPGR^{ORF15} (Table 9).

| | wtRPGR^{ORF15} | coRPGR^{ORF15} |
|---|---|---|
| Deletions (mean [range]) | 1.5 (0-4) | nil |
| Insertions (mean [range]) | 0.5 (0-1) | nil |
| Point mutations (mean | 17.8 (9-33) | nil |
| [range]) | | |
| Total (mean [range]) | 19.75 (9-38) | nil |

Key parameters including the Phred quality scores Q20, Q30, and Q40 (Q20 indicates a base call accuracy of 99%, Q30 of 99.9%, and Q40 of 99.99%) (Table 10), mean confidence and number of expected errors were significantly weaker in wtRPGR^{ORF15} versus coRPGR^{ORF15} (Table 11). Data are shown as mean±standard deviation, p-values were corrected for multiple comparison using the false discovery rate (FDR) correction method.

| [%] of Base Calls | | | |
|---|---|---|---|
| with a Confidence | | | p-Value [FDR |
| Level of at Least | wtRPGR^{ORF15} | coRPGR^{ORF15} | Corrected] |
| 99% (Phred Q20) | 91.3 ± 1.0 | 96.6 ± 0.9 | 0.0005 |
| 99.9% (Phred Q30) | 83.1 ± 2.6 | 90.5 ± 2.6 | 0.0044 |
| 99.99% (Phred Q40) | 73.6 ± 3.8 | 82.3 ± 3.0 | 0.0054 |

| | | | p-Value [FDR |
|---|---|---|---|
| | wtRPGR^{ORF15} | coRPGR^{ORF15} | Corrected] |
| Confidence Mean | 49.1 ± 1.2 | 52.4 ± 1.0 | 0.0044 |
| Expected Errors | 82.9 ± 25.1 | 14.4 ± 5.1 | 0.0023 |

Final proof for the superior sequence fidelity of coRPGRORF15 was given by the National Genetics Reference Laboratory (NGRL) in Manchester. After exchanging the CAG promoter region (1527 bp) for the much smaller human rhodopsin kinase promoter (199 bp) to aid the recombinant production of AAV, as well as localization to photoreceptor cells, both constructs (RK.coRPGR^{ORF15} and RK.wtRPGR^{ORF15}), along with appropriate primers, were sent to the NGRL and personnel left masked as to the identity of the sequences. After running 34 sequence reactions on RK.wtRPGR^{ORF15} as template, the cumulative data showed 74 ambiguous nucleotide calls (eg, equal signal for guanine and adenine) and 6 potential insertion/deletion mutations (4 potential insertions and 2 potential deletions), all found in the purine-rich ORF15 region, the mutational hotspot of RPGR^{ORF15}. In contrast, the coRPGRORF15 construct was sequenced with at least 2 times coverage, with exactly half the number of sequence reactions and no mutations found in the plasmid.

Codon-Optimised RPGR Yields Higher Expression Levels then Wild Type RPGR

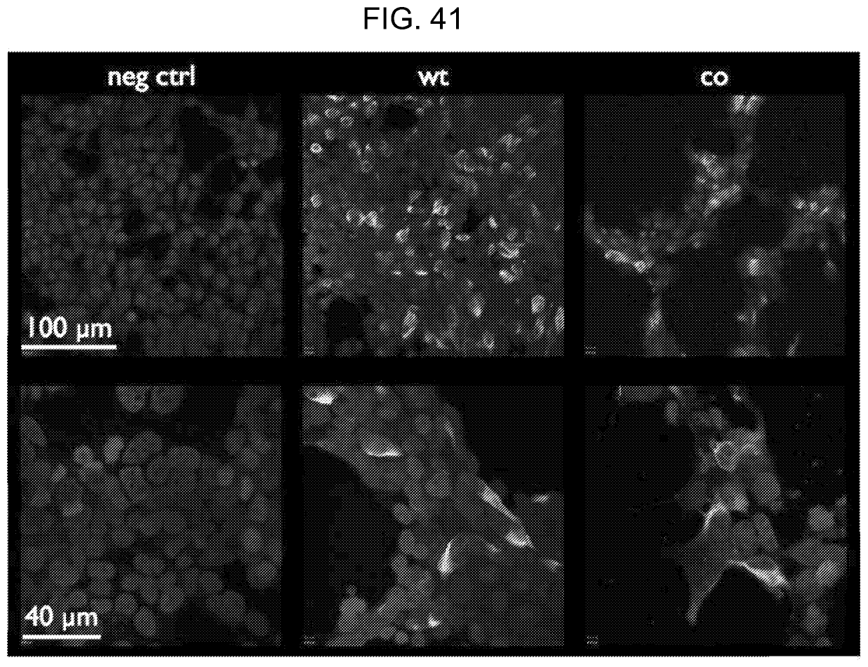

In order to analyse the effect of an increased codon adaptation index (CAI) on the expression levels of RPGR^{ORF15}, transfection experiments were performed on HEK293T cells using the CAG.coRPGR^{ORF15} and CAG.wtRPGR^{ORF15} plasmid constructs in head-to-head comparisons. HEK293T cells are of human origin, and therefore, share the species-specific codon frequency distribution, which served as basis for the optimisation for Homo sapiens. It was hypothesized that cells transfected with CAG.coRPGR^{ORF15} produce more RPGR^{ORF15} than cells transfected with the wild type construct CAG.wtRPGR^{ORF15}. To test this hypothesis, several experimental avenues were taken in order to quantify RPGR^{ORF15} in transfected cells. First, HEK293T cells were transfected with CAG.coRPGR^{ORF15} and CAG.wtRPGR^{ORF15} plasmid constructs and processed for immunocytochemistry (ICC) to establish whether the transgene detection could be detected by antibody binding. FIG. 41 shows representative images from such an experiment, where cells were transfected with medium only (neg ctrl), CAG.wtRPGR^{ORF15} (wt), or CAG.coRPGR^{ORF15} (co), and stained with anti-RPGR.

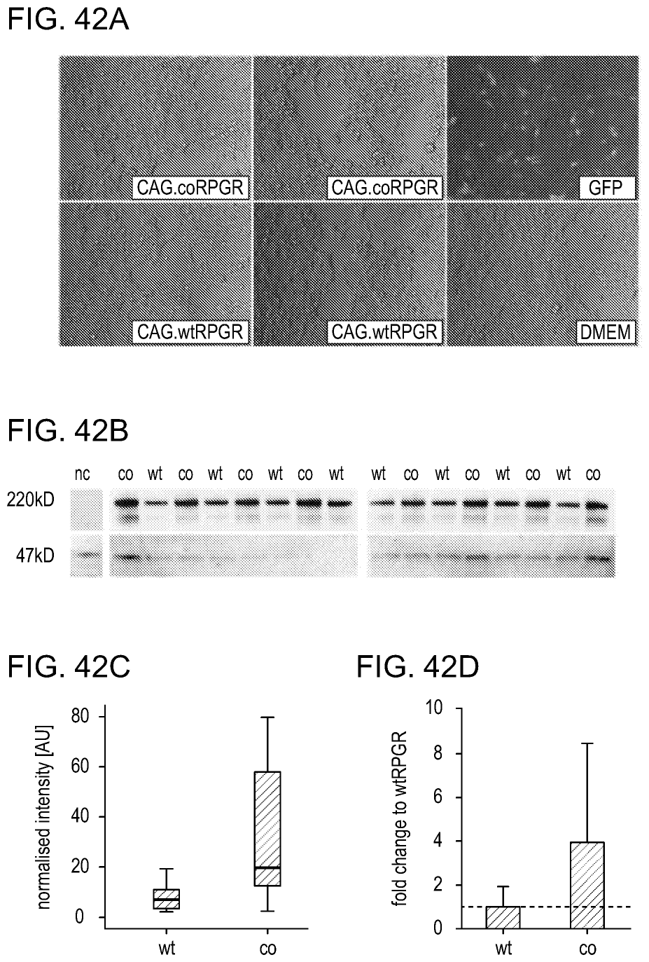

Western blot analysis was used to assess expression levels in whole cell lysate from transfected HEK293T cells. Four independent 6-well plate transfections, each with a technical replicate for wt- and coRPGR, produced a total n of 8 per construct. Aliquots from these lysates were run on 2 gels in parallel and mean signal intensities of resulting bands compared (FIG. 42). The Shapiro-Wilk test retained the null-hypothesis for normality of the data sets (p=0.06 to 0.19) and 1-way ANOVA showed the statistical significance (p=0.01, n=8) of the difference between the mean signal intensity reflecting the codon-optimised construct=32.0±8.28 arbitrary units [AU] (mean±standard error) and the signal from cell transfected with the wild type construct=8.11±1.63 AU.

Fluorescence-activated cell sorting (FACS) was also used to measure expression levels of RPGR^{ORF15} in transfected HEK293T cells. In a similar setup as mentioned above, 3 independent experiments with 6-well plates were conducted, each with 3 technical replicates of wells with HEK293T cells transfected with either CAG.wtRPGR^{ORF15}, CAG.coRPGR^{ORF15}, CAG.eGFP (as positive control for transfection) or media only (as negative control).

Fluorescence-activated cell sorting (FACS) was also used to measure expression levels of RPGR^{ORF15} in transfected HEK293T cells. In a similar setup as mentioned above, 3 independent experiments with 6-well plates were conducted, each with 3 technical replicates of wells with HEK293T cells transfected with either CAG.wtRPGR^{ORF15}, CAG.coRPGR^{ORF15}, CAG.eGFP (as positive control for transfection) or media only (as negative control).

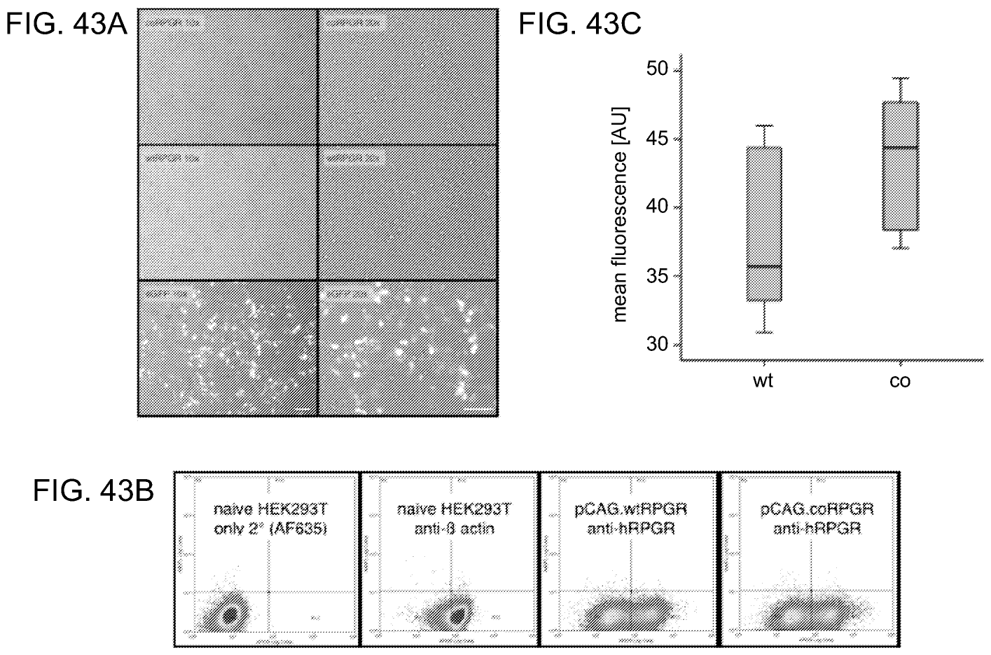

Cells transfected with CAG.eGFP showed eGFP expression at time of harvest, indicating that the transfection was successful and that the cells had enough time to produce a plasmid-encoded transgene. After the ICC protocol, these cells were used to set the lower end of the FACS gating for fluorescence in the far-red range, as they were incubated with secondary antibody only. The positive controls (naïve HEK293T cells exposed to rabbit anti-β-actin and donkey anti-rabbit with conjugated Alexa-Fluor 635) were then used to define the upper end of the fluorescence gate setting. Cells transfected with the CAG.coRPGR^{ORF15} construct showed higher fluorescence intensity then the cells transfected with the wild type construct, CAG.wtRPGR^{ORF15}, (FIG. 43). The Shapiro-Wilk test rejected the null-hypothesis for normality of the data sets (p <0.05) and the Kruskal Wallis non-parametric test demonstrated a robust statistical difference between the cohorts (p <0.01, n=9).

### Example 6: Microperimetry Measurement of Therapeutic Efficacy within and Near the Macula

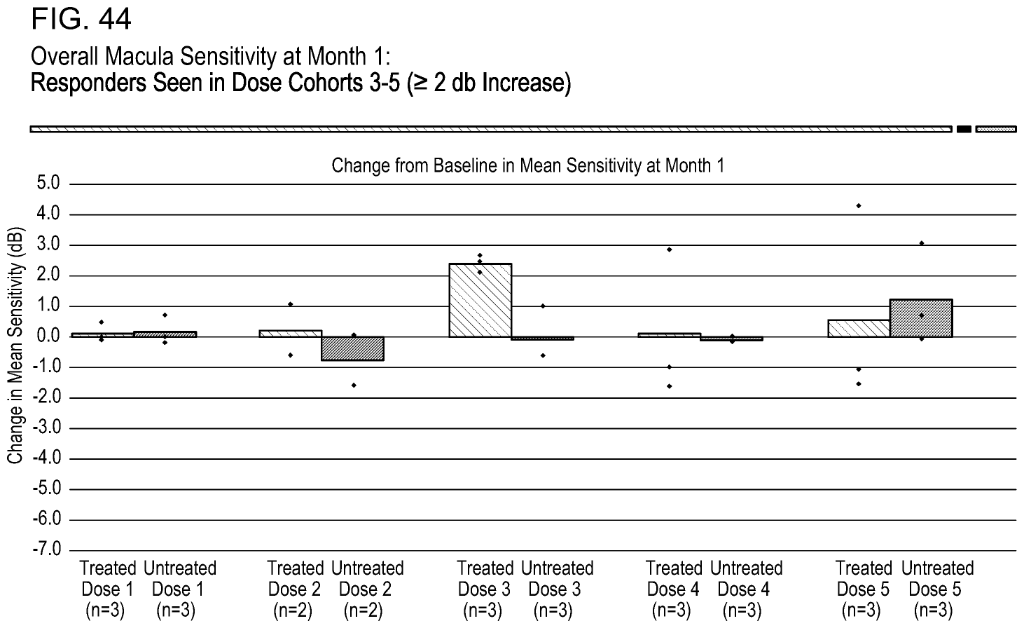

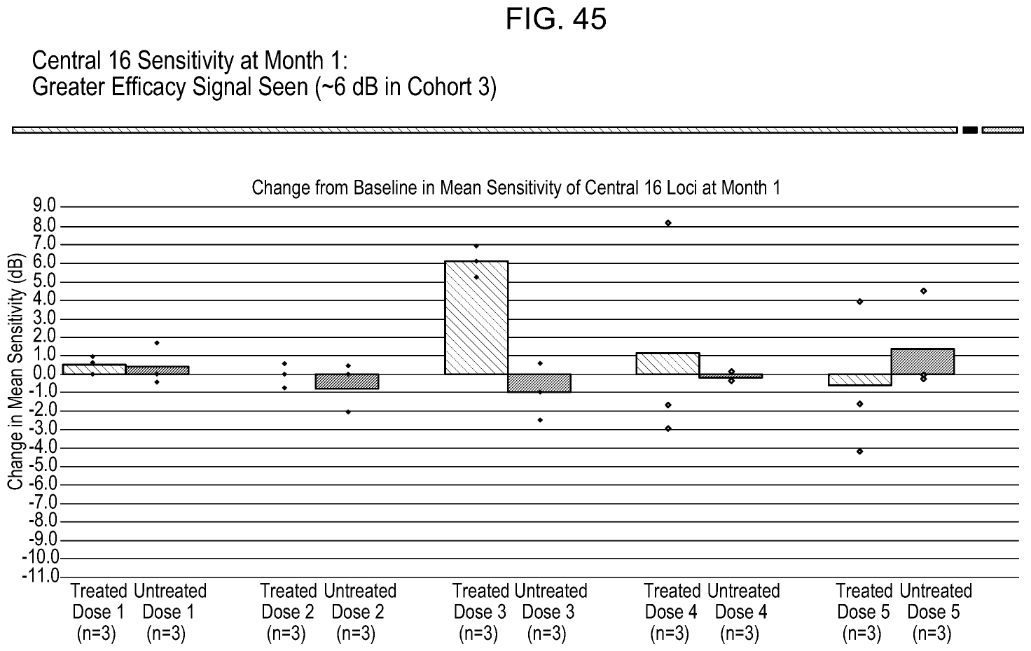

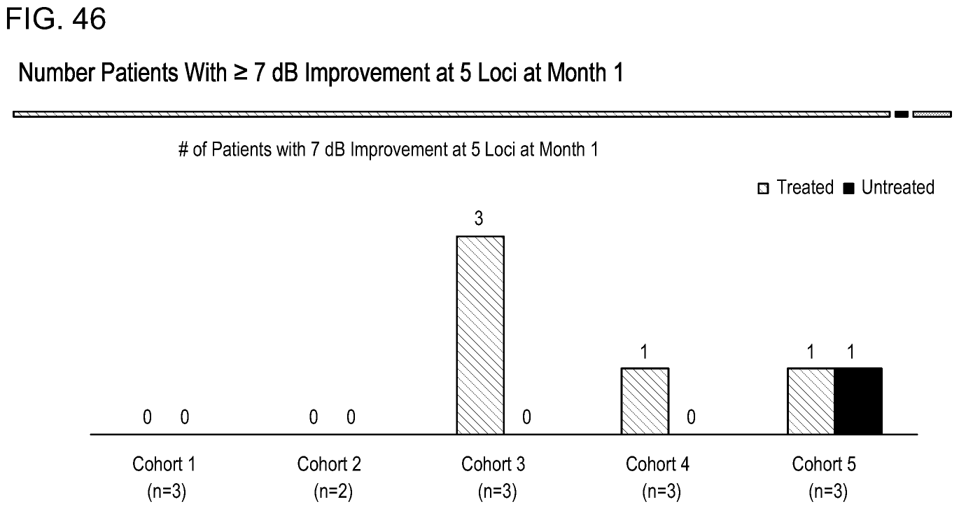

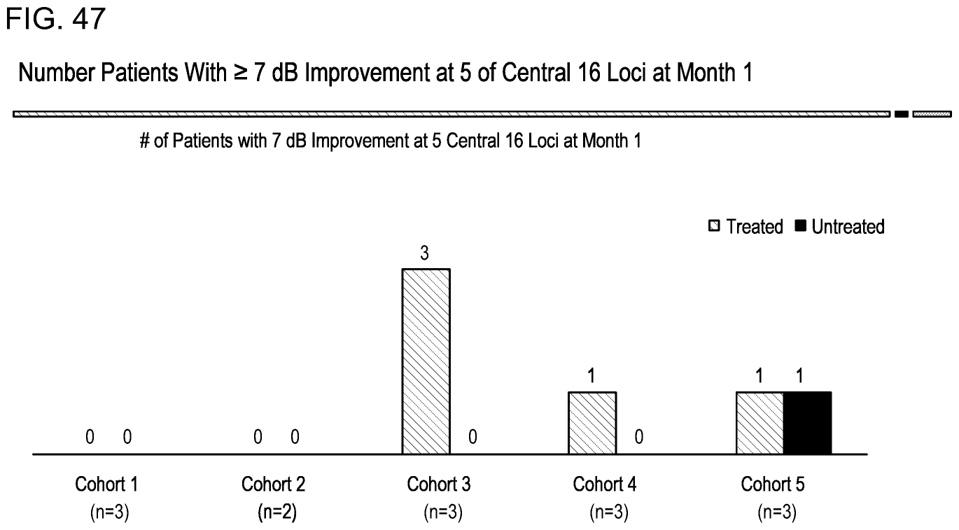

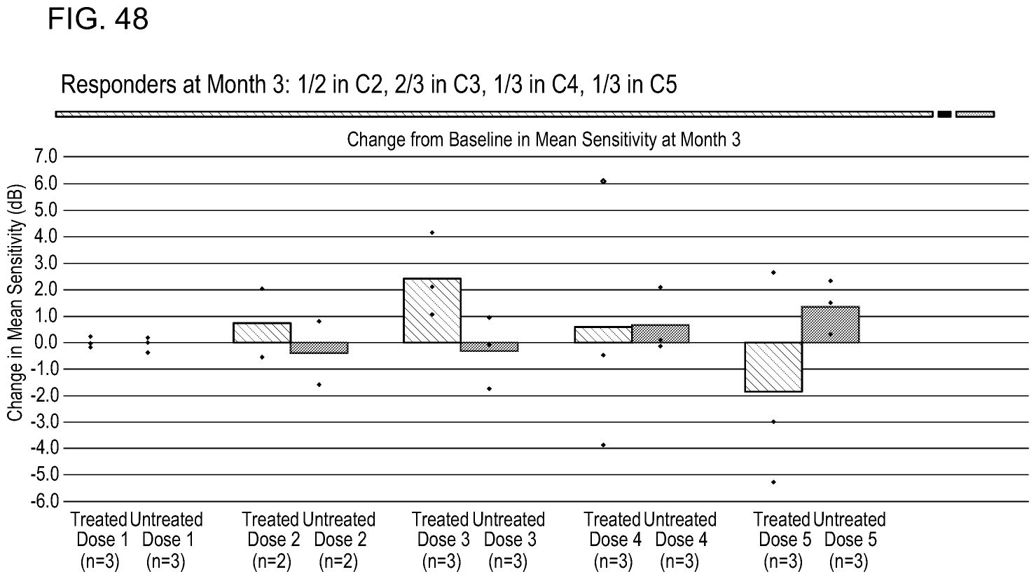

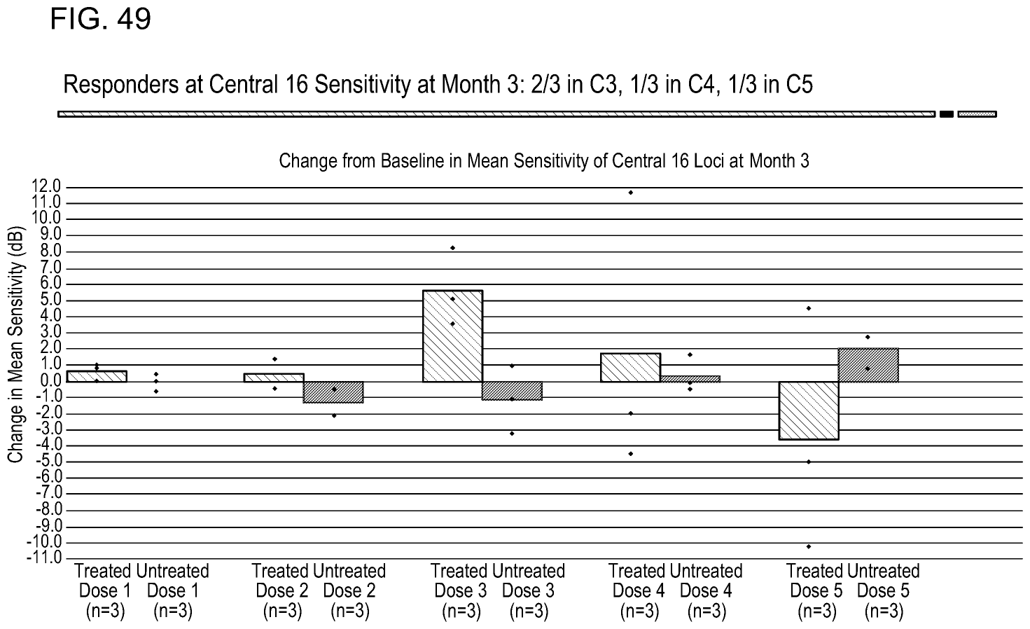

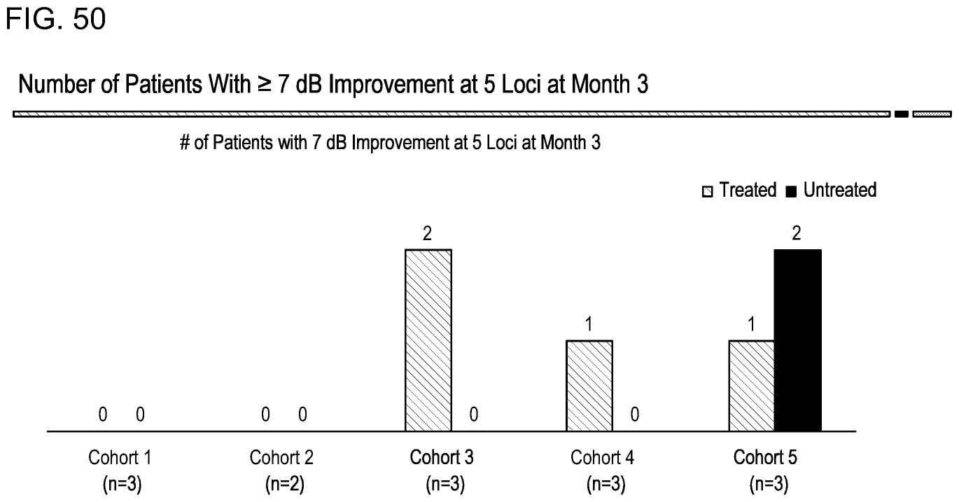

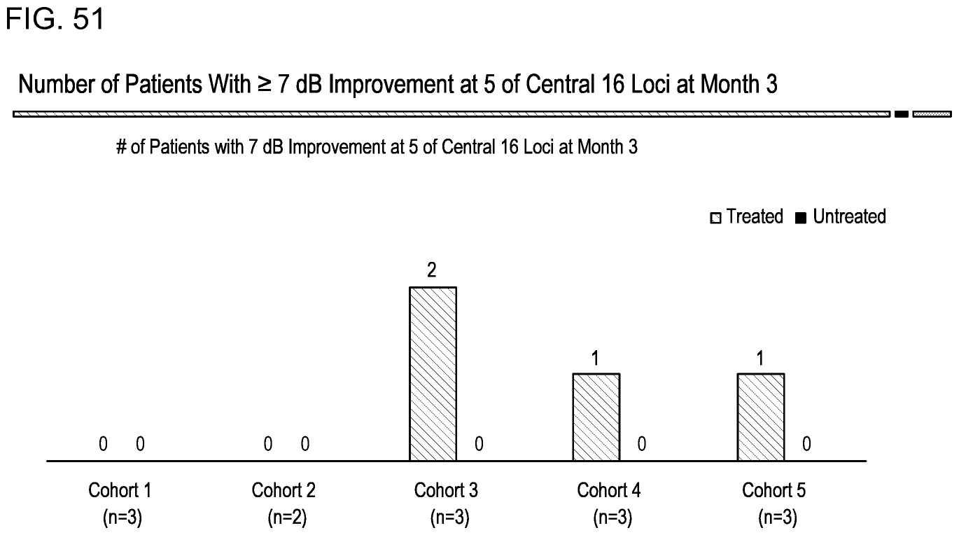

Subjects were treated with a composition of the disclosure comprising an AAV-coRPGR^{ORF15} particle. Prior to treatment a baseline microperimetry measurement of all 68 loci was taken. Following treatment at various timepoints, a follow-up microperimetry measurement of all 68 loci was taken. FIGS. 44-51 provide the results of this study in which both the entire field of 68 loci and a central 16 set of loci are evaluated for therapeutic efficacy.

### Example 7: OCT Measurement of Therapeutic Efficacy as Shown by Retinal Thickness

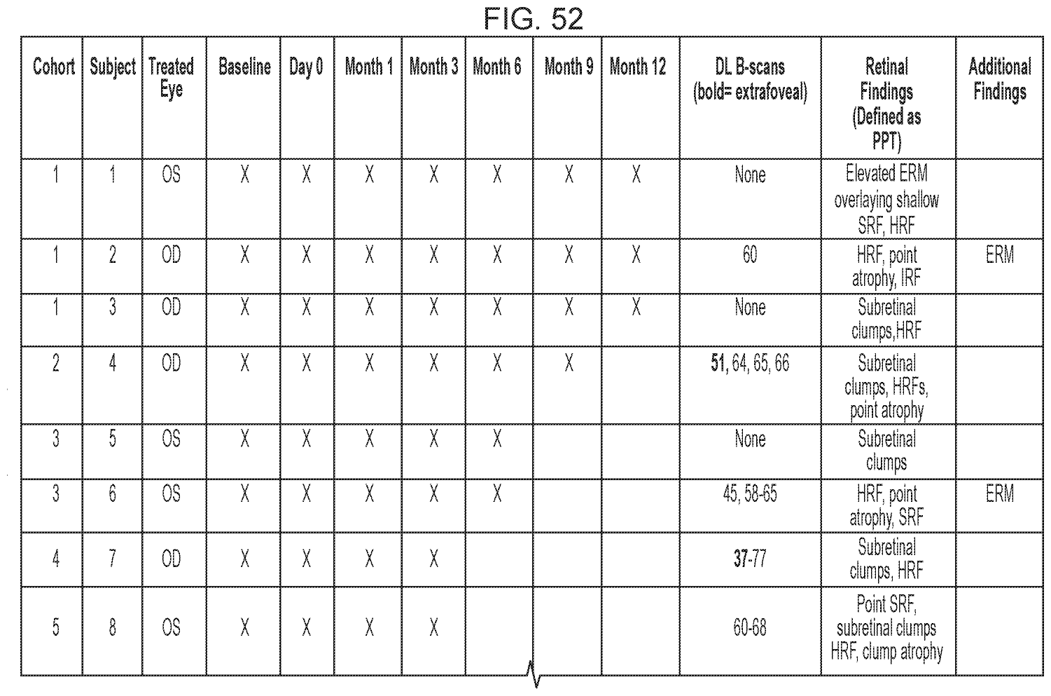

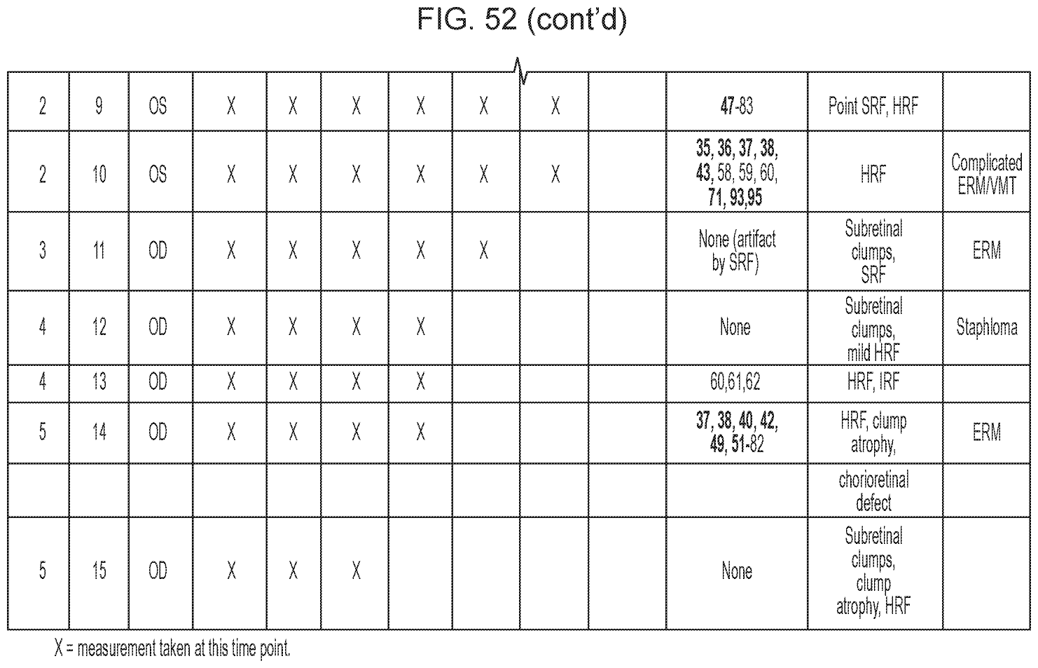

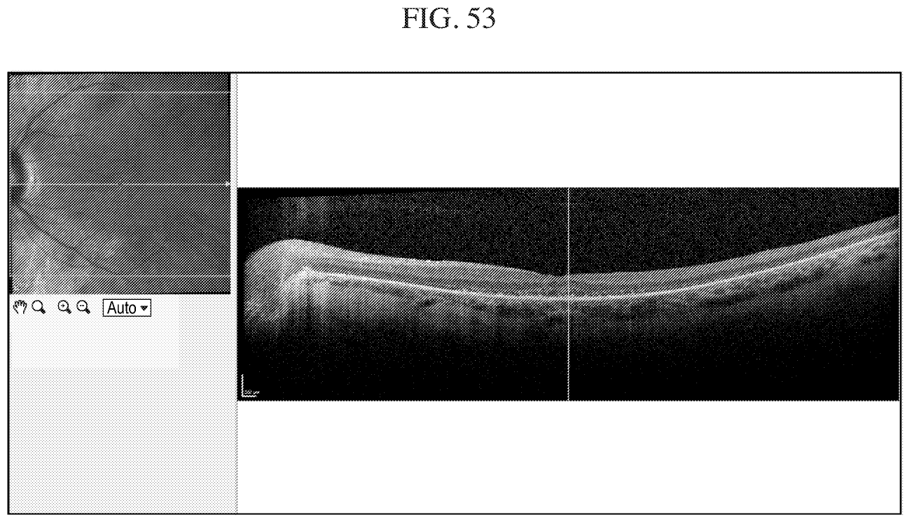

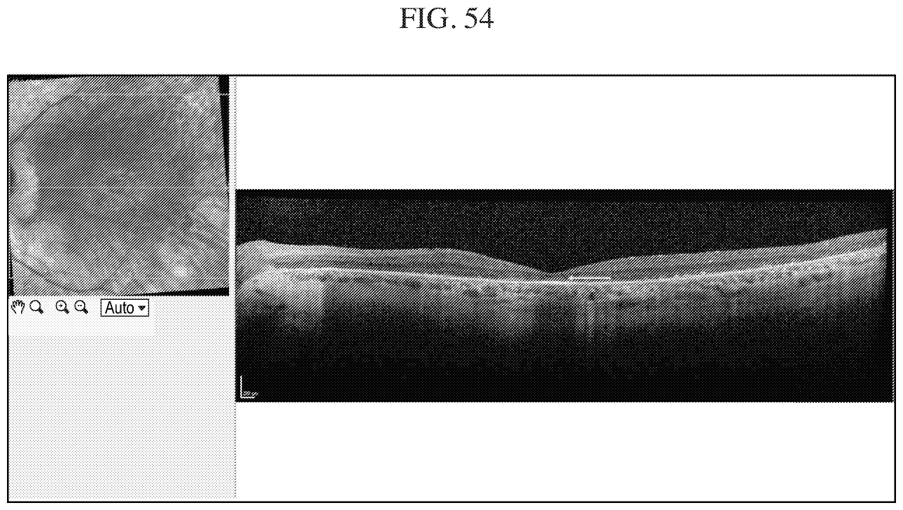

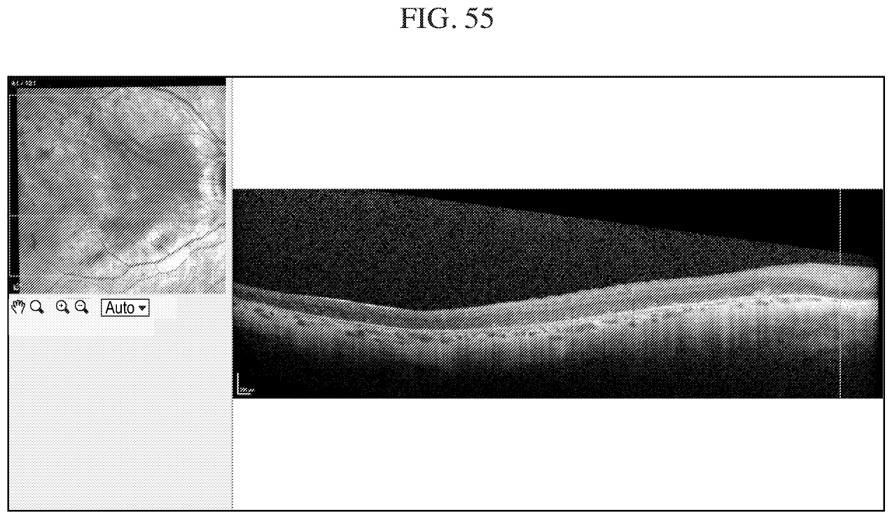

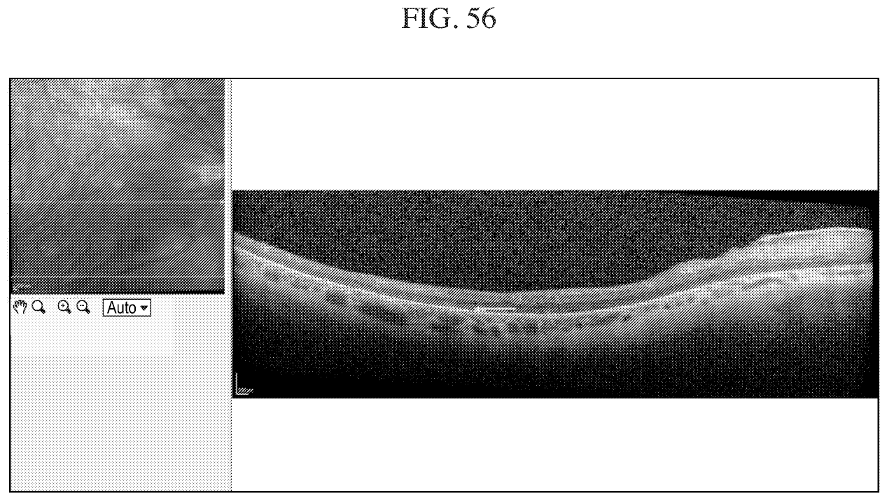

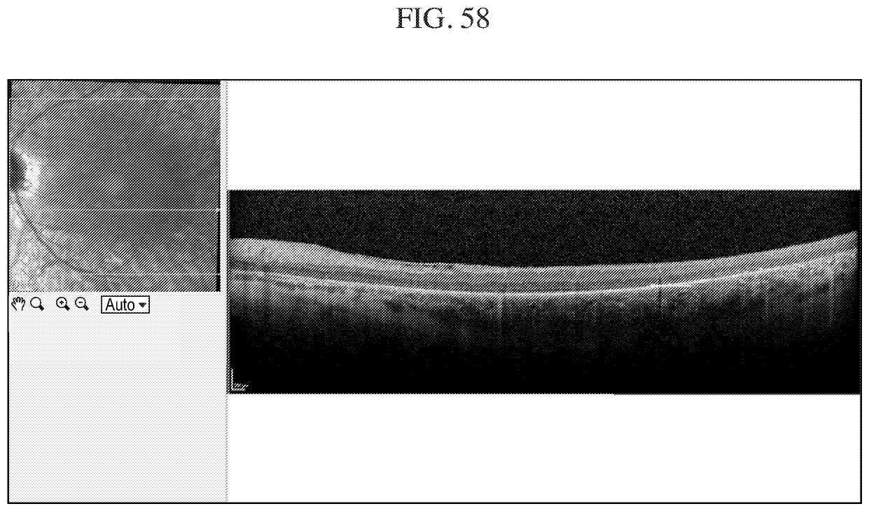

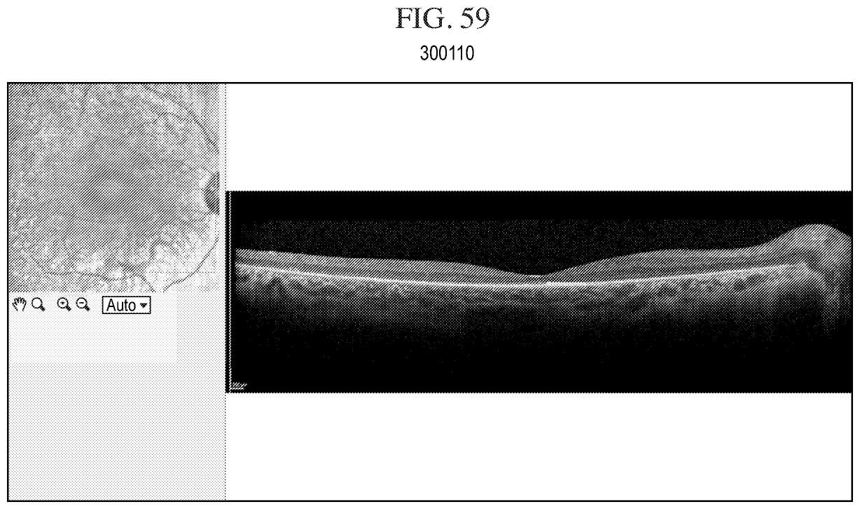

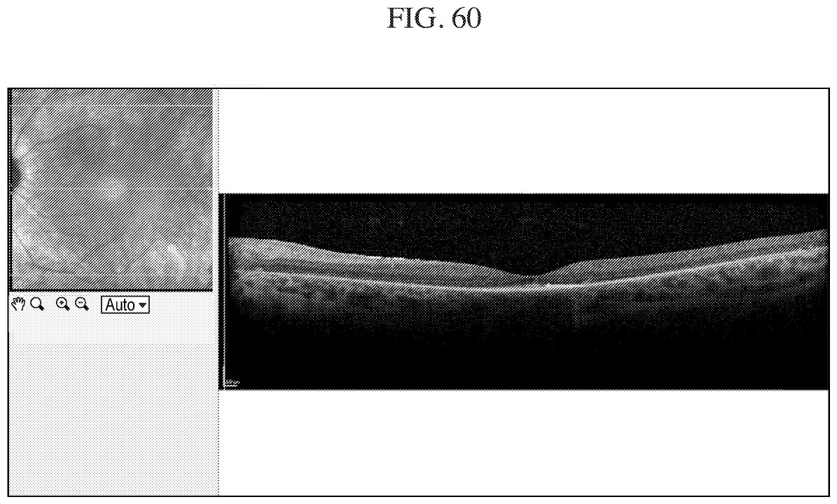

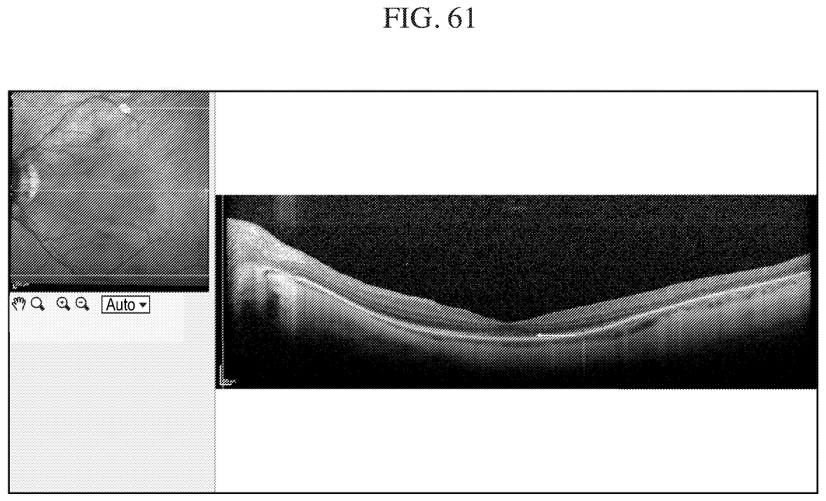

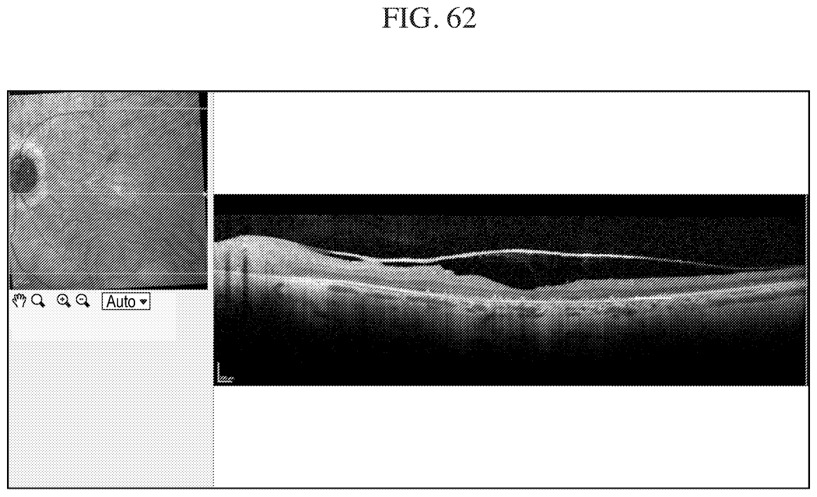

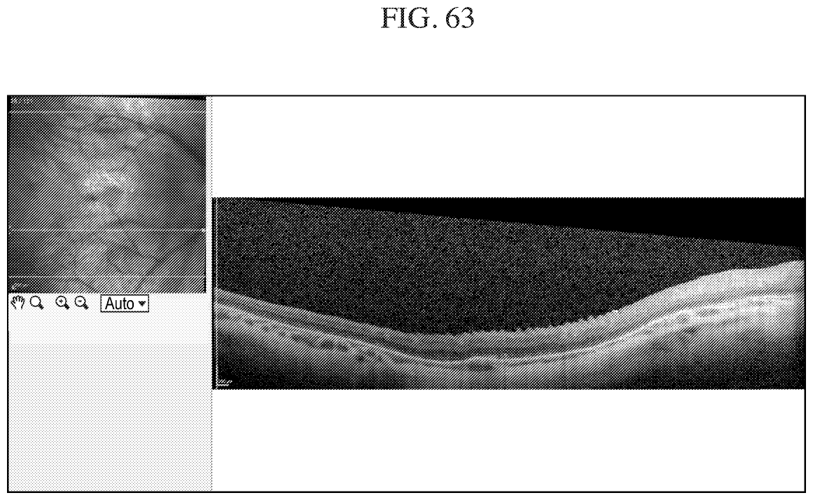

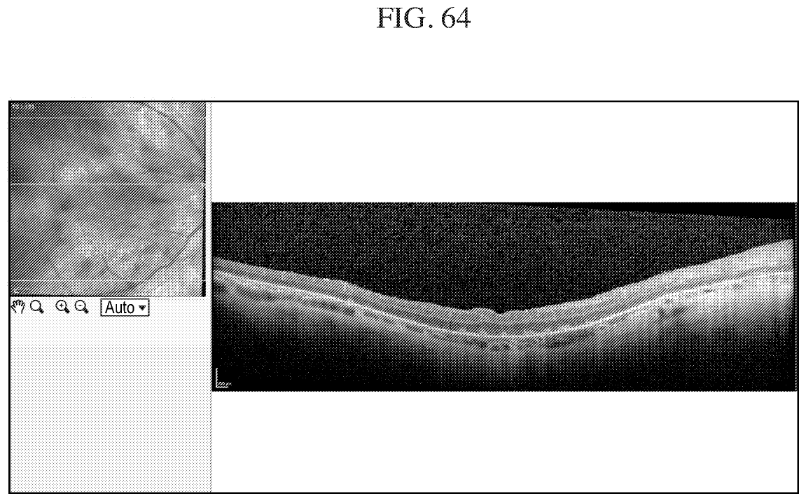

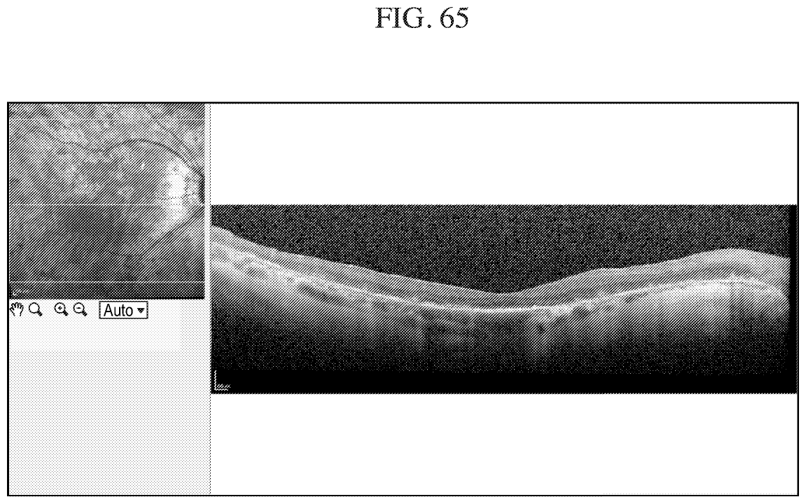

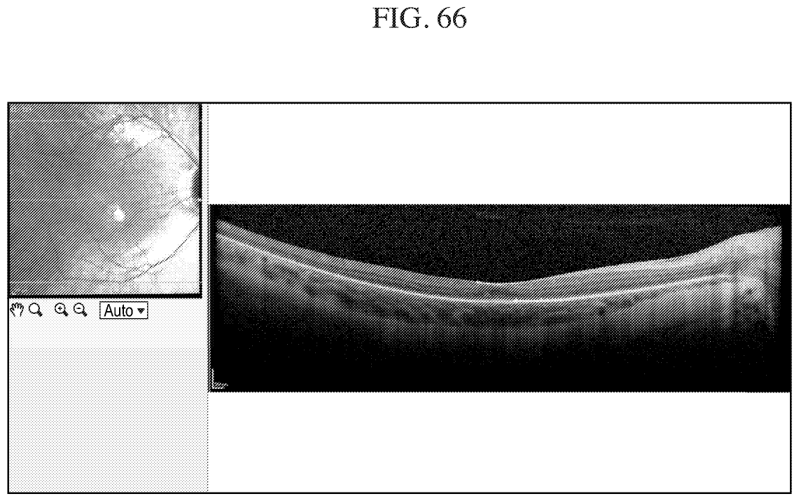

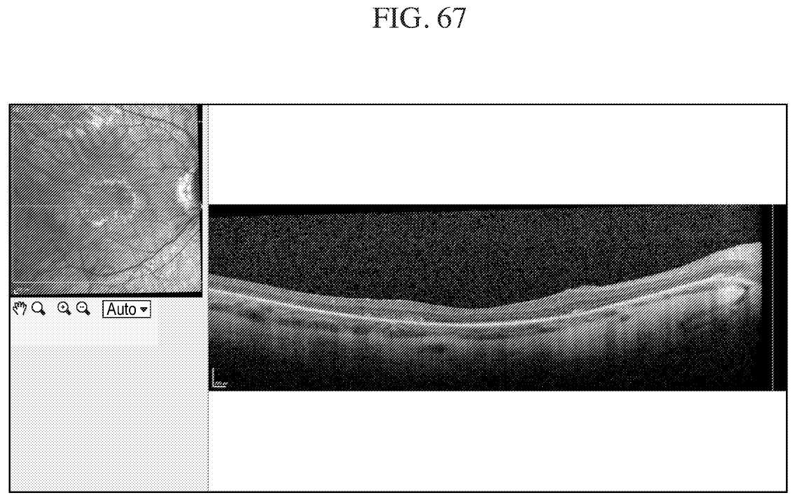

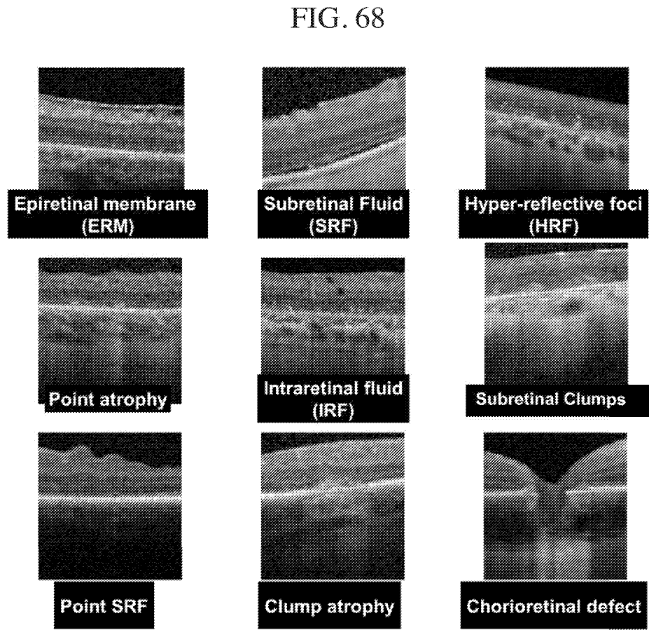

Results of the Xirius analysis reveal an improved therapeutic outcome of participants receiving treatment as evidenced by the appearance of a double line of retinal thickness by OCT analysis. The data demonstrating this finding are provided in FIGS. 52-68.

### Example 8: Clinical Trial of Gene Therapy for Retinitis Pigmentosa

### 6.0 Study Objectives and Endpoints

### 6.1 Objective

The objective of the study is to evaluate the safety, tolerability and efficacy of a single sub-retinal injection of AAV8-RPGR in subjects with XLRP.

### Endpoints

### Primary Efficacy Endpoint

The primary efficacy endpoint is the proportion of study eyes with ≥7 dB improvement from baseline at ≥5 of the 16 central loci of the 10-2 grid assessed by Macular Integrity Assessment (MAIA) microperimetry at 12 months.

### Safety Endpoint

The primary safety endpoint is the incidence of TEAEs over a 12-month period.

### Secondary Endpoints

Proportion of study eyes with ≥7 dB improvement from baseline at ≥5 of 16 central loci of the 10-2 grid assessed by MAIA microperimetry at 1, 2, 3, 6, and 9 months
Proportion of study eyes with ≥7 dB improvement from baseline at ≥5 of 68 loci of the 10-2 grid assessed by MAIA microperimetry at 1, 2, 3, 6, 9, and 12 months
Change from baseline in microperimetry at 1, 2, 3, 6, 9, and 12 months
Change from baseline in BCVA at 1, 2, 3, 6, 9, and 12 months
Change from baseline in visual field assessed by Octopus 900 perimeter at 1, 2, 3, 6, 9, and 12 months

### Exploratory Endpoints

Change from baseline in multi-luminance mobility test (MLMT) at 6 and 12 months
Change from baseline in the 25-item Visual Function Questionnaire (VFQ-25) at 3 and 12 months (in adults only)
Change from baseline in SD-OCT at 1, 2, 3, 6, 9, and 12 months
Change from baseline in fundus autofluorescence at 1, 2, 3, 6, 9, and 12 months
Change from baseline in other anatomic and functional outcomes at 1, 3, 6, 9, and 12 months

### Investigational Plan

### Dose Expansion, Version 9

Subjects are randomized in a 1:1:1 allocation ratio to a high-dose group (2.5×10\hat{ }11 gp), a low-dose group (5×10\hat{ }10 gp), and an untreated group. Within the treated groups, the sponsor, investigator and subject will be masked (i.e. double-masked) to the assigned dose. To further minimise potential bias of the treated and non-treated eye evaluations, all subjective ophthalmic assessments at the Screening/Baseline Visit (Visit 1) and from Month 3 (Visit 6) onwards will be conducted by a masked assessor.

Study data will be collected for both eyes of each subject. Since treatment requires an invasive surgical procedure under general anaesthesia, the sponsor, investigator and the subject will be unmasked to the study procedure (i.e., vitrectomy and sub-retinal injection), however within the treated groups, the sponsor, investigator and subject will be masked to the assigned dose. To further minimise potential bias of the treated and non-treated eye evaluations, all subjective ophthalmic assessments at the Screening/Baseline Visit (Visit 1) and from Month 3 (Visit 6) onwards will be conducted by a masked assessor.

### Inclusion Criteria

1. Subject/parent/legal guardian (if applicable) is willing and able to provide informed consent/assent for participation in the study
2. Are male, ≥10 years of age, and able to comply and adequately perform all study assessments
3. Documentation of a pathogenic mutation in the RPGR gene
4. Have a BCVA in both eyes that meets the following criteria:
   Better than or equal to BCVA of 34 ETDRS letters (equivalent to better than or equal to 6/60 or 20/200 Snellen acuity).
5. Mean total retinal sensitivity in the study eye as assessed by microperimetry ≥0.1 dB and ≤8 dB

### Exclusion Criteria:

Subjects are not eligible for study participation if they meet any of the following exclusion criteria:
1. Have a history of amblyopia in either eye
2. Are unwilling to use barrier contraception methods (if applicable), or abstain from sexual intercourse, for a period of 3 months following treatment with AAV8-RPGR
3. Have any other significant ocular or non-ocular disease/disorder which, in the opinion of the investigator, may put the subjects at risk because of participation in the study, may influence the results of the study, may influence the subject's ability to perform study diagnostic tests, or impact the subject's ability to participate in the study. This would include, but is not limited to, the following:
   a. clinically significant cataract
   b. contraindication to oral corticosteroid
   c. Unsuitability for retinal surgery
4. Have participated in another research study involving an investigational product in the past 12 weeks or received a gene/cell-based therapy at any time previously (including, but not limited to, Intelligent Retinal Implant System implantation, ciliary neurotrophic factor therapy, nerve growth factor therapy).

### Study Treatment

Subjects are assigned to 1 of the following: high-dose (2.5×10\hat{ }11 gp), low-dose (5×10\hat{ }10 gp), or an untreated control arm. The study drug is the same as in Example 3. 9.5

### Randomisation

### Study Masking

Ophthalmic assessments used as efficacy endpoints (BCVA, LLVA, microperimetry, contrast sensitivity and VFQ-25) are conducted by appropriately qualified masked assessors. For the immediate post-operative visits, masking of the assessors will not be viable as clinical signs of surgery will be apparent (i.e., redness, swelling). Therefore, unmasked assessors perform all ophthalmic assessments at Visit 3 (Day 1), Visit 4 (Day 7), Visit 5 (Month 1), and Visit 5.9 (Month 2). From Visit 6 (Month 3) onwards, masked assessors are used, as signs of surgery will have dissipated and it should not be possible clinically to differentiate between those subjects that have not undergone surgery, and those subjects that have undergone surgery and received active treatment.

Masked Assessments at Month 3, 6, 9 and 12 Post-Treatment with AAV8-RPGR
Best-corrected visual acuity
Low-luminance visual acuity
Microperimetry
Contrast sensitivity
25-Item Visual Function Questionnaire

### 9.8 Concomitant Therapy

Subjects are prescribed a course of oral corticosteroids. In addition, at the time of surgery, subjects (adult and pediatric) may be treated with up to 1 mL of triamcinolone, 40 mg/mL solution, which must be administered via a deep sub-Tenon approach.

For adults, 60 mg of oral prednisone/prednisolone are prescribed for the initial 21 days (starting 3 days prior to surgery), followed by a weekly taper as follows, for a total of 9 weeks of treatment:

Day −3 through day 17 (21 days): 60 mg by mouth once daily

Day 18 through day 24 (7 days): 50 mg by mouth once daily

Day 25 through day 31 (7 days): 40 mg by mouth once daily

Day 32 through day 38 (7 days): 30 mg by mouth once daily

Day 39 through day 45 (7 days): 20 mg by mouth once daily

Day 46 through day 52 (7 days): 10 mg by mouth once daily

Day 53 through day 59 (7 days): 5 mg by mouth once daily.

If at the Month-2 visit (Visit 5.9), inflammation is observed, corticosteroid therapy should be re-initiated, via oral and/or intraocular route, based on the clinical condition of the subject, and the judgement of the investigator.

For pediatric subjects, oral prednisolone/prednisone is started 3 days prior to surgery. The starting dose will be based on kilogram weight of the subject, up to a maximum of 60 mg starting dose (rounded to the nearest 1 mg). Subsequent doses will have multipliers to provide the appropriate taper over an additional 6 weeks, for a total of 9 weeks of treatment. See tapering regimen for pediatric subjects below:

Day −3 through day 17 (21 days): Starting Dose (SD) 1 mg/kg by mouth/once daily (maximum dose of 60 mg once daily)

Day 18 through day 24 (7 days): SD×0.83 mg by mouth once daily

Day 25 through day 31 (7 days): SD×0.67 mg by mouth once daily

Day 32 through day 38 (7 days): SD×0.5 mg by mouth once daily

Day 39 through day 45 (7 days): SD×0.33 mg by mouth once daily

Day 46 through day 52 (7 days): SD×0.17 mg by mouth once daily

Day 53 through day 59 (7 days): SD×0.08 mg by mouth once daily

If at the Month-2 visit (Visit 5.9), inflammation is observed, corticosteroid therapy should be reinitiated, via oral and/or intraocular route, based on the clinical condition of the subject, and the judgement of the investigator.

### Assessment of Efficacy

### Best-Corrected Visual Acuity

To evaluate changes in VA over the study period, BCVA is assessed for both eyes using the ETDRS VA chart.

The BCVA test is performed prior to pupil dilation, and distance refraction should be carried out before BCVA is measured. Initially, letters are read at a distance of 4 metres from the chart. If <20 letters are read at 4 metres, testing at 1 metre should be performed. BCVA is to be reported as number of letters read correctly by the subject.

at the Screening/Baseline Visit, eyes will be eligible for the study if they have a BCVA better then or equal to 34 ETDRS letters.

For BCVA, assessors will be appropriately qualified for conducting the assessment.

if the BCVA value at Visit 1 (Screening/Baseline) is ≥±10 letter gain or loss in the study eye compared to the previous XOLARIS study visit (if applicable), then BCVA must be repeated an additional 2 times, resulting in a total of 3 BCVA measures at Visit 1. To facilitate the additional BCVA measures this visit should be conducted over 2 days, with BCVA measured twice on Day 1 and once on Day 2 (prior to pupil dilation). All 3 BCVA values must be recorded in the eCRF. The highest score will be used to determine subject eligibility.

If the BCVA value at Visit 1 (Screening/Baseline) is <±10 letter difference in the study eye compared to the previous XOLARIS study visit, then BCVA will be collected once and will not be repeated.

If subject was not previously in XOLARIS study, BCVA assessments at baseline must be performed in triplicate.

### Spectral Domain Optical Coherence Tomography (SD-OCT)

SD-OCT is performed as in Example 3

### Fundus Autofluorescence

Fundus autofluorescence images are taken as in Example 3.

### MAIA Microperimetry

MAIA Microperimetry is performed as in Example 3.

### Visual Field Testing (Perimetry)

Visual fields is assessed in both eyes. Visual fields will be assessed in triplicate over a 2-day period at Visit 1 for all subjects. Visual fields are assessed using the Octopus 900 perimeter.

### Contrast Sensitivity

Contrast sensitivity is measured as in Example 3.

### Low Luminance Visual Acuity

Low luminance visual acuity is measured as in Example 3.

### Multi-Luminance Mobility Test

MLMT is be conducted at Visit 1 (Screening/Baseline), Visit 7 (Month 6), and Visit 9 (Month 12). Assessments include the time to navigate the course, the number of collisions with obstacles, and the ability to navigate under different lighting conditions.

### Visual Function Questionnaire

Adult subjects complete the VFQ-25 at Visit 1 (Screening/Baseline), Visit 6 (Month 3), and Visit 9 (Month 12) or the ET Visit, if applicable.

### Assessment of Safety

Safety assessments are performed as in Example 312.2.4

### Efficacy Analyses

Efficacy assessments are ocular in nature and therefore are tabulated by eye (Study Eye and Fellow Eye). Efficacy data will be summarised using descriptive statistics.

Improvement in retinal sensitivity and change from baseline in retinal sensitivity are tabulated by visit and by eye.

The proportion of eyes with improved retinal sensitivity, for both the center grid (i.e., the central 16 loci) and the entire grid (i.e., all 68 loci), are compared between study arms (high dose vs untreated; low dose vs untreated) using the Fisher Exact-Boschloo test with a Berger-Boos correction of beta=0.001 (Berger 1994). In addition, the difference in proportions between study arms is presented with its corresponding 95% CI calculated using the method of Miettinen and Nurminen (Miettinen 1985).

Change from baseline in mean sensitivity, in both the center grid and the entire grid, is compared between study arms using an ANCOVA model including baseline value and study arm (high dose, low-dose, and untreated) as covariates. The difference in means between study arms, and its 95% CI, will be derived from the same ANCOVA model.

### INCORPORATION BY REFERENCE

Every document cited herein, including any cross referenced or related patent or application is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

### OTHER EMBODIMENTS

While particular embodiments of the disclosure have been illustrated and described, various other changes and modifications can be made without departing from the spirit and scope of the disclosure. The scope of the appended claims includes all such changes and modifications that are within the scope of this disclosure.

What is claimed is:

1. A composition comprising a plurality of recombinant adeno associated virus of serotype 8 (rAAV8) particles,
wherein each rAAV8 of the plurality of rAAV8 particles is non-replicating, and
wherein each rAAV8 of the plurality of rAAV8 particles comprises a polynucleotide comprising, from 5′ to 3′:
(a) a sequence encoding a 5′ inverted terminal repeat (ITR);
(b) a sequence encoding a G protein-coupled receptor kinase 1 (GRK1) promoter;
(c) a sequence encoding a retinitis pigmentosa GTPase regulator ORF15 isoform (RPGR^{ORF15});
(d) a sequence encoding a polyadenylation (polyA) signal;
(e) a sequence encoding a 3′ ITR; and
wherein the composition comprises
(i) between 1.0×10¹⁰ vector genomes (vg) per milliliter (mL) and 1×10¹³ vg/mL, inclusive of the endpoints;
(ii) between 1.25×10¹² DNase resistant particles (DRP) per milliliter (mL) and 1.0×10¹³ DRP/mL; or
(ii) between 5×10¹⁰ genome particles (gp) and 5×10¹² gp, inclusive of the endpoints.

2. The composition of claim 1, wherein the composition comprises between 1.25×10¹² vg/mL and 1×10¹³ vg/mL, inclusive of the endpoints.

3. The composition of claim 1, wherein the composition comprises 1×10¹² vg/mL.

4. The composition of claim 1, wherein the composition comprises 2.5×10¹² vg/mL.

5. The composition of claim 1, wherein the composition comprises 5×10¹² vg/mL.

6. The composition of claim 1, wherein the composition comprises 5×10⁹ gp, 1×10¹⁰ gp, 5×10¹⁰ gp, 1×10¹¹ gp, 2.5×10¹¹ gp 5×10¹¹ gp, 1.25×10¹² gp, 2.5×10¹² gp, 5×10¹² gp, or 1×10¹³.

7. The composition of any one of claims 1-6, further comprising a pharmaceutically acceptable carrier.

8. The composition of claim 7, wherein the pharmaceutically acceptable carrier comprises Tris, MgCl₂, and NaCl.

9. The composition of claim 8, wherein the pharmaceutically acceptable carrier comprises 20 mM Tris, 1 mM MgCl₂, and 200 mM NaCl at pH 8.0.

10. The composition of claim 8 or 9, wherein the pharmaceutically acceptable carrier further comprises poloxamer 188 at 0.001%.

11. The composition of any one of claims 1-10, wherein the sequence encoding the GRK1 promoter comprises or consists of the sequence of:
(SEQ ID NO: 1)

| | |
|---|---|
| 1 | gggccccaga agcctggtgg ttgtttgtcc ttctcagggg |
| | aaaagtgagg cggccccttg |
| 61 | gaggaagggg ccgggcagaa tgatctaatc ggattccaag |
| | cagctcaggg gattgtcttt |
| 121 | ttctagcacc ttcttgccac tcctaagcgt cctccgtgac |
| | cccggctggg atttagcctg |
| 181 | gtgctgtgtc agccccggg. |

12. The composition of claim 11, wherein the sequence encoding RPGR^{ORF15} comprises or consists of a nucleotide sequence encoding the RPGR^{ORF15} amino acid sequence of:
(SEQ ID NO: 2)

| | |
|---|---|
| 1 | MREPEELMPD SGAVFTFGKS KFAENNPGKF WFKNDVPVHL |
| | SCGDEHSAVV TGNNKLYMFG |
| 61 | SNNWGQLGLG SKSAISKPTC VKALKPEKVK LAACGRNHTL |
| | VSTEGGNVYA TGGNNEGQLG |
| 121 | LGDTEERNTF HVISFFTSEH KIKQLSAGSN TSAALTEDGR |
| | LFMWGDNSEG QIGLKNVSNV |
| 181 | CVPQQVTIGK PVSWISCGYY HSAFVTTDGE LYVFGEPENG |
| | KLGLPNQLLG NHRTPQLVSE |
| 241 | IPEKVIQVAC GGEHTVVLTE NAVYTFGLGQ FGQLGLGTFL |
| | FETSEPKVIE NIRDQTISYI |
| 301 | SCGENHTALI TDIGLMYTFG DGRHGKLGLG LENFTNHFIP |
| | TLCSNFLRFI VKLVACGGCH |
| 361 | MVVFAAPHRG VAKEIEFDEI NDTCLSVATF LPYSSLTSGN |
| | VLQRTLSARM RRRERERSPD |
| 421 | SFSMRRTLPP IEGTLGLSAC FLPNSVFPRC SERNLQESVL |
| | SEQDLMQPEE PDYLLDEMTK |
| 481 | EAEIDNSSTV ESLGETTDIL NMTHIMSLNS NEKSLKLSPV |
| | QKQKKQQTIG ELTQDTALTE |
| 541 | NDDSDEYEEM SEMKEGKACK QHVSQGIFMT QPATTIEAFS |
| | DEEVEIPEEK EGAEDSKGNG |
| 601 | IEEQEVEANE ENVKVHGGRK EKTEILSDDL TDKAEVSEGK |
| | AKSVGEAEDG PEGRGDGTCE |
| 661 | EGSSGAEHWQ DEEREKGEKD KGRGEMERPG EGEKELAEKE |
| | EWKKRDGEEQ EQKEREQGHQ |
| 721 | KERNQEMEEG GEEEHGEGEE EEGDREEEEE KEGEGKEEGE |
| | GEEVEGEREK EEGERKKEER |
| 781 | AGKEEKGEEE GDQGEGEEEE TEGRGEEKEE GGEVEGGEVE |
| | EGKGEREEEE EEGEGEEEEG |
| 841 | EGEEEEGEGE EEEGEGKGEE EGEEGEGEEE GEEGEGEGEE |
| | EEGEGEGEEE GEGEGEEEEG |
| 901 | EGEGEEEGEG EGEEEEGEGK GEEEGEEGEG EGEEEEGEGE |
| | GEDGEGEGEE EEGEWEGEEE |
| 961 | EGEGEGEEEG EGEGEEGEGE GEEEEGEGEG EEEEGEEEGE |
| | EEGEGEEEGE GEGEEEEEGE |
| 1021 | VEGEVEGEEG EGEGEEEEGE EEGEEREKEG EGEENRRNRE |
| | EEEEEEGKYQ ETGEEENERQ |
| 1081 | DGEEYKKVSK IKGSVKYGKH KTYQKKSVTN TQGNGKEQRS |
| | KMPVQSKRLL KNGPSGSKKF |
| 1141 | WNNVLPHYLE LK. |

13. The composition of claim 12, wherein the sequence encoding the RPGR^{ORF15} amino acid sequence comprises a codon optimized sequence.

14. The composition of claim 13, wherein the sequence encoding RPGR^{ORF15} comprises or consists of the nucleotide sequence of:
(SEQ ID NO: 3)

| | |
|---|---|
| 1 | atgagagagc cagaggagct gatgccagac agtggagcag |
| | tgtttacatt cggaaaatct |
| 61 | aagttcgctg aaaataaccc aggaaagttc tggtttaaaa |
| | acgacgtgcc cgtccacctg |
| 121 | tcttgtggcg atgagcatag tgccgtggtc actgggaaca |
| | ataagctgta catgttcggg |
| 181 | tccaacaact ggggacagct ggggctggga tccaaatctg |
| | ctatctctaa gccaacctgc |
| 241 | gtgaaggcac tgaaacccga gaaggtcaaa ctggccgctt |
| | gtggcagaaa ccacactctg |
| 301 | gtgagcaccg agggcgggaa tgtctatgcc accggaggca |
| | acaatgaggg acagctggga |
| 361 | ctgggggaca ctgaggaaag gaataccttt cacgtgatct |
| | ccttctttac atctgagcat |
| 421 | aagatcaagc agctgagcgc tggctccaac acatctgcag |
| | ccctgactga ggacgggcgc |
| 481 | ctgttcatgt ggggagataa ttcagagggc cagattgggc |
| | tgaaaaacgt gagcaatgtg |
| 541 | tgcgtccctc agcaggtgac catcggaaag ccagtcagtt |
| | ggatttcatg tggctactat |
| 601 | catagcgcct tcgtgaccac agatggcgag ctgtacgtct |
| | ttggggagcc cgaaaacgga |
| 661 | aaactgggcc tgcctaacca gctgctgggc aatcaccgga |
| | caccccagct ggtgtccgag |
| 721 | atccctgaaa aagtgatcca ggtcgcctgc gggggagagc |
| | atacagtggt cctgactgag |
| 781 | aatgctgtgt ataccttcgg actgggccag tttggccagc |
| | tggggctggg aaccttcctg |
| 841 | tttgagacat ccgaaccaaa agtgatcgag aacattcgcg |
| | accagactat cagctacatt |
| 901 | tcctgcggag agaatcacac cgcactgatc acagacattg |
| | gcctgatgta tacctttggc |
| 961 | gatggacgac acgggaagct gggactggga ctggagaact |
| | tcactaatca ttttatcccc |
| 1021 | accctgtgtt ctaacttcct gcggttcatc gtgaaactgg |
| | tcgcttgcgg cgggtgtcac |
| 1081 | atggtggtct tcgctgcacc tcataggggc gtggctaagg |
| | agatcgaatt tgacgagatt |
| 1141 | aacgatacat gcctgagcgt ggcaactttc ctgccataca |
| | gctccctgac ttctggcaat |
| 1201 | gtgctgcaga gaaccctgag tgcaaggatg cggagaaggg |
| | agagggaacg ctctcctgac |
| 1261 | agtttctcaa tgcgacgaac cctgccacct atcgagggaa |
| | cactgggact gagtgcctgc |
| 1321 | ttcctgccta actcagtgtt tccacgatgt agcgagcgga |
| | atctgcagga gtctgtcctg |
| 1381 | agtgagcagg atctgatgca gccagaggaa cccgactacc |
| | tgctggatga gatgaccaag |
| 1441 | gaggccgaaa tcgacaactc tagtacagtg gagtccctgg |
| | gcgagactac cgatatcctg |
| 1501 | aatatgacac acattatgtc actgaacagc aatgagaaga |
| | gtctgaaact gtcaccagtg |
| 1561 | cagaagcaga agaaacagca gactattggc gagctgactc |
| | aggacaccgc cctgacagag |
| 1621 | aacgacgata gcgatgagta tgaggaaatg tccgagatga |
| | aggaaggcaa agcttgtaag |
| 1681 | cagcatgtca gtcaggggat cttcatgaca cagccagcca |
| | caactattga ggctttttca |
| 1741 | gacgaggaag tggagatccc cgaggaaaaa gagggcgcag |
| | aagattccaa ggggaatgga |
| 1801 | attgaggaac aggaggtgga agccaacgag gaaaatgtga |
| | aagtccacgg aggcaggaag |
| 1861 | gagaaaacag aaatcctgtc tgacgatctg actgacaagg |
| | ccgaggtgtc cgaaggcaag |
| 1921 | gcaaaatctg tcggagaggc agaagacgga ccagagggac |
| | gaggggatgg aacctgcgag |
| 1981 | gaaggctcaa gcggggctga gcattggcag gacgaggaac |
| | gagagaaggg cgaaaaggat |
| 2041 | aaaggccgcg gggagatgga acgacctgga gagggcgaaa |
| | aagagctggc agagaaggag |
| 2101 | gaatggaaga aaagggacgg cgaggaacag gagcagaaag |
| | aaagggagca gggccaccag |
| 2161 | aaggagcgca accaggagat ggaagagggc ggcgaggaag |
| | agcatggcga gggagaagag |
| 2221 | gaagagggcg atagagaaga ggaagaggaa aaagaaggcg |
| | aagggaagga ggaaggagag |
| 2281 | ggcgaggaag tggaaggcga gagggaaaag gaggaaggag |
| | aacggaagaa agaggaaaga |
| 2341 | gccggcaaag aggaaaaggg cgaggaagag ggcgatcagg |
| | gcgaaggcga ggaggaagag |
| 2401 | accgagggcc gcggggaaga gaaagaggag ggaggagagg |
| | tggagggcgg agaggtcgaa |
| 2461 | gagggaaagg gcgagcgcga agaggaagag gaagagggcg |
| | agggcgagga agaagagggc |
| 2521 | gagggggaag aagaggaggg agagggcgaa gaggaagagg |
| | gggagggaaa gggcgaagag |
| 2581 | gaaggagagg aaggggaggg agaggaagag ggggaggagg |
| | gcgaggggga aggcgaggag |
| 2641 | gaagaaggag agggggaagg cgaagaggaa ggcgaggggg |
| | aaggagagga ggaagaaggg |
| 2701 | gaaggcgaag gcgaagagga gggagaagga gagggggagg |
| | aagaggaagg agaagggaag |
| 2761 | ggcgaggagg aaggcgaaga gggagagggg gaaggcgagg |
| | aagaggaagg cgagggcgaa |
| 2821 | ggagaggacg gcgagggcga gggagaagag gaggaagggg |
| | aatgggaagg cgaagaagag |
| 2881 | gaaggcgaag gcgaaggcga agaagagggc gaaggggagg |
| | gcgaggaggg cgaaggcgaa |
| 2941 | ggggaggaag aggaaggcga aggagaaggc gaggaagaag |
| | agggagagga ggaaggcgag |
| 3001 | gaggaaggag agggggagga ggagggagaa ggcgagggcg |
| | aagaagaaga agagggagaa |
| 3061 | gtggagggcg aagtcgaggg ggaggaggga gaaggggaag |
| | gggaggaaga agagggcgaa |
| 3121 | gaagaaggcg aggaaagaga aaaagaggga gaaggcgagg |
| | aaaaccggag aaatagggaa |
| 3181 | gaggaggaag aggaagaggg aaagtaccag gagacaggcg |
| | aagaggaaaa cgagcggcag |
| 3241 | gatggcgagg aatataagaa agtgagcaag atcaaaggat |
| | ccgtcaagta cggcaagcac |
| 3301 | aaaacctatc agaagaaaag cgtgaccaac acacagggga |
| | atggaaaaga gcagaggagt |
| 3361 | aagatgcctg tgcagtcaaa acggctgctg aagaatggcc |
| | catctggaag taaaaaattc |
| 3421 | tggaacaatg tgctgcccca ctatctggaa ctgaaataa. |

15. The composition of any one of claims 1-14, wherein the sequence encoding the polyA signal comprises a bovine growth hormone (BGH) polyA sequence.

16. The composition of claim 15, wherein the sequence encoding the BGH polyA signal comprises the nucleotide sequence of:
(SEQ ID NO: 4)

| | |
|---|---|
| 1 | tcgctgatca gcctcgactg tgccttctag ttgccagcca |
| | tctgttgttt gcccctcccc |
| 61 | cgtgccttcc ttgaccctgg aaggtgccac tcccactgtc |
| | ctttcctaat aaaatgagga |
| 121 | aattgcatcg cattgtctga gtaggtgtca ttctattctg |
| | gggggtgggg tggggcagga |
| 181 | cagcaagggg gaggattggg aagacaatag caggcatgct |
| | ggggatgcgg tgggctctat |
| 241 | ggcttctgag gcggaaagaa ccagctgggg. |

17. The composition of any one of claims 1-16, wherein the sequence encoding the 5′ ITR is derived from a 5′ITR sequence of an AAV of serotype 2 (AAV2).

18. The composition of any one of claims 1-16, wherein the sequence encoding the 5′ ITR comprises a sequence that is identical to a sequence of a 5′ITR of an AAV2.

19. The composition of any one of claims 1-16, wherein the sequence encoding the 5′ ITR comprises or consists of the nucleotide sequence of:
(SEQ ID NO: 5)
CTGCGCGCTCGCTCGCTCACTGAGGCCGCCCGGGCGTCGGGCGACCTTTG
GTCGCCCGGCCTCAGTGAGCGAGCGAGCGCGCAGAGAGGGAGTGGCCAAC
TCCATCACTAGGGGTTCCT.

20. The composition of any one of claims 1-19, wherein the sequence encoding the 3′ ITR is derived from a 3′ITR sequence of an AAV2.

21. The composition of any one of claims 1-19, wherein the sequence encoding the 3′ ITR comprises a sequence that is identical to a sequence of a 3′ITR of an AAV2.

22. The composition of any one of claims 1-21, wherein the sequence encoding the 3′ ITR comprises or consists of the nucleotide sequence of:
(SEQ ID NO: 6)
AGGAACCCCTAGTGATGGAGTTGGCCACTCCCTCTCTGCGCGCTCGCTCG
CTCACTGAGGCCGGGCGACCAAAGGTCGCCCGACGCCCGGGCTTTGCCCG
GGCGGCCTCAGTGAGCGAGCGAGCGCGCAG.

23. The composition of any one of claims 1-22, wherein the polynucleotide further comprises a Kozak sequence.

24. The composition of claim 23, wherein the Kozak sequence comprises or consists of the nucleotide sequence of GGCCACCATG (SEQ ID NO:7).

25. The composition of claim of any one of claims 1-24, wherein the polynucleotide comprises or consists of the sequence of:
(SEQ ID NO: 8)

| | |
|---|---|
| 1 | CTGCGCGCTC GCTCGCTCAC TGAGGCCGCC CGGGCGTCGG |
| | GCGACCTTTG GTCGCCCGGC |
| 61 | CTCAGTGAGC GAGCGAGCGC GCAGAGAGGG AGTGGCCAAC |
| | TCCATCACTA GGGGTTCCTG |
| 121 | CGGCAATTCA GTCGATAACT ATAACGGTCC TAAGGTAGCG |
| | ATTTAAATAC GCGCTCTCTT |
| 181 | AAGGTAGCCC CGGGACGCGT CAATTGGGGC CCCAGAAGCC |
| | TGGTGGTTGT TTGTCCTTCT |
| 241 | CAGGGGAAAA GTGAGGCGGC CCCTTGGAGG AAGGGGCCGG |
| | GCAGAATGAT CTAATCGGAT |
| 301 | TCCAAGCAGC TCAGGGGATT GTCTTTTTCT AGCACCTTCT |
| | TGCCACTCCT AAGCGTCCTC |
| 361 | CGTGACCCCG GCTGGGATTT AGCCTGGTGC TGTGTCAGCC |
| | CCGGGGCCAC CATGAGAGAG |
| 421 | CCAGAGGAGC TGATGCCAGA CAGTGGAGCA GTGTTTACAT |
| | TCGGAAAATC TAAGTTCGCT |
| 481 | GAAAATAACC CAGGAAAGTT CTGGTTTAAA AACGACGTGC |
| | CCGTCCACCT GTCTTGTGGC |
| 541 | GATGAGCATA GTGCCGTGGT CACTGGGAAC AATAAGCTGT |
| | ACATGTTCGG GTCCAACAAC |
| 601 | TGGGGACAGC TGGGGCTGGG ATCCAAATCT GCTATCTCTA |
| | AGCCAACCTG CGTGAAGGCA |
| 661 | CTGAAACCCG AGAAGGTCAA ACTGGCCGCT TGTGGCAGAA |
| | ACCACACTCT GGTGAGCACC |
| 721 | GAGGGCGGGA ATGTCTATGC CACCGGAGGC AACAATGAGG |
| | GACAGCTGGG ACTGGGGGAC |
| 781 | ACTGAGGAAA GGAATACCTT TCACGTGATC TCCTTCTTTA |
| | CATCTGAGCA TAAGATCAAG |
| 841 | CAGCTGAGCG CTGGCTCCAA CACATCTGCA GCCCTGACTG |
| | AGGACGGGCG CCTGTTCATG |
| 901 | TGGGGAGATA ATTCAGAGGG CCAGATTGGG CTGAAAAACG |
| | TGAGCAATGT GTGCGTCCCT |
| 961 | CAGCAGGTGA CCATCGGAAA GCCAGTCAGT TGGATTTCAT |
| | GTGGCTACTA TCATAGCGCC |
| 1021 | TTCGTGACCA CAGATGGCGA GCTGTACGTC TTTGGGGAGC |
| | CCGAAAACGG AAAACTGGGC |
| 1081 | CTGCCTAACC AGCTGCTGGG CAATCACCGG ACACCCCAGC |
| | TGGTGTCCGA GATCCCTGAA |
| 1141 | AAAGTGATCC AGGTCGCCTG CGGGGGAGAG CATACAGTGG |
| | TCCTGACTGA GAATGCTGTG |
| 1201 | TATACCTTCG GACTGGGCCA GTTTGGCCAG CTGGGGCTGG |
| | GAACCTTCCT GTTTGAGACA |
| 1261 | TCCGAACCAA AAGTGATCGA GAACATTCGC GACCAGACTA |
| | TCAGCTACAT TTCCTGCGGA |
| 1321 | GAGAATCACA CCGCACTGAT CACAGACATT GGCCTGATGT |
| | ATACCTTTGG CGATGGACGA |
| 1381 | CACGGGAAGC TGGGACTGGG ACTGGAGAAC TTCACTAATC |
| | ATTTTATCCC CACCCTGTGT |
| 1441 | TCTAACTTCC TGCGGTTCAT CGTGAAACTG GTCGCTTGCG |
| | GCGGGTGTCA CATGGTGGTC |
| 1501 | TTCGCTGCAC CTCATAGGGG CGTGGCTAAG GAGATCGAAT |
| | TTGACGAGAT TAACGATACA |
| 1561 | TGCCTGAGCG TGGCAACTTT CCTGCCATAC AGCTCCCTGA |
| | CTTCTGGCAA TGTGCTGCAG |
| 1621 | AGAACCCTGA GTGCAAGGAT GCGGAGAAGG GAGAGGGAAC |
| | GCTCTCCTGA CAGTTTCTCA |
| 1681 | ATGCGACGAA CCCTGCCACC TATCGAGGGA ACACTGGGAC |
| | TGAGTGCCTG CTTCCTGCCT |
| 1741 | AACTCAGTGT TTCCACGATG TAGCGAGCGG AATCTGCAGG |
| | AGTCTGTCCT GAGTGAGCAG |
| 1801 | GATCTGATGC AGCCAGAGGA ACCCGACTAC CTGCTGGATG |
| | AGATGACCAA GGAGGCCGAA |
| 1861 | ATCGACAACT CTAGTACAGT GGAGTCCCTG GGCGAGACTA |
| | CCGATATCCT GAATATGACA |
| 1921 | CACATTATGT CACTGAACAG CAATGAGAAG AGTCTGAAAC |
| | TGTCACCAGT GCAGAAGCAG |
| 1981 | AAGAAACAGC AGACTATTGG CGAGCTGACT CAGGACACCG |
| | CCCTGACAGA GAACGACGAT |
| 2041 | AGCGATGAGT ATGAGGAAAT GTCCGAGATG AAGGAAGGCA |
| | AAGCTTGTAA GCAGCATGTC |
| 2101 | AGTCAGGGGA TCTTCATGAC ACAGCCAGCC ACAACTATTG |
| | AGGCTTTTTC AGACGAGGAA |
| 2161 | GTGGAGATCC CCGAGGAAAA AGAGGGCGCA GAAGATTCCA |
| | AGGGGAATGG AATTGAGGAA |
| 2221 | CAGGAGGTGG AAGCCAACGA GGAAAATGTG AAAGTCCACG |
| | GAGGCAGGAA GGAGAAAACA |
| 2281 | GAAATCCTGT CTGACGATCT GACTGACAAG GCCGAGGTGT |
| | CCGAAGGCAA GGCAAAATCT |
| 2341 | GTCGGAGAGG CAGAAGACGG ACCAGAGGGA CGAGGGGATG |
| | GAACCTGCGA GGAAGGCTCA |
| 2401 | AGCGGGGCTG AGCATTGGCA GGACGAGGAA CGAGAGAAGG |
| | GCGAAAAGGA TAAAGGCCGC |
| 2461 | GGGGAGATGG AACGACCTGG AGAGGGCGAA AAAGAGCTGG |
| | CAGAGAAGGA GGAATGGAAG |
| 2521 | AAAAGGGACG GCGAGGAACA GGAGCAGAAA GAAAGGGAGC |
| | AGGGCCACCA GAAGGAGCGC |
| 2581 | AACCAGGAGA TGGAAGAGGG CGGCGAGGAA GAGCATGGCG |
| | AGGGAGAAGA GGAAGAGGGC |
| 2641 | GATAGAGAAG AGGAAGAGGA AAAAGAAGGC GAAGGGAAGG |
| | AGGAAGGAGA GGGCGAGGAA |
| 2701 | GTGGAAGGCG AGAGGGAAAA GGAGGAAGGA GAACGGAAGA |
| | AAGAGGAAAG AGCCGGCAAA |
| 2761 | GAGGAAAAGG GCGAGGAAGA GGGCGATCAG GGCGAAGGCG |
| | AGGAGGAAGA GACCGAGGGC |
| 2821 | CGCGGGGAAG AGAAAGAGGA GGGAGGAGAG GTGGAGGGCG |
| | GAGAGGTCGA AGAGGGAAAG |
| 2881 | GGCGAGCGCG AAGAGGAAGA GGAAGAGGGC GAGGGCGAGG |
| | AAGAAGAGGG CGAGGGGGAA |
| 2941 | GAAGAGGAGG GAGAGGGCGA AGAGGAAGAG GGGGAGGGAA |
| | AGGGCGAAGA GGAAGGAGAG |
| 3001 | GAAGGGGAGG GAGAGGAAGA GGGGGAGGAG GGCGAGGGGG |
| | AAGGCGAGGA GGAAGAAGGA |
| 3061 | GAGGGGGAAG GCGAAGAGGA AGGCGAGGGG GAAGGAGAGG |
| | AGGAAGAAGG GGAAGGCGAA |
| 3121 | GGCGAAGAGG AGGGAGAAGG AGAGGGGGAG GAAGAGGAAG |
| | GAGAAGGGAA GGGCGAGGAG |
| 3181 | GAAGGCGAAG AGGGAGAGGG GGAAGGCGAG GAAGAGGAAG |
| | GCGAGGGCGA AGGAGAGGAC |
| 3241 | GGCGAGGGCG AGGGAGAAGA GGAGGAAGGG GAATGGGAAG |
| | GCGAAGAAGA GGAAGGCGAA |
| 3301 | GGCGAAGGCG AAGAAGAGGG CGAAGGGGAG GGCGAGGAGG |
| | GCGAAGGCGA AGGGGAGGAA |
| 3361 | GAGGAAGGCG AAGGAGAAGG CGAGGAAGAA GAGGGAGAGG |
| | AGGAAGGCGA GGAGGAAGGA |
| 3421 | GAGGGGGAGG AGGAGGGAGA AGGCGAGGGC GAAGAAGAAG |
| | AAGAGGGAGA AGTGGAGGGC |
| 3481 | GAAGTCGAGG GGGAGGAGGG AGAAGGGGAA GGGGAGGAAG |
| | AAGAGGGCGA AGAAGAAGGC |
| 3541 | GAGGAAAGAG AAAAAGAGGG AGAAGGCGAG GAAAACCGGA |
| | GAAATAGGGA AGAGGAGGAA |
| 3601 | GAGGAAGAGG GAAAGTACCA GGAGACAGGC GAAGAGGAAA |
| | ACGAGCGGCA GGATGGCGAG |
| 3661 | GAATATAAGA AAGTGAGCAA GATCAAAGGA TCCGTCAAGT |
| | ACGGCAAGCA CAAAACCTAT |
| 3721 | CAGAAGAAAA GCGTGACCAA CACACAGGGG AATGGAAAAG |
| | AGCAGAGGAG TAAGATGCCT |
| 3781 | GTGCAGTCAA AACGGCTGCT GAAGAATGGC CCATCTGGAA |
| | GTAAAAAATT CTGGAACAAT |
| 3841 | GTGCTGCCCC ACTATCTGGA ACTGAAATAA GAGCTCCTCG |
| | AGGCGGCCCG CTCGAGTCTA |
| 3901 | GAGGGCCCTT CGAAGGTAAG CCTATCCCTA ACCCTCTCCT |
| | CGGTCTCGAT TCTACGCGTA |
| 3961 | CCGGTCATCA TCACCATCACCATTGAGTTT AAACCCGCTG |
| | ATCAGCCTCG ACTGTGCCTT |
| 4021 | CTAGTTGCCA GCCATCTGTT GTTTGCCCCT CCCCCGTGCC |
| | TTCCTTGACC CTGGAAGGTG |
| 4081 | CCACTCCCAC TGTCCTTTCCTAATAAAATG AGGAAATTGC |
| | ATCGCATTGT CTGAGTAGGT |
| 4141 | GTCATTCTAT TCTGGGGGGT GGGGTGGGGC AGGACAGCAA |
| | GGGGGAGGAT TGGGAAGACA |
| 4201 | ATAGCAGGCA TGCTGGGGAT GCGGTGGGCT CTATGGCTTC |
| | TGAGGCGGAA AGAACCAGAT |
| 4261 | CCTCTCTTAA GGTAGCATCG AGATTTAAAT TAGGGATAAC |
| | AGGGTAATGG CGCGGGCCGC |
| 4321 | AGGAACCCCT AGTGATGGAG TTGGCCACTCCCTCTCTGCG |
| | CGCTCGCTCG CTCACTGAGG |
| 4381 | CCGGGCGACC AAAGGTCGCCCGACGCCCGGGCTTTGCCCG |
| | GGCGGCCTCA GTGAGCGAGC |
| 4441 | GAGCGCGCAG. |

26. The composition of any one of claims 1-25, wherein the polynucleotide further comprises a sequence encoding a woodchuck posttranslational regulatory element (WPRE).

27. The composition claim 26, wherein the sequence encoding the WPRE comprises a nucleotide sequence of:
(SEQ ID NO: 9)

| | |
|---|---|
| 1 | atcaacctct ggattacaaa atttgtgaaa gattgactgg |
| | tattcttaac tatgttgctc |
| 61 | cttttacgct atgtggatac gctgctttaa tgcctttgta |
| | tcatgctatt gcttcccgta |
| 121 | tggctttcat tttctcctcc ttgtataaat cctggttgct |
| | gtctctttat gaggagttgt |
| 181 | ggcccgttgt caggcaacgt ggcgtggtgt gcactgtgtt |
| | tgctgacgca acccccactg |
| 241 | gttggggcat tgccaccacc tgtcagctcc tttccgggac |
| | tttcgctttc cccctcccta |
| 301 | ttgccacggc ggaactcatc gccgcctgcc ttgcccgctg |
| | ctggacaggg gctcggctgt |
| 361 | tgggcactga caattccgtg gtgttgtcgg ggaaatcatc |
| | gtcctttcct tggctgctcg |
| 421 | cctgtgttgc cacctggatt ctgcgcggga cgtccttctg |
| | ctacgtccct tcggccctca |
| 481 | atccagcgga ccttccttcc cgcggcctgc tgccggctct |
| | gcggcctctt ccgcgtcttc |
| 541 | gccttcgccc tcagacgagt cggatctccc tttgggccgc |
| | ctccccgc. |

28. The composition of any one of claims 1-27, wherein each of the rAAV8 particles comprise a viral Rep protein isolated or derived from an AAV serotype 8 (AAV8) Rep protein.

29. The composition of any one of claims 1-28, wherein each of the rAAV8 particles comprise a viral Cap protein isolated or derived from an AAV serotype 8 (AAV8) Cap protein.

30. A device, comprising the composition of any one of claims 1-29.

31. The device of claim 30, wherein the device comprises a microdelivery device.

32. The device of claim 31, wherein the microdelivery device comprises a microneedle.

33. The device of claim 32, wherein the microneedle is suitable for subretinal delivery.

34. The device of claim 33, wherein the device comprises a volume of at least 50 μL.

35. The device of claim 32, wherein the microdelivery device comprises a microcatheter.

36. The device of claim 35, wherein the device is suitable for suprachoroidal delivery.

37. The device of claim 36, wherein the device comprises a volume of at least 50 μL.

38. A method of treating Retinitis Pigmentosa in a subject in need thereof, comprising administering to the subject a therapeutically effective amount of the composition of any one of claims 1-29.

39. A method of treating Retinitis Pigmentosa in a subject in need thereof, comprising administering to the subject a therapeutically effective amount of a composition, wherein the administration is performed using the device of any one of claims 30-38.

40. The method of claim 38 or 39, wherein administering the therapeutically effective amount of the composition improves a sign of Retinitis Pigmentosa in the subject.

41. The method of claim 40, wherein the sign of Retinitis Pigmentosa comprises a degeneration of an ellipsoid zone (EZ) when compared to a healthy EZ.

42. The method of claim 41, wherein the degeneration of the EZ comprises a reduction in photoreceptor cell density, a reduction in number of photoreceptor cilia, or a combination thereof, when compared to a healthy EZ.

43. The method of claim 41 or 42, wherein the degeneration of the EZ comprises a reduction of a width and/or area of the EZ when compared to a healthy EZ,

44. The method of any one of claims 41-43, wherein the degeneration of the EZ comprises a reduction of a length of the EZ when compared to a healthy EZ, wherein the length comprises a distance along one or more of the anterior to posterior (A/P) axis, the dorsal to ventral (D/V) axis or the medial to lateral (M/L) axis of the eye; and/or wherein the degeneration of the EZ comprises a reduction of a area of the EZ when compared to a healthy EZ, wherein the area comprises a π time the square of the distance along one or more of the anterior to posterior (A/P) axis, the dorsal to ventral (D/V) axis or the medial to lateral (M/L) axis of the eye.

45. The method of any one of claims 41-44, wherein the healthy EZ comprises an EZ of an age and gender matched individual who does not have either a sign or symptom of Retinitis Pigmentosa.

46. The method of claim 45, wherein the age and gender matched individual who does not have either a sign or symptom of Retinitis Pigmentosa does not have a risk factor for developing Retinitis Pigmentosa.

47. The method of any one of claims 41-44, wherein the healthy EZ comprises a predetermined control or threshold.

48. The method of claim 47, wherein the predetermined control or threshold comprises an average or mean value determined from measurements of a plurality of healthy EZ from a plurality of individuals.

49. The method of claim 47, wherein the plurality of individuals are age and gender matched to the subject.

50. The method of any one of claims 41-44, wherein the healthy EZ comprises an unaffected eye of the subject.

51. The method of claim 50, wherein the unaffected eye does not have a detectable sign of Retinitis Pigmentosa.

52. The method of claim 51, wherein the unaffected eye does not have detectable degeneration of the EZ.

53. The method of claim 40, wherein the sign of Retinitis Pigmentosa comprises a degeneration of an ellipsoid zone (EZ) when compared to a baseline EZ.

54. The method of claim 53, wherein the baseline EZ comprises a measurement of the degeneration of the subject's EZ prior to administration of the composition.

55. The method of claim 54, wherein the measurement of the degeneration of the subject's EZ comprises a determination of a number of living or viable photoreceptors in a portion of the EZ, a number of cilia in a portion of the EZ, a width of a portion of the EZ, a length of the EZ along one or more axes in a portion of the EZ, an area of a portion of the EZ, or any combination thereof.

56. The method of any one of claims 43-55, wherein administering the therapeutically effective amount of the composition improves a sign or a symptom of Retinitis Pigmentosa, wherein the sign of Retinitis Pigmentosa comprises the degeneration of an ellipsoid zone (EZ) when compared to a healthy EZ or a baseline EZ and wherein the improvement comprises increasing the width of the EZ between 1 μm and 20 μm, inclusive of the endpoints and/or increasing the width of the EZ between 0.8 μm and 320 μm, inclusive of the endpoints.

57. The method of claim 56, wherein the improvement comprises increasing the width of the EZ between 3 μm and 15 μm, inclusive of the endpoints and/or increasing the width of the EZ between 7 μm and 180 μm, inclusive of the endpoints.

58. The method of any one of claims 43-55, wherein the improvement comprises increasing the width and/or area of the EZ by 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100% or any percentage in between, when compared to a baseline EZ.

59. The method of any one of claims 43-58, wherein the improvement comprises increasing the width of the EZ uniformly across one or more sector(s) of the eye.

60. The method of any one of claims 43-58, wherein the improvement comprises increasing the width of the EZ non-uniformly across one or more sector(s) of the eye, wherein the increased width is maximal at the macula or within one or more central sector(s) and wherein the increased width is minimal at one or more peripheral sector(s).

61. The method of any one of claims 43-60, wherein the improvement comprises increasing the length of the EZ along the A/P axis.

62. The method of any one of claims 43-61, wherein the improvement comprises increasing the length of the EZ along the D/V axis.

63. The method of any one of claims 43-62, wherein the improvement comprises increasing the length of the EZ along the M/L axis.

64. The method of any one of claims 59-63, wherein the improvement comprises increasing the length and/or area of the EZ by 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100% or any percentage in between, when compared to a baseline EZ.

65. The method of any one of claims 41-64, wherein administering the therapeutically effective amount of the composition reduces a rate of further degeneration or inhibits further degeneration of the EZ when compared to a baseline EZ.

66. The method of claim 65, wherein following administration of the composition, a number of living or viable photoreceptors in a portion of the EZ, a number of cilia in a portion of the EZ, a width of a portion of the EZ, a length of the EZ along one or more axes in a portion of the EZ more axes in a portion of the EZ, an area of a portion of the EZ, or any combination thereof is equal to the number of living or viable photoreceptors in the portion of the EZ, the number of cilia in the portion of the EZ, the width of the portion of the EZ, the length of the EZ along one or more axes in the portion of the EZ or any combination thereof when compared to a baseline EZ.

67. The method of any one of claims 43-66, wherein a width or a length of a portion of the EZ of the subject or a width or a length of a portion of a healthy EZ is measured using optical coherence tomography (OCT).

68. The method of any one of claims 40 and 45-55, wherein the sign of Retinitis Pigmentosa comprises a reduction of a level of retinal sensitivity compared to a healthy level of retinal sensitivity.

69. The method of claim 68, wherein the level of retinal sensitivity is measured using microperimetry.

70. The method of claim 69, wherein the measuring the level of retinal sensitivity comprises:
(a) generating an image of a fundus of an eye of the subject;
(b) projecting a grid of points onto the image of (a);
(c) stimulating the eye at each point on the grid of (b) with light, wherein each subsequent stimulus has a greater intensity than a previous stimulus;
(d) repeating step (c) at least once;
(e) determining for each point on the grid of (b) a minimum threshold value, wherein the minimum threshold value is an intensity of light from (c) at which the subject can first perceive the light; and
(f) converting the minimum threshold value from (e) from asb to decibels (dB), wherein a maximum intensity of light equals 0 dB and a minimum intensity of light equals a maximum dB value of a dB scale.

71. The method of claim 70, wherein the stimulating step of (c) comprises a light stimulus having a range from approximately 4 to 1000 apostilb (asb).

72. The method of claim 70 or 71, wherein the grid comprises at least 37 points.

73. The method of claim 72, wherein the grid comprises or consists of 68 points.

74. The method of any one of claims 70-73, wherein the points are evenly spaced over a circle having a diameter that covers 10° of the eye.

75. The method of claim 74, wherein the circle is centered on the macula.

76. The method of any one of claims 69-75, wherein measuring the level of retinal sensitivity further comprises averaging the minimum threshold value at each point in the grid of (b) to produce a mean retinal sensitivity.

77. The method of any one of claims 69-76, wherein the healthy level of retinal sensitivity is determined using an age and gender matched individual who does not have either a sign or symptom of Retinitis Pigmentosa.

78. The method of claim 77, wherein the age and gender matched individual who does not have either a sign or symptom of Retinitis Pigmentosa does not have a risk factor for developing Retinitis Pigmentosa.

79. The method of any one of claims 69-76, wherein the healthy level of retinal sensitivity is determined using a predetermined control or threshold.

80. The method of claim 79, wherein the predetermined control or threshold comprises an average or mean value determined from measurements of a plurality of healthy levels of retinal sensitivity from a plurality of individuals.

81. The method of claim 80, wherein the plurality of individuals are age and gender matched to the subject.

82. The method of any one of claims 69-81, wherein the healthy level of retinal sensitivity is measured from an unaffected eye of the subject.

83. The method of claim 82, wherein the unaffected eye does not have a detectable sign of Retinitis Pigmentosa.

84. The method of claim 83, wherein the unaffected eye does not have detectable reduction in a level of retinal sensitivity.

85. The method of claim 84, wherein the sign of Retinitis Pigmentosa comprises a reduction of a level of retinal sensitivity when compared to a baseline level of retinal sensitivity.

86. The method of claim 85, wherein the baseline level of retinal sensitivity comprises a measurement of a level of retinal sensitivity of the subject prior to administration of the composition.

87. The method of any one of claims 79-86, wherein administering the therapeutically effective amount of the composition restores retinal sensitivity of the subject when compared to a healthy level of retinal sensitivity.

88. The method of claim 87, wherein restoring retinal sensitivity comprises an increase in a mean retinal sensitivity in a portion of the retina when compared to a healthy level of retinal sensitivity.

89. The method of claim 88, wherein a mean retinal sensitivity in a portion of the retina of the subject equals a mean retinal sensitivity in the portion of the retina in the healthy level of retinal sensitivity.

90. The method of any one of claims 85-89, wherein administering the therapeutically effective amount of the composition improves retinal sensitivity of the subject when compared to a healthy or baseline level of retinal sensitivity.

91. The method of claim 90, wherein improving retinal sensitivity comprises an increase in a mean retinal sensitivity in a portion of the retina when compared to a healthy or baseline level of retinal sensitivity.

92. The method of claim 91, wherein improving retinal sensitivity comprises an increase in a level of mean retinal sensitivity of between 1 and 30 decibels (dB), inclusive of the endpoints.

93. The method of claim 92, wherein improving retinal sensitivity comprises an increase in a level of mean retinal sensitivity of between 1 and 15 dB, inclusive of the endpoints.

94. The method of claim 93, wherein improving retinal sensitivity comprises an increase in a level of mean retinal sensitivity of between 2 to 10 dB, inclusive of the endpoints.

95. The method of claim 91, wherein improving retinal sensitivity comprises an increase in a level of mean retinal sensitivity of 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100% or any percentage in between in a level of mean retinal sensitivity when compared to a healthy or baseline level of retinal sensitivity.

96. The method of claim 91, wherein the increase in a level of mean retinal sensitivity occurs in at least 1, 2, 3, 4, 5, 6, 7, 8, 9, 10, 15, 20, 25, 30, 35 or any number of points in between within a microperimetery grid.

97. The method of claim 96, wherein the increase in a level of mean retinal sensitivity occurs in at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100% or any percentage in between in within a microperimetery grid.

98. The method of any one of claims 85-89, wherein administering the therapeutically effective amount of the composition inhibits further reduction or prevents loss of retinal sensitivity of the subject when compared to a baseline level of retinal sensitivity.

99. The method of claim 98, wherein a level retinal sensitivity in the subject following administration of the composition equals the baseline level of retinal sensitivity

100. A method of preventing Retinitis Pigmentosa in a subject, comprising administering to the subject a prophylactically effective amount of the composition of any one of claims 1-29, wherein the subject is at risk of developing Retinitis Pigmentosa.

101. The method of claim 100, wherein the subject has a risk factor for developing Retinitis Pigmentosa.

102. The method of claim 101, wherein the factor comprises one or more of a genetic marker, a family history of Retinitis Pigmentosa, a symptom of Retinitis Pigmentosa or a combination thereof.

103. The method of claim 102, wherein the symptom of Retinitis Pigmentosa comprises a reduction or loss of visual acuity.

104. The method of claim 102, wherein the visual acuity relates to night vision, peripheral vision, color vision or any combination thereof.

105. The method of any one of claims 40-104, wherein the composition is administered by a subretinal route.

106. The method of claim 105, wherein the composition is administered by a subretinal injection or infusion.

107. The method of claim 106, wherein the composition is administered by a subretinal injection and wherein the injection comprises a volume of 100 μL or up to 100 μL.

108. The method of claim 106 or 107, wherein the subretinal injection comprises two-step injection.

109. The method of claim 108, wherein the two-step injection comprises:
(a) inserting a microneedle between a photoreceptor cell layer and a retinal pigment epithelial (RPE) layer in an eye of the subject;
(b) injecting a solution between the photoreceptor cell layer and a retinal pigment epithelial layer in the eye of the subject in an amount sufficient to partially detach the retina from the RPE to form a bleb; and
(c) injecting the composition into the bleb of (b).

110. The method of claim 109, wherein the solution comprises a balanced salt solution.

111. The method of any one of claims 40-105, wherein the composition is administered by a suprachoroidal route.

112. The method of claim 111, wherein the composition is administered by a suprachoroidal injection or infusion.

113. The method of claim 111 or 112, wherein the composition is administered by a suprachoroidal injection and wherein the injection comprises a volume of between 50 and 1000 μL, inclusive of the endpoints.

114. The method of claim 113, wherein the injection comprises a volume of between 50 and 300 μL, inclusive of the endpoints.

115. The method of any one of claims 111-114, wherein the suprachoroidal injection comprises:
(a) contacting a hollow end of a microdelivery device and a suprachoroidal space of an eye of the subject, wherein the hollow end comprises an opening; and
(b) flowing the composition through the hollow end of the microdelivery device to introduce the composition into the suprachoroidal space.

116. The method of claim 115, wherein the hollow end of the microdelivery device pierced a sclera,
wherein the hollow end of the microdelivery device or an extension thereof traversed a portion of a suprachoroidal space, and/or
wherein the hollow end of the microdelivery device traversed a choroid at least once.

117. The method of claim 90, wherein improving retinal sensitivity comprises an increase in sensitivity of at least 5 dB, at least 6 dB, at least 7 dB, at least 8 dB, at least 9 dB, or at least 10 dB in at least 5 points in the central 16 points of a 68 point grid.

118. The method of claim 117, wherein improving retinal sensitivity comprises an increase in sensitivity of at least 7 dB in at least 5 points in the central 16 points of a 68 point grid.
